(19) United States
(12) Patent Application Publication
(10) Pub. No.: US 2026/0258029 A1
(43) Pub. Date: Sep. 3, 2026

(54) CHROMANE AMIDINE MONOBACTAM COMPOUNDS FOR THE TREATMENT OF BACTERIAL INFECTIONS

(71) Applicant: Merck Sharp & Dohme LLC, Rahway, NJ (US)

(72) Inventors: Helen Y. Chen, Florham Park, NJ (US); Shuzhi Dong, Plainsboro, NJ (US); Zhiyong Hu, Livingston, NJ (US); Jing Su, Scotch Plains, NJ (US); Tao Yu, Edison, NJ (US); Yong Zhang, West Windsor, NJ (US)

(73) Assignee: Merck Sharp & Dohme LLC, Rahway, NJ (US)

(21) Appl. No.: 18/992,378

(22) PCT Filed: Jul. 13, 2023
(86) PCT No.: PCT/US2023/027577

§ 371 Date: Jan. 8, 2025

Related U.S. Application Data
(60) Provisional application No. 63/390,122, filed on Jul. 18, 2022.

(51) Int. Cl. C07D 417/14; A61K 31/424; A61K 31/431; A61K 31/5517; A61P 31/04

(52) U.S. Cl. CPC C07D 417/14; A61K 31/424; A61K 31/431; A61K 31/5517; A61P 31/04

(57) ABSTRACT

The present invention relates to monobactam compounds of Formula (I):(Formula (I)) and pharmaceutically acceptable salts thereof. The present invention also relates to compositions which comprise a monobactam compound of structural formula I or a pharmaceutically acceptable salt thereof, and a pharmaceutically acceptable carrier. The invention further relates to methods for treating a bacterial infection comprising administering to the patient a therapeutically effective amount of a compound of structural formula I, either alone or in combination with a therapeutically effective amount of a second beta-lactam antibiotic.

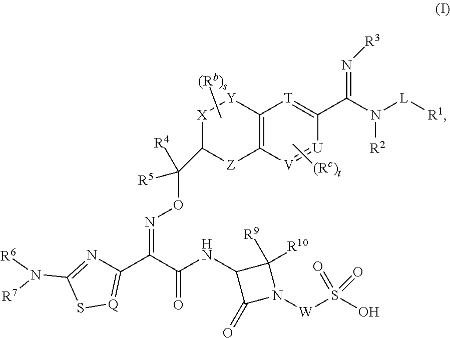

### FIELD OF THE INVENTION

This invention relates to novel monobactam compounds, processes for their preparation and their use as therapeutic agents. In particular, the invention relates to monobactam compounds useful as antibiotic agents for the treatment of bacterial infections.

### BACKGROUND OF THE INVENTION

The introduction of antibiotics for treatment of bacterial infections is one of the great medical achievements of the 20^{th} century. Over the past few decades, however, bacteria resistant to multiple antibiotics have begun to emerge throughout the world, threatening the effectiveness of antibiotic therapy. In the United States alone, at least 23,000 people each year die as a direct result of infections caused by antibiotic-resistant bacteria, and numerous others die from pre-existing conditions exacerbated by similar infections. Antibiotic Resistance Threats in the United States, 2013, Centers for Disease Control, Atlanta, Georgia. New antibiotics are needed to combat the current and future threat of multidrug resistant bacteria.

β-lactams are the most widely used antibiotics for treatment of serious bacterial infections. These include carbapenems, cephalosporins, penicillins, and monobactams. As has been observed for other antibiotic classes, resistance to β-lactams has emerged. For most Gram-negative bacteria, this resistance is primarily driven by the expression of β-lactamases, enzymes that hydrolyze β-lactam compounds. There are 4 different classes of β-lactamases (A, B, C, and D) capable of hydrolyzing overlapping but distinct subsets of β-lactams (Drawz and Bonomo, Clin. Micro. Rev., 2010, 23:160-201). While the class B β-lactamases, also known as metallo 3-lactamases (MBLs), are not the most prevalent β-lactamases found in the clinic, the frequency and distribution of their expression is on the rise and represent a significant medical threat because (i) MBLs have the ability to hydrolze all β-lactams except monobactams, and (ii) unlike the class A and C β-lactamases, there are no inhibitors available for the MBLs.

Aztreonam, a monobactam, was first approved in the U.S in 1986 for the treatment of aerobic Gram-negative bacterial infections and remains the only monobactam in use in the U.S. today. However, aztreonam has poor activity against Pseudomonas and Acinetobacter strains. Because monobactams are inherently resistant to hydrolysis by MBLs, several companies have begun developing novel monobactam compounds for the treatment of infections caused by Gram-negative bacteria. Monobactam compounds comprising a siderophore moiety are disclosed in WO 2007/065288, WO2012/073138, J Medicinal Chemistry 56: 5541-5552 (2013), and Bioorganic and Medicinal Chemistry Letters 22:5989 (2012).

WO 2019/070492 discloses chromane monobactam compounds for treating bacterial infections. WO2017/106064 discloses biaryl monobactam compounds and their use to treat bacterial infections. WO 2013/110643 discloses novel amidine substituted monobactam derivatives and their use as antimicrobial reagents. WO 2015/103583 discloses monobactam derivatives useful for treating infectious disease which is bacterial infection. U.S. Patent Application Publication No US 2015/0045340 and No. US 2014/0275007 disclose oxamazin monobactams and their use as antibacterial agents. U.S. Patent Application Publication No. US 2015/0266867 discloses novel monobactam compounds for the use as antibacterial agents.

The need for new antibiotics to overcome multidrug resistance continues. Compounds disclosed in this invention are designed to fill this medical need, through administration either on their own or in combination with a suitable β-lactamase inhibitor.

### SUMMARY OF THE INVENTION

The invention relates to the design and synthesis of monobactam analogs, a novel class of highly potent antibiotics effective against a broad range of Gram-negative bacteria. These compounds and their pharmaceutically acceptable salts may be useful as therapeutic agents for clinical treatment of various infections caused by Gram-negative bacteria, including strains that are multidrug resistant. The compounds can be used alone or in combination with a suitable β-lactamase inhibitor. The present invention includes the compounds of Formula I:

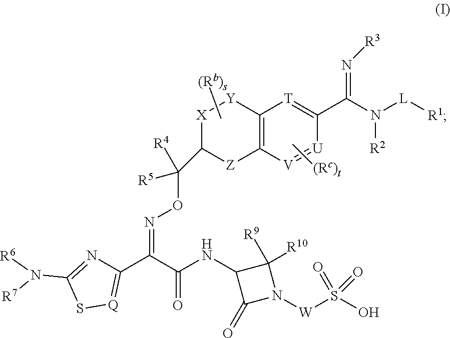

and pharmaceutically acceptable salts thereof.

The present invention also relates to a pharmaceutical composition for treating a bacterial infection in a subject, including infection with multidrug resistant Gram-negative bacterial strains, comprising a monobactam compound of structural formula I or a pharmaceutically acceptable salt thereof, and a pharmaceutically acceptable carrier, diluent or excipient.

The Compounds of Formula (I), also referred to herein as the “monobactam compounds”, and pharmaceutically acceptable salts thereof can be useful, for example, for inhibiting the growth of Gram-negative bacterial strains, including but not limited to, Pseudomonas, Klebsiella and Acinetobacter strains, including Pseudomonas aeruginosa, Klebsiella pneumoniae and Acinetobacter baumannii, and/or for treating or preventing the clinical manifestations thereof in a patient.

The present invention is also directed to methods of treating Gram-negative bacterial infections in a subject in need of treatment thereof, comprising administering to the subject an effective amount of a monobactam compound of the invention. In specific embodiments of the invention, the method includes administration of a beta lactamase inhibitor compound. Embodiments, sub-embodiments and features of the present invention are either further described in or will be apparent from the ensuing description, examples and appended claims.

### DETAILED DESCRIPTION OF THE INVENTION

The present invention is concerned with novel compounds of structural Formula I:

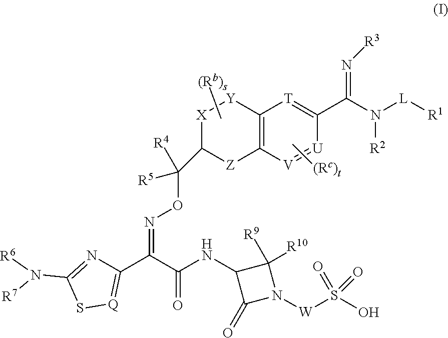

or a pharmaceutically acceptable salt thereof, wherein:
T is CH, or N, provided that no more than two of T, U and V are N;
U is CH, or N;
V is CH or N;
X is selected from the group consisting of
   1) O, and
   2) CH₂;
Y is selected from the group consisting of:
   1) O,
   2) NR⁸,
   3) S, and
   4) CH₂,
provided that when Y is O, NR⁸ or S then X is not O;
Z is
   1) O,
   2) S,
   3) CH₂, or
   4) NH,
provided that when Z is O, S or NH, then X is not O;
W is selected from the group consisting of:
   1) bond, and
   2) O;
Q is selected from the group consisting of:
   1) N, and
   2) CR⁸;
L is selected from the group consisting of:
   1) —C₁₋₆alkyl-,
   2) —C₁₋₆alkyl-O—,
   3) —C₁₋₆alkyl-O—C₁₋₆alkyl-,
   4) —C₁₋₆alkyl-S—,
   5) —C₁₋₆alkyl-S—C₁₋₆alkyl-,
   6) —C₁₋₆alkyl-N(R^{m})—, and
   7) —C₁₋₆alkyl-N(R^{m})—C₁₋₆alkyl-,
wherein alkyl is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl;
R¹ is selected from the group consisting of:
   1) —C₃₋₁₂cycloalkyl,
   2) —C₃₋₁₂cycloalkenyl,
   3) C₂₋₁₁cycloheteroalkyl,
   4) C₂₋₁₁cycloheteroalkenyl,
   5) aryl, and
   6) heteroaryl,
wherein cycloalkyl, cycloalkenyl, cycloheteroalkyl, cycloheteroalkenyl, aryl and heteroaryl are unsubstituted or substituted with one to five substituents selected from R^{a};
R² is selected from the group consisting of:
   1) hydrogen,
   2) C₁₋₆alkyl,
   3) C₁₋₆alkyl-OR⁴, and
   4) C₁₋₆alkyl-NHR⁴,
wherein alkyl is unsubstituted or substituted with one to three halogens;
R³ is selected from the group consisting of:
   1) hydrogen, and
   2) OH;
R⁴ is selected from the group consisting of:
   1) hydrogen,
   2) C₁₋₃alkyl, and
   3) C₃cycloalkyl,
wherein alkyl and cycloalkyl are unsubstituted or substituted with one to three halogens or OC₁₋₃alkyl;
R⁵ is selected from the group consisting of:
   1) —CO₂H, and
   2) tetrazole;
R⁶ and R⁷ are selected from the group consisting of:
   1) hydrogen, and
   2) C₁₋₆alkyl,
wherein alkyl is unsubstituted or substituted with one to three halogens, provided that at least one of R⁶ and R⁷ is hydrogen;
R⁸ is independently selected from the group consisting of:
   1) hydrogen,
   2) C₁₋₄alkyl,
   3) halogen, and
   4) C₃₋₇cycloalkyl,
wherein alkyl and cycloalkyl are unsubstituted or substituted with one to three substituents selected from: —OH, halogen, NH₂, and —OC₁₋₃alkyl;
R⁹ and R¹⁰ are selected from the group consisting of:
   1) hydrogen, and
   2) C₁₋₆alkyl,
wherein alkyl is unsubstituted or substituted with one to three substituents selected from: halogen, OH, —OC₁₋₃alkyl, —NHC(O)C₁₋₃alkyl, —C(O)NHC₁₋₃alkyl, NHC₁₋₃alkyl, and SC₁₋₃alkyl, provided that one or both of R⁹ and R¹⁰ are C₁₋₆alkyl,
or alternatively R⁹ and R¹⁰ together with the carbon to which they are attached form a monocyclic C₃₋₅cycloalkyl or a monocyclic C₂₋₅cycloheteroalkyl, wherein cycloalkyl and cycloheteroalkyl are unsubstituted or substituted with one to three substituents independently selected from halogen, —OH and —OC₁₋₃alkyl;
each R^{a} is independently selected from the group consisting of:
   1) halogen,
   2) —C₁₋₆alkyl,
   3) —C₀₋₆alkyl-O—C₁₋₆alkyl,
   4) —C₀₋₆alkyl-OH,
   5) —C₀₋₆alkyl S(O)ᵣR^{j},
   6) —C₀₋₆alkyl S(O)ᵣNR^{k}R^{l},
   7) —C₀₋₆alkyl C(O)Rⁱ,
   8) —C₀₋₆alkyl OC(O)Rⁱ,
   9) —C₀₋₆alkyl C(O)ORⁱ,
   10) —C₀₋₆alkyl CN,
   11) —C₀₋₆alkyl C(O)NR^{k}R^{l},
   12) —C₀₋₆alkyl C(NH)NR^{k}R^{l},
   13) —C₀₋₆alkylNR^{k}R^{l},
   14) —C₀₋₆alkyl N(R^{k})(C(O)Rⁱ),
   15) —C₀₋₆alkyl N(R^{k})(C(O)OR^{h}),
   16) —C₀₋₆alkyl N(R^{k})(C(O)NR^{f}R^{g}), and
   17) —C₀₋₆alkyl N(R^{k})(S(O)ᵥR^{j}),
wherein alkyl is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆ alkylNH(C₁₋₃alkyl);
each R^{b} is independently selected from the group consisting of:
   1) hydrogen,
   2) C₁₋₆alkyl,
   3) C₀₋₆alkyl-O—C₁₋₆alkyl,
   4) C₀₋₆alkyl-OH,
   5) C₀₋₆alkyl-S(O)ᵤR^{d},
   6) C₁₋₆alkyl-C(O—N(R^{e})₂,
   7) C₁₋₆alkylN(R^{e})C(O)R^{e},
   8) C₀₋₆alkyl-N(R^{e})₂, and
   9) halogen,
wherein alkyl is unsubstituted or substituted with one to three halogens, or wherein two R^{b} substituents together with the atoms they are attached to can cyclize to form a 3 to 6 membered ring;
each R^{e} is independently selected from the group consisting of:
   1) hydrogen,
   2) C₁₋₆alkyl,
   3) C₀₋₆alkyl-O—C₁₋₆alkyl,
   4) C₀₋₆alkyl-OH,
   5) C₀₋₆alkyl-S(O)ᵥR^{f},
   6) C₀₋₆alkyl-S(O)ᵥN(R^{g})₂,
   7) C₁₋₆alkyl C(O)—N(R^{g})₂,
   8) C₁₋₆alkylN(R^{g})C(O)R^{g},
   9) C₀₋₆alkyl-N(R^{g})₂, and
   10) halogen,
wherein alkyl is unsubstituted or substituted with one to three halogens;
each R^{d} is independently selected from the group consisting of:
   1) hydrogen, and
   2) —C₁₋₆ alkyl,
wherein each alkyl is unsubstituted or substituted with one to three halogens;
each R^{e} is independently selected from the group consisting of:
   1) hydrogen, and
   2) —C₁₋₆ alkyl,
wherein each alkyl is unsubstituted or substituted with one to three halogens;
each R^{f} is independently selected from the group consisting of:
   1) hydrogen, and
   2) —C₁₋₆alkyl,
wherein each alkyl is unsubstituted or substituted with one to three halogens;
each R^{g} is independently selected from the group consisting of:
   1) hydrogen, and
   2) —C₁₋₆ alkyl,
wherein each alkyl is unsubstituted or substituted with one to three halogens;
each R^{h} is independently selected from the group consisting of:
   1) hydrogen, and
   2) —C₁₋₆ alkyl,
wherein each alkyl is unsubstituted or substituted with one to three halogens;
each Rⁱ is —C₁₋₆ alkyl, wherein each alkyl is unsubstituted or substituted with one to three halogens;
each R^{j} is independently selected from the group consisting of:
   1) hydrogen,
   2) OH, and
   3) —C₁₋₆ alkyl,
wherein each alkyl is unsubstituted or substituted with one to three halogens;
each R^{k} is independently selected from the group consisting of:
   1) hydrogen, and
   2) —C₁₋₆ alkyl,
wherein each alkyl is unsubstituted or substituted with one to three halogens;
each R^{l} is independently selected from the group consisting of:
   1) hydrogen, and
   2) —C₁₋₆ alkyl,
wherein each alkyl is unsubstituted or substituted with one to three halogens;
each R^{m} is independently selected from the group consisting of:
   1) hydrogen, and
   2) —C1-6 alkyl,
wherein each alkyl is unsubstituted or substituted with one to three halogens;
each r is independently 0, 1 or 2;
each s is independently 0, 1, 2, 3, 4 or 5;
each t is independently 0, 1, 2 or 3;
each u is independently selected from 0, 1 or 2; and
each v is independently selected from 0, 1 or 2.

The invention relates to novel monobactam analogs, a class of highly potent antibiotics effective against a broad range of Gram-negative bacteria. These compounds have utility as therapeutic agents for clinical treatment of various infections caused by Gram-negative bacteria, including strains that are multidrug resistant, and for the treatment or prevention of the clinical pathologies associated therewith.

In each of the various embodiments of the compounds of the invention described herein, each variable including those of Formula (I), and the various embodiments thereof, is selected independently of the others unless otherwise indicated.

The present invention includes the compounds of Formula (I), and the individual diastereoisomers, enantiomers, and epimers of the compounds of Formula (I), and mixtures of diastereoisomers and/or enantiomers thereof including racemic mixtures. The present invention also encompasses any solvates, hydrates, stereoisomers, and tautomers of the compounds of Formula (I), and of any pharmaceutically acceptable salts thereof.

In one embodiment of the present invention, T is CH or N, provided that no more than two of T, U and V are N; U is CH or N; and V═CH or N.

In another embodiment of the present invention, T is CH or N, provided that no more than two of T, U and V are N. In a class of this embodiment, T is CH or N. In another class of this embodiment, T is CH. In another class of this embodiment, T is N.

In another embodiment of the present invention, U is CH or N. In a class of this embodiment, U is CH. In another class of this embodiment, U is N.

In another embodiment of the present invention, V═CH or N. In a class of this embodiment, V is CH. In another class of this embodiment, V is N.

In another embodiment of the present invention, T, U and V are CH.

In another embodiment of the present invention, W is a bond or O. In a class of this embodiment, W is a bond. In another class of this embodiment, W is O.

In another embodiment of the present invention, Q is N or CR⁸. In a class of this embodiment, Q is N. In another class of this embodiment, Q is CR⁸. In another class of this embodiment, Q is CH₂.

In another embodiment of the present invention, X is O or CH₂. In a class of this embodiment, X is O. In another class of this embodiment, X is CH₂.

In another embodiment, Y is O, NR⁸, S or CH₂, provided that when Y is O, NR⁸ or S, then X is not O. In another embodiment, Y is O, NR⁸, S or CH₂, provided that when Y is O, NR⁸ or S, then X is CH₂.

In another embodiment, Y is O, NR⁸, S or CH₂. In a class of this embodiment, Y is O or CH₂. In another class of this embodiment, Y is NR⁸ or S. In another class of this embodiment, Y is O. In another class of this embodiment, Y is NR⁸. In another class of this embodiment, Y is S. In another class of this embodiment, Y is CH₂.

In another embodiment, Z is O, S, CH₂ or NH, provided that when Z is O, S or NH, then X is not O. In a class of this embodiment, Z is O, S, CH₂, or NH. In another class of this embodiment, Z is O or CH₂. In another class of this embodiment, Z is S or NH. In another class of this embodiment, Z is S. In another class of this embodiment, Z is CH₂. In another class of this embodiment, Z is NH. In another class of this embodiment, Z is O.

In another embodiment of this invention, L is selected from the group consisting of: —C₁₋₆alkyl-, —C₁₋₆alkyl-O—, —C₁₋₆alkyl-O—C₁₋₆alkyl-, —C₁₋₆alkyl-S—, —C₁₋₆alkyl-S—C₁₋₆alkyl-, —C₁₋₆alkyl-N(R^{m})—, and —C₁₋₆alkyl-N(R^{m})—C₁₋₆alkyl-, wherein alkyl is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl. In a class of this embodiment, L is selected from the group consisting of: —C₁₋₆alkyl-, —C₁₋₆alkyl-O—, —C₁₋₆alkyl-O—C₁₋₆alkyl-, —C₁₋₆alkyl-S—, —C₁₋₆alkyl-S—C₁₋₆alkyl-, —C₁₋₆alkyl-NR^{m}—, and —C₁₋₆alkyl-NR^{m}—C₁₋₆alkyl-, wherein alkyl is unsubstituted or substituted with one to three substituents selected from: C₁₋₃alkyl.

In another embodiment of this invention, L is selected from the group consisting of: —C₁₋₆alkyl-, —C₁₋₆alkyl-O—, and —C₁₋₆alkyl-O—C₁₋₆alkyl-, wherein alkyl is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl. In a class of this embodiment, L is selected from the group consisting of: —C₁₋₆alkyl-, —C₁₋₆alkyl-O—, and —C₁₋₆alkyl-O—C₁₋₆alkyl-, wherein alkyl is unsubstituted or substituted with one to three substituents selected from: C₁₋₃alkyl.

In another embodiment of this invention, L is selected from the group consisting of —C₁₋₆alkyl-, and —C₁₋₆alkyl-O—, wherein alkyl is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl.

In another embodiment of this invention, L is selected from the group consisting of —C₁₋₆alkyl-, and —C₁₋₆alkyl-O—, wherein alkyl is unsubstituted or substituted with one to three substituents selected from: C₁₋₃alkyl. In another class of this embodiment, L is selected from the group consisting of —CH₂—, —CH₂CH₂—, —CH₂CH₂CH₂—, and —CH₂CH₂O—, wherein L is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl. In another class of this embodiment, L is selected from the group consisting of: —CH₂—, —CH₂CH₂—, —CH₂CH₂CH₂—, and —CH₂CH₂O—, wherein L is unsubstituted or substituted with one to three substituents selected from: C₁₋₃alkyl. In another class of this embodiment, L is selected from the group consisting of: —CH₂—, —CH₂CH₂—, —CH₂CH₂CH₂—, —CH₂CH₂O—, and —CH(CH₃)—CH₂—.

In another embodiment of this invention, L is —C₁₋₆alkyl-, wherein alkyl is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl. In a class of this embodiment, L is —C₁₋₆alkyl-, wherein alkyl is unsubstituted or substituted with one to three substituents selected from: C₁₋₃alkyl. In another class of this embodiment, L is selected from the group consisting of: —CH₂—, —CH₂CH₂—, and —CH₂CH₂CH₂—, wherein L is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl. In another class of this embodiment, L is selected from the group consisting of —CH₂—, —CH₂CH₂—, and —CH₂CH₂CH₂—, wherein L is unsubstituted or substituted with one to three substituents selected from: C₁₋₃alkyl.

In another embodiment of this invention, L is —C₁₋₆alkyl-O—, wherein alkyl is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl. In a class of this embodiment, L is —C₁₋₆alkyl-O—, wherein alkyl is unsubstituted or substituted with one to three substituents selected from: C₁₋₃alkyl. In another class of this embodiment, L is —CH₂CH₂O—, wherein L is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl. In another class of this embodiment, L is —CH₂CH₂O—, wherein L is unsubstituted or substituted with one to three substituents selected from: C₁₋₃alkyl.

In another embodiment of the present invention, R¹ is selected from the group consisting of: —C₃₋₁₂cycloalkyl, —C₃₋₁₂cycloalkenyl, C₂₋₁₁cycloheteroalkyl, C₂₋₁₁cycloheteroalkenyl, aryl, and heteroaryl, wherein cycloalkyl, cycloalkenyl, cycloheteroalkyl, cycloheteroalkenyl, aryl and heteroaryl are unsubstituted or substituted with one to five substituents selected from R.

In another embodiment of the present invention, R¹ is selected from the group consisting of: —C₃₋₁₂cycloalkyl, C₂₋₁₁cycloheteroalkyl, aryl, and heteroaryl, wherein cycloalkyl, cycloheteroalkyl, aryl and heteroaryl are unsubstituted or substituted with one to five substituents selected from R^{a}.

In another embodiment of the present invention, R¹ is selected from the group consisting of: —C₃₋₁₂cycloalkyl, C₂₋₁₁cycloheteroalkyl, and aryl, wherein cycloalkyl, cycloheteroalkyl and aryl are unsubstituted or substituted with one to five substituents selected from R^{a}. In a class of this embodiment, R¹ is selected from the group consisting of cyclopropane, cyclobutane, cyclohexane, bicyclo[1.1.1]pentane, spiro[3.3]heptane, azetidine, pyrrolidine, piperidine, morpholine, diazabicyclo[2.2.1]heptane, and 2,3-dihydroindene, wherein R¹ is unsubstituted or substituted with one to five substituents selected from R^{a}.

In another embodiment of the present invention, R¹ is aryl, wherein aryl is unsubstituted or substituted with one to five substituents selected from R^{a}. In a class of this embodiment, R¹ is 2,3-dihydroindene, wherein R¹ is unsubstituted or substituted with one to five substituents selected from R.

In another embodiment of the present invention, R¹ is selected from the group consisting of: C₃₋₁₂cycloalkyl, and C₂₋₁₁cycloheteroalkyl, wherein cycloalkyl and cycloheteroalkyl are unsubstituted or substituted with one to five substituents selected from R^{a}. In a class of this embodiment, R¹ is selected from the group consisting of cyclopropane, cyclobutane, cyclohexane, bicyclo[1.1.1]pentane, spiro[3.3]heptane, azetidine, pyrrolidine, piperidine, morpholine, and diazabicyclo[2.2.1]heptane, wherein R¹ unsubstituted or substituted with one to five substituents selected from R^{a}. In another class of this embodiment, R¹ is selected from the group consisting of cyclopropane, cyclobutane, bicyclo[1.1.1]pentane, spiro[3.3]heptane, azetidine, and pyrrolidine, wherein R¹ unsubstituted or substituted with one to five substituents selected from R^{a}.

In another embodiment of the present invention, R¹ is C₃₋₁₂cycloalkyl, wherein cycloalkyl is unsubstituted or substituted with one to five substituents selected from R^{a}. In a class of this embodiment, R¹ is selected from the group consisting of cyclopropane, cyclobutane, cyclohexane, bicyclo[1.1.1]pentane, and spiro[3.3]heptane, wherein R¹ unsubstituted or substituted with one to five substituents selected from R^{a}. In another class of this embodiment, R¹ is selected from the group consisting of: cyclopropane, cyclobutane, bicyclo[1.1.1]pentane, and spiro[3.3]heptane, wherein R¹ unsubstituted or substituted with one to five substituents selected from R^{a}.

In another embodiment of the present invention, R¹ is C₂₋₁₁cycloheteroalkyl, wherein cycloheteroalkyl is unsubstituted or substituted with one to five substituents selected from R^{a}. In a class of this embodiment, R¹ is selected from the group consisting of azetidine, pyrrolidine, piperidine, morpholine, diazabicyclo[2.2.1]heptane, wherein R¹ unsubstituted or substituted with one to five substituents selected from R^{a}. In another class of this embodiment, R¹ is selected from the group consisting of azetidine, and pyrrolidine, wherein R¹ unsubstituted or substituted with one to five substituents selected from R^{a}.

In another embodiment of the present invention, R² is selected from the group consisting of: hydrogen, —C₁₋₆alkyl, —C₁₋₆alkyl-OR⁴, and —C₁₋₆alkyl-NHR⁴, wherein alkyl is unsubstituted or substituted with one to three halogens. In a class of this embodiment, R² is selected from the group consisting of: hydrogen and C₁₋₆alkyl, wherein alkyl is unsubstituted or substituted with one to three halogens. In another class of this embodiment, R² is C₁₋₃alkyl, wherein alkyl is unsubstituted or substituted with one to three halogens. In another class of this embodiment, R² is C₁₋₃alkyl. In another class of this embodiment, R² is hydrogen.

In another embodiment of the present invention, R³ is selected from the group consisting of: hydrogen, and OH. In a class of this embodiment, R³ is OH. In another class of this embodiment, R³ is hydrogen.

In another embodiment of the present invention, R⁴ is selected from the group consisting of: hydrogen, —C₁₋₃alkyl, and C₃cycloalkyl, wherein alkyl and cycloalkyl are unsubstituted or substituted with one to three halogens or OC₁₋₃alkyl.

In another embodiment of the present invention, R⁴ is selected from the group consisting of: hydrogen, and C₁₋₃alkyl, wherein alkyl is unsubstituted or substituted with one to three halogens or OC₁₋₃alkyl. In a class of this embodiment, R⁴ is selected from the group consisting of: hydrogen, and C₁₋₃alkyl. In another class of this embodiment, R⁴ is hydrogen.

In another class of this embodiment, R⁴ is C₁₋₃alkyl, wherein alkyl is unsubstituted or substituted with one to three halogens or OC₁₋₃alkyl. In another class of this embodiment, R⁴ is C₁₋₃alkyl. In a subclass of this class, R⁴ is —CH₃.

In another class of the present invention, R⁴ is selected from the group consisting of C₁₋₃alkyl, and C₃cycloalkyl, wherein alkyl and cycloalkyl are unsubstituted or substituted with one to three halogens or OC₁₋₃alkyl. In a class of this embodiment, R⁴ is selected from the group consisting of C₁₋₃alkyl, and C₃cycloalkyl.

In another class of the present invention, R⁴ is cyclopropyl, wherein cyclopropyl is unsubstituted or substituted with one to three halogens or OC₁₋₃alkyl.

In another embodiment of the present invention, R⁵ is selected from the group consisting of: —CO₂H, and tetrazole. In a class of this embodiment, R⁵ is tetrazole. In another class of this embodiment, R⁵ is —CO₂H.

In another embodiment of the present invention, R⁶ and R⁷ are selected from the group consisting of hydrogen, and C₁₋₆alkyl, wherein alkyl is unsubstituted or substituted with one to three halogens, provided that at least one of R⁶ and R⁷ is hydrogen.

In another embodiment, R⁶ is independently selected from the group consisting of: hydrogen, and C₁₋₆alkyl, wherein alkyl is unsubstituted or substituted with one to three halogens, provided that at least one of R⁶ and R⁷ is hydrogen.

In another embodiment, R⁶ is independently selected from the group consisting of: hydrogen, and C₁₋₆alkyl, wherein alkyl is unsubstituted or substituted with one to three halogens. In a class of this embodiment, R⁶ is C₁₋₆alkyl, wherein alkyl is unsubstituted or substituted with one to three halogens. In another class of this embodiment, R⁶ is C₁₋₆alkyl. In another class of this embodiment, R⁶ is hydrogen.

In another embodiment, R⁷ is independently selected from the group consisting of: hydrogen, and C₁₋₆alkyl, wherein alkyl is unsubstituted or substituted with one to three halogens, provided that at least one of R⁶ and R⁷ is hydrogen.

In another embodiment, R⁷ is independently selected from the group consisting of: hydrogen, and C₁₋₆alkyl, wherein alkyl is unsubstituted or substituted with one to three halogens. In a class of this embodiment, R⁷ is C₁₋₆alkyl, wherein alkyl is unsubstituted or substituted with one to three halogens. In another class of this embodiment, R⁷ is C₁₋₆alkyl. In another class of this embodiment, R⁷ is hydrogen.

In another embodiment of the present invention, R⁸ is independently selected from the group consisting of: hydrogen, C₁₋₄alkyl, halogen, and C₃-C₇cycloalkyl, wherein alkyl and cycloalkyl are unsubstituted or substituted with one to three substituents selected from: —OH, halogen, NH₂, and —OC₁₋₃alkyl. In a class of this embodiment, R⁸ is independently selected from the group consisting of: hydrogen, C₁₋₄alkyl, and halogen, wherein C₁-C₄ alkyl is unsubstituted or substituted with one to three substituents selected from: —OH, halogen, NH₂, and —OC₁₋₃alkyl. In another class of this embodiment, R⁸ is independently selected from the group consisting of: hydrogen, and C₁₋₄alkyl, wherein C₁-C₄ alkyl is unsubstituted or substituted with one to three substituents selected from: —OH, halogen, NH₂, and —OC₁₋₃alkyl. In another class of this embodiment, R⁸ is C₁₋₄alkyl, wherein C₁-C₄ alkyl is unsubstituted or substituted with one to three substituents selected from: —OH, halogen, NH₂, and —OC₁₋₃alkyl. In another class of this embodiment, R⁸ is independently selected from the group consisting of hydrogen, and C₁₋₄alkyl, In another class of this embodiment, R⁸ is hydrogen.

In another embodiment of the present invention, R⁹ and R¹⁰ are selected from the group consisting of hydrogen, and C₁₋₆alkyl, wherein alkyl is unsubstituted or substituted with one to three substituents selected from: halogen, OH, —OC₁₋₃alkyl, —NHC(O)C₁₋₃alkyl, —C(O)NHC₁₋₃alkyl, NHC₁₋₃alkyl, and SC₁₋₃alkyl, provided that one or both of R⁹ and R¹⁰ are C₁₋₆alkyl, or alternatively R⁹ and R¹⁰ together with the carbon to which they are attached form a monocyclic C₃₋₅cycloalkyl or a monocyclic C₂₋₅cycloheteroalkyl, wherein cycloalkyl and cycloheteroalkyl are unsubstituted or substituted with one to three substituents independently selected from halogen, —OH and —OC₁₋₃alkyl. In a class of this embodiment, R⁹ and R¹⁰ are selected from the group consisting of hydrogen, and C₁₋₆alkyl, wherein alkyl is unsubstituted or substituted with one to three substituents selected from: halogen, OH, —OC₁₋₃alkyl, —NHC(O)C₁₋₃alkyl, —C(O)NHC₁₋₃alkyl, NHC₁₋₃alkyl, and SC₁₋₃alkyl, provided that one or both of R⁹ and R¹⁰ are C₁₋₆alkyl. In another class of this embodiment, R⁹ and R¹⁰ are selected from the group consisting of: hydrogen, —CH₃, and —CH₂CH₃. In another class of this embodiment, R⁹ and R¹⁰ are selected from C₁₋₆alkyl, wherein alkyl is unsubstituted or substituted with one to three substituents selected from: halogen, OH, —OC₁₋₃alkyl, —NHC(O)C₁₋₃alkyl, —C(O)NHC₁₋₃alkyl, NHC₁₋₃alkyl, or SC₁₋₃alkyl, provided that one or both of R⁹ and R¹⁰ are C₁₋₆alkyl. In another class of this embodiment, R⁹ and R¹⁰ are selected from C₁₋₆alkyl. In another class of this embodiment, R⁹ and R¹⁰ are selected from: —CH₃ and —CH₂CH₃. In another class of this embodiment, R⁹ and R¹⁰ are each —CH₂CH₃. In another class of this embodiment, R⁹ and R¹⁰ are each —CH₃.

In another embodiment of the present invention, R⁹ is independently C₁₋₆alkyl, wherein alkyl is unsubstituted or substituted with one to three substituents selected from: halogen, OH, —OC₁₋₃alkyl, —NHC(O)C₁₋₃alkyl, —C(O)NHC₁₋₃alkyl, NHC₁₋₃alkyl, and SC₁₋₃alkyl, or alternatively R⁹ and R¹⁰ together with the carbon to which they are attached form a monocyclic C₃₋₅cycloalkyl or a monocyclic C₂₋₅cycloheteroalkyl, wherein cycloalkyl and cycloheteroalkyl are unsubstituted or substituted with one to three substituents independently selected from halogen, —OH and —OC₁₋₃alkyl. In another class of this embodiment, R⁹ is independently selected from the group consisting of: C₁₋₆alkyl, wherein alkyl is unsubstituted or substituted with one to three substituents selected from: halogen, OH, —OC₁₋₃alkyl, —NHC(O)C₁₋₃alkyl, —C(O)NHC₁₋₃alkyl, NHC₁₋₃alkyl, and SC₁₋₃alkyl.

In another class of this embodiment, R⁹ is independently selected from the group consisting of C₁₋₆alkyl. In another class of this embodiment, R⁹ is independently selected from the group consisting of: —CH₃, and —CH₂CH₃. In another class of this embodiment, R⁹ is —CH₂CH₃. In another class of this embodiment, R⁹ is —CH₃.

In another embodiment of the present invention, R¹⁰ is independently C₁₋₆alkyl, wherein alkyl is unsubstituted or substituted with one to three substituents selected from: halogen, OH, —OC₁₋₃alkyl, —NHC(O)C₁₋₃alkyl, —C(O)NHC₁₋₃alkyl, NHC₁₋₃alkyl, and SC₁₋₃alkyl, or alternatively R⁹ and R¹⁰ together with the carbon to which they are attached form a monocyclic C₃₋₅cycloalkyl or a monocyclic C₂₋₅cycloheteroalkyl, wherein cycloalkyl and cycloheteroalkyl are unsubstituted or substituted with one to three substituents independently selected from halogen, —OH and —OC₁₋₃alkyl. In another class of this embodiment, R¹⁰ is independently selected from the group consisting of: C₁₋₆alkyl, wherein alkyl is unsubstituted or substituted with one to three substituents selected from: halogen, OH, —OC₁₋₃alkyl, —NHC(O)C₁₋₃alkyl, —C(O)NHC₁₋₃alkyl, NHC₁₋₃alkyl, and SC₁₋₃alkyl. In another class of this embodiment, R¹⁰ is independently selected from the group consisting of C₁₋₆alkyl. In another class of this embodiment, R¹⁰ is independently selected from the group consisting of —CH₃, and —CH₂CH₃. In another class of this embodiment, R¹⁰ is —CH₂CH₃. In another class of this embodiment, R¹⁰ is —CH₃.

In another embodiment of the present invention, each R^{a} is independently selected from the group consisting of halogen, —C₁₋₆alkyl, —C₀₋₆alkyl-O—C₁₋₆alkyl, —C₀₋₆alkyl-OH, —C₀₋₆alkyl S(O)ᵣR^{j}, —C₀₋₆alkyl S(O)ᵣNR^{k}R^{l}, —C₀₋₆alkyl C(O)Rⁱ, —C₀₋₆alkyl OC(O)Rⁱ, —C₀₋₆alkyl C(O)ORⁱ, —C₀₋₆alkyl CN, —C₀₋₆alkyl C(O)NR^{k}R^{l}, —C₀₋₆alkyl C(NH)NR^{k}R^{l}, —C₀₋₆alkylNR^{k}R^{l}, —C₀₋₆alkyl N(R^{k})(C(O)Rⁱ), —C₀₋₆alkyl N(R^{k})(C(O)OR^{h}), —C₀₋₆alkyl N(R^{k})(C(O)NR^{f}R^{h}), and —C₀₋₆alkyl N(R^{k})(S(O)_{y}Rⁱ), wherein alkyl is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl).

In another embodiment of the present invention, each R^{a} is independently selected from the group consisting of halogen, —C₁₋₆alkyl, —C₀₋₆alkyl-O—C₁₋₆alkyl, —C₀₋₆alkyl-OH, —C₀₋₆alkyl C(O)Rⁱ, —C₀₋₆alkyl OC(O)Rⁱ, —C₀₋₆alkyl C(O)ORⁱ, —C₀₋₆alkyl CN, —C₀₋₆alkyl C(O)NR^{k}R^{l}, —C₀₋₆alkyl C(NH)NR^{k}R^{l}, and —C₀₋₆alkylNR^{k}R^{l}, wherein alkyl is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl).

In another embodiment of the present invention, each R^{a} is independently selected from the group consisting of: halogen, —C₁₋₆alkyl, —C₀₋₆alkyl-O—C₁₋₆alkyl, —C₀₋₆alkyl-OH, —C₀₋₆alkyl CN, and —C₀₋₆alkylNR^{k}R^{l}, wherein alkyl is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl).

In another embodiment of the present invention, each R^{a} is independently selected from the group consisting of: halogen, —C₁₋₆alkyl, —C₀₋₆alkyl-O—C₁₋₆alkyl, —C₀₋₆alkyl-OH, and —C₀₋₆alkylNR^{k}R^{l}, wherein alkyl is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl). In a class of this embodiment, each R^{a} is independently selected from the group consisting of: F, CH₃, —OCH₃, —CH₂OCH₃, —CH₂CH₂OCH₃, —CH₂OH, —NH₂, —CH₂NH₂, and —CH₂CH₂NH₂, wherein R^{a} is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl). In another class of this embodiment, each R^{a} is independently selected from the group consisting of: F, CH₃, —OCH₃, —CH₂OCH₃, —CH₂CH₂OCH₃, —CH₂OH, —NH₂, —CH₂NH₂, and —CH₂CH₂NH₂.

In another embodiment of the present invention, each R^{a} is independently selected from the group consisting of: halogen, —C₀₋₆alkyl-OH, and —C₀₋₆alkylNR^{k}R^{l}, wherein R^{a} is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl). In a class of this embodiment, each R^{a} is independently selected from the group consisting of: halogen, —C₀₋₆alkyl-OH, and —C₀₋₆alkylNR^{k}R^{l}. In another class of this embodiment, each R^{a} is independently selected from the group consisting of: F, —CH₂OH, —NH₂, —CH₂NH₂, and —CH₂CH₂NH₂, wherein R^{a} is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl). In another class of this embodiment, each R^{a} is independently selected from the group consisting of F, —CH₂OH, —NH₂, —CH₂NH₂, and —CH₂CH₂NH₂.

In another embodiment of the present invention, each R^{a} is independently selected from the group consisting of: halogen, —C₁₋₆alkyl, —C₀₋₆alkyl-O—C₁₋₆alkyl, —C₀₋₆alkyl-OH, —C₀₋₆alkyl S(O)ᵣR^{J}, —C₀₋₆alkyl S(O)ᵣNR^{k}R^{l}, —C₀₋₆alkyl C(O)Rⁱ, —C₀₋₆alkyl OC(O)Rⁱ, —C₀₋₆alkyl C(O)ORⁱ, —C₀₋₆alkyl CN, —C₀₋₆alkyl C(O)NR^{k}R^{l}, —C₀₋₆alkyl C(NH)NR^{k}R^{l}, —C₀₋₆alkylNR^{k}R^{l}, —C₀₋₆alkyl N(R^{k})(C(O)Rⁱ), —C₀₋₆alkyl N(R^{k})(C(O)OR^{h}), —C₀₋₆alkyl N(R^{k})(C(O)NR^{f}R^{g}), and —C₀₋₆alkyl N(R^{k})(S(O)ᵥR^{j}), wherein alkyl is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl).

In another embodiment of the present invention, each R^{a} is independently selected from the group consisting of halogen, —C₁₋₆alkyl, —C₀₋₆alkyl-O—C₁₋₆alkyl, —C₀₋₆alkyl-OH, and —C₀₋₆alkylNR^{k}R^{l}, wherein alkyl is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl). In a class of this embodiment, each R^{a} is independently selected from the group consisting of: halogen, —C₁₋₆alkyl, —C₀₋₆alkyl-O—C₁₋₆alkyl, —C₀₋₆alkyl-OH, and —C₀₋₆alkylNR^{k}R^{l}, wherein alkyl is unsubstituted or substituted with one to three substituents selected from: halogen, OH, —OC₁₋₃alkyl, and —C₁₋₃alkyl. In another class of this embodiment, each R^{a} is independently selected from the group consisting of: F, —CH₃, —OCH₃, —CH₂OCH₃, —(CH₂)₂OCH₃, —OH, —CH₂OH, —(CH₂)₂OH, —(CH₂)₃OH, —CH(OH)CH₂OH, —CH₂CH(OH)CH₂OH, —NH₂, —CH₂NH₂, —(CH₂)₂NH₂, —C(CH₃)₂NH₂, —(CH₂)₃NH₂, —NH(CH₃), —CH₂NH(CH₃), and —CH₂CH(OH)CH₂NH₂.

In another embodiment of the present invention, each R^{a} is halogen. In a class of this embodiment, each R^{a} is F.

In another embodiment of the present invention, each R^{a} is independently selected from the group consisting of —C₀₋₆alkyl-O—C₁₋₆alkyl, wherein alkyl is unsubstituted or substituted with one to three substituents selected from: halogen, OH, and —C₁₋₃alkyl. In a class of this embodiment, each R^{a} is independently selected from the group consisting of —OCH₃, —CH₂OCH₃, and —(CH₂)₂OCH₃.

In another embodiment of the present invention, each R^{a} is independently selected from the group consisting of —C₁₋₆alkyl, —C₀₋₆alkyl-OH, and —C₀₋₆alkylNR^{k}R^{l}, wherein alkyl is unsubstituted or substituted with one to three substituents selected from: halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl). In a class of this embodiment, each R^{a} is independently selected from the group consisting of —C₁₋₆alkyl, —C₀₋₆alkyl-OH, and —C₀₋₆alkylNR^{k}R^{l}, wherein alkyl is unsubstituted or substituted with one to three substituents selected from: halogen, OH, and —C₁₋₃alkyl. In another class of this embodiment, each R^{a} is independently selected from the group consisting of —CH₃, —OH, —CH₂OH, —(CH₂)₂OH, —(CH₂)₃OH, —CH(OH)CH₂OH, —CH₂CH(OH)CH₂OH, —NH₂, —CH₂NH₂, —(CH₂)₂NH₂, —C(CH₃)₂NH₂, —(CH₂)₃NH₂, —NH(CH₃), —CH₂NH(CH₃), and —CH₂CH(OH)CH₂NH₂. In another class of this embodiment, each R^{a} is independently selected from the group consisting of: —CH₃, —OH, —NH₂, —CH₂NH₂, and —CH₂CH(OH)CH₂NH₂.

In another embodiment of the present invention, each R^{a} is independently selected from the group consisting of: —C₁₋₆alkyl, wherein alkyl is unsubstituted or substituted with one to three substituents selected from: halogen, OH, —OC₁₋₃alkyl, and —C₁₋₃alkyl. In a class of this embodiment, each R^{a} is —CH₃.

In another embodiment of the present invention, each R^{a} is —C₀₋₆alkyl-OH, wherein alkyl is unsubstituted or substituted with one to three substituents selected from: halogen, OH, —OC₁₋₃alkyl, and —C₁₋₃alkyl. In a class of this embodiment, each R^{a} is independently selected from the group consisting of: —OH, —CH₂OH, —(CH₂)₂OH, —(CH₂)₃OH, —CH(OH)CH₂OH, and —CH₂CH(OH)CH₂OH.

In another embodiment of the present invention, each R^{a} is —C₀₋₆alkylNR^{k}R^{l}, wherein alkyl is unsubstituted or substituted with one to three substituents selected from: halogen, OH, —OC₁₋₃alkyl, and —C₁₋₃alkyl. In a class of this embodiment, each R^{a} is independently selected from the group consisting of: —NH₂, —CH₂NH₂, —(CH₂)₂NH₂, —C(CH₃)₂NH₂, —(CH₂)₃NH₂, —NH(CH₃), —CH₂NH(CH₃), and —CH₂CH(OH)CH₂NH₂.

In another embodiment of the present invention, each R^{b} is independently selected from the group consisting of: hydrogen, —C₁₋₆alkyl, —C₀₋₆alkyl-O—C₁₋₆alkyl, —C₀₋₆alkyl-OH, —C₀₋₆alkyl-S(O)ᵤR^{d}, —C₁₋₆alkyl-C(O—N(R^{e})₂, —C₁₋₆alkylN(R^{e})C(O)R^{e}, —C₀₋₆alkyl-N(R^{e})₂, and halogen, wherein alkyl is unsubstituted or substituted with one to three halogens, and wherein two R^{b} substituents together with the atoms they are attached to can cyclize to form a monocyclic C₃₋₆cycloalkyl or a monocyclic C₂₋₆cycloheteroalkyl ring.

In another embodiment of the present invention, each R^{b} is independently selected from the group consisting of: hydrogen, —C₁₋₆alkyl, —C₀₋₆alkyl-O—C₁₋₆alkyl, —C₀₋₆alkyl-OH, and halogen, wherein alkyl is unsubstituted or substituted with one to three halogens, and wherein two R^{b} substituents together with the atoms they are attached to can cyclize to form a monocyclic C₃₋₆cycloalkyl or a monocyclic C₂₋₆cycloheteroalkyl ring.

In another embodiment of the present invention, each R^{b} is independently selected from the group consisting of: hydrogen, —C₁₋₆alkyl, —C₀₋₆alkyl-O—C₁₋₆alkyl, —C₀₋₆alkyl-OH, and halogen, wherein alkyl is unsubstituted or substituted with one to three halogens, and wherein two R^{b} substituents together with the atoms they are attached to can cyclize to form a monocyclic C₃₋₆cycloalkyl or a monocyclic C₂₋₆cycloheteroalkyl ring.

In another embodiment of the present invention, each R^{b} is independently selected from the group consisting of: hydrogen, C₁₋₆alkyl, and halogen, wherein alkyl is unsubstituted or substituted with one to three halogens, and wherein two R^{b} substituents together with the atoms they are attached to can cyclize to form a 3 to 6 membered ring. In a class of this embodiment, each R^{b} is independently selected from the group consisting of hydrogen, and —C₁₋₆alkyl, wherein alkyl is unsubstituted or substituted with one to three halogens, and wherein two R^{b} substituents together with the atoms they are attached to can cyclize to form a 3 to 6 membered ring. In another class of embodiment, each R^{b} is C₁₋₆alkyl, wherein alkyl is unsubstituted or substituted with one to three halogens, and wherein two R^{b} substituents together with the atoms they are attached to can cyclize to form a 3 to 6 membered ring. In another class of this embodiment, each R^{b} is C₁₋₆alkyl, wherein alkyl is unsubstituted or substituted with one to three halogens. In another class of this embodiment, each R^{b} is hydrogen.

In another embodiment of the present invention, each R^{c} is independently selected from the group consisting of: hydrogen, —C₁₋₆alkyl, —CO—6alkyl-O—C₁₋₆alkyl, —C₀₋₆alkyl-OH, —C₀₋₆alkyl-S(O)ᵥR^{f}, —C₀₋₆alkyl-S(O)ᵥN(R^{g})₂, —C₁₋₆alkyl C(O)—N(R^{g})₂, —C₁₋₆alkylN(R^{g})C(O)R^{g}, —C₀₋₆alkyl-N(R^{g})₂, and halogen, wherein alkyl is unsubstituted or substituted with one to three halogens.

In another embodiment of the present invention, each R^{c} is independently selected from the group consisting of: hydrogen, —C₁₋₆alkyl, —C₀₋₆alkyl-O—C₁₋₆alkyl, —C₀₋₆alkyl-OH, and halogen, wherein alkyl is unsubstituted or substituted with one to three halogens.

In another embodiment of the present invention, each R is independently selected from the group consisting of: hydrogen, —C₁₋₆alkyl, —C₀₋₆alkyl-O—C₁₋₆alkyl, and halogen, wherein alkyl is unsubstituted or substituted with one to three halogens. In a class of this embodiment, each R^{c} is independently selected from the group consisting of: hydrogen, —C₁₋₆alkyl, —O—C₁₋₆alkyl, and halogen, wherein alkyl is unsubstituted or substituted with one to three halogens.

In another embodiment of the present invention, each R^{c} is independently selected from the group consisting of: hydrogen, —C₁₋₆alkyl, and —C₀₋₆alkyl-O—C₁₋₆alkyl, wherein alkyl is unsubstituted or substituted with one to three halogens. In a class of this embodiment, each R^{c} is independently selected from the group consisting of: hydrogen, —C₁₋₆alkyl, and —O—C₁₋₆alkyl, wherein alkyl is unsubstituted or substituted with one to three halogens. In another class of this embodiment, each R^{c} is independently selected from the group consisting of hydrogen, —CH₃, and —OCH₃.

In another embodiment of the present invention, each R^{c} is independently selected from the group consisting of: C₁₋₆alkyl, and C₀₋₆alkyl-O—C₁₋₆alkyl, wherein alkyl is unsubstituted or substituted with one to three halogens. In a class of this embodiment, each R^{c} is independently selected from the group consisting of: C₁₋₆alkyl, and —O—C₁₋₆alkyl, wherein alkyl is unsubstituted or substituted with one to three halogens. In another class of this embodiment, each R^{e} is independently selected from the group consisting of —CH₃, and —OCH₃.

In another embodiment of the present invention, each R^{c} is C₁₋₆alkyl, wherein alkyl is unsubstituted or substituted with one to three halogens. In a class of this embodiment, each R^{c} is —CH₃.

In another embodiment of the present invention, R^{d} is independently selected from the group consisting of hydrogen, and —C₁₋₆ alkyl, wherein each alkyl is unsubstituted or substituted with one to three halogens. In a class of this embodiment, R^{d} is —C₁₋₆ alkyl, wherein each alkyl is unsubstituted or substituted with one to three halogens. In another class of this embodiment, R^{d} is hydrogen.

In another embodiment of the present invention, R^{e} is independently selected from the group consisting of: hydrogen, and —C₁₋₆ alkyl, wherein each alkyl is unsubstituted or substituted with one to three halogens. In a class of this embodiment, R^{e} is —C₁₋₆ alkyl, wherein each alkyl is unsubstituted or substituted with one to three halogens. In another class of this embodiment, R^{e} is hydrogen.

In another embodiment of the present invention, R^{f} is independently selected from the group consisting of hydrogen, and —C₁₋₆ alkyl, wherein each alkyl is unsubstituted or substituted with one to three halogens. In a class of this embodiment, R^{f} is —C₁₋₆ alkyl, wherein each alkyl is unsubstituted or substituted with one to three halogens. In another class of this embodiment, R^{f} is hydrogen.

In another embodiment of the present invention, R^{g} is independently selected from the group consisting of hydrogen, and —C₁₋₆ alkyl, wherein each alkyl is unsubstituted or substituted with one to three halogens. In a class of this embodiment, R^{g} is —C₁₋₆ alkyl, wherein each alkyl is unsubstituted or substituted with one to three halogens. In another class of this embodiment, R^{g} is hydrogen.

In another embodiment of the present invention, R^{h} is independently selected from the group consisting of hydrogen, and —C₁₋₆ alkyl, wherein each alkyl is unsubstituted or substituted with one to three halogens. In a class of this embodiment, R^{h} is —C₁₋₆ alkyl, wherein each alkyl is unsubstituted or substituted with one to three halogens. In another class of this embodiment, R^{h} is hydrogen.

In another embodiment of the present invention, each Rⁱ is —C₁₋₆ alkyl, wherein each alkyl is unsubstituted or substituted with one to three halogens. In a class of this embodiment, each R¹ is —C₁₋₆ alkyl. In another class of this embodiment, each Rⁱ is —CH₃.

In another embodiment of the present invention, R^{j} is independently selected from the group consisting of: hydrogen, OH and —C₁₋₆ alkyl, wherein each alkyl is unsubstituted or substituted with one to three halogens. In a class of this embodiment, R^{j} is independently selected from the group consisting of: hydrogen, and —C₁₋₆ alkyl, wherein each alkyl is unsubstituted or substituted with one to three halogens. In another class of this embodiment, R^{j} is hydrogen or OH. In another class of this embodiment, R^{j} is OH. In another class of this embodiment, R^{j} is hydrogen. In another class of this embodiment, R^{j} is —C₁₋₆ alkyl, wherein each alkyl is unsubstituted or substituted with one to three halogens.

In another embodiment of the present invention, R^{k} is independently selected from the group consisting of: hydrogen, and —C₁₋₆ alkyl, wherein each alkyl is unsubstituted or substituted with one to three halogens. In a class of this embodiment, R^{k} is —C₁₋₆ alkyl, wherein each alkyl is unsubstituted or substituted with one to three halogens. In another class of this embodiment, R^{k} is hydrogen.

In another embodiment of the present invention, R^{l} is independently selected from the group consisting of: hydrogen, and —C₁₋₆ alkyl, wherein each alkyl is unsubstituted or substituted with one to three halogens. In a class of this embodiment, R^{l} is —C₁₋₆ alkyl, wherein each alkyl is unsubstituted or substituted with one to three halogens. In another class of this embodiment, R^{l} is hydrogen.

In another embodiment of the present invention, R^{m} is independently selected from the group consisting of: hydrogen, and —C₁₋₆ alkyl, wherein each alkyl is unsubstituted or substituted with one to three halogens. In a class of this embodiment, R^{m} is —C₁₋₆ alkyl, wherein each alkyl is unsubstituted or substituted with one to three halogens. In another class of this embodiment, R^{m} is hydrogen.

In another embodiment of the present invention, each r is independently 0, 1, or 2. In a class of this embodiment, r is 0 or 1. In another class of this embodiment, r is 1 or 2. In another class of this embodiment, r is 0 or 2. In another class of this embodiment, r is 0. In another class of this embodiment, r is 1. In another class of this embodiment, r is 2.

In another embodiment of the present invention, each s is independently 0, 1, 2, 3, 4 or 5. In a class of this embodiment, each s is independently 0, 1, 2, 3 or 4. In another class of this embodiment, each s is independently 0, 1, 2, or 3. In another class of this embodiment, each s is independently 1, 2, or 3. In another class of this embodiment, each s is independently 1 or 3. In another class of this embodiment, s is 0 or 1. In another class of this embodiment, s is 1 or 2. In another class of this embodiment, s is 0 or 2. In another class of this embodiment, s is 0. In another class of this embodiment, s is 1. In another class of this embodiment, s is 2. In another class of this embodiment, s is 3. In another class of this embodiment, s is 4. In another class of this embodiment, s is 5.

In another embodiment of the present invention, each t is independently 0, 1, 2, or 3. In a class of this embodiment, t is 0, 1, or 2. In another class of this embodiment, t is 0 or 1. In another class of this embodiment, t is 1 or 2. In another class of this embodiment, t is 0 or 2. In another class of this embodiment, t is 0. In another class of this embodiment, t is 1. In another class of this embodiment, t is 2. In another class of this embodiment, t is 3.

In another embodiment of the present invention, each u is independently 0, 1, or 2. In a class of this embodiment, u is 0 or 1. In another class of this embodiment, u is 1 or 2. In another class of this embodiment, u is 0 or 2. In another class of this embodiment, u is 0. In another class of this embodiment, u is 1. In another class of this embodiment, u is 2.

In another embodiment of the present invention, each v is independently 0, 1, or 2. In a class of this embodiment, v is 0 or 1. In another class of this embodiment, v is 1 or 2. In another class of this embodiment, v is 0 or 2. In another class of this embodiment, v is 0. In another class of this embodiment, v is 1. In another class of this embodiment, v is 2.

In another embodiment of the present invention, the invention relates to compounds of structural formula Ia:

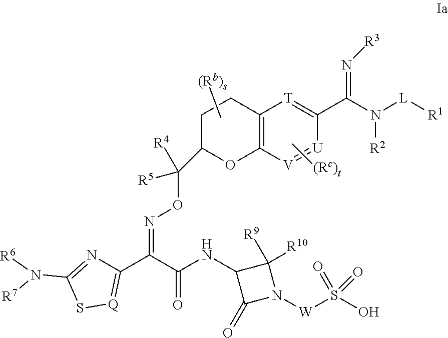

or a pharmaceutically acceptable salt thereof.

In another embodiment of the present invention, the invention relates to compounds of structural formula Ib:

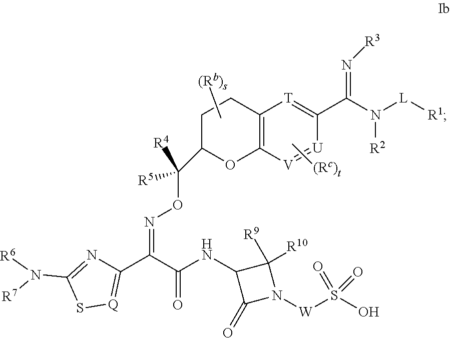

or a pharmaceutically acceptable salt thereof.

In another embodiment of the present invention, the invention relates to compounds of structural formula Ic:

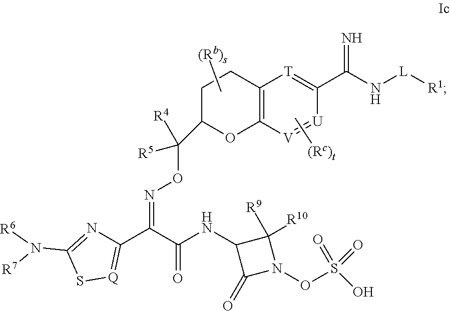

or a pharmaceutically acceptable salt thereof.

In another embodiment of the present invention, the invention relates to compounds of structural formula Id:

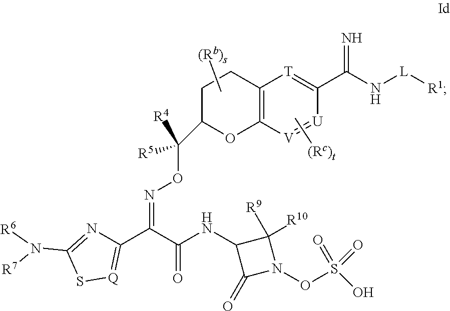

or a pharmaceutically acceptable salt thereof.

In another embodiment of the present invention, the invention relates to compounds of structural formula Ie:

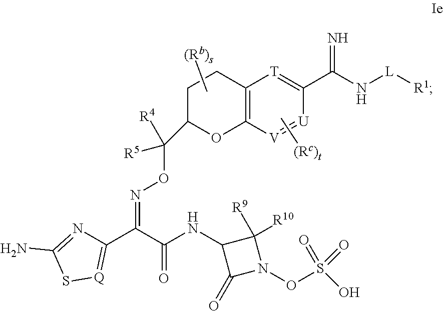

or a pharmaceutically acceptable salt thereof.

In another embodiment of the present invention, the invention relates to compounds of structural formula If:

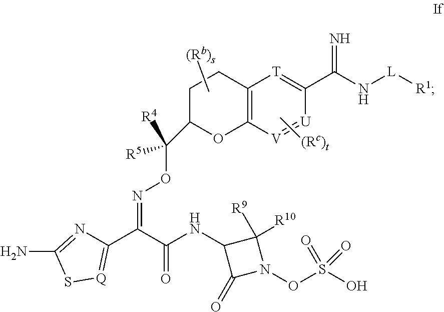

or a pharmaceutically acceptable salt thereof.

In another embodiment of the present invention, the invention relates to compounds of structural formula Ig:

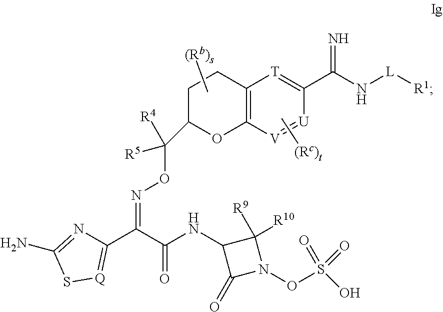

or a pharmaceutically acceptable salt thereof.

In another embodiment of the present invention, the invention relates to compounds of structural formula Ih:

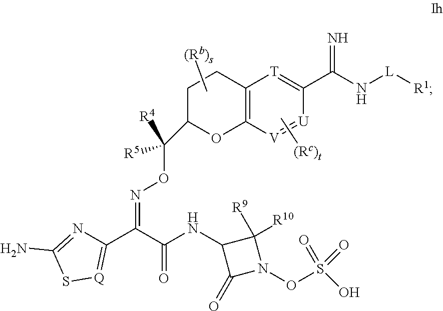

or a pharmaceutically acceptable salt thereof.

In another embodiment of the present invention, the invention relates to compounds of structural formula II:

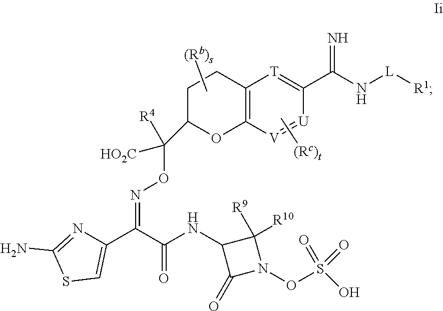

or a pharmaceutically acceptable salt thereof.

In another embodiment of the present invention, the invention relates to compounds of structural formula Ij:

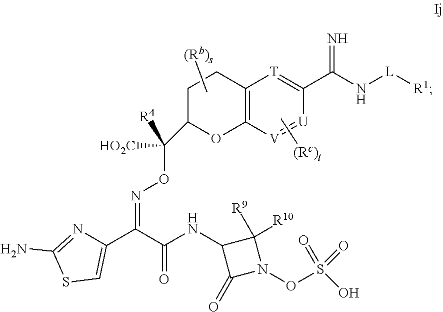

or a pharmaceutically acceptable salt thereof.

In another embodiment of the present invention, the invention relates to compounds of structural formula Ik:

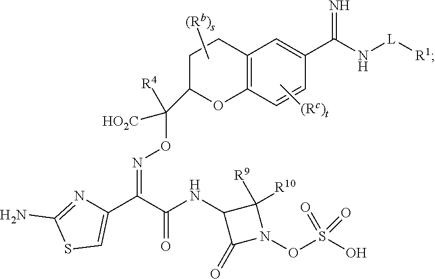

or a pharmaceutically acceptable salt thereof.

In another embodiment of the present invention, the invention relates to compounds of structural formula II:

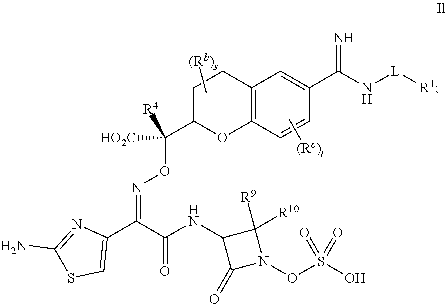

or a pharmaceutically acceptable salt thereof.

In another embodiment of the present invention, the invention relates to compounds of structural formula Im:

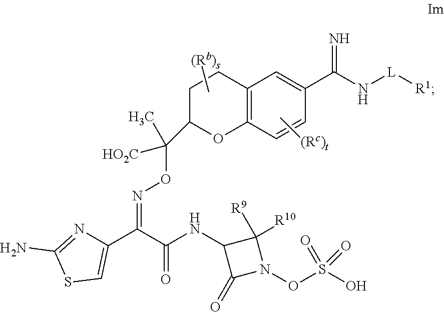

or a pharmaceutically acceptable salt thereof.

In another embodiment of the present invention, the invention relates to compounds of structural formula In:

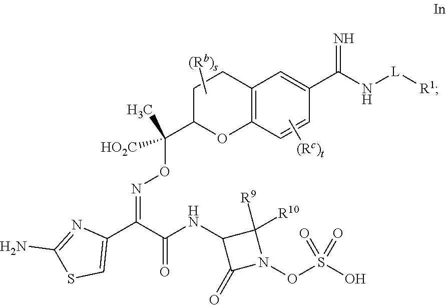

or a pharmaceutically acceptable salt thereof.

The compound of structural formula I includes the compounds of structural formulas Ia, Ib, Ic, Id, Ie, If, Ig, Ih, Ii, Ij, Ik, Il, Im and In, and pharmaceutically acceptable salts, hydrates and solvates thereof.

Another embodiment of the present invention relates to compounds of structural formula I wherein:
T is CH;
U is CH;
V is CH;
X is CH₂;
Y is O or CH₂;
Z is O or CH₂;
W is bond or O;
Q is CR⁸;
L is selected from the group consisting of:
   1) —C₁₋₆alkyl-, and
   2) —C₁₋₆alkyl-O—,
wherein alkyl is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl;
R¹ is selected from the group consisting of:
   1) —C₃₋₁₂cycloalkyl,
   2) C₂₋₁₁cycloheteroalkyl, and
   3) aryl,
wherein cycloalkyl, cycloheteroalkyl and aryl are unsubstituted or substituted with one to five substituents selected from R^{a};
R² is hydrogen;
R³ is hydrogen;
R⁴ is selected from the group consisting of:
   1) C₁₋₃alkyl, and
   2) C₃cycloalkyl,
wherein alkyl and cycloalkyl are unsubstituted or substituted with one to three substituents selected from: halogen and OC₁₋₃alkyl;
R⁵ is —CO₂H or tetrazole;
R⁶ is hydrogen;
R⁷ is hydrogen;
R⁸ is hydrogen;
R⁹ is C₁₋₆alkyl, and
R¹⁰ is C₁₋₆alkyl;
or a pharmaceutically acceptable salt thereof.
R^{a}, R^{b}, R^{c}, R^{d}, R^{e}, R^{f}, R^{g}, R^{h}, Rⁱ, R^{j}, R^{k}, R^{l}, R^{m}, r, s, t, u, and v are as defined above;
or a pharmaceutically acceptable salt thereof.

Another embodiment of the present invention relates to compounds of structural formula I wherein:
T is CH;
U is CH;
V is CH;
X is CH₂;
Y is CH₂;
Z is O;
W is O;
Q is CR⁸;
L is —C₁₋₆alkyl-, wherein alkyl is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl;
R¹ is selected from the group consisting of:
   1) —C₃₋₁₂cycloalkyl, and
   2) C₂₋₁₁cycloheteroalkyl,
wherein cycloalkyl and cycloheteroalkyl are unsubstituted or substituted with one to five substituents selected from R^{a};
R² is hydrogen;
R³ is hydrogen;
R⁴ is C₁₋₃alkyl;
R⁵ is —CO₂H;
R⁶ is hydrogen;
R⁷ is hydrogen;
R⁸ is hydrogen;
R⁹ is C₁₋₆alkyl;
R¹⁰ is C₁₋₆alkyl; and
R^{a}, R^{b}, R^{c}, R^{d}, R^{e}, R^{f}, R^{g}, R^{h}, Rⁱ, R^{j}, R^{k}, R^{l}, R^{m}, r, s, t, u, and v are as defined above; or a pharmaceutically acceptable salt thereof.

Illustrative, but non-limiting, examples of the compounds of the present invention are the following compounds:
1) (S)-2-((R)-6-(N-(((1r,4R)-4-amino-1-fluorocyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
2) (S)-2-((R)-6-(N-(((1s,4S)-4-amino-1-fluorocyclohexyl)-methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)-oxy)propanoic acid;
3) (S)-2-((R)-6-(N-((3-amino-bicyclo[1.1.1]pentan-1-yl)-methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)-amino)oxy)propanoic acid;
4) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(piperidin-4-ylmethyl)-carbamimidoyl)chroman-2-yl)propanoic acid;
5) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N—(((R)-pyrrolidin-3-yl)methyl)-carbamimidoyl)chroman-2-yl)propanoic acid;
6) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N—(((S)-pyrrolidin-3-yl)methyl)-carbamimidoyl)chroman-2-yl)propanoic acid;
7) (S)-2-((R)-6-(N-(((1r,3R)-3-aminocyclobutyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)propanoic acid;
8) (S)-2-((R)-6-(N-(((1s,3S)-3-aminocyclobutyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)propanoic acid;
9) (S)-2-((((Z)-1-(2-aminothiazol-4-yl)-2-(((S)-2,2-dimethyl-4-oxo-1-(sulfooxy)azetidin-3-yl)amino)-2-oxo-ethylidene)-amino)oxy)-2-((R)-6-(N-(2-((S)-pyrrolidin-2-yl)-ethyl)-carbamimidoyl)chroman-2-yl)propanoic acid;
10) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N-(2-((R)-pyrrolidin-2-yl)ethyl)-carbamimidoyl)chroman-2-yl)propanoic acid;
11) (S)-2-((R)-6-(N-(3-(1-aminocyclopropyl)propyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)propanoic acid;
12) (S)-2-((R)-6-(N-(2-(1-aminocyclopropyl)ethyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2 -oxoethylidene)amino)oxy)-propanoic acid;
13) (S)-2-((R)-6-(N-(2-(1-aminocyclobutyl)ethyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)propanoic acid;
14) (S)-2-((R)-6-(N-(2-(4-aminopiperidin-1-yl)ethyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
15) (S)-2-((R)-6-(N-(((1r,4R)-4-aminocyclohexyl)methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)amino)oxy)propanoic acid;
16) (S)-2-((R)-6-(N-(((1s,4S)-4-aminocyclohexyl)methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)-amino)oxy)propanoic acid;
17) (S)-2-((R)-6-(N-(4-(aminomethyl)benzyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxo-ethylidene)amino)oxy)-propanoic acid;
18) (S)-2-((R)-6-(N-(3-(aminomethyl)benzyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxo-ethylidene)amino)oxy)-propanoic acid;
19) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(azetidin-3-ylmethyl)-carbamimidoyl)chroman-2-yl)propanoic acid;
20) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-((4-(methoxymethyl)-piperidin-4-yl)methyl)-carbamimidoyl)chroman-2-yl)propanoic acid;
21) (S)-2-((R)-6-(N-(((1S,3R)-3-amino-2,2-dimethylcyclobutyl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)propanoic acid;
22) (S)-2-((R)-6-(N-(((2R,4r,6R)-6-aminospiro[3.3]heptan-2-yl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
23) (S)-2-((R)-6-(N-(((2S,4s,6S)-6-aminospiro[3.3]heptan-2-yl)methyl)carbamimidoyl)-chroman- 2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
24) (S)-2-((R)-6-(N-(((2R,4r,6R)-6-amino-2-fluorospiro[3.3]heptan-2-yl)methyl)-carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
25) (S)-2-((R)-6-(N-(((2S,4s,6S)-6-amino-2-fluorospiro[3.3]heptan-2-yl)methyl)-carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
26) (S)-2-((R)-6-(N-(((1s,3S)-3-amino-1-methylcyclobutyl)-methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)-amino)oxy)propanoic acid;
27) (S)-2-((R)-6-(N-(((1r,3R)-3-amino-1-methylcyclobutyl)methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)amino)oxy)propanoic acid;
28) (S)-2-((R)-6-(N-(((1s,3S)-3-(aminomethyl)cyclobutyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
29) (S)-2-((R)-6-(N-(((1r,3R)-3-(aminomethyl)-cyclobutyl)methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)amino)oxy)propanoic acid;
30) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(2-(piperidin-4-yl)ethyl)-carbamimidoyl)chroman-2-yl)propanoic acid;
31) tert-butyl (S)-2-((((Z)-1-(2-((tert-butoxycarbonyl)amino)thiazol-4-yl)-2-(((S)-2,2-dimethyl-4-oxo-1-(sulfooxy)azetidin-3-yl)amino)-2-oxoethylidene)amino)oxy)-2-((R)-6-(N—(((R)-morpholin-2-yl)methyl)carbamimidoyl)chroman-2-yl)propanoate;
32) tert-butyl (S)-2-((((Z)-1-(2-((tert-butoxycarbonyl)amino)thiazol-4-yl)-2-(((S)-2,2-dimethyl-4-oxo-1-(sulfooxy)azetidin-3-yl)amino)-2-oxoethylidene)amino)oxy)-2-((R)-6-(N—(((S)-morpholin-2-yl)methyl)carbamimidoyl)chroman-2-yl)propanoate;
33) (S)-2-((R)-6-(N-(2-((1R,4R)-2,5-diazabicyclo[2.2.1]heptan-2-yl)ethyl)carbamimidoyl)-chroman- 2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
34) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(3-((S)-pyrrolidin-2-yl)propyl)-carbamimidoyl)chroman-2-yl)propanoic acid;
35) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(3-((R)-pyrrolidin-2-yl)propyl)-carbamimidoyl)chroman-2-yl)propanoic acid;
36) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(3-((S)-azetidin-2-yl)propyl)-carbamimidoyl)chroman-2-yl)propanoic acid;
37) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(3-((R)-azetidin-2-yl)propyl)-carbamimidoyl)chroman-2-yl)propanoic acid;
38) (S)-2-((R)-6-(N-(((1s,4S)-4-(aminomethyl)cyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
39) (S)-2-((R)-6-(N-(((1r,4R)-4-(aminomethyl)cyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)-oxy)propanoic acid;
40) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N-(2-((S)-pyrrolidin-3-yl)ethyl)-carbamimidoyl)chroman-2-yl)propanoic acid;
41) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N-(2-((R)-pyrrolidin-3-yl)ethyl)-carbamimidoyl)chroman-2-yl)propanoic acid;
42) (S)-2-((((Z)-1-(2-aminothiazol-4-yl)-2-(((S)-2,2-dimethyl-4-oxo-1-(sulfooxy)azetidin-3-yl)amino)-2-oxo-ethylidene)amino)oxy)-2-((R)-6-(N-(2-(azetidin-3-yl)-ethyl)-carbamimidoyl)chroman-2-yl)propanoic acid;
43) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N-(2-(3-methylazetidin-3-yl)ethyl)-carbamimidoyl)chroman-2-yl)propanoic acid;
44) (S)-2-((((Z)-1-(2-aminothiazol-4-yl)-2-(((S)-2,2-dimethyl-4-oxo-1-(sulfooxy)azetidin-3 -yl)amino)-2-oxoethylidene)-amino)oxy)-2-((R)-6-(N-(2-((S)-azetidin-2-yl)ethyl)-carbamimidoyl)chroman-2-yl)propanoic acid;
45) (S)-2-((((Z)-1-(2-aminothiazol-4-yl)-2-(((S)-2,2-dimethyl-4-oxo-1-(sulfooxy)azetidin-3-yl)amino)-2-oxo-ethylidene)amino)oxy)-2-((R)-6-(N-(2-((R)-azetidin-2-yl)ethyl)-carbamimidoyl)chroman-2-yl)propanoic acid;
46) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N—((S)-1-(azetidin-3-yl)propan-2-yl)carbamimidoyl)chroman-2-yl)propanoic acid;
47) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N—((R)-1-(azetidin-3-yl)propan-2-yl)carbamimidoyl)chroman-2-yl)propanoic acid;
48) (S)-2-((R)-6-(N—(((S)-1-amino-2,3-dihydro-1H-inden-5-yl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
49) (S)-2-((R)-6-(N—(((R)-1-amino-2,3-dihydro-1H-inden-5-yl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
50) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(2-(((S)-pyrrolidin-3-yl)oxy)ethyl)-carbamimidoyl)chroman-2-yl)propanoic acid;
51) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4S)-2-(hydroxymethyl)-piperidin-4-yl)methyl)carbamimidoyl)chroman-2-yl)propanoic acid;
52) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4R)-2-(hydroxymethyl)-piperidin-4-yl)methyl)carbamimidoyl)chroman-2-yl)propanoic acid;
53) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4R)-2-(hydroxymethyl)-piperidin-4-yl)methyl)carbamimidoyl)chroman-2-yl)propanoic acid;
54) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4S)-2-(hydroxymethyl)-piperidin-4-yl)methyl)carbamimidoyl)chroman-2-yl)propanoic acid;
55) (S)-2-((R)-6-(N-(((1S,2R)-2 -(aminomethyl)cyclopropyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
56) (S)-2-((R)-6-(N-(((1R,2S)-2-(aminomethyl)cyclopropyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
57) (S)-2-((R)-6-(N-(((1s,4S)-4-amino-4-methylcyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
59) (S)-2-((R)-6-(N-(((1r,4R)-4-amino-4-methylcyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
59) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-((4-(2-methoxyethyl)piperidin-4-yl)methyl)carbamimidoyl)chroman-2-yl)propanoic acid;
60) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4R)-2-(methoxymethyl)-piperidin-4-yl)methyl)carbamimidoyl)chroman-2-yl)propanoic acid;
61) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4S)-2-(methoxymethyl)-piperidin-4-yl)methyl)carbamimidoyl)chroman-2-yl)propanoic acid;
62) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4R)-2-(methoxymethyl)-piperidin-4-yl)methyl)carbamimidoyl)chroman-2-yl)propanoic acid;
63) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4S)-2-(methoxymethyl)-piperidin-4-yl)methyl)carbamimidoyl)chroman-2-yl)propanoic acid;
64) (S)-2-((R)-6-(N-(((1R,2S)-2-(2-aminoethyl)cyclopropyl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
65) (S)-2-((R)-6-(N-(((1S,2R)-2-(2-aminoethyl)cyclopropyl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
66) (S)-2-((R)-6-(N-(2-((1S,2R)-2-(aminomethyl)cyclopropyl)ethyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
67) (S)-2-((R)-6-(N-(2-((1R,2S)-2-(aminomethyl)cyclopropyl)ethyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
68) (S)-2-((R)-6-(N-(((1r,4R)-4-amino-1-methoxycyclohexyl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
69) (S)-2-((R)-6-(N-(((1s,4S)-4-amino-1-methoxycyclohexyl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
70) (S)-2-((R)-6-(N-(((1s,3S)-3-amino-3-(hydroxymethyl)cyclobutyl)methyl)-carbamimidoyl)-chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)amino)oxy)propanoic acid; and
71) (S)-2-((R)-6-(N-(((1r,3R)-3-amino-3-(hydroxymethyl)cyclobutyl)methyl)-carbamimidoyl)-chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)amino)oxy)propanoic acid;
   or a pharmaceutically acceptable salt thereof.

Other embodiments of the present invention include the following:
(a) A pharmaceutical composition comprising an effective amount of a compound of Formula (I) as defined herein, or a pharmaceutically acceptable salt thereof, and a pharmaceutically acceptable carrier.
(b) The pharmaceutical composition of (a), further comprising a second compound, wherein the second compound is a beta-lactamase inhibitor.
(c) The pharmaceutical composition of (b), wherein the second compound is selected from the group consisting of: relebactam, tazobactam, clavulanic acid, sulbactam, avibactam, taniborbactam, nacubactam, vaborbactam, zidebactam, durlobactam, enmetazobactam, and xeruborbactam, or a pharmaceutically acceptable salt thereof.
(d) A pharmaceutical composition comprising (i) a compound of Formula (I), or a pharmaceutically acceptable salt thereof, and (ii) a second compound, wherein the second compound is an beta-lactamase inhibitor compound, wherein the compound of Formula (I), and the second compound are each employed in an amount that renders the combination effective for treating or preventing bacterial infection.
(e) The combination of (d), wherein the second compound is selected from the group consisting of: relebactam, tazobactam, clavulanic acid, sulbactam, avibactam, taniborbactam, nacubactam, vaborbactam, zidebactam, durlobactam, enmetazobactam, and xeruborbactam, or a pharmaceutically acceptable salt thereof.
(f) A method for treating a bacterial infection in a subject which comprises administering to a subject in need of such treatment an effective amount of a compound of Formula (I), or a pharmaceutically acceptable salt thereof.
(g) A method for preventing and/or treating a bacterial infection which comprises administering to a subject in need of such treatment a pharmaceutical composition comprising an effective amount of a compound of Formula (I), or a pharmaceutically acceptable salt thereof, and a pharmaceutically acceptable carrier.
(h) A method for treating a bacterial infection which comprises administering to a subject in need of such treatment a therapeutically effective amount of the composition of (a), (b), (c), (d), or (e).
(i) The method of treating a bacterial infection as set forth in (f), (g), or (h), wherein the bacterial infection is due to Gram negative bacteria.
(j) The method of treating a bacterial infection as set forth in (f), (g), (h), or (i), wherein the bacterial infection is due to Pseudomonas aeruginosa or Acinetobacter baumannii.

The present invention also includes a compound of Formula (I), or a pharmaceutically acceptable salt thereof, (i) for use in, (ii) for use as a medicament for, or (iii) for use in the preparation (or manufacture) of a medicament for, medicine or treating bacterial infection, including infection with a multidrug resistant bacterial strain. In these uses, the compounds of the present invention can optionally be employed in combination with one or more second therapeutic agents including relebactam, tazobactam, clavulanic acid, sulbactam, avibactam, taniborbactam, nacubactam, vaborbactam, zidebactam, durlobactam, enmetazobactam, and xeruborbactam, or a pharmaceutically acceptable salt thereof.

Additional embodiments of the invention include the pharmaceutical compositions, combinations and methods set forth in (a)-(j) above and the uses set forth in the preceding paragraph, wherein the compound of the present invention employed therein is a compound of one of the embodiments, sub-embodiments, classes or sub-classes described above. The compound may optionally be used in the form of a pharmaceutically acceptable salt in these embodiments.

In the embodiments of the compounds and salts provided above, it is to be understood that each embodiment may be combined with one or more other embodiments, to the extent that such a combination provides a stable compound or salt and is consistent with the description of the embodiments. It is further to be understood that the embodiments of compositions and methods provided as (a) through (j) above are understood to include all embodiments of the compounds and/or salts, including such embodiments as result from combinations of embodiments.

Additional embodiments of the present invention include each of the pharmaceutical compositions, combinations, methods and uses set forth in the preceding paragraphs, wherein the compound of the present invention or its salt employed therein is substantially pure. With respect to a pharmaceutical composition comprising a compound of Formula (I) or its salt and a pharmaceutically acceptable carrier and optionally one or more excipients, it is understood that the term “substantially pure” is in reference to a compound of Formula (I) or its salt per se; i.e., the purity of this active ingredient in the composition.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “-O-alkyl,” etc.

As used herein, and throughout this disclosure, the following terms, unless otherwise indicated, shall be understood to have the following meanings:

The term “β-lactamase inhibitor” refers to a compound which is capable of inhibiting enzyme activity from β-lactamases. As used herein, inhibiting β-lactamase activity means inhibiting the activity of a class A, C, and/or D β-lactamase. For antimicrobial applications inhibition at a 50% inhibitory concentration is preferably achieved at or below about 100 micrograms/mL, or at or below about 50 micrograms/mL, or at or below about 25 micrograms/mL. The terms “class A”, “class B”, “class C”, and “class D” β-lactamases are understood by those skilled in the art and are described in S. G. Waley, β-lactamase: mechanisms of action, in The Chemistry of β-Lactams, M. I. Page, Ed.; Chapman and Hall, London, (1992) 198-228.

The term “metallo-β-lactamase” denotes a metalloprotein capable of inactivating a β-lactam antibiotic. The β-lactamase can be an enzyme which catalyzes the hydrolysis of the β-lactam ring of a β-lactam antibiotic. Of particular interest herein are microbial metallo-β-lactamases. The metallo-β-lactamase can be, for example, a zinc metallo-β-lactamase. β-Lactamases of interest include those disclosed in, e.g., S. G. Waley, β-lactamase: mechanisms of action, in The Chemistry of β-Lactams, M. I. Page, Ed.; Chapman and Hall, London, (1992) 198-228. ρ-Lactamases of particular interest herein include metallo-β-lactamases of Escherichia coli (such as New Delhi Metallo-β-lactamase, NDM), Serratia marcescens (such as IMP), and Klebsiella spp. (such as Verona integron-encoded metallo-β-lactamase, VIM).). Additional metallo-β-lactamases of interest herein include SPM-, GIM-, SIM-, KHM-, AIM-, DIM-, SMB-, TMB-, and FIM-type enzymes.

The term “antibiotic” refers to a compound or composition which decreases the viability of a microorganism, or which inhibits the growth or proliferation of a microorganism. The phrase “inhibits the growth or proliferation” means increasing the generation time (i.e., the time required for the bacterial cell to divide or for the population to double) by at least about 2-fold. Preferred antibiotics are those which can increase the generation time by at least about 10-fold or more (e.g., at least about 100-fold or even indefinitely, as in total cell death). As used in this disclosure, an antibiotic is further intended to include an antimicrobial, bacteriostatic, or bactericidal agent. Examples of antibiotics include penicillins, cephalosporins and carbapenems.

The term “β-lactam antibiotic” refers to a compound with antibiotic properties that contains a β-lactam functionality. Non-limiting examples of β-lactam antibiotics include penicillins, cephalosporins, penems, carbapenems, and monobactams.

The term “about”, when modifying the quantity (e.g., kg, L, or equivalents) of a substance or composition, or the value of a physical property, or the value of a parameter characterizing a process step (e.g., the temperature at which a process step is conducted), or the like refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of ±0.1, 0.2, 0.3, 0.4, 0.5, 1.0, 2.0, 3.0, 4.0, or 5.0 of the appropriate unit. In certain embodiments, “about” can mean a variation of ±1%, 2%, 3%, 4%, 5%, 10%, or 20%.

Another embodiment of the present invention is a compound of Formula (I), or a pharmaceutically acceptable salt thereof, as originally defined or as defined in any of the foregoing embodiments, sub-embodiments, aspects, classes or sub-classes, wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a product containing a compound of Formula (I) or its salt (e.g., the product isolated from a reaction mixture affording the compound or salt) consists of the compound or salt. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest level of purity governs. A compound or salt of 100% purity is one which is free of detectable impurities as determined by a standard method of analysis.

With respect to a compound of the invention which has one or more asymmetric centers and can occur as mixtures of stereoisomers, a substantially pure compound can be either a substantially pure mixture of the stereoisomers or a substantially pure individual diastereomer or enantiomer unless expressly depicted otherwise. The present invention encompasses all stereoisomeric forms of the compounds of Formula (I). Unless a specific stereochemistry is indicated, the present invention is meant to comprehend all such isomeric forms of these compounds. Centers of asymmetry that are present in the compounds of Formula (I) can all independently of one another have (R) configuration or (S) configuration.

When bonds to the chiral carbon are depicted as straight lines in the structural Formulas of the invention, it is understood that both the (R) and (S) configurations of the chiral carbon, and hence both enantiomers and mixtures thereof, are embraced within the Formula. Similarly, when a compound name is recited without a chiral designation for a chiral carbon, it is understood that both the (R) and (S) configurations of the chiral carbon, and hence individual enantiomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula (I) or it can be done on a final racemic product. Absolute stereochemistry may be determined by X-ray crystallography of crystalline products or crystalline intermediates which are derivatized, if necessary, with a reagent containing a stereogenic center of known configuration. Where compounds of this invention are capable of tautomerization, all individual tautomers as well as mixtures thereof are included in the scope of this invention. Unless a particular isomer, salt, solvate (including hydrates) or solvated salt of such racemate, enantiomer, diastereomer or tautomer is indicated, the present invention includes all such isomers, as well as salts, solvates (including hydrates) and solvated salts of such racemates, enantiomers, diastereomers and tautomers and mixtures thereof.

### Definitions

“Ac” is acetyl, which is CH₃C(═O)—.

“Alkyl” means saturated carbon chains which may be linear or branched or combinations thereof, unless the carbon chain is defined otherwise. Other groups having the prefix “alk”, such as alkoxy and alkanoyl, also may be linear or branched, or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like.

“Aryl” means a monocyclic, bicyclic or fused carbocyclic aromatic ring or ring system containing carbon atoms, wherein at least one of the rings is aromatic. The term aryl also encompasses an aryl group, as defined above, which is fused to an aryl, cycloalkyl, cycloalkenyl, cycloheteroalkyl, cycloheteroalkenyl or heteroaryl ring. Examples of aryl include phenyl and naphthyl. In one embodiment of the present invention, aryl is phenyl. In another embodiment of the present invention, aryl is dihydroindene. In another embodiment of the present invention, aryl is 2,3-dihydroindene.

“Cycloalkyl,” as used herein, refers to a saturated monocyclic ring or bicyclic, tricyclic, fused, spirocyclic or bridged ring system comprising 3 to 14 carbon atoms. The cycloalkyl ring system contains more than one ring, the rings can be joined via a ring carbon. Examples of cycloalkyl include cyclopropyl, cyclobutyl, cyclopentyl, cyclohexyl, cycloheptyl, indanyl, and the like. In one embodiment of the present invention, cycloalkyl is selected from: cyclopropane, cyclobutane, cyclopentane, and cyclohexane. In another embodiment, cycloalkyl is cyclopropane. In another embodiment, cycloalkyl is cyclobutane. In another embodiment, cycloalkyl is cyclopentane. In another embodiment, cycloalkyl is cyclohexane. In another embodiment of the present invention, cycloalkyl is selected from: cyclopropane, cyclobutane, cyclohexane, bicyclo[1.1.1]pentane, and spiro[3.3]heptane. In another embodiment of the present invention, cycloalkyl is selected from: cyclopropane, cyclobutane, bicyclo[1.1.1]pentane, and spiro[3.3]heptane. In another embodiment of the present invention, cycloalkyl is selected from: cyclopropane, cyclobutane, and cyclohexane. In another embodiment of the present invention, cycloalkyl is selected from: cyclopropane and cyclobutane. In another embodiment of the present invention, C₃₋₁₂cycloalkyl is selected from: cyclopropane, cyclobutane, cyclohexane, bicyclo[1.1.1]pentane, and spiro[3.3]heptane. In another embodiment of the present invention, C₃₋₁₂cycloalkyl is selected from: cyclopropane, cyclobutane, bicyclo[1.1.1]pentane, and spiro[3.3]heptane. In another embodiment of the present invention, C₃₋₁₂cycloalkyl is selected from: cyclopropane, cyclobutane, and cyclohexane. In another embodiment of the present invention, C₃₋₁₂cycloalkyl is selected from: cyclopropane and cyclobutane.

“Cycloalkenyl” means a monocyclic ring or bicyclic, spirocyclic, fused or bridged carbocyclic ring system having a specified number of carbon atoms containing at least one double bond. Examples of cycloalkenyl include cyclopropenyl, cyclobutenyl, cyclopentenyl, cycloheptenyl, and the like.

“Cycloheteroalkyl,” as used herein, refers to a saturated monocyclic ring or bicyclic, tricyclic, spirocyclic, fused or bridged ring system comprising 3 to 14 ring atoms, wherein from 1 to 4 of the ring atoms are independently N, NH, S (including SO and SO₂) and 0, and the remainder of the ring atoms are carbon atoms. When a heterocycloalkyl contains two or more rings, the rings may be fused, bridged or spirocyclic. The cycloheteroalkyl group can be joined via a ring carbon or ring nitrogen atom (if present). Where the ring or ring system contains one or more N atoms, the N can be in the form of quaternary amine. The nitrogen or sulfur atom of the heterocycloalkyl (if present) can be optionally oxidized to the corresponding N-oxide, S-oxide or S,S-dioxide. The cycloheteroalkyl ring may be substituted on the ring carbons and/or the ring nitrogen or sulfur. Examples of cycloheteroalkyl include, oxetanyl, piperidyl, pyrrolidinyl, piperazinyl, morpholinyl, thiomorpholinyl, thiazolidinyl, 1,4-dioxanyl, tetrahydrofuranyl, delta-lactam, delta-lactone, silacyclopentane, silapyrrolidine, pyrrolidinyl, azetidinyl, piperidine, piperazine, azepane, azocane, morpholine, thiomorpholine, and the like. In one embodiment of the present invention, cycloheteroalkyl is selected from azetidine, pyrrolidine, piperidine, morpholine, and diazabicyclo[2.2.1]heptane. In another embodiment of the present invention, cycloheteroalkyl is selected from azetidine, pyrrolidine, piperidine, and morpholine. In another embodiment of the present invention, cycloheteroalkyl is selected from azetidine, and pyrrolidine. In another embodiment of the present invention, C₂₋₁₁cycloheteroalkyl is selected from: azetidine, pyrrolidine, piperidine, morpholine, and diazabicyclo[2.2.1]heptane. In another embodiment of the present invention. C₂₋₁₁cycloheteroalkyl is selected from azetidine, pyrrolidine, piperidine, and morpholine. In another embodiment of the present invention, C₂₋₁₁cycloheteroalkyl is selected from azetidine, and pyrrolidine.

“Cycloheteroalkenyl” means a monocyclic ring or bicyclic, fused, spirocyclic or bridged ring system comprising 3 to 14 ring atoms and containing at least one double bond and at least one heteroatom. Examples of cycloheteroalkenyl include dihydropyran and dihydrofuran, and the like.

“Heteroaryl” means a monocyclic ring or bicyclic or fused ring system containing 5-14 ring atoms containing at least one ring heteroatom selected from N, NH, S (including SO and SO₂) and O, wherein at least one of the heteroatom containing rings is aromatic. The term heteroaryl encompasses a heteroaryl group, as defined above, which is fused to an aryl, cycloalkyl, cycloalkenyl, cycloheteroalkyl, cycloheteroalkenyl or heteroaryl ring. In the case of a heteroaryl ring system where one or more of the rings are saturated or partially saturated and contain one or more N atoms, the N can be in the form of quaternary amine. Any nitrogen atom of a heteroaryl can be optionally oxidized to the corresponding N-oxide. The heteroaryl group can be optionally substituted by one or more ring system substituents which may be the same or different. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzisoxazolyl, benzoxazolyl, benzothiazolyl, benzimidazolyl, benzopyrazolyl, benzofuranyl, benzothiophenyl (including S-oxide and dioxide), benzotriazolyl, furo(2,3-b)pyridyl, quinolyl, indolyl, isoquinolyl, quinazolinyl, dibenzofuranyl, and the like. In one embodiment of the present invention, heteroaryl is selected from: pyridine.

“Halogen” includes fluorine, chlorine, bromine and iodine. In one embodiment, halogen is fluorine, chorine, bromine or iodine. In another embodiment, halogen is fluorine or chlorine. In another embodiment, halogen is chlorine, fluorine or iodine. In another embodiment, halogen is fluorine. In another embodiment, halogen is chlorine. In another embodiment, halogen is bromine. In another embodiment, halogen is iodine.

“Me” represents methyl.

“Oxo” means an oxygen atom connected to another atom by a double bond and represents “═O”.

“Quatemary salt” means a cation formed by four covalent bonds to nitrogen. When any variable (e.g., R¹, R^{a}, etc.) occurs more than one time in any constituent or in Formula (I), its definition on each occurrence is independent of its definition at every other occurrence. Also, combinations of substituents and/or variables are permissible only if such combinations result in stable compounds. A squiggly line across a bond in a substituent variable represents the point of attachment.

“Saturated” means containing only single bonds.

“Unsaturated” means containing at least one double or triple bond. In one embodiment, unsaturated means containing at least one double bond. In another embodiment, unsaturated means containing at least one triple bond.

When any variable (e.g., R^{l}, R^{a}, etc.) occurs more than one time in any constituent or in formula I, its definition on each occurrence is independent of its definition at every other occurrence. Also, combinations of substituents and/or variables are permissible only if such combinations result in stable compounds. A squiggly line across a bond in a substituent variable represents the point of attachment.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to:

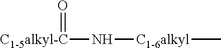

The phrase “pharmaceutically acceptable” is employed herein to refer to those compounds, materials, compositions, salts and/or dosage forms which are, using sound medical judgment, and following all applicable government regulations, safe and suitable for administration to a human being or an animal.

Compounds of Formula I may contain one or more asymmetric centers and can thus occur as racemates and racemic mixtures, single enantiomers, diastereomeric mixtures and individual diastereomers. The present invention is meant to encompass all such isomeric forms of the compounds of Formula I.

The independent syntheses of optical isomers and diastereoisomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration or sufficient heavy atoms to make an absolute assignment.

If desired, racemic mixtures of the compounds may be separated so that the individual enantiomers are isolated. The separation can be carried out by methods well-known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of Formula I.

In the compounds of general formula I, the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of structural formula I. For example, different isotopic forms of hydrogen (H) include protium (H), deuterium (H), and tritium (³H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Tritium is radioactive and may therefore provide for a radiolabeled compound, useful as a tracer in metabolic or kinetic studies. Isotopically-enriched compounds within structural formula I, can be prepared without undue experimentation by conventional techniques well known to those skilled in the art or by processes analogous to those described in the Schemes and Examples herein using appropriate isotopically-enriched reagents and/or intermediates.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

A “stable” compound is a compound which can be prepared and isolated and whose structure and properties remain or can be caused to remain essentially unchanged for a period of time sufficient to allow use of the compound for the purposes described herein (e.g., therapeutic administration to a subject). The compounds of the present invention are limited to stable compounds embraced by Formula (I).

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different. When a group, e.g., C₁-C₈ alkyl, is indicated as being substituted, such substitutions can also occur where such group is part of a larger substituent, e.g., —C₁-C₆alkyl-C₃-C₇cycloalkyl and —C₁-C₈alkyl-aryl.

Unless expressly stated to the contrary in a particular context, any of the various cyclic rings and ring systems described herein may be attached to the rest of the compound at any ring atom (i.e., any carbon atom or any heteroatom) provided that a stable compound results.

Unless expressly stated to the contrary, all ranges cited herein are inclusive. For example, a heteroaromatic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. Similarly, C₁-C₆ when used with a chain, for example an alkyl chain, means that the chain can contain 1, 2, 3, 4, 5 or 6 carbon atoms. It also includes all ranges contained therein including C₁-C₅, C₁-C₄, C₁-C₃, C₁-C₂, C₂-C₆, C₃-C₆, C₄-C₆, C₅-C₆, and all other possible combinations.

It should also be noted that any carbon as well as heteroatom with unsatisfied valences in the text, schemes, examples and tables herein is assumed to have the sufficient number of hydrogen atom(s) to satisfy the valences.

The compounds of the present invention have at least one asymmetric center and can have one or more additional centers as a result of the presence of certain substituents and/or substituent patterns. Accordingly, compounds of the invention can occur as mixtures of stereoisomers, or as individual diastereomers, or enantiomers. All isomeric forms of these compounds, whether individually or in mixtures, are within the scope of the present invention.

The term “compound” refers to the free compound and, to the extent they are stable, any hydrate or solvate thereof. A hydrate is the compound complexed with water, and a solvate is the compound complexed with an organic solvent.

As indicated above, the compounds of the present invention can be employed in the form of pharmaceutically acceptable salts. It will be understood that, as used herein, the compounds of the instant invention can also include the pharmaceutically acceptable salts, and also salts that are not pharmaceutically acceptable when they are used as precursors to the free compounds or their pharmaceutically acceptable salts or in other synthetic manipulations.

The term “Drug resistant” means, in connection with a Gram-negative bacterial strain, a strain which is no longer susceptible to at least one previously effective drug; which has developed the ability to withstand antibiotic attack by at least one previously effective drug. “Multi-drug resistant” means a strain that is no longer susceptible to two or more previously effective drugs; which has developed the ability to withstand antibiotic attack by two or more previously effective drugs. A drug resistant strain may relay that ability to withstand to its progeny. This resistance may be due to random genetic mutations in the bacterial cell that alters its sensitivity to a single drug or to different drugs.

The term “pharmaceutically acceptable salt” refers to a salt which possesses the effectiveness of the parent compound and which is not biologically or otherwise undesirable (e.g., is neither toxic nor otherwise deleterious to the recipient thereof). The term “pharmaceutically acceptable salt” refers to salts prepared from pharmaceutically acceptable non-toxic bases or acids including inorganic or organic bases and inorganic or organic acids.

Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, ascorbate, adipate, alginate, aspirate, benzenesulfonate, benzoate, bicarbonate, bisulfate, bitartrate, borate, bromide, butyrate, camphorate, camphorsulfonate, camsylate, carbonate, chloride, clavulanate, citrate, cyclopentane propionate, diethylacetic, digluconate, dihydrochloride, dodecylsulfanate, edetate, edisylate, estolate, esylate, ethanesulfonate, formate, formic, fumarate, gluceptate, glucoheptanoate, gluconate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, undeconate, valerate and the like. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethylamine, 2-diethylaminoethanol, 2-dimethylaminoethanol, ethanolamine, ethylamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylanine, tripropylamine, tromethamine, and the like. Also, included are the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

These salts can be obtained by known methods, for example, by mixing a compound of the present invention with an equivalent amount and a solution containing a desired acid, base, or the like, and then collecting the desired salt by filtering the salt or distilling off the solvent. The compounds of the present invention and salts thereof may form solvates with a solvent such as water, ethanol, or glycerol. The compounds of the present invention may form an acid addition salt and a salt with a base at the same time according to the type of substituent of the side chain.

As set forth above, the present invention includes pharmaceutical compositions comprising a compound of Formula I of the present invention, optionally one other active components (e.g., a β-lactamase inhibitor), and a pharmaceutically acceptable carrier. The characteristics of the carrier will depend on the route of administration. By “pharmaceutically acceptable” is meant that the ingredients of the pharmaceutical composition must be compatible with each other, do not interfere with the effectiveness of the active ingredient(s), and are not deleterious (e.g., toxic) to the recipient thereof. Thus, compositions according to the invention may, in addition to the inhibitor, contain diluents, fillers, salts, buffers, stabilizers, solubilizers, and other materials well known in the art.

Also as set forth above, the present invention includes a method for treating a bacterial infection which comprises administering to a subject in need of such treatment a therapeutically effective amount of a compound of Formula (I), or a pharmaceutically acceptable salt thereof, optionally in combination with a β-lactamase inhibitor. The term “subject” (or, alternatively, “patient”) as used herein refers to an animal, preferably a mammal, most preferably a human, who has been the object of treatment, observation or experiment. The term “administration” and variants thereof (e.g., “administering” a compound) in reference to a compound of Formula (I) mean providing the compound, or a pharmaceutically acceptable salt thereof, to the individual in need of treatment. When a compound or a salt thereof is provided in combination with one or more other active agents (e.g., a β-lactamase inhibitor), “administration” and its variants are each understood to include provision of the compound or its salt and the other agents at the same time or at different times. When the agents of a combination are administered at the same time, they can be administered together in a single composition or they can be administered separately.

It is understood that a “combination” of active agents can be a single composition containing all of the active agents or multiple compositions each containing one or more of the active agents. In the case of two active agents a combination can be either a single composition comprising both agents or two separate compositions each comprising one of the agents; in the case of three active agents a combination can be either a single composition comprising all three agents, three separate compositions each comprising one of the agents, or two compositions one of which comprises two of the agents and the other comprises the third agent; and so forth.

The compositions and combinations of the present invention are suitably administered in effective amounts. The term “effective amount” as used herein means the amount of active compound sufficient to inhibit bacterial growth and thereby elicit the response being sought (i.e., an “inhibition effective amount”) in a cell, tissue, system, animal or human. In one embodiment, the effective amount is a “therapeutically effective amount” for the alleviation of the symptoms of the disease or condition being treated (e.g., the healing of conditions associated with bacterial infection, and/or bacterial drug resistance). In another embodiment, the effective amount is a “prophylactically effective amount” for prophylaxis of the symptoms of the disease or condition being prevented. When the active compound (i.e., active ingredient) is administered as the salt, references to the amount of active ingredient are to the free acid or free base form of the compound.

The administration of a composition of the present invention is suitably parenteral, oral, sublingual, transdermal, topical, intranasal, intratracheal, intraocular, or intrarectal, wherein the composition is suitably formulated for administration by the selected route using formulation methods well known in the art, including, for example, the methods for preparing and administering formulations described in chapters 39, 41, 42, 44 and 45 in Remington—The Science and Practice of Pharmacy, 21^{st} edition, 2006. In one embodiment, compounds of the invention are administered intravenously in a hospital setting. In another embodiment, administration is oral in the form of a tablet or capsule or the like. When administered systemically, a therapeutic composition is for example, suitably administered at a sufficient dosage to attain a blood level of inhibitor of at least about 1 microgram/mL, and in additional embodiment at least about 10 micrograms/mL, and at least about 25 micrograms/mL. For localized administration, much lower concentrations than this may be effective, and much higher concentrations may be tolerated.

Intravenous administration of a compound of the invention can be conducted by reconstituting a powdered form of the compound with an acceptable solvent. Suitable solvents include, for example, saline solutions (e.g., 0.9% Sodium Chloride Injection) and sterile water (e.g., Sterile Water for Injection, Bacteriostatic Water for Injection with methylparaben and propylparaben, or Bacteriostatic Water for Injection with 0.9% benzyl alcohol). The powdered form of the compound can be obtained by gamma-irradiation of the compound or by lyophilization of a solution of the compound, after which the powder can be stored (e.g., in a sealed vial) at or below room temperature until it is reconstituted. The concentration of the compound in the reconstituted IV solution can be, for example, in a range of from about 0.1 mg/mL to about 20 mg/mL.

The present invention also includes a method for inhibiting bacterial growth which comprises administering to a bacterial cell culture, or to a bacterially infected cell culture, tissue, or organism, an inhibition effective amount of a compound of Formula (I). Additional embodiments of the invention include the bacterial growth inhibiting method just described, wherein the compound of the present invention employed therein is a compound of one of the embodiments, sub-embodiments or classes described above. The compound may optionally be used in the form of a pharmaceutically acceptable salt in these embodiments. The method can involve administration of a compound of Formula (I) to an experimental cell culture in vitro to prevent the growth of β-lactam resistant bacteria. The method can alternatively involve administration of a compound of Formula I to an animal, including a human, to prevent the growth of β-lactam resistant bacteria in vivo. In these cases the compound of Formula (I) is typically co-administered with a β-lactamase inhibitor.

The methods of the presently disclosed subject matter are useful for treating these conditions in that they inhibit the onset, growth, or spread of the condition, cause regression of the condition, cure the condition, or otherwise improve the general well-being of a subject afflicted with, or at risk of, contracting the condition. Thus, in accordance with the presently disclosed subject matter, the terms “treat”, “treating”, and grammatical variations thereof, as well as the phrase “method of treating”, are meant to encompass any desired therapeutic intervention, including but not limited to a method for treating an existing infection in a subject, and a method for the prophylaxis (i.e., preventing) of infection, such as in a subject that has been exposed to a microbe as disclosed herein or that has an expectation of being exposed to a microbe as disclosed herein.

Compounds of the invention can be employed for the treatment, prophylaxis or inhibition of bacterial growth or infections due to bacteria that are resistant to β-lactam antibiotics. More particularly, the bacteria can be metallo-β-lactamase positive strains that are highly resistant to β-lactam antibiotics. The terms “slightly resistant” and “highly resistant” are well-understood by those of ordinary skill in the art (see, e.g., Payne et al., Antimicrobial Agents and Chemotherapy 38:767-772 (1994); Hanaki et al., Antimicrobial Agents and Chemotherapy 30:11.20-11.26 (1995)). For the purposes of this invention, bacterial strains which are highly resistant to imipenem are those against which the MIC of imipenem is >16 μg/mL, and bacterial strains which are slightly resistant to imipenem are those against which the MIC of imipenem is >4 μg/mL.

The compounds wherein R⁴ is C₁₋₃alkyl, such as CH₃, or cyclopropyl and R⁵ is CO₂H or tetrazole have the unexpected benefit of stability compared to compounds wherein R⁴ is hydrogen and R⁵ is CO₂H or tetrazole.

Compounds of the invention can be used in combination with a β-lactamase inhibitor for the treatment of infections caused by β-lactamase producing strains, in addition to those infections which are subsumed within the antibacterial spectrum of the antibiotic agent. Examples of β-lactamase producing bacteria are Pseudomonas aeruginosa, Pseudomonas putida, Enterobacter cloacae, Klebsiella pneumoniae, Klebsiella oxytoca, Escherichia coli, Serratia marcescens, Enterobacter aerogenes, Enterobacter asburiae, Citrobacter freundii, Proteus mirabilis, Morganella morganii, Providencia rettgeri, Stenotrophomonas maltophilia and Acinetobacter baumannii.

It is generally advantageous to use a compound of Formula (I) in admixture or conjunction with a β-lactamase inhibitor, or a prodrug thereof. It is advantageous to use a compound of Formula I in combination with a class A and C β-lactamase inhibitor because of the class B β-lactamase resistant properties of the compounds. It is also advantageous to use a compound of Formula I in combination with one or more Class A, C, or D β-lactamase inhibitors to further limit β-lactam susceptibility. As already noted, the compound of Formula I and the β-lactamase inhibitor can be administered separately (at the same time or as different times) or in the form of a single composition containing both active ingredients.

Relebactam, tazobactam, clavulanic acid, sulbactam, avibactam, taniborbactam, nacubactam, vaborbactam, zidebactam, durlobactam, emnetazobactam, xeruborbactam, and other β-lactamase and metallo-β-lactamase inhibitors suitable for use in the present invention include those known to show inhibitory activity to β-lactamases.

### Abbreviations

Ambient is room temperature; aq. is aqueous; ACN is acetonitrile; AcOH is acetic acid; Bn is benzyl; BOC (or Boc) is t-butyloxycarbonyl; BOC₂O is di-tert-butyl dicarbonate; BuBr is butyl bromide; CBZ (or Cbz) is carbobenzoxy (alternatively, benzyloxycarbonyl); CBZ-Cl is benzyloxycarbonyl chloride; CDCl3 is deuterated chloroform; CV or cv is column volume(s); D₂O is deuterium oxide; DBU is 1,8-diazabicyclo[5.4.0]undec-7-ene; DCC is dicyclohexyl carbodiimide; DCE is dichloroethane; DCM is dichloromethane; DEAD is diethyl azodicarboxylate; (DHQD)₂AQN is 1,4-bis[(5-ethyl-1-azabicyclo[2.2.2]octan-2-yl)-(6-methoxyquinolin-4-yl)methoxy]anthracene-9,10-dione; DIAD is diisopropyl azodicarboxylate; DIEA or DIPEA is diisopropylethylamine; DMA is dimethylacetamide; DMAP is 4-dimethylaminopyridine or N,N-dimethylamino-pyridine; DME is dimethoxyethane; DMF is N,N-dimethylformamide; DMSO is dimethyl sulfoxide; DPPA is diphenylphosphoryl azide; EDC is 1-ethyl-3-(3-dimethylaminopropyl) carbodiimide; eq. or equiv. is equivalent(s); Et is ethyl; Et₃N is triethyl amine; Et₂O is diethyl ether; EA or EtOAc is ethyl acetate; EtOH is ethanol; g is gram(s); FA is formic acid; h or hr or hrs is hour(s); HATU is 1-[Bis(dimethylamino)methylene]-1H-1,2,3-triazolo[4,5-b]pyridinium 3-oxid hexafluorophosphate; hex is hexane; HMDS is hexamethyl-disilazide; HPLC is high-performance liquid chromatography; Int is intermediate; IPA is isopropyl alcohol; L or 1 is liter(s); LC/MS or LC-MS is liquid chromatography/mass spectrometry; LDA is lithium diisopropylamide; LiHMDS is lithium hexamethyl-disilazide M is molar; min is minute(s); mg is milligram(s); ml, mL or ML is milliliter(s); Me is methyl; MeCN is acetonitrile; MeOH is methanol; Mel is methyl iodide; MPLC is medium pressure liquid chromatography; MTBE is methyl tert-butyl ether; N is normal; NaBH(OAc)₃ is sodium triacetoxyborohydride; NBS is N-bromo-succinimide; NCS is N-chlorosuccinimide; NEt₃ is trietheyl amine; NMR is nuclear magnetic resonance; MS is mass spectrometry; MW is molecular weight; Pd/C is palladium on carbon; PdCl₂(dppf)₂ is [1,1′ bis(diphenyl-phosphino)-ferrocene]dichloropalladium(II); di-t-BuDPPF-PdCl₂ is 1,1′-bis(di-tert-butylphosphino)ferrocene palladium dichloride; Pd(AcO)₂ is palladium (II) acetate; PE is petroleum ether; PG is protective group; Ph is phenyl; Ph₃P is triphenyl phosphine; RP is reverse phase; RP-HPLC is reverse-phase high-performance liquid chromatography; rt, r.t., R.T. or RT is room temperature; sat'd is saturated; SFC is super critical fluid chromatography; tBu is tert-butyl; tBuOH is tert-butyl alcohol; TBAF is tetrabutylammonium fluoride; TB is tert-butyldimethylsilyl; TBS-Cl is tert-butyldimethylsilyl chloride; TBDPSCl is tert-Butyldiphenyl chlorosilane; t-BuOH is tert-butyl alcohol; TEA is triethylamine; TFA is trifluoroacetic acid; THF is tetrahydrofuran; TLC is thin layer chromatography; TMS is trimethylsilyl; TMS-Cl is trimethylsilyl chloride; and wt % is weight percentage.

### Methods for Making the Compounds of Formula (I):

The compounds disclosed herein can be prepared and tested according to the following reaction schemes and Examples, or modifications thereof, using readily available starting materials, reagents and conventional synthesis procedures. In these reactions, it is also possible to make use of variations which are themselves known to those of ordinary skill in this art, but are not mentioned here in greater detail. Furthermore, other methods for preparing compounds disclosed herein will be readily apparent to the person of ordinary skill in the art in light of the following reaction scheme and Examples. Unless otherwise indicated, all variables are as defined above. The following examples illustrate the invention and its practice. The examples are not to be construed as limitations on the scope or spirit of the invention. In these examples, all temperatures are degrees Celsius unless otherwise noted, and “room temperature” refers to a temperature in a range of from about 20° C. to about 25° C. Reactions sensitive to moisture or air were performed under nitrogen using anhydrous solvents and reagents. The progress of reactions can be determined by either analytical thin layer chromatography (TLC) performed with E. Merck precoated TLC plates or AnHui Liangchen Guiyuan Co., Ltd., silica gel 60F-254 or GF254, layer thickness 0.25 mm or liquid chromatography-mass spectrum (LC-MS). For HPLC/MS data of intermediates, unless otherwise specified, two main HPLC conditions used were as follows: 1) LC1 (SHIMADZU C18 Xtimate 3 μm 2.1×30 mm column with gradient 10:90-80:20 v/v CH₃CN/H₂O+v 0.0375% TFA over 0.9 min then hold at 80:20 v/v CH₃CN/H₂O+v 0.0375% TFA for 0.6 min; flow rate 1.2 mL/min, UV wavelength 220 & 254 nm); and 2) LC2 (Agilent C18 Xtimate 3 μm 2.1×30 mm column with gradient 10:90-80:20 v/v CH₃CN/H₂O+v 0.0375% TFA over 3.0 min then hold at 80:20 v/v CH₃CN/H₂O+v 0.0375% TFA for 0.5 min; flow rate 0.8 mL/min, UV wavelength 220 & 254 nm). For HPLC/MS data of final products, two main HPLC conditions used were as follows: 1) LC1: Agilent Poroshell 120 EC-C18 1.9 um 3.0×30 mm column with gradient 5:95-80:20 v/v CH₃CN (v 0.0375% TFA)/H₂O (v 0.0188% TFA) over 1.2 min then 80:20-95:5 v/v CH₃CN (v 0.0375% TFA)/H₂O (v 0.0188% TFA) for 1.3 min; flow rate 1.5 mL/min, UV wavelength 220 & 254 nm); and 2) LC2: Agilent Poroshell 120 EC-C18 1.9 um 3.0×30 mm column with gradient 0:100-30:70 v/v CH₃CN (v 0.0375% TFA)/H₂O (v 0.0188% TFA) over 1.2 min then 30:70-95:5 v/v CH₃CN (v 0.0375% TFA)/H₂O (v 0.0188% TFA) for 1.3 min; flow rate 1.5 m L/min, UV wavelength 220 & 254 nm); Mass analysis was performed with electrospray ionization in positive ion detection mode. ¹H NMR spectra were recorded on Varian or Bruker instruments at 400-500 MHz. Concentration of solutions was carried out on a rotary evaporator under reduced pressure or bubbled with nitrogen stream (a pipette head connecting to the end of nitrogen tube inserted in the mixture) until volatiles were removed completely or by lyophilization. Silica gel chromatography was performed on pre-packed silica gel columns using a commercial MPLC system. The names of compounds in the Examples were generated in Chemdraw™.

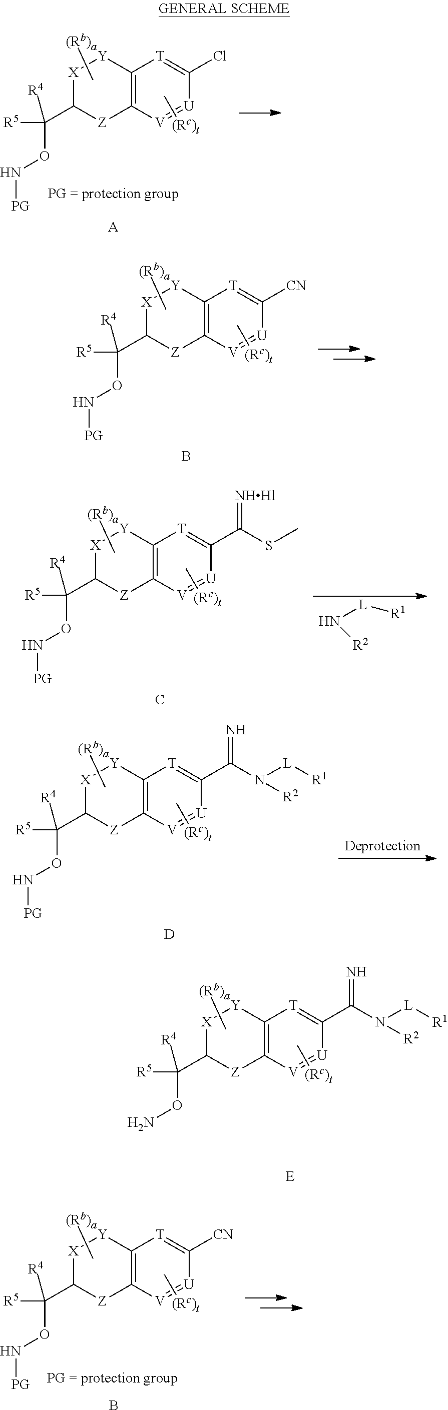

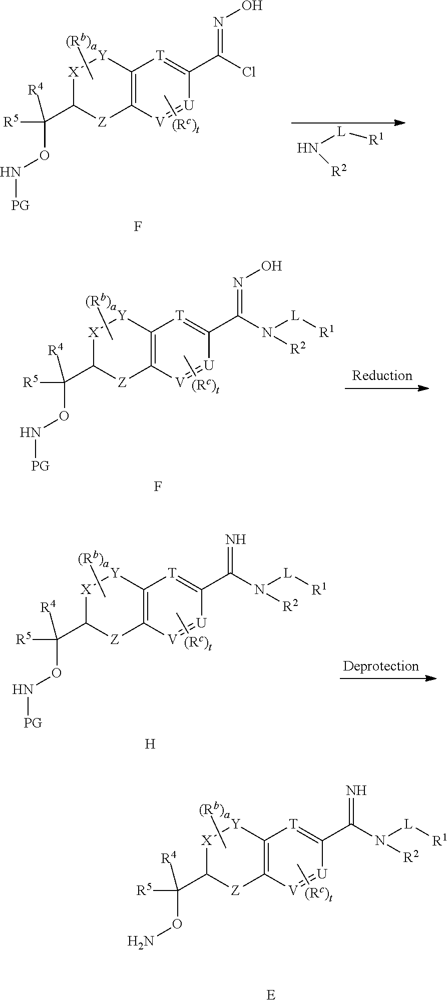

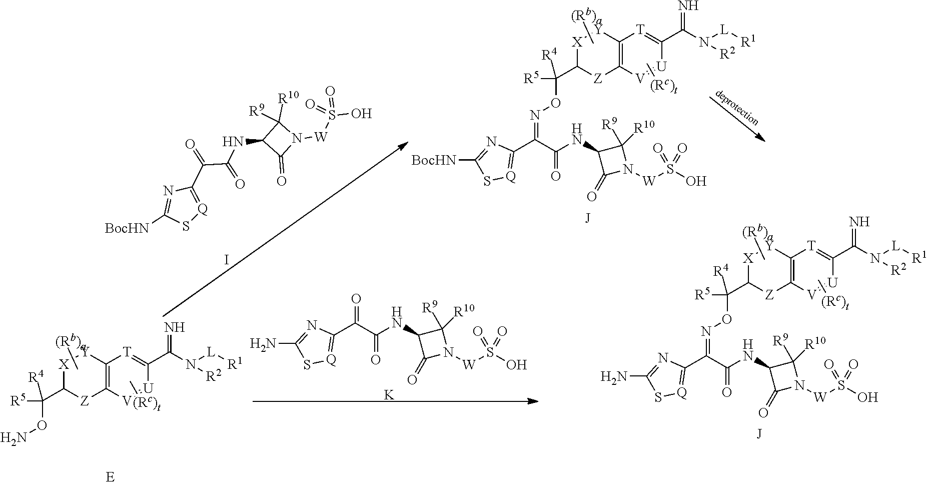

The chroman chloride A was converted via cynation reaction to intermediate B, which underwent functional group manipulations to provide intermediate C. Amination of intermediate C afforded intermediate D, which was then deprotected to give compound E. Alternatively, intermediate B was converted to intermediate F through functional group manipulation. Animation of intermediate F, followed by the reduction of hydroxyamidine G and removal of protecting group (PG) in intermediate H provided compound E. The condensation reaction of compound E with intermediate I afforded intermediate J, whose protecting group was removed to give the final product. Alternatively, compound E was coupled with intermediate K to afford the final product directly.

### Example 1: Preparation of Intermediate 1c

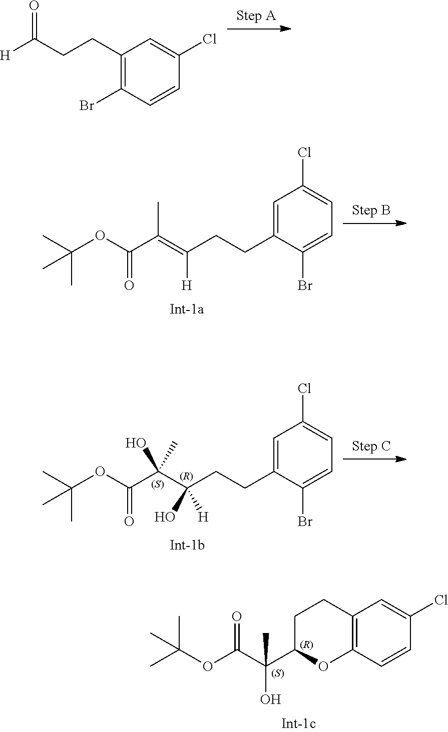

Step A—Synthesis of Intermediate-1α To a solution of tert-butyl 2-(diethoxyphosphoryl)-propanoate (2150.0 g, 8.06 mol) in THF (8.4 L) stirred at ambient temperature, was added NaH (339.3 g, 8.48 mol, 60% purity) in several portions. The mixture was stirred at 30-40° C. for 3 h. Then a solution of 3-(2-bromo-5-chlorophenyl)propanal (2100.0 g, 8.48 mol) in THF (4.2 L) was added dropwise to the above mixture at 30-50° C. After the addition, the mixture was stirred at 20-40° C. for 1 h, then poured into ice water (10 L), and diluted with EtOAc (10 L). The organic layer was separated, and the aqueous phase was extracted with EtOAc (3 L). The combined organic layers were washed with brine (10 L), and concentrated under reduced pressure. The resulting residue was purified by column chromatography on silica gel eluting with petroleum ether:ethyl acetate (1:0-10:1) to give intermediate 1a. ¹H-NMR (400 MHz, CDCl3): δ 7.47 (d, J=8.5 Hz, 1H), 7.22 (d, J=2.6 Hz, 1H), 7.07 (dd, J=8.5, 2.6 Hz, 1H), 6.71 (td, J=7.5, 1.5 Hz, 1H), 2.93-2.71 (m, 2H), 2.63-2.37 (m, 2H), 1.78 (d, J=1.3 Hz, 3H), 1.52 (s, 9H).

Step B—Synthesis of Intermediate 1b Into a 50 L 4-necked round-bottom flask, purged and maintained with an inert atmosphere of nitrogen, were added K₂CO₃ (2188 g, 15.84 mol), potassium ferricyanide (5218 g, 15.84 mol), tetraoxodipotassium osmium (38.1 g, 0.105 mol), (DHQD)₂AQN (90.1 g, 0.105 mol), and a solution of intermediate 1a (1900 g, 5.28 mol) in tert-butanol/water (19 L/19 L). The resulting mixture was stirred at room temperature for 2 days. Then the reaction mixture was extracted with ethyl acetate and the organic layers were combined and dried over anhydrous sodium sulfate. The resulting solids were filtered out. The filtrate was concentrated under vacuum, and the resulting residue was purified by a silica gel column eluting with ethyl acetate/petroleum ether (1/100-1/10) to give intermediate 1b. LC-MS: m/z 417.0 [M+Na]⁺.

Step C—Synthesis of Intermediate 1c To a mixture of Cs₂CO₃ (2317.2 g, 7112.01 mmol) and Pd(AcO)₂ (39.9 g, 177.80 mmol) in toluene (14 L) was slowly added 2-(di-tert-butylphosphino)biphenyl (106.1 g, 355.60 mmol) over 30 min. Then intermediate 1b (1400 g, 3556.01 mmol) was added, and the reaction mixture was stirred for 20 h at 90° C. Then the reaction mixture was cooled to room temperature with a water/ice bath. The resulting solids were filtered off, and the filtrate was concentrated under reduced pressure. The resulting residue was purified on a silica gel column eluting with ethyl acetate/petroleum ether (1/100-1/5) to give intermediate 1c. ¹H-NMR (400 MHz, CDCl3): δ 7.07-7.00 (m, 2H), 6.67 (d, J=8.3 Hz, 1H), 4.14 (dd, J=11.0, 2.4 Hz, 1H), 3.03-2.60 (m, 2H), 2.23-1.86 (m, 2H), 1.53 (s, 9H), 1.41 (s, 3H).

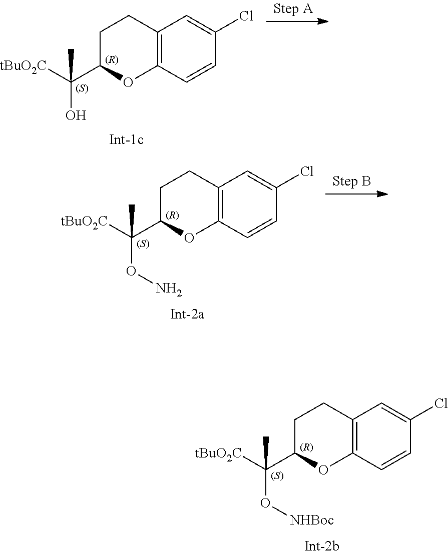

Step A—Synthesis of Intermediate 2a To a solution of intermediate 1c (460 g, 1.47 mol) in toluene (4.8 L) was added sodium hydride (60 wt. %, 70.8 g, 1.77 mol) in several batches at 27° C. The mixture was stirred at 27° C. for 1 h. Then a solution of amino 2,4,6-trimethylbenzene-1-sulfonate (380.4 g, 1.77 mol) in DCM (1.2 L) was added dropwise with stirring at 27° C. The reaction mixture was stirred at 27° C. for 2 h, then quenched by the addition of water (2 L) and extracted with MTBE (2×2 L). The organic layers were combined, dried over anhydrous Na₂SO₄ and filtered. The filtrate was concentrated under vacuum. The resulting residue was purified on a silica gel column eluting with EtOAc/PE (1:10) to give intermediate 2a. ¹H-NMR (400 MHz, CDCl3): δ 7.08-6.96 (m, 2H), 6.96 (overlap, 1H), 6.77 (d, J=9.4 Hz, 1H), 4.20 (dd, J=11.4, 1.9 Hz, 1H), 2.99-2.62 (m, 2H), 2.06 (ddt, J=13.6, 5.9, 2.1 Hz, 1H), 1.87 (dtd, J=13.6, 12.0, 5.8 Hz, 1H), 1.54 (s, 9H), 1.53 (s, 3H).

Step B—Synthesis of Intermediate 2b A mixture of 2a (500 g, 1525.27 mmol) and di-tert-butyl dicarbonate (399.00 g, 1828.18 mmol) in ethyl alcohol (5 L) was stirred for 5 h at 50° C., then concentrated under vacuum. The resulting crude product was purified by slurrying with hexanes. The solids were collected by filtration to afford intermediate 2b. ¹H NMR (400 MHz, CDCl3): δ 7.53 (s, 1H), 6.99 (t, 2H), 6.68 (t, 1H), 4.21 (q, 1H), 2.82 (t, 2H), 2.17-2.12 (m, 2H), 1.55-1.45 (m, 21H).

### Example 3: Preparation of Intermediates 3a and 3c

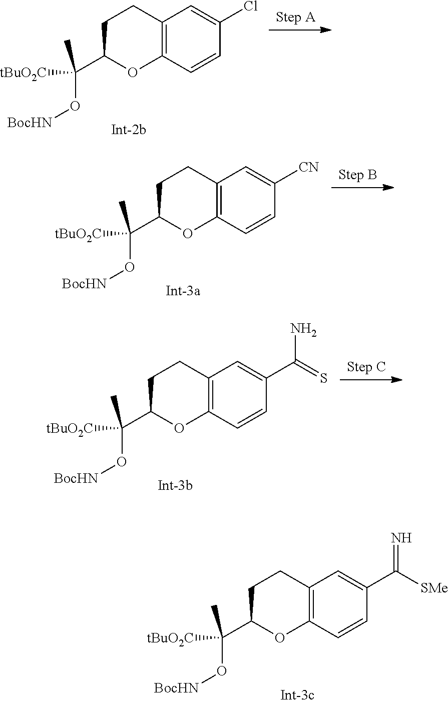 [SMILES: CC(C)(C)OC(=O)NO[C@](C)(C(=O)OC(C)(C)C)[C@H]1CCc2cc(Cl)ccc2O1 ; CC(C)(C)OC(=O)NO[C@](C)(C(=O)OC(C)(C)C)[C@H]1CCc2cc(C#N)ccc2O1 ; CC(C)(C)OC(=O)NO[C@](C)(C(=O)OC(C)(C)C)[C@H]1CCc2cc(C(N)=S)ccc2O1 ; CSC(=N)c1ccc2c(c1)CC[C@H]([C@](C)(ONC(=O)OC(C)(C)C)C(=O)OC(C)(C)C)O2]

Step A—Synthesis of Intermediate 3a To a mixture of intermediate 2b (8.0 g, 18.69 mmol), potassium hexacyanoferrate(II) trihydrate (3.95 g, 9.35 mmol), sodium carbonate (0.248 g, 2.337 mmol), and chloro(2-dicyclohexylphosphino-2′,4′,6′-tri-i-propyl-1,1′-biphenyl)(2′-amino-1,1′-biphenyl-2-yl) palladium(II) (1.471 g, 1.869 mmol) were added ACN (64 mL) and water (60 mL), both of which had been sparged with nitrogen for 1 h. The reaction vessel was evacuated and filled with nitrogen before sealing. Then the reaction was heated at 80° C. and stirred for 2 h. The reaction was then partitioned between ethyl acetate and water. The aqueous layer was back-extracted with ethyl acetate, and the combined organic layers were washed with brine, dried over anhydrous sodium sulfate, and filtered through a Celite™ pad. The resulting filtrate was concentrated in vacuo to give a crude residue, which was purified via silica gel chromatography (ISCO 220 g; 0-70% EtOAc/hexanes to give the title compound. LC-MS: m/z 419.2 [M+H]⁺.

Step B—Synthesis of Intermediate 3b To a mixture of intermediate 3a (4.81 g, 11.49 mmol), MgCl₂ (1.641 g, 17.24 mmol), and NaSH (1.933 g, 34.5 mmol) was added nitrogen-sparged anhydrous DMF (20.5 mL) under an atmosphere of nitrogen. The reaction mixture was evacuated and filled with nitrogen before capping and stirring at ambient temperature for 21 h. Then the reaction was cooled to 0° C. and quenched with saturated aqueous NH₄Cl and water. The resulting mixture was extracted with ethyl acetate. The aqueous layer was back-extracted with ethyl acetate and the combined organic layers were washed with brine, dried over anhydrous sodium sulfate, and filtered. The filtrate was concentrated in vacuo to give a crude residue, which was purified via silica gel chromatography (ISCO, 220 g; 0-100% EtOAc/hexanes) to give the title compound. LC-MS: m/z 453.2 [M+H]⁺.

Step C—Synthesis of Intermediate 3c To a solution of intermediate 3b (4.85 g, 10.72 mmol) in anhydrous ether (35 mL) was added iodomethane (0.804 mL, 12.86 mmol) at ambient temperature. The reaction was sealed and stirred for 24 h. Then the ether supernatant was decanted off The resulting oil was triturated with ether (15 mL), then dried under high vacuum to give intermediate 3c. The decanted ether layers were also combined and concentrated in vacuo. To the resulting residue was added 1:1 hexanes/ether (30 mL), and the resulting solid material was collected by filtration, washed with 1:1 hexanes/Et₂O (20 mL) and dried under vacuum to give an additional amount of intermediate 3c. The combined crude product was used in subsequent reactions without further purification. LC-MS: m/z 467.3 [M+H]⁺.

### Example 4: Preparation of Intermediate 4

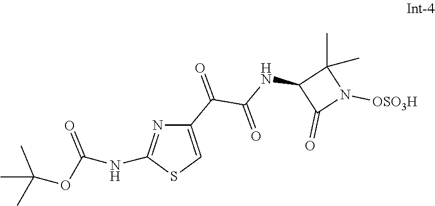

Intermediate 4 was prepared using the method described in Patent Publication No: WO 2017/106064.

### Example 5: Preparation of Intermediate 5

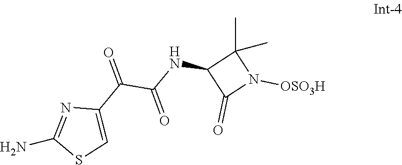

A solution of Intermediate 4 (10 g, 21.5 mmol) and CH₂Cl₂ (43 mL) was cooled to 0° C. Then TFA (86 mL, 1116 mmol) was added via syringe. The reaction mixture was stirred at 0° C. for 5 h, then concentrated under vacuum without heating to give a residue (~20 mL of total volume). DCM (100 mL) was added to the residue and the mixture was concentrated under vacuum without heating to ~20 mL total volume. This process was repeated four times to drive out most of the TFA. Finally, the solvent was removed completely under vacuum. To the resulting residue was added water (100 mL). After stirred for 30 min, the mixture was filtered, and the filter cake was rinsed with water (1 volume of cake) and then collected. The filter cake was dissolved in 1:1 acetonitrile/water (50 mL), and the mixture was lyophilized overnight to provide intermediate 5. LC-MS: m/z 365.3 [M+H]⁺. ¹H NMR (500 MHz, DMSO-d₆) δ: 9.67 (d, J=7.7 Hz, 1H), 7.88 (s, 1H), 7.57 (s, 2H), 4.59 (d, J=7.9 Hz, 1H), 1.44 (s, 3H), 1.25 (s, 3H).

### Example 6: Preparation of Intermediate 6c

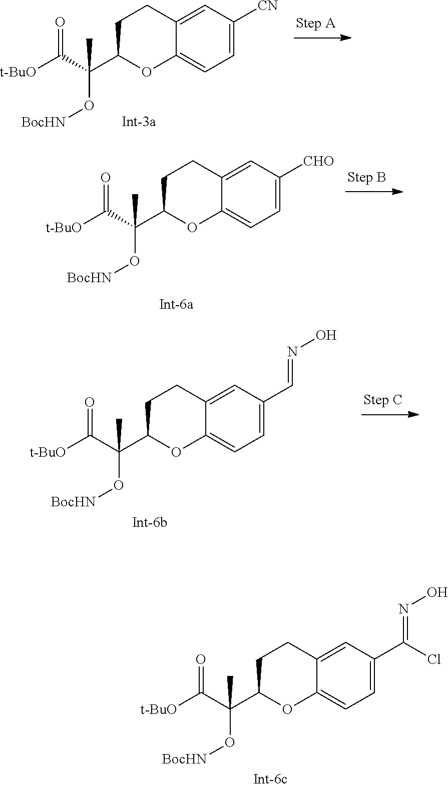 [SMILES: CC(C)(C)OC(=O)NO[C@](C)(C(=O)OC(C)(C)C)[C@H]1CCc2cc(C#N)ccc2O1 ; CC(C)(C)OC(=O)NO[C@](C)(C(=O)OC(C)(C)C)[C@H]1CCc2cc(C=O)ccc2O1 ; CC(C)(C)OC(=O)NO[C@](C)(C(=O)OC(C)(C)C)[C@H]1CCc2cc(/C=N/O)ccc2O1 ; CC(C)(C)OC(=O)NO[C@](C)(C(=O)OC(C)(C)C)[C@H]1CCc2cc(/C(Cl)=N/O)ccc2O1]

Step A—Synthesis of Intermediate 6a To a solution of intermediate 3a (0.98 g, 2.342 mmol) in a pre-mixed solution of AcOH (3 mL)/pyridine (6 mL)/water (3 mL), were added sodium hypophosphite monohydrate (1.986 g, 18.73 mmol) and Raney nickel (1.45 g). The resulting mixture was stirred at 70° C. for 12 h. Then the mixture was diluted with 100 mL of 50% EtOAc/hexanes and filtered. The filtrate was washed with 100 mL of water (3×), then dried over anhydrous MgSO₄, and concentrated under vacuum. The resulting residue was purified by silica gel chromatography (ISCO, 80 g column, gradient elution with 0~100% EtOAc/hexanes) to afford intermediate 6a. LC-MS (ESI) m/z: 422.3 [M+H]⁺.

Step B—Synthesis of Intermediate 6b To a mixture of intermediate 6a (300 mg, 0.712 mmol) in EtOH (3 mL) and water (3 mL) was added TEA (0.149 mL, 1.068 mmol), followed by hydroxylamine hydrochloride (74.2 mg, 1.068 mmol). The reaction mixture was stirred at 25° C. for 3 h, then diluted with water (40 mL) and extracted with ethyl acetate (80 mL×3). The combined organic layers were washed with brine (40 mL), dried over anhydrous Na₂SO₄, filtered, and the filtrate was concentrated in vacuo. The resulting residue was purified by preparative TLC plate (SiO₂, 30% ethyl acetate in petroleum ether) to give intermediate 6b. LC-MS (ESI) m/z: 459.0 [M+Na]⁺.

Step C—Synthesis of Intermediate 6c To a solution of intermediate 6b (500 mg, 1.145 mmol) in DMF (3.4 mL) was added 1-chloropyrrolidine-2,5-dione (199 mg, 1.489 mmol). The reaction was stirred at ambient temperature for 45 min. Then the reaction mixture was diluted with 15 mL of Et₂O/hexanes (2:1), and washed with 20 mL of water (2×). The organic layer dried over anhydrous Na₂SO₄, and then filtered. The filtrate was concentrated under vacuum to give crude intermediate 6c, which was used in subsequent reactions without further purification. LC-MS (ESI) m/z: 471.3 [M+H]⁺.

### Example 7: Preparation of Compound 1

### (S)-2-((R)-6-(N-(((1r,4R)-4-amino-1-fluorocyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

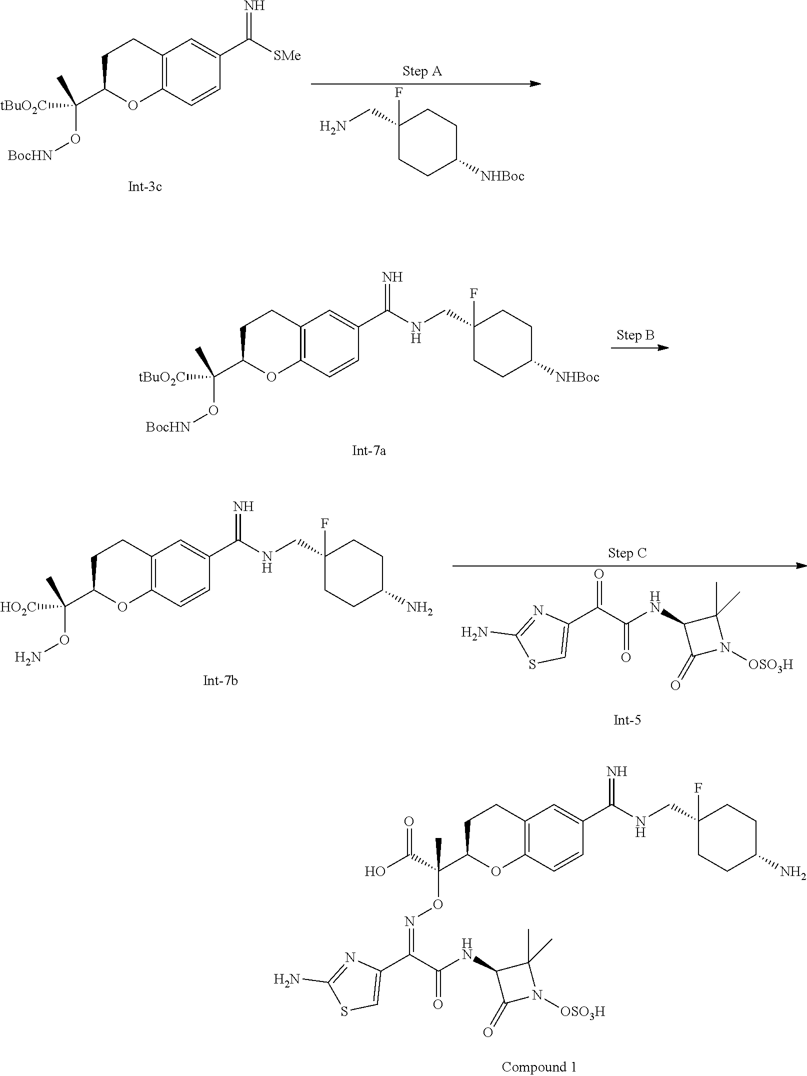

Step A—Synthesis of Intermediate 7a To a vial containing a mixture of 1,1-dimethylethyl N-[trans-4-(aminomethyl)-4-fluorocyclohexyl]carbamate (129.7 mg, 0.386 mmol) in anhydrous acetonitrile (1 mL) was added a solution of intermediate 3c (0.11 g, 0.238 mmol) and acetic acid (0.044 mL, 0.771 mmol) in anhydrous acetonitrile (1 mL). The reaction mixture was heated at 65° C. for 2 h. Then the reaction was cooled to ambient temperature and purified on a reverse phase HPLC (ISCO C18Aq 50 g; product elutes at 68% ACN+0.05% TFA/water+0.05% TFA) with gradient elution 0-100% ACN+0.05% TFA/water+0.05% TFA. The desired fractions were collected and concentrated in vacuo. The aqueous residue was partitioned between brine and ethyl acetate, and further back-extracted with ethyl acetate. The combined organic layers were dried over anhydrous sodium sulfate, and concentrated in vacuo to give the title compound. LC-MS: m/z 665.4 [M+H]⁺.

Step B—Synthesis of Intermediate 7b To a vial containing intermediate 7a (0.1139 g, 0.171 mmol) was added 2:1 trifluoroacetic acid/anhydrous dichloromethane (2 mL) at ambient temperature. The reaction was stirred for 16.5 h, then a solution of 4:1 MeOH/toluene (10 mL) was added to the reaction and the solution was concentrated in vacuo. The resulting residue was azeotroped with 4:1 MeOH/toluene (10 mL) and then dried under high vacuum to give the title compound. LC-MS: m/z 409.3 [M+H]⁺.

Step C—Synthesis of Compound 1 To a vial charged with intermediate 7b (0.171 mmol), intermediate 5 (79 wt %, 0.129 g, 0.199 mmol), and powdered molecular sieves 4A (325 mesh particle; 0.100 g, dried under high vacuum with heat) was added anhydrous dimethylacetamide (1.2 mL) at ambient temperature. The hazy reaction mixture was stirred for 18 h, then filtered through a Celite™ pad, which was then washed with MeOH. The filtrate was concentrated in vacuo, and the resulting residue was cooled to 0° C. With stirring, added DCM (6 mL) was slowly added to the residue resulting in precipitation of a yellow solid. The solid was collected by centrifugation (4000 rpm). The supernatant was decanted and the insoluble yellow solid was triturated with DCM (3 mL). Centrifugation and decanting of supernatant were repeated to give a crude solid, which was purified by RP HPLC (XSelect CSH Prep C18; 5 uM OBD; 50×250 mm; 0%-13% ACN/(water+0.16% TFA) over 11 min.; isocratic at 13% ACN/(water+0.16% TFA) for 14 min.). The product fractions were collected, concentrated n vacuo to remove acetonitrile, and the aqueous layer was directly loaded onto an Amberchrom CG161M column (26 g), washed with 9 CV of (water+0.1% FA), and eluted off with 3 CV of 100% (ACN+0.1% FA) followed by 3 CV of 50% (ACN+0.1% FA)/(water+0.1% FA). The desired fractions were collected, concentrated in vacuo, and the aqueous residue was lyophilized to give the title compound as the formic acid salt. LC-MS: m/z 755.3 [M+H]⁺. ¹H NMR (400 MHz, 4:1 D2O/d-DMSO) δ7.38-7.31 (m, 2H), 6.87-6.77 (m, 2H), 4.56 (s, 1H), 4.34 (d, J=11.0 Hz, 1H), 3.66 (bs, 1H), 3.60 (bs, 1H), 3.37-3.29 (m, 1H), 2.80-2.67 (m, 2H), 2.08-1.73 (m, 7H), 1.71-1.49 (m, 3H), 1.60-1.52 (m, 2H), 1.45 (s, 3H), 1.35 (s, 3H), 1.18 (s, 3H).

### Example 8: Preparation of Compounds 2 and 3

Starting from the appropriate commercially available mono-Boc protected diamines, the following compounds were prepared as formic acid salts according to Example 7:

| | | | LCMS |
|---|---|---|---|
| Compound | Structure | ¹H NMR | [M + H]⁺ |
| 2 | 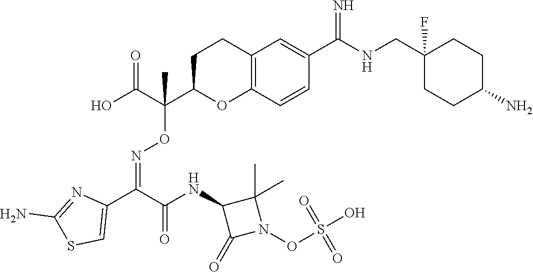 [SMILES: CC1(C)[C@H](NC(=O)/C(=N\O[C@](C)(C(=O)O)[C@H]2CCc3cc(C(=N)NC[C@]4(F)CC[C@@H](N)CC4)ccc3O2)c2csc(N)n2)C(=O)N1OS(=O)(=O)O] | ¹H NMR (400 MHz, 4:1 D₂O/d- DMSO) 7.37-7.32 (m, 2H), 6.89- 6.79 (m, 2H), 4.56 (s, 1H), 4.37 (dd, J = 11.2, 1.9 Hz, 1H), 3.57 (s, 1H), 3.52 (s, 1H), 3.18- 3.10 (m, 1H), 2.81-2.69 (m, 2H), 2.07-1.95 (m, 3H), 1.90-1.81 (m, 2H), 1.74-1.49 (m, 5H), 1.47 (s, 3H), 1.35 (s, 3H), 1.17 (s, 3H). | 755.3 |
| | (S)-2-((R)-6-(N-(((1s,4S)-4- | | |
| | amino-1-fluorocyclohexyl)- | | |
| | methyl)carbamimidoyl)chroman- | | |
| | 2-y1)-2-((((Z)-1-(2-aminothiazol- | | |
| | 4-y1)-2-(((S)-2,2-dimethyl-4-oxo- | | |
| | 1-(sulfooxy)azetidin-3-yl)amino)- | | |
| | 2-oxoethylidene)amino)- | | |
| | oxy)propanoic acid | | |
| 3 | 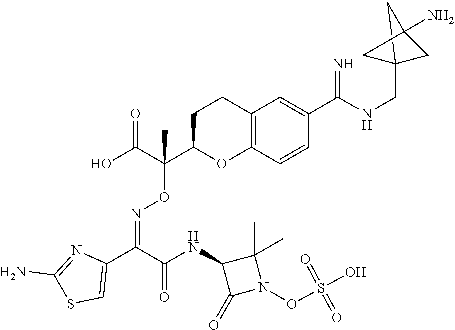 [SMILES: CC1(C)[C@H](NC(=O)/C(=N\O[C@](C)(C(=O)O)[C@H]2CCc3cc(C(=N)NCC45CC(N)(C4)C5)ccc3O2)c2csc(N)n2)C(=O)N1OS(=O)(=O)O] | ¹H NMR (400 MHz, 4:1 D₂O/d- DMSO) δ 7.37-7.31 (m, 2H), 6.86 (d, J = 8.5 Hz, 1H), 6.81 (s, 1H), 4.58 (s, 1H), 4.37 (d, J = 11.1 Hz, 1H), 3.60 (s, 2H), 2.83- 2.68 (m, 2H), 2.01 (s, 7H), 1.77- 1.60 (m, 1H), 1.47 (s, 3H), 1.36 (s, 3H), 1.18 (s, 3H). | 721.5 |
| | (S)-2-((R)-6-(N-((3-amino- | | |
| | bicyclo[1.1.1]pentan-1-yl)- | | |
| | methyl)carbamimidoyl)chroman- | | |
| | 2-yl)-2-((((Z)-1-(2-aminothiazol- | | |
| | 4-yl)-2-(((S)-2,2-dimethyl-4-oxo- | | |
| | 1-(sulfooxy)-azetidine-3- | | |
| | yl)amino)-2-oxoethylidene)- | | |
| | amino)oxy)propanoic acid | | |

### Example 9: Preparation of Intermediate 8c

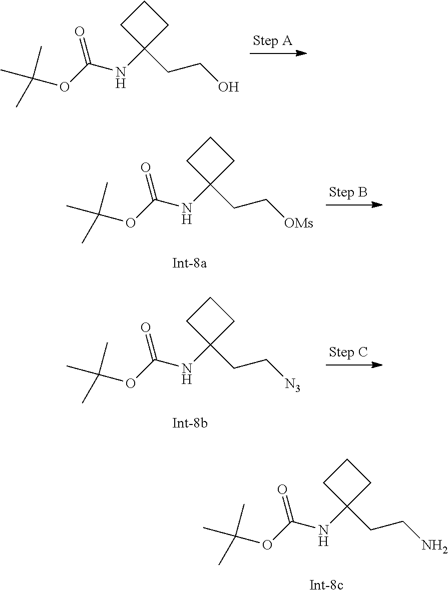 [SMILES: CC(C)(C)OC(=O)NC1(CCO)CCC1 ; CC(C)(C)OC(=O)NC1(CCOS(C)(=O)=O)CCC1 ; CC(C)(C)OC(=O)NC1(CCN=[N+]=[N-])CCC1 ; CC(C)(C)OC(=O)NC1(CCN)CCC1]

Step A—Synthesis of Intermediate 8a Methanesulfonyl chloride (0.216 mL, 2.79 mmol) was added to a stirred solution of tert-butyl N-[1-(2-hydroxyethyl)cyclobutyl]carbamate (500 mg, 2.322 mmol) and triethylamine (0.483 ml, 3.48 mmol) in THF (10 mL) at 0° C. The ice bath was removed, and the reaction was stirred at room temperature for 1 h, then quenched with saturated aqueous NaHCO₃ solution, and extracted with EtOAc. The combined organic layers were washed with brine, and dried over anhydrous MgSO₄. The drying agent was removed via filtration, and the filtrate concentrated under vacuum to give crude intermediate 8a, which was used in the next reaction without further purification. TLC: Rf=0.6 EtOAc/Hexane (1/1), KMnO₄ Stain.

Step B—Synthesis of Intermediate 8b Sodium azide (241 mg, 3.71 mmol) was added to a stirred solution of intermediate 8a (680 mg, 2.318 mmol) in DMF (10 mL) at room temperature. The reaction mixture was stirred at 70° C. overnight, then quenched with saturated aqueous NaHCO₃ solution, and extracted with EtOAc. The combined organic layers were washed with brine (2×), and dried over anhydrous MgSO₄. The drying agent was removed via filtration, and the filtrate was concentrated under vacuum to give crude intermediate 8b, which was used in the next reaction without further purification. TLC: Rf=0.7 EtOAc/Hexane (1/1), KMnO₄ Stain.

Step C—Synthesis of Intermediate 8c Intermediate 8b (140 mg, 0.583 mmol) was dissolved in ethanol (5 mL) and Pd/C (30 mg, 10%, 50% moisture) was added. The mixture was hydrogenated at room temperature under a H₂ balloon for 1 h. Then the catalyst was removed by filtration, and the filtrate was concentrated under vacuum to give crude intermediate 8c, which was used without further purification. TLC: Rf=0.0 EtOAc/Hexane (1/1), KMnO₄ Stain.

### Example 10: Preparation of Compound 4

### (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(piperidin-4-ylmethyl)carbamimidoyl)chroman-2-yl)propanoic Acid

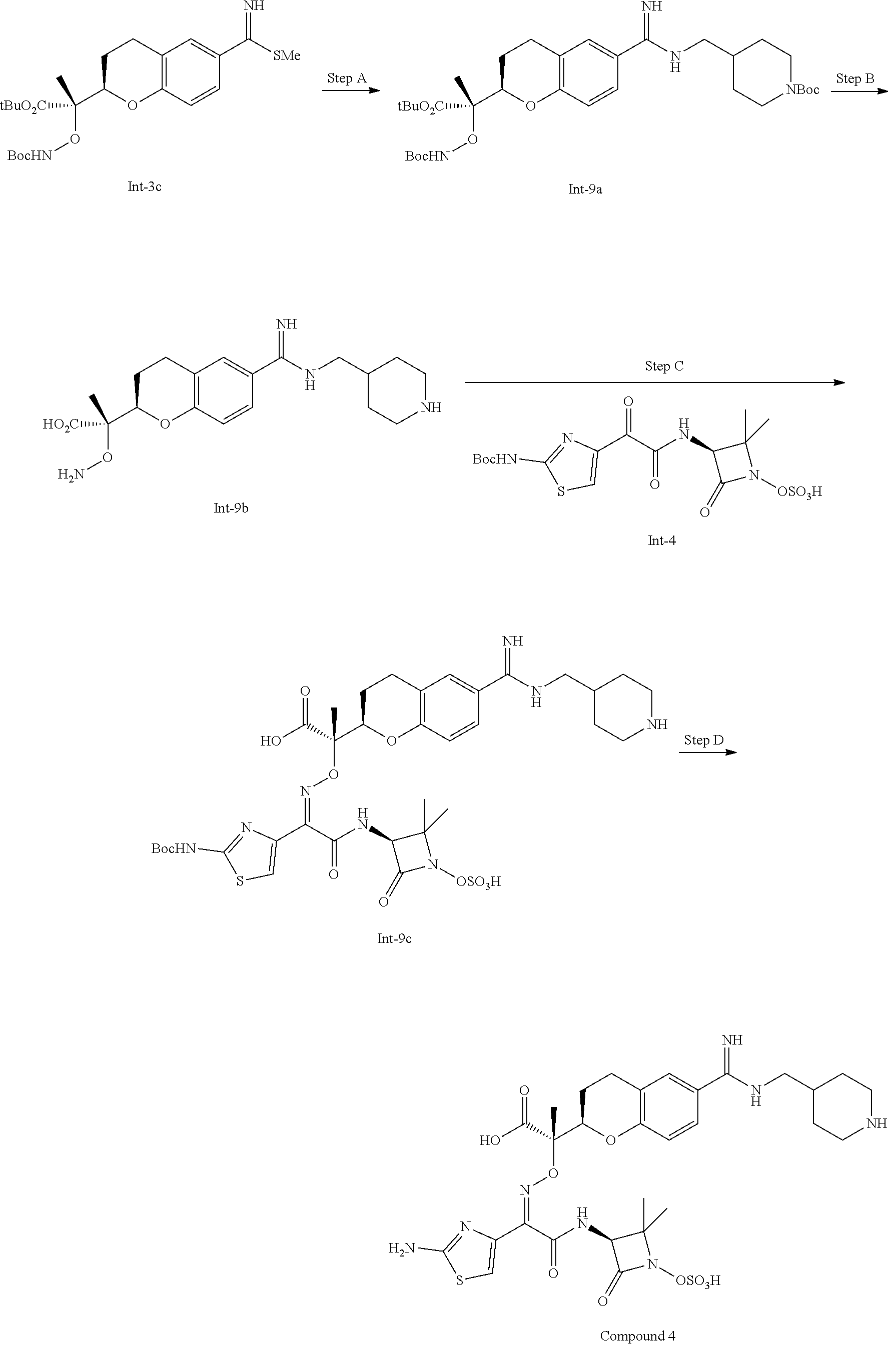

Step A—Synthesis of Intermediate 9a To a vial containing a mixture of 1-boc-4-(aminomethyl)-piperidine (90.7 uL, 0.429 mmol) and intermediate 3c (0.100 g, 0.214 mmol) was added a solution of potassium acetate (0.631 mg, 0.643 mmol) and acetic acid (0.07 mL, 1.286 mmol) in anhydrous MeOH (2 mL). The reaction was heated at 70° C. for 20 minutes, then cooled to room temperature, diluted with EtOAc (2 mL), and washed with saturated aqueous NaHCO₃ (1 mL). The organic layer was collected, dried over anhydrous Na₂SO₄, filtered, and the filtrate was concentrated in vacuo. The resulting residue was purified on a reverse phase HPLC (XSelect CSH Prep C18, 5 uM OBD, 30×150 mm; gradient elution with 35%-70% of ACN+0.05% TFA/water+0.05% TFA) to give the title compound. LC-MS [M+1]: m/z 633.6.

Step B—Synthesis of Intermediate 9b A mixture of intermediate 9a (0.0901 g, 0.144 mmol) and 2:1 trifluoroacetic acid/anhydrous dichloromethane (1.4 mL) at ambient temperature was stirred for 16.5 h. Then a solution of 4:1 MeOH/toluene (2 mL) was added to the reaction, and the solution was concentrated in vacuo. Repeated azeotroping of the resulting residue with 4:1 MeOH/toluene (2 mL) followed by drying the residue in vacuo to give the title compound. LC-MS [M+1]: m/z 377.25.

Step C—Synthesis of Intermediate 9c To a flask charged with intermediate 9b (0.239 mmol) and intermediate 4 (0.0105 g, 0.226 mmol) was added anhydrous methanol (2.4 mL) at ambient temperature. The reaction was stirred at room temperature for 3 h, and then concentrated in vacuo. The resulting residue was purified by a reverse phase HPLC (XSelect CSH Prep C18, 5 uM OBD, 30×150 mm; gradient elution with 12%-42% of ACN+0.05% TFA/water+0.05% TFA) to give the title compound. LC-MS [M+1]: m/z 823.9.

Step D—Synthesis of Compound 4 A solution of intermediate 9c (0.0737 g, 0.090 mmol) in 2:1 anhydrous DCM/TFA (0.9 mL) was stirred at ambient temperature for 1 h. The reaction was cooled to 0° C. and MTBE (3 mL) was added with stirring. The resulting mixture was sonicated and then centrifuged (4000 rpm) to collect the insoluble solids. The supernatant was decanted off, and the remaining solid was triturated with MTBE. The solid was isolated by centrifugation and dried in vacuo, then purified by reverse-phase chromatography (Isco C18 Aq 30 g Gold column; gradient elution with 0%-40% ACN+0.1% FA/water+0.1% FA). The desired fractions were collected and lyophilized to give the title compound as its formic acid salt. LC-MS [M+1]: m/z 723.4. ¹HNMR (400 MHz, 4:1 D₂O/d-DMSO) δ7.38-7.31 (m, 2H), 6.85 (d, J=8.5 Hz, 1H), 6.79 (s, 1H), 4.56 (s, 1H), 4.35 (d, J=9.6 Hz, 1H), 3.33 (d, J=13.0 Hz, 2H), 3.25 (d, J=6.9 Hz, 2H), 2.87 (t, J=11.9 Hz, 2H), 2.77-2.67 (m, 2H), 2.04-1.96 (m, 2H), 1.89 (s, 2H), 1.71-1.59 (m, 1H), 1.45 (s, 3H), 1.44-1.36 (m, 2H), 1.36 (s, 3H), 1.18 (s, 3H).

### Example 11: Preparation of Compounds 5 to 13

Starting from the appropriate commercially available mono-Boc protected diamines or di-Boc protected triamines, the following compounds were prepared as formic acid salts according to Example 10.

| | | | LCMS |
|---|---|---|---|
| Compound | Structure | ¹H NMR | [M + H]⁺ |
| 5 | 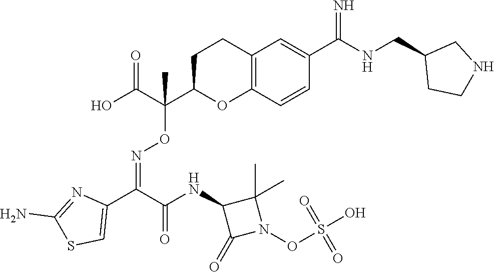 [SMILES: CC1(C)[C@H](NC(=O)/C(=N\O[C@](C)(C(=O)O)[C@H]2CCc3cc(C(=N)NC[C@@H]4CCNC4)ccc3O2)c2csc(N)n2)C(=O)N1OS(=O)(=O)O] | ¹H NMR (400 MHz, 4:1 D₂O/d- DMSO) δ: 7.36 (s, 1H), 7.33 (d, J = 8.6 Hz, 1H), 6.84 (d, J = 8.6 Hz, 1H), 6.81 (s, 1H), 4.57 (s, 1H), 4.34 (d, J = 10.4 Hz, 1H), 3.49-3.27 (m, 4H), 3.25-3.14 (m, 1H), 2.93 (dd, J = 11.9, 8.6 Hz, 1H), 2.77- 2.66 (m, 3H), 2.17 (dd, J = 13.0, 5.2 Hz, 1H), 2.02 (d, J = 11.6 Hz, 1H), 1.75-1.61 (m, 2H), 1.46 (s, 3H), 1.36 (s, 3H), 1.17 (s, 3H). | 709.5 |
| | (S)-2-((((Z)-1-(2-aminothiazol- | | |
| | 4-y1)-2-(((S)-2,2-dimethyl-4- | | |
| | oxo-1-(sulfooxy)azetidin-3- | | |
| | yl)amino)-2-oxoethylidene)- | | |
| | amino)oxy)-2-((R)-6-(N-(((R)- | | |
| | pyrrolidin-3-yl)methyl)- | | |
| | carbamimidoyl)chroman-2- | | |
| | yl)propanoic acid | | |
| 6 | 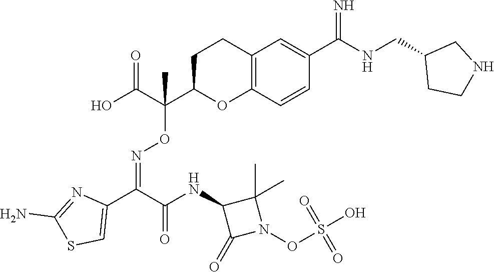 [SMILES: CC1(C)[C@H](NC(=O)/C(=N\O[C@](C)(C(=O)O)[C@H]2CCc3cc(C(=N)NC[C@H]4CCNC4)ccc3O2)c2csc(N)n2)C(=O)N1OS(=O)(=O)O] | ¹H NMR (400 MHz, 4:1 D₂O/d- DMSO) δ: 7.36 (s, 1H), 7.35-7.29 (d, J = 8.5 Hz, 1H), 6.85 (d, J = 8.5 Hz, 1H), 6.80 (s, 1H), 4.57 (s, 1H), 4.35 (d, J = 9.4 Hz, 1H), 3.48- 3.37 (m, 3H), 3.36-3.28 (m, 1H), 3.25-3.14 (m, 1H), 2.94 (dd, J = 12.0, 8.6 Hz, 1H), 2.79-2.66 (m, 3H), 2.18 (td, J = 12.7, 7.3 Hz, 1H), 2.02 (d, J = 13.2 Hz, 1H), 1.74-1.61 (m, 2H), 1.46 (s, 3H), 1.36 (s, 3H), 1.18 (s, 3H). | 709.5 |
| | (S)-2-((((Z)-1-(2-aminothiazol- | | |
| | 4-y1)-2-(((S)-2,2-dimethyl-4- | | |
| | oxo-1-(sulfooxy)azetidin-3- | | |
| | yl)amino)-2-oxoethylidene)- | | |
| | amino)oxy)-2-((R)-6-(N-(((S)- | | |
| | pyrrolidin-3-y1)methyl)- | | |
| | carbamimidoyl)chroman-2- | | |
| | yl)propanoic acid | | |
| 7 | 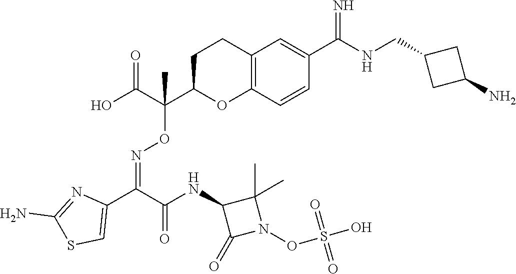 [SMILES: CC1(C)[C@H](NC(=O)/C(=N\O[C@](C)(C(=O)O)[C@H]2CCc3cc(C(=N)NC[C@H]4C[C@H](N)C4)ccc3O2)c2csc(N)n2)C(=O)N1OS(=O)(=O)O] | ¹H NMR (500 MHz, 400 uL D₂O/100 uL CD₃CN) δ: 7.45-7.37 (m, 2H), 6.93-6.89 (m, 2H), 4.48- 4.41 (m, 1H), 3.95-3.87 (m, 1H), 3.50 (d, J = 10 Hz, 2H), 2.84 (br s, 3H), 2.41-2.21 (m, 4H), 2.10 (br s, 1H), 2.01-1.95 (m, 1H), 1.78 (br s, 1H), 1.52 (s, 3H), 1.49 (s, 3H), 1.26 (s, 3H). | 709.4 |
| | (S)-2-((R)-6-(N-(((1r,3R)-3- | | |
| | aminocyclobutyl)methyl)carbami- | | |
| | midoyl)chroman-2-yl)-2-((((Z)- | | |
| | 1-(2-aminothiazol-4-yl)-2-(((S)- | | |
| | 2,2-dimethyl-4-oxo-1- | | |
| | (sulfooxy)azetidin-3-yl)amino)- | | |
| | 2-oxoethylidene)- | | |
| | amino)oxy)propanoic acid | | |
| 8 | 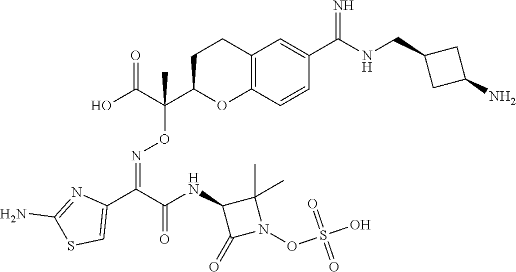 [SMILES: CC1(C)[C@H](NC(=O)/C(=N\O[C@](C)(C(=O)O)[C@H]2CCc3cc(C(=N)NC[C@H]4C[C@@H](N)C4)ccc3O2)c2csc(N)n2)C(=O)N1OS(=O)(=O)O] | ¹H NMR (500 MHz, 400 uL D₂O/100 uL CD₃CN) δ: 7.47-7.42 (m, 2H), 6.96-6.92 (m, 2H), 4.48 (d, J = 15 Hz, 1H), 3.76-3.68 (m, 1H), 3.46 (d, J = 5 Hz, 2H), 2.93-2.81 (m, 2H), 2.60-2.49 (m, 3H), 2.15 (br s, 1H), 2.03-1.99 (m, 1H), 1.98- 1.90 (m, 2H), 1.80 (br s, 1H), 1.52 (s, 3H), 1.49 (s, 3H), 1.29 (s, 3H). | 709.5 |
| | (S)-2-((R)-6-(N-(((1s,3S)-3- | | |
| | aminocyclobutyl)methyl)carbami- | | |
| | midoyl)chroman-2-y1)-2-((((Z)- | | |
| | 1-(2-aminothiazol-4-y1)-2-(((S)- | | |
| | 2,2-dimethyl-4-oxo-1- | | |
| | (sulfooxy)azetidin-3-yl)amino)- | | |
| | 2-oxoethylidene)- | | |
| | amino)oxy)propanoic acid | | |
| 9 | 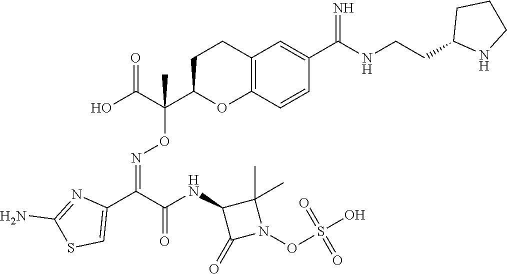 [SMILES: CC1(C)[C@H](NC(=O)/C(=N\O[C@](C)(C(=O)O)[C@H]2CCc3cc(C(=N)NCC[C@@H]4CCCN4)ccc3O2)c2csc(N)n2)C(=O)N1OS(=O)(=O)O] | ¹H NMR (500 MHz, 400 uL D₂O/100 uL CD₃CN) δ: 7.32-7.27 (m, 2H), 6.82-6.77 (m, 2H), 4.54 (s, 1H), 4.37-4.34 (m, 1H), 3.57-3.51 (m, 1H), 3.42-3.39 (m, 2H), 3.21- 3.12 (m, 2H), 2.70 (br s, 2H), 2.19- 2.09 (m, 2H), 2.02-1.83 (m, 3H), 1.75-1.55 (m, 3H), 1.45 (s, 3H), 1.32 (s, 3H), 1.10 (s, 3H). | 723.5 |
| | (S)-2-((((Z)-1-(2-aminothiazol- | | |
| | 4-yl)-2-(((S)-2,2-dimethyl-4- | | |
| | oxo-1-(sulfooxy)azetidin-3- | | |
| | yl)amino)-2-oxo-ethylidene)- | | |
| | amino)oxy)-2-((R)-6-(N-(2-((S)- | | |
| | pyrrolidin-2-yl)-ethyl)- | | |
| | carbamimidoyl)chroman-2- | | |
| | yl)propanoic acid | | |
| 10 | 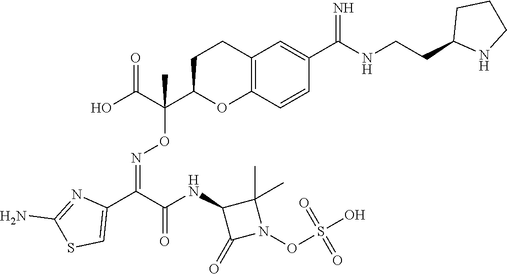 [SMILES: CC1(C)[C@H](NC(=O)/C(=N\O[C@](C)(C(=O)O)[C@H]2CCc3cc(C(=N)NCC[C@H]4CCCN4)ccc3O2)c2csc(N)n2)C(=O)N1OS(=O)(=O)O] | ¹H NMR (500 MHz, 400 uL D₂O/100 uL CD₃CN) δ: 7.32-7.26 (m, 2H), 6.79-6.77 (m, 2H), 4.53 (s, 1H), 4.37-4.34 (m, 1H), 3.57-3.51 (m, 1H), 3.42-3.39 (m, 2H), 3.21- 3.18 (m, 2H), 2.70 (br s, 2H), 2.20- 2.09 (m, 2H), 2.01-1.83 (m, 3H), 1.75-1.54 (m, 3H), 1.45 (s, 3H), 1.32 (s, 3H), 1.10 (s, | 723.4 |
| | (S)-2-((((Z)-1-(2-aminothiazol- | | |
| | 4-yl)-2-(((S)-2,2-dimethyl-4- | | |
| | oxo-1-(sulfooxy)azetidin-3- | | |
| | yl)amino)-2-oxoethylidene)- | | |
| | amino)oxy)-2-((R)-6-(N-(2-((R)- | | |
| | pyrrolidin-2-yl)ethyl)- | | |
| | carbamimidoyl)chroman-2- | | |
| | yl)propanoic acid | | |
| 11 | 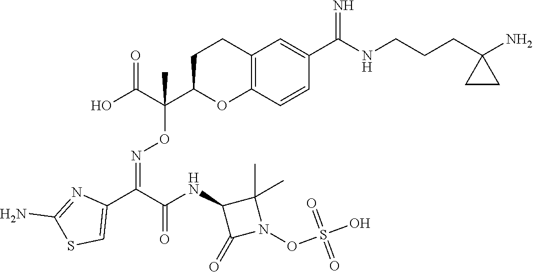 [SMILES: CC1(C)[C@H](NC(=O)/C(=N\O[C@](C)(C(=O)O)[C@H]2CCc3cc(C(=N)NCCCC4(N)CC4)ccc3O2)c2csc(N)n2)C(=O)N1OS(=O)(=O)O] | ¹H NMR (500 MHz, 400 uL D₂O/100 uL CD₃CN) δ: 7.34-7.30 (m, 2H), 6.83-6.81 (m, 2H), 4.58 (s, 1H), 4.38-4.35 (m, 1H), 3.35-3.32 (m, 2H), 2.74 (br s, 2H), 2.04-2.00 (m, 2H), 1.93-1.90 (m, 1H), 1.79- 1.66 (m, 3H), 1.47 (s, 3H), 1.36 (s, 3H), 1.16 (s, 3H), 0.86-0.83 (m, 2H), 0.73-0.70 (m, 2H). | 723.5 |
| | (S)-2-((R)-6-(N-(3-(1- | | |
| | aminocyclopropyl)propyl)carba- | | |
| | mimidoyl)chroman-2-yl)-2- | | |
| | ((((Z)-1-(2-aminothiazol-4-yl)-2- | | |
| | (((S)-2,2-dimethyl-4-oxo-1- | | |
| | (sulfooxy)azetidin-3-yl)amino)- | | |
| | 2-oxoethylidene)- | | |
| | amino)oxy)propanoic acid | | |
| 12 | 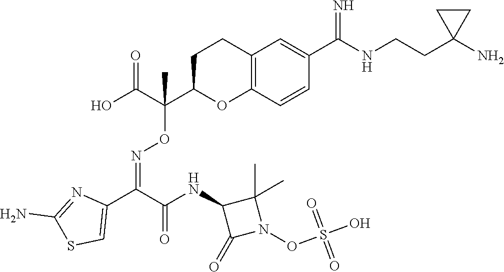 [SMILES: CC1(C)[C@H](NC(=O)/C(=N\O[C@](C)(C(=O)O)[C@H]2CCc3cc(C(=N)NCCC4(N)CC4)ccc3O2)c2csc(N)n2)C(=O)N1OS(=O)(=O)O] | ¹H NMR (500 MHz, 400 uL D₂O/100 uL CD₃CN) δ: 7.37-7.32 (m, 2H), 6.86-6.82 (m, 2H), 4.59 (s, 1H), 4.39-4.36 (m, 1H), 3.51-3.48 (m, 2H), 2.76 (br s, 2H), 2.07-2.03 (m, 2H), 1.93-1.92 (m, 1H), 1.72 (br s, 1H), 1.48 (s, 3H), 1.39 (s, 3H), 1.19 (s, 3H), 0.96-0.93 (m, 2H), 0.83-0.80 (m, 2H). | 709.5 |
| | (S)-2-((R)-6-(N-(2-(1- | | |
| | aminocyclopropyl)ethyl)carbami- | | |
| | midoyl)chroman-2-yl)-2-((((Z)- | | |
| | 1-(2-aminothiazol-4-yl)-2-(((S)- | | |
| | 2,2-dimethyl-4-oxo-1- | | |
| | (sulfooxy)azetidin-3-yl)amino)- | | |
| | 2-oxoethylidene)amino)oxy)- | | |
| | propanoic acid | | |
| 13 | 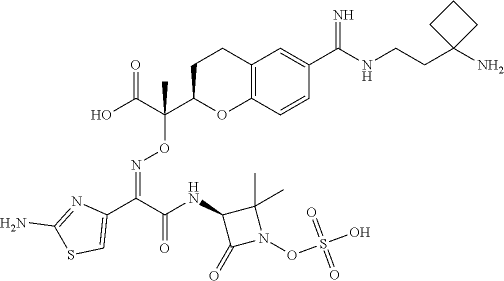 [SMILES: CC1(C)[C@H](NC(=O)/C(=N\O[C@](C)(C(=O)O)[C@H]2CCc3cc(C(=N)NCCC4(N)CCC4)ccc3O2)c2csc(N)n2)C(=O)N1OS(=O)(=O)O] | ¹H NMR (500 MHz, 400 uL D₂O/100 uL CD₃CN) δ: 7.62-7.58 (m, 2H), 7.11-7.09( m, 2H), 4.64- 4.62 (m, 1H), 3.72-3.69 (m, 2H), 3.01 (br s, 2H), 2.49-2.38 (m, 3H), 2.32-2.29 (m, 1H), 2.18-2.16 (m, 3H), 2.11-2.05 (m, 1H), 1.96 (br s, 1H), 1.73 (s, 3H), 1.62 (s, 3H), 1.51 (s, 2H), 1.42 (s, 3H) | 723.6 |
| | (S)-2-((R)-6-(N-(2-(1- | | |
| | aminocyclobutyl)ethyl)carbami- | | |
| | midoyl)chroman-2-yl)-2-((((Z)- | | |
| | 1-(2-aminothiazol-4-yl)-2-(((S)- | | |
| | 2,2-dimethyl-4-oxo-1- | | |
| | (sulfooxy)azetidin-3-yl)amino)- | | |
| | 2-oxoethylidene)- | | |
| | amino)oxy)propanoic acid | | |

### (S)-2-((R)-6-(N-(2-(4-aminopiperidin-1-yl)ethyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

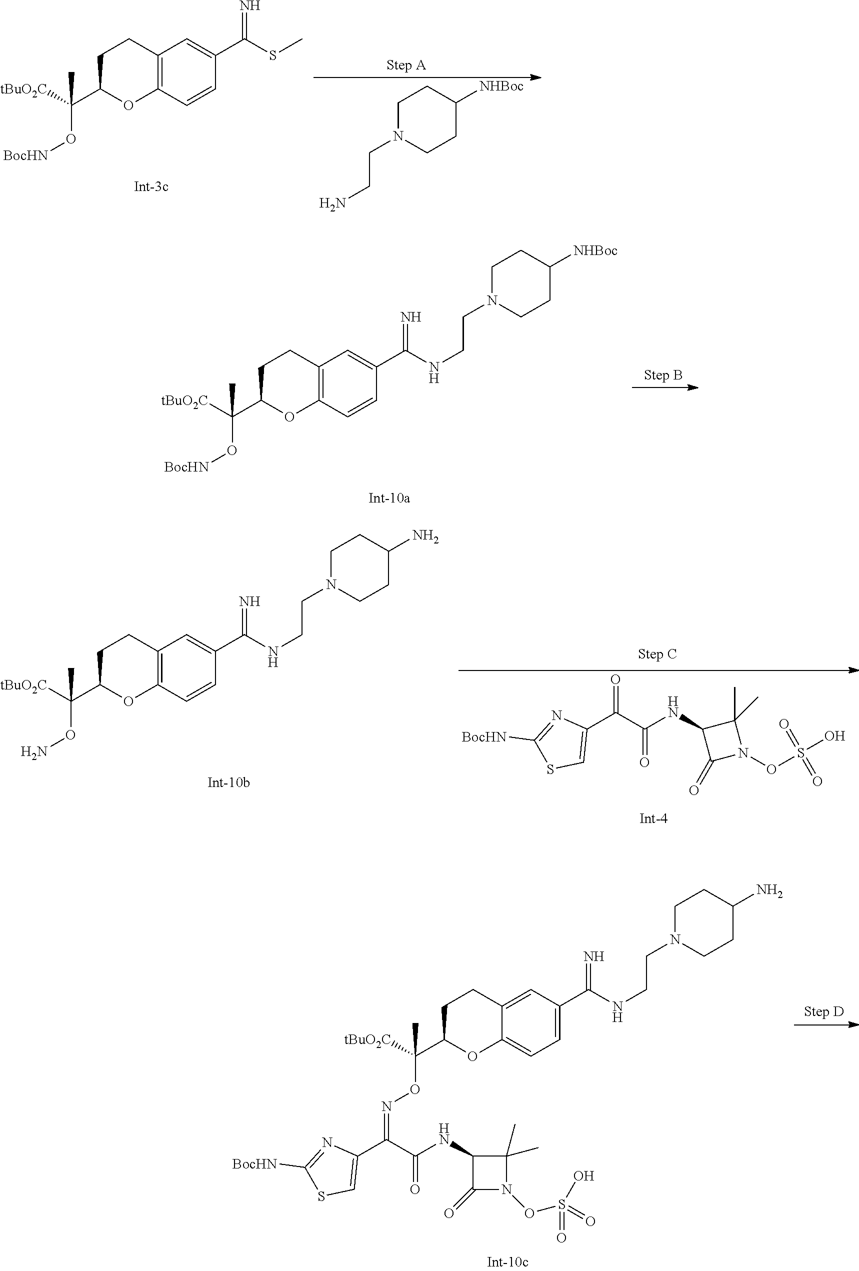 [SMILES: CSC(=N)c1ccc2c(c1)CC[C@H]([C@](C)(ONC(=O)OC(C)(C)C)C(=O)OC(C)(C)C)O2 ; CC(C)(C)OC(=O)NC1CCN(CCN)CC1 ; CC(C)(C)OC(=O)NO[C@](C)(C(=O)OC(C)(C)C)[C@H]1CCc2cc(C(=N)NCCN3CCC(NC(=O)OC(C)(C)C)CC3)ccc2O1 ; CC(C)(C)OC(=O)[C@@](C)(ON)[C@H]1CCc2cc(C(=N)NCCN3CCC(N)CC3)ccc2O1 ; CC(C)(C)OC(=O)Nc1nc(C(=O)C(=O)N[C@@H]2C(=O)N(OS(=O)(=O)O)C2(C)C)cs1 ; CC(C)(C)OC(=O)Nc1nc(/C(=N/O[C@](C)(C(=O)OC(C)(C)C)[C@H]2CCc3cc(C(=N)NCCN4CCC(N)CC4)ccc3O2)C(=O)N[C@@H]2C(=O)N(OS(=O)(=O)O)C2(C)C)cs1]

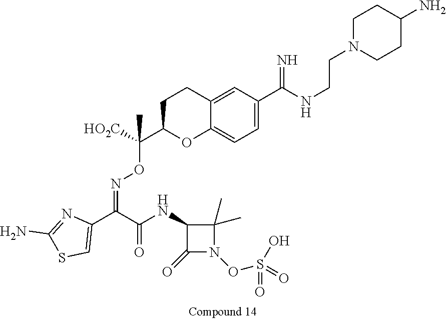 [SMILES: CC1(C)[C@H](NC(=O)/C(=N\O[C@](C)(C(=O)O)[C@H]2CCc3cc(C(=N)NCCN4CCC(N)CC4)ccc3O2)c2csc(N)n2)C(=O)N1OS(=O)(=O)O]

Step A—Synthesis of Intermediate 10a To a solution of intermediate 3c (200 mg, 0.429 mmol) in MeOH (2 mL) was added tert-butyl(1-(2-aminoethyl)piperidin-4-yl)carbamate (104 mg, 0.429 mmol), potassium acetate (126 mg, 1.286 mmol) and acetic acid (103 mg, 1.715 mmol) at 20° C. The mixture was stirred at 80° C. under nitrogen for 20 min. Then the solvent was removed under reduced pressure to give crude intermediate 10a, which was used in the next step without further purification. LC-MS (ESI) m/z: 662.4 [M+H]⁺.

Step B—Synthesis of Intermediate 10b To a solution of intermediate 10a (362 mg, 0.547 mmol) in DCM (4 mL) was added TFA (2 mL). The reaction was stirred at 20° C. for 1.5 h. Then the reaction mixture was concentrated at 15° C. under reduced pressure to give crude intermediate 10b, which was used in the next step without further purification. LC-MS (ESI) m/z: 462.2 [M+H]⁺.

Step C—Synthesis of Intermediate 10c To a solution of intermediate 10b (252 mg, 0.546 mmol) in MeOH (4 mL) was added intermediate 4 (203 mg, 0.437 mmol) at 20 CC. The reaction was stirred at 28° C. for 2 h. Then the reaction mixture was concentrated in vacuo to give crude intermediate 10c, which was used in the next step without further purification. LC-MS (ESI) m/z: 908.4 [M+H]⁺.

Step D—Synthesis of Compound 14 To a solution of intermediate 10c (496 mg, 0.546 mmol) in DCM (2 mL) was added TFA (4 mL). The reaction was stirred at 20° C. for 30 min. Then the solvent was removed with a nitrogen flow, and the resulting residue was purified by a reverse phase HPLC (Column: Boston Uni C18 40*150*5 um; Condition: water (0.1% TFA)-ACN; Begin B 0%, End B 30%; Gradient Time (min) 10; 100% B Hold Time (min) 2; FlowRate (mL/min) 60; Injections 2) to afford compound 14 as its TFA salt form. This material was further purified by a reverse phase HPLC (Column: Welch Xtimate C18 150*25 mm*5 um;

Condition: water (0.225% FA)-ACN; Begin B 0%, End B 13%; Gradient Time (min) 15; 100% B Hold Time (min) 2; FlowRate (mL/min) 25; Injections 2) then lyophilized to afford compound 14 as its formic acid salt form. LC-MS (ESI) m/z: 752.3 [M+H]⁺. ¹H NMR (400 MHz, DMSO-d₆) δ: 7.45-7.39 (m, 2H), 6.92 (d, J=8.6 Hz, 1H), 6.74 (s, 1H), 4.59 (s, 1H), 4.45-4.35 (m, 1H), 4.02-3.99 (m, 1H), 3.48-3.40 (m, 2H), 3.06-2.86 (m, 3H), 2.83-2.76 (m, 2H), 2.65-2.58 (m, 2H), 2.11-1.95 (m, 3H), 1.88-1.78 (m, 2H), 1.69-1.57 (m, 1H), 1.48 (s, 3H), 1.37 (s, 3H), 1.22 (s, 3H).

### Example 13: Preparation of Compounds 15 to 20

Starting from intermediate 3c, the following compounds 15 to 20 were analogously prepared as formic acid salts according to Example 12 with the exception of substituting tert-butyl (1-(2-aminoethyl)piperidin-4-yl)carbamate) with the appropriate amine in Step A:

| Com- | | | LCMS |
|---|---|---|---|
| pound | Structure | ¹H NMR | [M + H]⁺ |
| 15 | 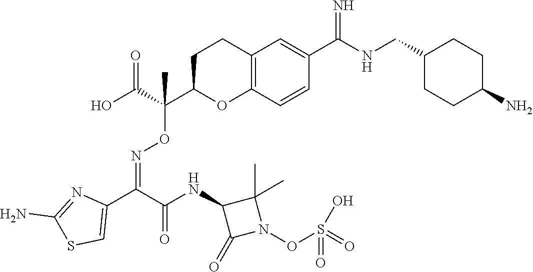 | ¹H NMR (CD₃CN + D₂O, 400 MHz) δ: 7.41-7.34 (m, 2H), 6.89 (d, J = 8.2 Hz, 1H), 6.80 (s, 1H), 4.60 (s, 1H), 4.37 (s, 1H), 3.19 (d, J = 7.0 Hz, 2H), 3.09-3.00 (m, 1H), 2.87-2.72 (m, 2H), 2.10- 1.96 (m, 3H), 1.84 (br d, J = 13.7 Hz, 2H), 1.69 (br d, J = 8.6 Hz, 2H), 1.49 (s, 3H), 1.41 (s, 3H), 1.38-1.26 (m, 2H), 1.24 (s, 3H), 1.14-1.00 (m, 2H) | 736.8 |
| | (S)-2-((R)-6-(N-(((1r,4R)-4- | | |
| | aminocyclohexyl)methyl)carba- | | |
| | mimidoyl)chroman-2-yl)-2- | | |
| | ((((Z)-1-(2-aminothiazol-4-yl)- | | |
| | 2-(((S)-2,2-dimethyl-4-oxo-1- | | |
| | (sulfooxy)-azetidin-3- | | |
| | yl)amino)-2- | | |
| | oxoethylidene)amino)oxy )pro- | | |
| | panoic acid | | |
| 16 | 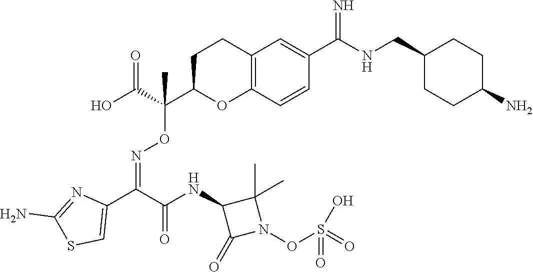 [SMILES: CC1(C)[C@H](NC(=O)/C(=N\O[C@](C)(C(=O)O)[C@H]2CCc3cc(C(=N)NC[C@H]4CC[C@@H](N)CC4)ccc3O2)c2csc(N)n2)C(=O)N1OS(=O)(=O)O] | ¹H NMR (CD₃CN + D₂O, 400 MHz) δ: 7.41-7.35 (m, 2H), 6.89 (d, J = 8.2 Hz, 1H), 6.79 (s, 1H), 4.60 (s, 1H), 4.39 (br d, J = 11.7 Hz, 1H), 3.27 (br d, J = 7.4 Hz, 3H), 2.78 (br s, 2H), 2.08 (br d, J = 11.7 Hz, 2H), 1.78-1.61 (m, 8H), 1.49 (s, 3H), 1.46 (br s, 1H), 1.42 (s, 3H), 1.24 (s, 3H). | 736.9 |
| | (S)-2-((R)-6-(N-(((1s,4S)-4- | | |
| | aminocyclohexyl)methyl)carba- | | |
| | mimidoyl)chroman-2-yl)-2- | | |
| | ((((Z)-1-(2-aminothiazol-4-yl)- | | |
| | 2-(((S)-2,2-dimethyl-4-oxo-1- | | |
| | (sulfooxy)-azetidin-3- | | |
| | yl)amino)-2- | | |
| | oxoethylidene)amino)oxy) | | |
| | propanoic acid | | |
| 17 | 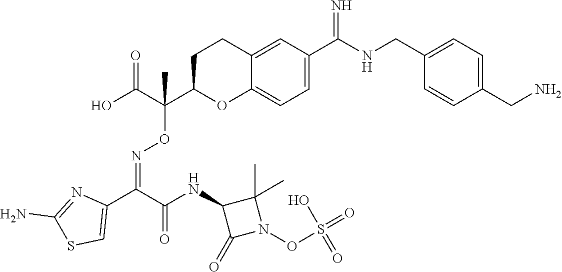 [SMILES: CC1(C)[C@H](NC(=O)/C(=N\O[C@](C)(C(=O)O)[C@H]2CCc3cc(C(=N)NCc4ccc(CN)cc4)ccc3O2)c2csc(N)n2)C(=O)N1OS(=O)(=O)O] | ¹H NMR (400 MHz, DMSO-d₆) δ: 7.51-7.39 (m, 6H), 6.97 (d, J = 8.6 Hz, 1H), 6.75 (s, 1H), 4.61 (s, 3H), 4.46-4.41 (m, 1H), 4.00 (s, 2H), 2.83-2.71 (m, 2H), 2.07-1.98 (m, 1H), 1.63-1.52 (m, 1H), 1.49 (s, 3H), 1.39 (s, 3H), 1.23 (s, 3H). | 745.2 |
| | (S)-2-((R)-6-(N-(4- | | |
| | (aminomethyl)benzyl)carbami- | | |
| | midoyl)chroman-2-y1)-2- | | |
| | ((((Z)-1-(2-aminothiazol-4-yl)- | | |
| | 2-(((S)-2,2-dimethyl-4-oxo-1- | | |
| | (sulfooxy)-azetidin-3- | | |
| | yl)amino)-2-oxo- | | |
| | ethylidene)amino)oxy) | | |
| | propanoic acid | | |
| 18 | 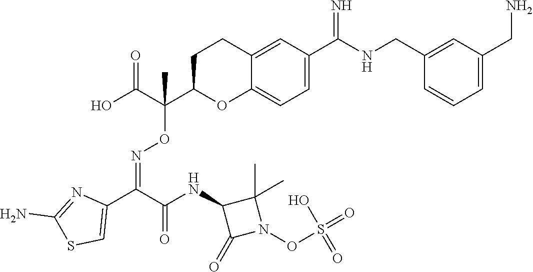 [SMILES: CC1(C)[C@H](NC(=O)/C(=N\O[C@](C)(C(=O)O)[C@H]2CCc3cc(C(=N)NCc4cccc(CN)c4)ccc3O2)c2csc(N)n2)C(=O)N1OS(=O)(=O)O] | ¹H NMR (400 MHz, DMSO-d₆) δ: 7.57-7.48 (m, 2H), 7.45-7.36 (m, 4H), 6.96 (d, J = 8.6 Hz, 1H), 6.75 (s, 1H), 4.61 (s, 3H), 4.43 (br d, J = 11.7 Hz, 1H), 4.02 (s, 2H), 2.88-2.72 (m, 2H), 2.03-1.93 (m, 1H), 1.62-1.50 (m, 1H), 1.48 (s, 3H), 1.39 (s, 3H), 1.23 (s, 3H). | 745.2 |
| | (S)-2-((R)-6-(N-(3- | | |
| | (aminomethyl)benzyl)carbami- | | |
| | midoyl)chroman-2-yl)-2- | | |
| | ((((Z)-1-(2-aminothiazol-4-yl)- | | |
| | 2-(((S)-2,2-dimethyl-4-oxo-1- | | |
| | (sulfooxy)-azetidin-3- | | |
| | yl)amino)-2-oxo- | | |
| | ethylidene)amino)oxy) | | |
| | propanoic acid | | |
| 19 | 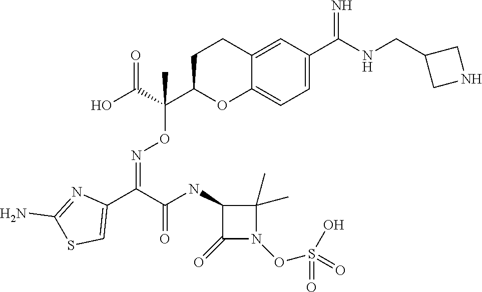 [SMILES: CC1(C)[C@H](NC(=O)/C(=N\O[C@](C)(C(=O)O)[C@H]2CCc3cc(C(=N)NCC4CNC4)ccc3O2)c2csc(N)n2)C(=O)N1OS(=O)(=O)O] | ¹H NMR (400 MHz, DMSO-d₆) δ: 7.40 (s, 1H), 7.35 (br d, J = 9.0 Hz, 1H), 6.91 (d, J = 8.6 Hz, 1H), 6.74 (s, 1H), 4.56 (s, 1H), 4.38 (br d, J = 11.7 Hz, 1H), 4.05-3.95 (m, 2H), 3.81-3.70 (m, 2H), 3.65-3.44 (m, 2H), 3.21-3.04 (m, 1H), 2.88- 2.66 (m, 2H), 2.05-1.95 (m, 1H), 1.68-1.46 (m, 1H), 1.48 (s, 3H), 1.37 (s, 3H), 1.21 (s, 3H). | 695.5 |
| | (S)-2-((((Z)-1-(2-aminothiazol- | | |
| | 4-y1)-2-(((S)-2,2-dimethyl-4- | | |
| | oxo-1-(sulfooxy)azetidin-3- | | |
| | yl)amino)-2- | | |
| | oxoethylidene)amino)oxy)-2- | | |
| | ((R)-6-(N-(azetidin-3- | | |
| | ylmethyl)- | | |
| | carbamimidoyl)chroman-2- | | |
| | yl)propanoic acid | | |
| 20 | 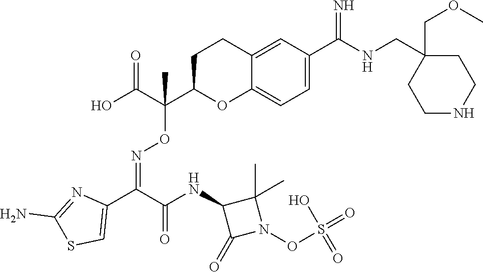 [SMILES: COCC1(CNC(=N)c2ccc3c(c2)CC[C@H]([C@](C)(O/N=C(\C(=O)N[C@@H]2C(=O)N(OS(=O)(=O)O)C2(C)C)c2csc(N)n2)C(=O)O)O3)CCNCC1] | ¹H NMR (400 MHz, CD₃CN) δ: 7.36-7.44 (m, 2H), 6.93 (d, J = 7.43 Hz, 1H), 6.77 (s, 1H), 4.61 (s, 1H), 4.42-4.36 (m, 1H), 3.40 (s, 2H), 3.33 (s, 2H), 3.31 (s, 3H), 3.02-3.24 (m, 4H), 2.86-2.68 (m, 2H), 2.12-2.01 (m, 1H), 1.78-1.62 (m, 5H), 1.49 (s, 3H), 1.43 (s, 3H), 1.26 (s, 3H). | 767.2 |
| | (S)-2-((((Z)-1-(2-aminothiazol- | | |
| | 4-y1)-2-(((S)-2,2-dimethyl-4- | | |
| | oxo-1-(sulfooxy)azetidin-3- | | |
| | yl)amino)-2- | | |
| | oxoethylidene)amino)oxy)-2- | | |
| | ((R)-6-(N-((4- | | |
| | (methoxymethyl)-piperidin-4- | | |
| | yl)methyl)- | | |
| | carbamimidoyl)chroman-2- | | |
| | yl)propanoic acid | | |

### Example 14: Preparation of Intermediate 11d

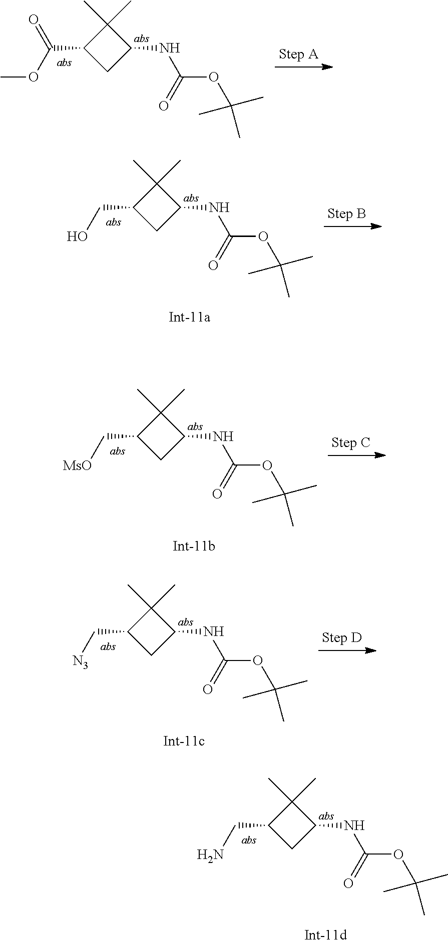 [SMILES: COC(=O)[C@H]1C[C@@H](NC(=O)OC(C)(C)C)C1(C)C ; CC(C)(C)OC(=O)N[C@@H]1C[C@H](CO)C1(C)C ; CC(C)(C)OC(=O)N[C@@H]1C[C@H](COS(C)(=O)=O)C1(C)C ; CC(C)(C)OC(=O)N[C@@H]1C[C@H](CN=[N+]=[N-])C1(C)C ; CC(C)(C)OC(=O)N[C@@H]1C[C@H](CN)C1(C)C]

Step A—Synthesis of Intermediate 11a Aluminum(III) lithium hydride (1.486 ml, 2.97 mmol) (2 N THF) was added to a stirred solution of methyl (1S,3R)-3-((tert-butoxycarbonyl)amino)-2,2-dimethylcyclobutane-1-carboxylate (510 mg, 1.982 mmol) in THF (15 mL) at −12° C. (ice/acetone bath), and the mixture was stirred at −10° C. for 45 min. Then the reaction was quenched with 1 N NaOH (20 mL), and extracted with EtOAc (2×50 mL). The combined organic layers were washed with brine, dried over anhydrous MgSO₄, and filtered. The filtrate was concentrated under vacuum to give crude intermediate 11a, which was used in the next reaction without further purification. TLC: Rf=0.3 EtOAc/Hexane (1/1), KMnO₄ Stain.

Step B—Synthesis of Intermediate 11b Methanesulfonyl chloride (0.185 ml, 2.386 mmol) was added to a stirred solution of crude intermediate 11a (456 mg, 1.988 mmol) and triethylamine (0.416 ml, 2.98 mmol) in THF (15 ml) at 0° C. The mixture was warmed to room temperature and stirred for 1 h. Then the reaction was quenched with saturated aqueous NaHCO₃ solution, and extracted with EtOAc. The combined organic layers were washed with brine, and dried over anhydrous MgSO₄. The drying agent was removed via filtration, and the filtrate was concentrated under vacuum to give crude intermediate 11b, which was used in the next reaction without further purification. TLC: Rf=0.5 EtOAc/Hexane (1/1), KMnO₄ Stain.

Step C—Synthesis of Intermediate 11c Sodium azide (258 mg, 3.97 mmol) was added to a stirred mixture of intermediate 11b (610 mg, 1.984 mmol) in DMF (10 mL) at room temperature, and the resulting mixture was stirred at 70° C. for 3 h. Then the reaction was quenched with saturated aqueous NaHCO₃ solution, and extracted with EtOAc. The combined organic layers were washed with brine, and dried over anhydrous MgSO₄. The drying agent was removed via filtration, and the filtrate was concentrated under vacuum. The residue was purified by silica gel chromatography (40 g) gradient eluting with 0-70% EtOAc/hexanes) to give intermediate 11c. TLC: Rf=0.8 EtOAc/Hexane (1/1), KMnO₄ Stain.

Step D—Synthesis of Intermediate 11d To a solution of intermediate 11c (200 mg, 0.786 mmol) in ethanol (5 mL) was added Pd/C (30 mg, 10 wt %, 50% moisture). The mixture was stirred under a H₂ balloon at room temperature for 1 h. Then the catalyst was removed by filtration and the filtrate was evaporated under vacuum to afford the crude intermediate 11d, which was used without further purification. TLC: Rf=0, EtOAc/Hexane (1/1), KMnO₄ Stain.

### Example 15: Preparation of Compound 21

### (S)-2-((R)-6-(N-(((1S,3R)-3-amino-2,2-dimethylcyclobutyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

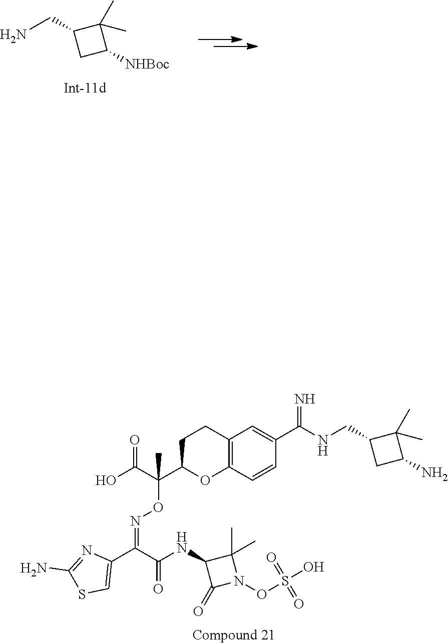 [SMILES: CC(C)(C)OC(=O)N[C@@H]1C[C@H](CN)C1(C)C ; CC1(C)[C@@H](CNC(=N)c2ccc3c(c2)CC[C@H]([C@](C)(O/N=C(\C(=O)N[C@@H]2C(=O)N(OS(=O)(=O)O)C2(C)C)c2csc(N)n2)C(=O)O)O3)C[C@H]1N]

Compound 21 was prepared as its formic acid salt from intermediate 11d according to the procedure described in Step A to Step D of Example 10.

LC-MS [M+H]⁺: m/z 737.6. ¹H NMR (500 MHz, 400 uL D₂O/100 uL CD₃CN) δ_{H} 7.27-7.23 (m, 2H), 6.82-6.77 (m, 2H), 4.54 (s, 1H), 4.38-4.35 (m, 1H), 3.40-3.24 (m, 3H), 2.70 (br s, 2H), 2.35-2.21 (m, 2H), 1.98 (br s, 1H), 1.74-1.67 (m, 2H), 1.45 (s, 3H), 1.32 (s, 3H), 1.09 (s, 3H), 1.07 (s, 3H), 1.04 (s, 3H).

### Example 16: Preparation of Intermediates 12b-2 and 12e-1

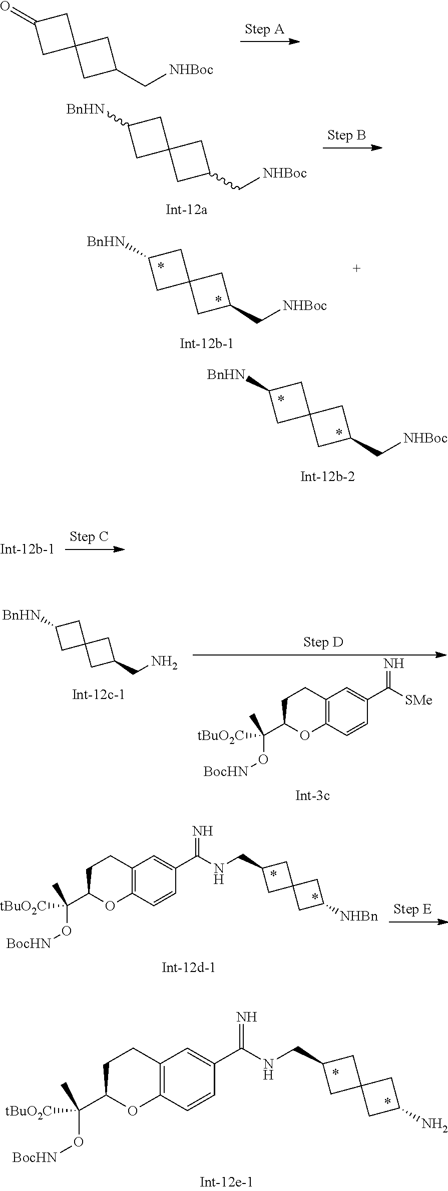

Step A—Synthesis of Intermediate 12a To a solution of tert-butyl ((6-oxospiro[3.3]heptan-2-yl)methyl)carbamate (500 mg, 2.089 mmol) in CH₂Cl₂ (8 ml) was added benzylamine (0.297 ml, 2.72 mmol) at ambient temperature. The mixture was stirred at ambient temperature for 10 minutes, then NaBH(OAc)₃ (886 mg, 4.18 mmol) and acetic acid (1.196 μl, 0.021 mmol) were added. The mixture was stirred for 3 h, then cooled to 0° C. and quenched with 1 N NaOH. The mixture was extracted with EtOAc. The combined organic layers were washed with brine, dried over anhydrous MgSO₄, and filtered. The filtrate was concentrated under vacuum, and the resulting residue was purified by silica gel chromatography with gradient elution of 0-100% EtOAc/hexanes to give intermediate 12a. LC-MS [M+1]: m/z 331.7.

Step B—Synthesis of Intermediate 12b-1 and 12b-2 Enantiomers of intermediate 12b (440 mg, 1.331 mmol) were separated by chiral SFC (Column: AS-H 21×250 mm; co-solvent: 10% EtOH+0.2% DIPA; 210 nm wavelength; injection volume 1.0 mL; flow rate 50 ml/min) to give intermediate 12b-1 and its enantiomer 12b-2. LC-MS [M+1]: m/z 331.4.

Step C—Synthesis of Intermediate 12c-1 Intermediate 12b-1 (100 mg, 0.303 mmol) was dissolved in DCM (5 mL) and TFA (0.6 ml) was added. The mixture was stirred at ambient temperature for 1 h. Then the mixture was evaporated under vacuum to afford the crude intermediate 12c-1. TLC (Rf=0, EtOAc/hexanes 1:1).

Step D—Synthesis of Intermediate 12d-1 To a vial containing intermediate 12c-1 (71 mg, 0.30 mmol) in anhydrous acetonitrile (4 mL) were added intermediate 3c (0.13 g, 0.279 mmol) and acetic acid (0.056 mL, 0.98 mmol). The reaction mixture was heated at 65° C. for 1 h. The reaction was cooled to ambient temperature and purified on reverse phase Isco Combiflash (100 g, 0~100% 0.05% TFA water/ACN) to give intermediate 12d-1. LC-MS [M+1]: m/z 649.6.

Step E—Synthesis of Intermediate 12e-1 Intermediate 12d-1 (130 mg, 0.2 mmol) was dissolved in ethanol (5 mL) and Pd/C (30 mg, 10%, 50% moisture) was added. The mixture was stirred under a H₂ balloon at room temperature for 1 h. Then the catalyst was removed by filtration, and the filtrate was evaporated to afford crude intermediate 12e-1. LC-MS [M+1]: m/z 559.5.

### Example 17: Preparation of Compound 22

### (S)-2-((R)-6-(N-(((2S,4s,6S)-6-aminospiro[3.3]heptan-2-yl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid or (S)-2-((R)-6-(N-(((2R,4r,6R)-6-aminospiro[3.3]heptan-2-yl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

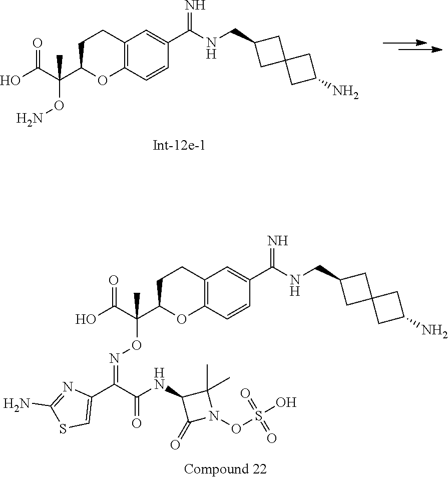 [SMILES: C[C@@](ON)(C(=O)O)[C@H]1CCc2cc(C(=N)NC[C@H]3CC4(C[C@@H](N)C4)C3)ccc2O1 ; CC1(C)[C@H](NC(=O)/C(=N\O[C@](C)(C(=O)O)[C@H]2CCc3cc(C(=N)NC[C@H]4CC5(C[C@@H](N)C5)C4)ccc3O2)c2csc(N)n2)C(=O)N1OS(=O)(=O)O]

Compound 22 was analogously prepared as its formic acid salt from intermediate 12e-1, using the procedure described in Steps C to D of Example 10. LC-MS [M+H]⁺: m/z 749.6. ¹H NMR (500 MHz, 400 uL D₂O/100 uL CD₃CN) δ_{H} 7.29-7.25 (m, 2H), 6.80-6.77 (m, 2H), 4.50 (s, 1H), 4.38-4.36 (m, 1H), 3.58-3.55 (m, 1H), 3.26 (d, J=10 Hz, 2H), 2.71 (br s, 2H), 2.53-2.33 (m, 3H), 2.23-2.12 (m, 2H), 2.05-1.96 (m, 3H), 1.78-1.69 (m, 3H), 1.44 (s, 3H), 1.32 (s, 3H), 1.11 (s, 3H).

### Example 18: Preparation of Compound 23

### (S)-2-((R)-6-(N-(((2R,4r,6R)-6-aminospiro[3.3]heptan-2-yl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid or (S)-2-((R)-6-(N-(((2S,4s,6S)-6-aminospiro[3.3]heptan-2-yl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

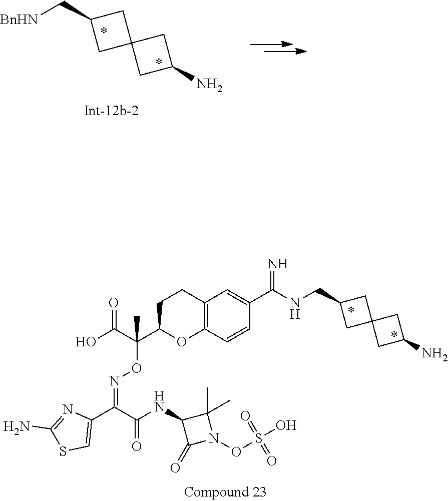

Compound 23 was analogously prepared as its formic acid salt from intermediate 12b-2, using the procedure described in Steps C to E of Example 16 and Steps C to D of Example 10. LC-MS [M+H]⁺: m/z 749.5. ¹H NMR (500 MHz, 400 uL D₂O/100 uL CD₃CN) 6H 7.28-7.25 (m, 2H), 6.80-6.76 (m, 2H), 4.51 (s, 1H), 4.38-4.35 (m, 1H), 3.60-3.53 (m, 1H), 3.26 (d, J=10 Hz, 2H), 2.71 (br s, 2H), 2.52-2.44 (m, 1H), 2.41-2.35 (m, 1H), 2.23-2.12 (m, 2H), 2.05-1.96 (m, 4H), 1.78-1.69 (m, 3H), 1.44 (s, 3H), 1.32 (s, 3H), 1.11 (s, 3H).

### Example 19: Preparation of Intermediate 13d

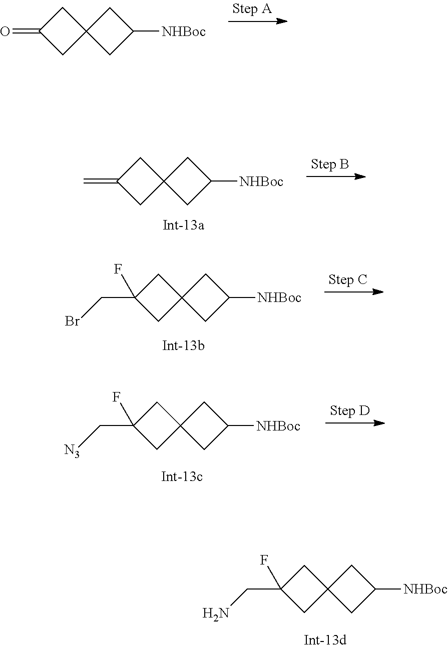 [SMILES: CC(C)(C)OC(=O)NC1CC2(CC(=O)C2)C1 ; C=C1CC2(C1)CC(NC(=O)OC(C)(C)C)C2 ; CC(C)(C)OC(=O)NC1CC2(C1)CC(F)(CBr)C2 ; CC(C)(C)OC(=O)NC1CC2(C1)CC(F)(CN=[N+]=[N-])C2 ; CC(C)(C)OC(=O)NC1CC2(C1)CC(F)(CN)C2]

Step A—Synthesis of Intermediate 13a To a mixture of methyltriphenylphosphonium bromide (5.07 g, 14.20 mmol) in DMSO (18 ml), stirred at room temperature, was added sodium hydride (0.568 g, 14.20 mmol) in one portion. The mixture was stirred at room temperature for 30 min. Then tert-butyl ((6-oxospiro[3.3]heptan-2-yl)methyl)carbamate (2 g, 8.88 mmol) was added in one portion, and the reaction was stirred at room temperature for 1.5 h. The reaction mixture was poured into a flask containing ice. Diethyl ether (150 mL) was added, followed by EtOAc (50 mL), and the mixture was stirred for 1 h. The aqueous layer was separated and extracted with 100 mL 1:1 Et₂O/EtOAc. The organic layers were combined, washed with brine, and dried over anhydrous magnesium sulfate. The drying agent was removed via, and the filtrate was concentrated under vacuum. The resulting residue was dissolved in a minimal amount of CH₂Cl₂ and loaded onto a dry Biotage 120 g silica column. The solvent was removed from the column via nitrogen stream. Gradient elution with 0% to 50% EtOAc in hexanes afforded the title compound. TLC: Rf=0.8 EtOAc/Hexane (1/1), KMnO₄ Stain.

Step B—Synthesis of Intermediate 13b To a stirred mixture of intermediate 13a (1.6 g, 7.16 mmol) in DCM (20 mL) was added triethylamine trihydrofluoride (2.92 mL, 17.91 mmol). The mixture was stirred at room temperature for 10 minutes, then a solution of N-bromosuccinimide (1.913 g, 10.75 mmol) in DCM (30 mL) was added dropwise, and the resulting mixture was stirred at room temperature for 3 h. The reaction mixture was partitioned between DCM and saturated sodium thiosulfate aqueous solution by stirring the resulting mixture for 1 h. The aqueous layer was separated and extracted with 100 mL 1:1 Et₂O/EtOAc. The organic layers were combined, washed with brine, and dried over anhydrous magnesium sulfate. The drying agent was removed via filtration, and the filtrate was concentrated under vacuum. The resulting residue was dissolved in a minimal amount of DCM and loaded onto a dry Biotage 120 g silica column. The solvent was removed from the column via a nitrogen stream. Gradient elution with 0% to 50% EtOAc in hexanes afforded intermediate 13b. TLC: Rf=0.6 EtOAc/Hexane (1/1), KMnO₄ Stain.

Step C—Synthesis of Intermediate 13c A mixture of intermediate 13b (1.93 g, 5.99 mmol) and sodium azide (0.506 g, 7.79 mmol) in DMSO (10 mL) was stirred at 120° C. overnight. The reaction was partitioned between ethyl acetate (150 mL) and brine (150 mL). The layers were separated and the organic layer was washed with brine, dried over anhydrous MgSO₄, filtered and evaporated to afford crude intermediate 13c, which was used in the next reaction without further purification. TLC: Rf=0.5 EtOAc/Hexane (1/1), KMnO₄ Stain.

Step D—Synthesis of Intermediate 13d To a stirred mixture of intermediate 13c (0.7 g, 2.462 mmol) in THF (12 mL) and water (10 mL) was added polymer-bound triphenylphosphine (0.965 g, 3.69 mmol) at room temperature. The mixture was stirred at room temperature for 16 h, and then filtered. The filtrate was concentrated under vacuum to afford crude intermediate 13d, which was used without further purification. TLC: Rf=0.1 EtOAc/Hexane (1/1), KMnO₄ Stain.

### Example 20: Preparation of Compounds 24 and 25

### (S)-2-((R)-6-(N-(((2R,4r,6R)-6-amino-2-fluorospiro[3.3]heptan-2-yl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid and (S)-2-((R)-6-(N-(((2S,4s,6S)-6-amino-2-fluorospiro[3.3]heptan-2-yl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

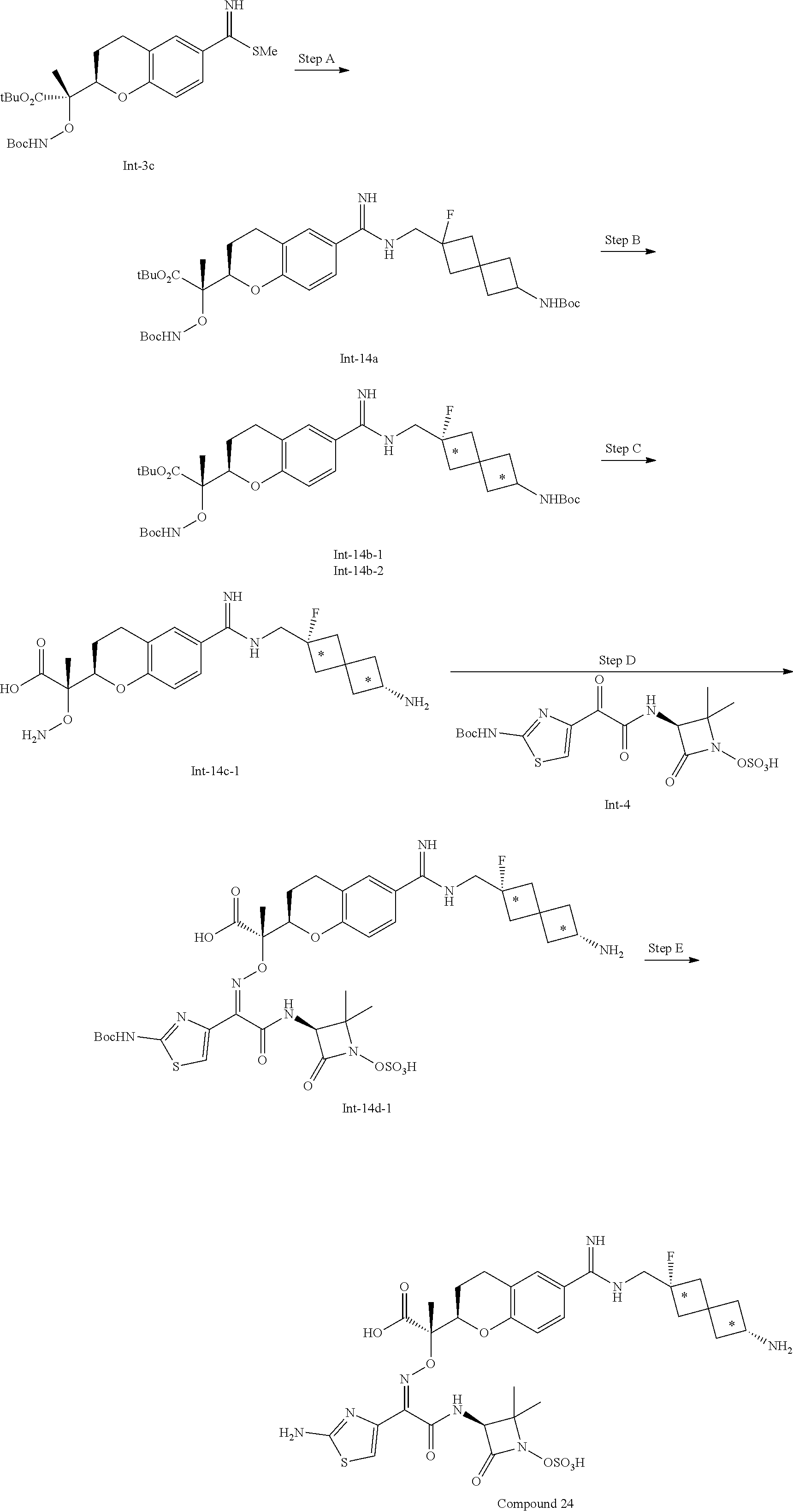

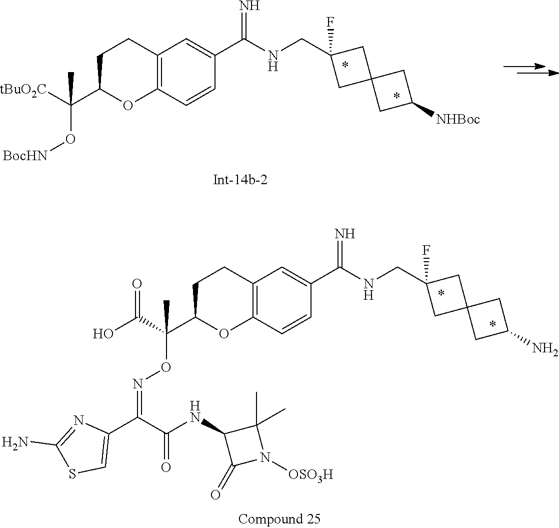

Step A—Synthesis of Intermediate 14a To a vial containing a mixture of intermediate 13d (266 mg, 1.03 mmol) in anhydrous acetonitrile (8 mL) were added intermediate 3c (0.3 g, 0.643 mmol) and acetic acid (0.129 mL, 2.25 mmol). The reaction mixture was heated at 65° C. for 4 h. The reaction mixture was cooled to ambient temperature and purified on reverse phase MPLC (Column: Isco, C-18, 50 g column; 0~100% water+0.05% TFA/ACN+0.05% TFA) to give intermediate 14a. LC-MS [M+1]: m/z 676.0.

Step B—Synthesis of Intermediate 14b-1 and Intermediate 14b-2 Diastereomers of intermediate 14a (400 mg, 0.591 mmol) were separated by chiral SFC (AS-H column 250 mm; co-solvent: 25% MeOH/ACN 1:1+0.2% DIPA; 210 nm wavelength; injection volume: 1.5 mL; flow rate 50 mL/min) to afford intermediate 14b-1 and its diastereomer 14b-2. LC-MS [M+1]: m/z 676.0.

Step C—Synthesis of Intermediate 14c-1 To a vial containing intermediate 14b-1 (0.17 g, 0.251 mmol) was added 2:1 trifluoroacetic acid/anhydrous dichloromethane (6 mL) at ambient temperature. The reaction was stirred for 16 h. Then a solution of 4:1 toluene/MeOH (10 mL) was added to the reaction, and the mixture was concentrated in vacuo. Repeated azeotroping of the resulting residue with 4:1 toluene/MeOH (10 mL). The resulting residue was then dried under high vacuum to give intermediate 14c-1, which was used in the next reaction without further purification. LC-MS [M+1]: m/z 420.0.

Step D—Synthesis of Intermediate 14d-1 To a vial charged with intermediate 14c-1 (0.106 g, 0.252 mmol) and intermediate 4 (0.117 g, 0.252 mmol) was added MeOH (3.0 ml) at ambient temperature. The reaction mixture was stirred at room temperature for 6 h, and then concentrated in vacuo to afford crude intermediate 14d-1, which was used in the next reaction without further purification. LC-MS [M+1]: m/z 867.4.

Step E—Synthesis of Compound 24 To a vial charged with intermediate 14d-1 (0.218 g, 0.252 mmol) was added 1:2 trifluoroacetic acid/anhydrous dichloromethane (6 mL) at ambient temperature. The reaction mixture was stirred at ambient temperature for 1 h and then cooled to 0° C. Ethyl ether (6 mL) was slowly added to the reaction mixture with stirring. The resulting precipitated solid was collected by centrifugation, and then purified by Gilson (Column: Isco, C18, 5 um, OBD 30×150 mm; 0~40% ACN+0.05% TFA/water+0.05% TFA, flow rate: 30 mL/min). The product fractions were collected, and concentrated in vacuo to remove acetonitrile. The remaining aqueous layer was directly loaded onto an Amberchrom CG161M column (26 g), washed with 9 CV of (water+0.1% FA), and eluted with 3 CV of 100% (AcCN+0.1% FA) followed by 3 CV of 50% (AcCN+0.1% FA)/(water+0.1% FA). The product containing fractions were collected, and concentrated in vacuo. The resulting aqueous residue was lyophilized to give compound 24 as its formic acid salt. LC-MS [M+1]: m/z 767.3. ¹H NMR (500 MHz, 400 uL D₂O/100 uL CD₃CN) δ: 7.47-7.44 (m, 2H), 6.97-6.93 (m, 2H), 4.51-4.48 (m, 1H), 3.76-3.71 (m, 1H), 3.65 (s, 1H), 2.90-2.83 (m, 2H), 2.53-2.40 (m, 5H), 2.32-2.13 (m, 3H), 2.02-1.99 (m, 3H), 1.82 (br s, 1H), 1.57 (s, 3H), 1.47 (s, 3H), 1.28 (s, 3H).

Compound 25 was analogously prepared as its formic acid salt from intermediate 14b-2 using the method described in Steps C to E of Example 20. LC-MS [M+1]: m/z 767.2. ¹H NMR (500 MHz, 400 uL D₂O/100 uL CD₃CN) δ: 7.48-7.45 (m, 2H), 6.99 (d, J=10 Hz, 1H), 6.91 (s, 1H), 4.51-4.48 (m, 1H), 3.80-3.72 (m, 2H), 3.67 (s, 1H), 2.90-2.84 (m, 2H), 2.54-2.42 (m, 5H), 2.34- 2.22 (m, 2H), 2.17 (br s, 1H), 2.03-2.00 (m, 2H), 1.80 (br s, 1H), 1.58 (s, 3H), 1.49 (s, 3H), 1.30 (s, 3H).

### Example 21: Preparation of Compounds 26 to 29

The following compounds were analogously prepared as formic acid salts using the method described in Example 20 starting from the appropriate amine:

| | | | LCMS |
|---|---|---|---|
| Compound | Structure | ¹H NMR | [M + H]⁺ |
| 26 | 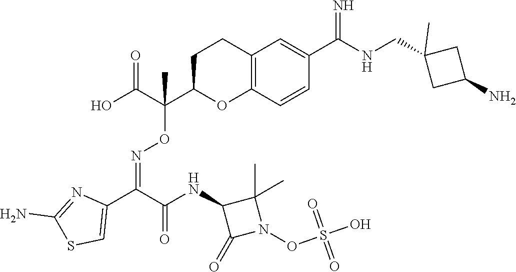 [SMILES: CC1(C)[C@H](NC(=O)/C(=N\O[C@](C)(C(=O)O)[C@H]2CCc3cc(C(=N)NC[C@]4(C)C[C@@H](N)C4)ccc3O2)c2csc(N)n2)C(=O)N1OS(=O)(=O)O] | ¹H NMR (500 MHz, 400 uL D₂O/100 uL CD₃CN) δ_{H} 7.46- 7.44 (m, 2H), 6.96-6.90 (m, 2H), 4.46-4.43 (m, 1H), 3.91- 3.83 (m, 1H), 3.41 (s, 2H), 2.85 (br s, 2H), 2.24-2.08 (m, 5H), 2.01-1.99 (m, 2H) 1.79 (br s, 1H), 1.56 (s, 3H), 1.48 (s, 3H), 1.29-1.27 (m, 6H). | 723.6 |
| | (S)-2-((R)-6-(N-(((1s,3S)-3- | | |
| | amino-1-methylcyclobutyl)- | | |
| | methyl)carbamimidoyl)chroman- | | |
| | 2-yl)-2-((((Z)-1-(2-aminothiazol- | | |
| | 4-yl)-2-(((S)-2,2-dimethyl-4-oxo- | | |
| | 1-(sulfooxy)-azetidin-3- | | |
| | yl)amino)-2-oxoethylidene)- | | |
| | amino)oxy)propanoic acid or (S)- | | |
| | 2-((R)-6-(N-(((1r,3R)-3-amino-1- | | |
| | methylcyclobutyl)methyl)carbami- | | |
| | midoyl)chroman-2-yl)-2-((((Z)-1- | | |
| | (2-aminothiazol-4-yl)-2-(((S)-2,2- | | |
| | dimethy1-4-oxo-1-(sulfooxy)- | | |
| | azetidin-3-yl)amino)-2- | | |
| | oxoethylidene)amino)oxy) | | |
| | propanoic acid | | |
| 27 | 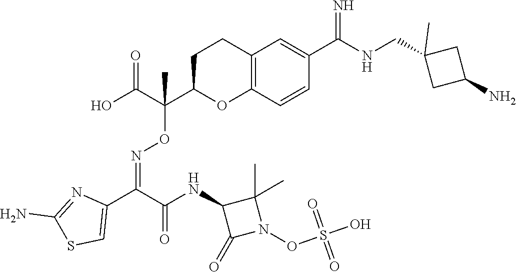 [SMILES: CC1(C)[C@H](NC(=O)/C(=N\O[C@](C)(C(=O)O)[C@H]2CCc3cc(C(=N)NC[C@]4(C)C[C@@H](N)C4)ccc3O2)c2csc(N)n2)C(=O)N1OS(=O)(=O)O] | ¹H NMR (500 MHz, 400 uL D₂O/100 uL CD₃CN) δ_{H} 7.56- 7.53 (m, 2H), 7.07 (d, J = 10 Hz, 1H), 7.01 (s, 1H), 4.80 (s, 1H), 4.59-4.56 (m, 1H), 3.95- 3.87 (m, 1H), 3.58 (s, 2H), 2.98 (br s, 2H), 2.58-2.52 (m, 2H), 2.26-2.11 (m, 4H), 1.91 (br s, 1H), 1.68 (s, 3H), 1.59 (s, 3H), 1.42 (s, 3H), 1.37 (s, 3H). | 723.5 |
| | (S)-2-((R)-6-(N-(((1s,3S)-3- | | |
| | amino-1-methylcyclobutyl)- | | |
| | methyl)carbamimidoyl)chroman- | | |
| | 2-yl)-2-((((Z)-1-(2-aminothiazol- | | |
| | 4-yl)-2-(((S)-2,2-dimethyl-4-oxo- | | |
| | 1-(sulfooxy)azetidin-3-yl)amino)- | | |
| | 2-oxoethylidene)amino)- | | |
| | oxy)propanoic acid or (S)-2-((R)- | | |
| | 6-(N-(((1r,3R)-3-amino-1- | | |
| | methylcyclobutyl)methyl)carbami- | | |
| | midoyl)chroman-2-yl)-2-((((Z)-1- | | |
| | (2-aminothiazol-4-yl)-2-(((S)-2,2- | | |
| | dimethyl-4-oxo-1-(sulfooxy)- | | |
| | azetidin-3-yl)amino)-2- | | |
| | oxoethylidene)amino)oxy) | | |
| | propanoic acid | | |
| 28 | 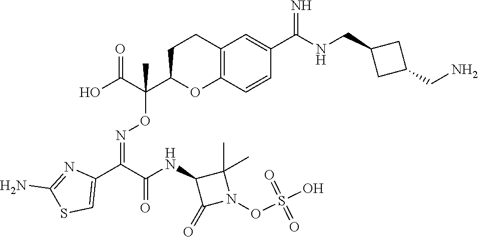 [SMILES: CC1(C)[C@H](NC(=O)/C(=N\O[C@](C)(C(=O)O)[C@H]2CCc3cc(C(=N)NC[C@H]4C[C@H](CN)C4)ccc3O2)c2csc(N)n2)C(=O)N1OS(=O)(=O)O] | ¹H NMR (500 MHz, 400 uL D₂O/100 uL CD₃CN) δ_{H} 7.29- 7.26 (m, 2H), 6.80-6.78 (m, 2H), 4.53 (s, 1H), 4.37-4.34 (m, 1H), 3.27 (d, J = 10 Hz, 2H), 2.86 (d, J = 10 Hz, 2H), 2.71 (br s, 2H), 2.53 (br s, 1H), 2.38 (br s, 1H), 2.19 (br s, 2H), 2.00 (br s, 1H), 1.70 (br s, 1H), 1.44 (m, 5H), 1.33 (s, 3H), 1.11 (s, 3H). | 723.5 |
| | (S)-2-((R)-6-(N-(((1s,3S)-3- | | |
| | (aminomethyl)cyclobutyl)methyl) | | |
| | carbamimidoyl)chroman-2-yl)-2- | | |
| | ((((Z)-1-(2-aminothiazol-4-yl)-2- | | |
| | (((S)-2,2-dimethyl-4-oxo-1- | | |
| | (sulfooxy)azetidin-3-yl)amino)-2- | | |
| | oxoethylidene)amino)oxy)propan- | | |
| | oic acid or (S)-2-((R)-6-(N- | | |
| | (((1r,3R)-3-(aminomethyl)- | | |
| | cyclobutyl)methyl)carbamimidoyl) | | |
| | chroman-2-yl)-2-((((Z)-1-(2- | | |
| | aminothiazol-4-yl)-2-(((S)-2,2- | | |
| | dimethyl-4-oxo-1-(sulfooxy)- | | |
| | azetidin-3-yl)amino)-2- | | |
| | oxoethylidene)amino)oxy) | | |
| | propanoic acid | | |
| 29 | 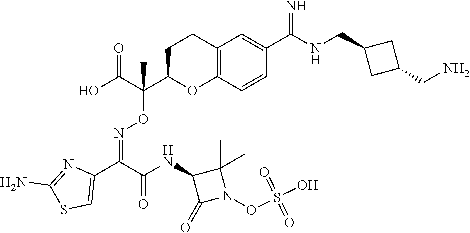 [SMILES: CC1(C)[C@H](NC(=O)/C(=N\O[C@](C)(C(=O)O)[C@H]2CCc3cc(C(=N)NC[C@H]4C[C@H](CN)C4)ccc3O2)c2csc(N)n2)C(=O)N1OS(=O)(=O)O] | ¹H NMR (500 MHz, 400 uL D₂O/100 uL CD₃CN) δ_{H} 7.29- 7.26 (m, 2H), 6.80-6.78 (m, 2H), 4.53 (s, 1H), 4.38-4.35 (m, 1H), 3.39 (d, J = 10 Hz, 2H), 2.97 (d, J = 10 Hz, 2H), 2.71 (br s, 2H), 2.66-2.49 (m, 2H), 2.00 (br s, 1H), 1.90-1.86 (m, 4H), 1.70 (br s, 1H), 1.45 (s, 3H), 1.32 (s, 3H), 1.11 (s, 3H). | 723.5 |
| | (S)-2-((R)-6-(N-(((1s,3S)-3- | | |
| | (aminomethyl)cyclobutyl)methyl) | | |
| | carbamimidoyl)chroman-2-yl)-2- | | |
| | ((((Z)-1-(2-aminothiazol-4-yl)-2- | | |
| | (((S)-2,2-dimethyl-4-oxo-1- | | |
| | (sulfooxy)azetidin-3-yl)amino)-2- | | |
| | oxoethylidene)amino)oxy)propan | | |
| | oic acid or (S)-2-((R)-6-(N- | | |
| | (((1r,3R)-3-(aminomethyl)- | | |
| | cyclobutyl)methyl)carbamimidoyl) | | |
| | chroman-2-yl)-2-((((Z)-1-(2- | | |
| | aminothiazol-4-yl)-2-(((S)-2,2- | | |
| | dimethyl-4-oxo-1-(sulfooxy)- | | |
| | azetidin-3-yl)amino)-2- | | |
| | oxoethylidene)amino)oxy)- | | |
| | propanoic acid | | |

### Example 22: Preparation of Compound 30

### (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(2-(piperidin-4-yl)ethyl)carbamimidoyl)chroman-2-yl)propanoic Acid

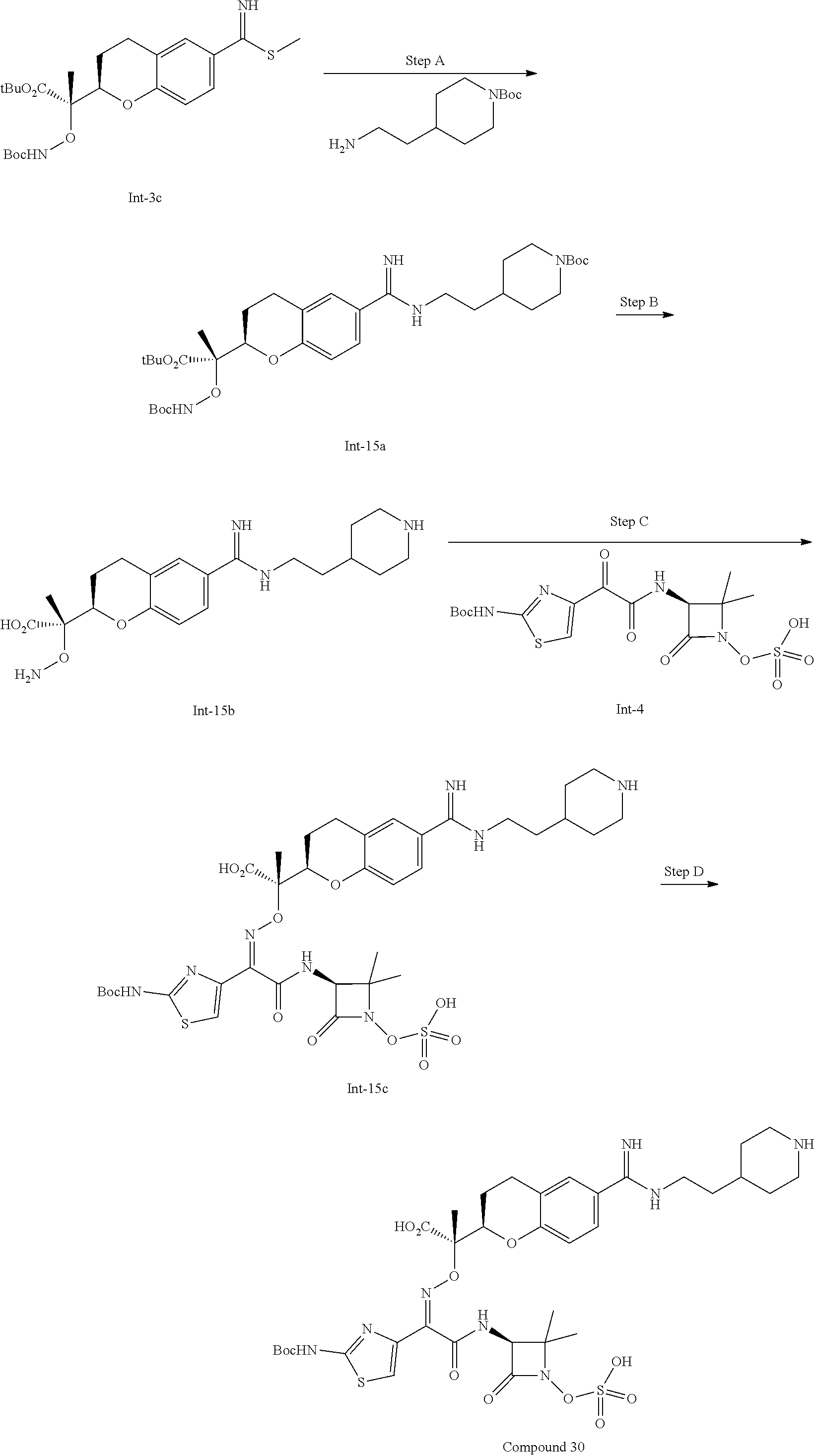

Step A—Synthesis of Intermediate 15a To a solution of tert-butyl 4-(2-aminoethyl)piperidine-1-carboxylate (80 mg, 0.350 mmol) and intermediate 3c (150 mg, 0.321 mmol) in EtOH (2 mL) was added acetic acid (0.1 mL, 1.747 mmol), followed by potassium acetate (63 mg, 0.642 mmol). The reaction was stirred at 80° C. for 1.5 h. Then the solvent was removed under vacuum, and the resulting residue was purified by column chromatography (SiO₂, DCM:MeOH=10:1) to afford intermediate 15a. LC-MS (ESI) m/z: 647.3 [M+H]⁺.

Step B—Synthesis of Intermediate 15b A solution of intermediate 15a (280 mg, 0.433 mmol) in TFA (4 mL) was stirred at 40° C. for 30 min. Then the solvent was removed with a nitrogen gas flow to afford crude intermediate 15b, which was used in the next reaction without further purification. LC-MS (ESI): m/z: 391.1 [M+H]⁺.

Step C—Synthesis of Intermediate 15c A solution of intermediate 4 (200 mg, 0.431 mmol) and intermediate 15b (169 mg, 0.433 mmol) in MeOH (3 mL) was stirred at 25° C. for 1.5 h. The reaction solution was concentrated under vacuum with the water bath temperature controlled below 30° C. to afford crude intermediate 15c, which was used in the next reaction without further purification. LC-MS (ESI) m/z: 837.2 [M+H]⁺.

Step D Synthesis of Compound 30 A solution of intermediate 15c (362 mg, 0.433 mmol) in 4 mL of 1:1 TFA/DCM was stirred at 25° C. for 30 min. Then the solvent was removed with a nitrogen gas flow. The resulting residue was dissolved in DMSO (3 mL) and purified by preparative-HPLC (Column: Phenomenex Gemini-NX C18 80*30 mm*5 um; Condition: water (0.1% TFA)-ACN; Begin B 2%, End B 32%; Gradient Time (min) 11; 100% B Hold Time (min) 2; FlowRate (mL/min) 60) to afford the crude product as its TFA salt form. The crude product was purified by preparative-HPLC (Column: Welch Xtimate C18 150*25 mm*5 um; Condition: water (0.225% FA)—ACN; Begin B 0%, End B18%; Gradient Time (min) 15; 100% B Hold Time (min) 1) to afford compound 30 as its formic acid salt form. LC-MS (ESI) m/z: 737.3 [M+H]⁺. ¹H NMR (400 MHz, DMSO-d₆) δ: 7.48-7.37 (m, 2H), 6.96 (d, J=8.2 Hz, 1H), 6.75 (s, 1H), 4.60 (s, 1H), 4.44 (br d, J=11.0 Hz, 1H), 3.35-3.27 (m, 2H), 3.24-3.16 (m, 2H), 2.90-2.71 (m, 4H), 2.10-2.03 (m, 1H), 1.82-1.73 (m, 2H), 1.65-1.53 (m, 4H), 1.50 (s, 3H), 1.39 (s, 3H), 1.31-1.24 (m, 2H), 1.23 (s, 3H).

### Example 23: Preparation of Compounds 31 and 32

### tert-butyl (S)-2-((((Z)-1-(2-((tert-butoxycarbonyl)amino)thiazol-4-yl)-2-(((S)-2,2-dimethyl-4-oxo-1-(sulfooxy)azetidin-3-yl)amino)-2-oxoethylidene)amino)oxy)-2-((R)-6-(N—(((R)-morpholin-2-yl)methyl)carbamimidoyl)chroman-2-yl)propanoate and tert-butyl (S)-2-((((Z)-1-(2-((tert-butoxycarbonyl)amino)thiazol-4-yl)-2-(((S)-2,2-dimethyl-4-oxo-1-(sulfooxy)azetidin-3-yl)amino)-2-oxoethylidene)amino)oxy)-2-((R)-6-(N—(((S)-morpholin-2-yl)methyl)carbamimidoyl)chroman-2-yl)propanoate

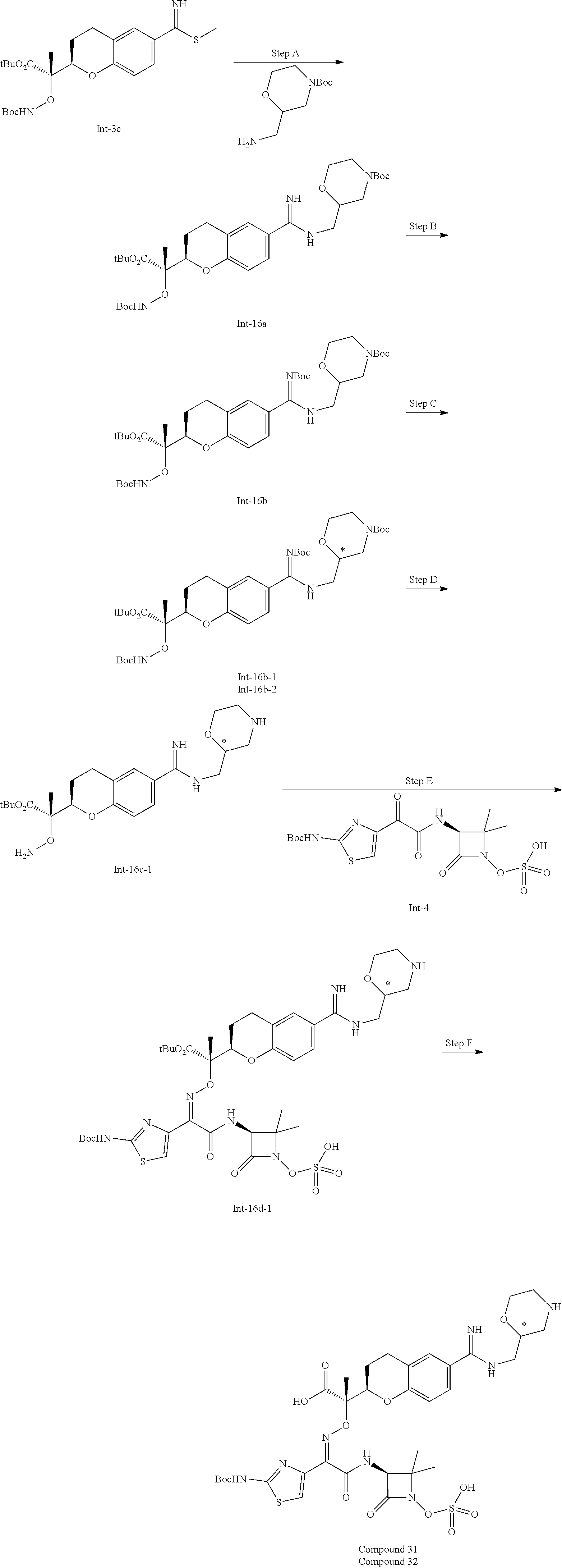

Step A—Synthesis of Intermediate 16a To a solution of tert-butyl 2-(aminomethyl)morpholine-4-carboxylate (391 mg, 1.808 mmol) and intermediate 3c (900 mg, 1.929 mmol) in MeOH (8 mL) was added acetic acid (0.4 mL, 6.99 mmol), followed by potassium acetate (350 mg, 3.57 mmol) at 23° C. The reaction was stirred at 83° C. for 20 min. Then the reaction mixture was concentrated under vacuum to afford crude intermediate 16a. This material was used in the next reaction without further purification. LC-MS (ESI) m/z: 635.4 [M+H]⁺.

Step B—Synthesis of Intermediate 16b To a solution of intermediate 16a (1.2 g, 1.890 mmol) in DCM (6 mL) was added Et₃N (1 mL, 7.17 mmol), followed by (BOC)₂O (1 mL, 4.31 mmol) at 12° C. The reaction was stirred at 25° C. for 16 h. Then the reaction was filtered and the filtrate was purified by flash silica gel chromatography (Biotage; 12 g Agela Silica Flash Column, Eluent of 15-33% EtOAc/Petroleum ether gradient @ 40 mL/min) to give intermediate 16b. LC-MS (ESI) m/z: 735.2 [M+H]⁺.

Step C—Synthesis of Intermediate 16b-1 and 16b-2 Enantiomers of intermediate 16b (860 mg, 1.170 mmol) were separated by chiral SFC (Column: DAICEL CHIRALPAK AD, 250 mm*30 mm, 10 um; Condition: 0.1% NH₃H₂O IPA; Begin B 30%, End B 30%; FlowRate (mL/min) 70; Injections 60) to afford intermediate 16b-1 first eluting isomer) and intermediate 16b-2 (second eluting isomer). LC-MS (ESI) m/z: 735.2 [M+H]⁺.

Step D—Synthesis of Intermediate 16c-1 A solution of intermediate 16b-1 (300 mg, 0.408 mmol) in 6 mL of TFA/DCM (1:5) was stirred at 16° C. for 13 h. Then the solvent was evaporated under a nitrogen gas flow to afford crude intermediate 16c-1, which was used in the next reaction without further purification. LC-MS (ESI) m/z: 435.2 [M+H]⁺.

Step E—Synthesis of Intermediate 16d-1 A solution of intermediate 16c-1 (177 mg, 0.407 mmol) and intermediate 4 (190 mg, 0.409 mmol) in MeOH (4 mL) was stirred at 25° C. for 1 h.

Then the reaction was concentrated under vacuum to afford crude intermediate 16d-1, which was used in the next reaction without further purification. LC-MS (ESI) m/z: 881.5 [M+H]⁺.

Step F—Synthesis of Compound 31 A solution of intermediate 16d-1 (300 mg, 0.341 mmol) in 4 mL of TFA/DCM (3:1) was stirred at 27° C. for 20 min. Then the reaction mixture was dried under a nitrogen gas flow. The resulting residue was dissolved in 3 mL of (3:1) DMSO/MeCN and purified by Prep-HPLC (Column: Boston Uni C18, 40*150*5 um; Condition: water (0.1% TFA)-ACN; Begin B 1, End B 31; Gradient Time (min) 10; 100% B Hold Time (min) 2; FlowRate (mL/min) 60) to afford the product as its TFA salt, which was further purified by Prep-HPLC (Column: Welch Xtimate C18, 150*25 mm*5 um; Condition: water (0.225% FA)-ACN; Begin B 0, End B 18; Gradient Time (min) 15; 100% B Hold Time (min) 2; FlowRate (mL/min) 25) to afford compound 31 as its formic acid salt. LC-MS (ESI) m/z: 725.4 [M+H]⁺. ¹H NMR (400 MHz, DMSO-d₆) δ: 7.46-7.30 (m, 2H), 6.91 (d, J=8.6 Hz, 1H), 6.74 (s, 1H), 4.56 (s, 1H), 4.37 (br d, J=11.3 Hz, 1H), 3.92 (br s, 2H), 3.66 (br t, J=11.3 Hz, 1H), 3.57-3.35 (m, 2H), 3.24 (br d, J=12.5 Hz, 1H), 3.11 (br d, J=12.1 Hz, 1H), 3.01-2.88 (m, 1H), 2.85-2.65 (m, 3H), 2.06-1.95 (m, 1H), 1.67-1.51 (m, 1H), 1.48 (s, 3H), 1.37 (s, 3H), 1.22 (s, 3H).

Compound 32 was analogously prepared as its formic acid salt from intermediate 16b-2 following Steps D to F of Example 23. LC-MS (ESI) m/z: 725.4 [M+H]⁺. ¹H NMR (400 MHz, DMSO-d₆) δ: 7.38 (br s, 1H), 7.31 (br d, J=7.4 Hz, 1H), 6.88 (br d, J=8.6 Hz, 1H), 6.72 (s, 1H), 4.52 (s, 1H), 4.37 (br d, J=10.6 Hz, 1H), 3.94-3.82 (m, 2H), 3.76-3.64 (m, 1H), 3.53-3.34 (m, 2H), 3.27-3.18 (m, 1H), 3.16-3.06 (m, 1H), 3.01-2.89 (m, 1H), 2.86-2.63 (m, 3H), 2.12-1.95 (m, 1H), 1.65-1.51 (m, 1H), 1.48 (s, 3H), 1.35 (s, 3H), 1.20 (s, 3H).

### Example 24: Preparation of Compound 33

### (S)-2-((R)-6-(N-(2-((1R,4R)-2,5-diazabicyclo[2.2.1]heptan-2-yl)ethyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

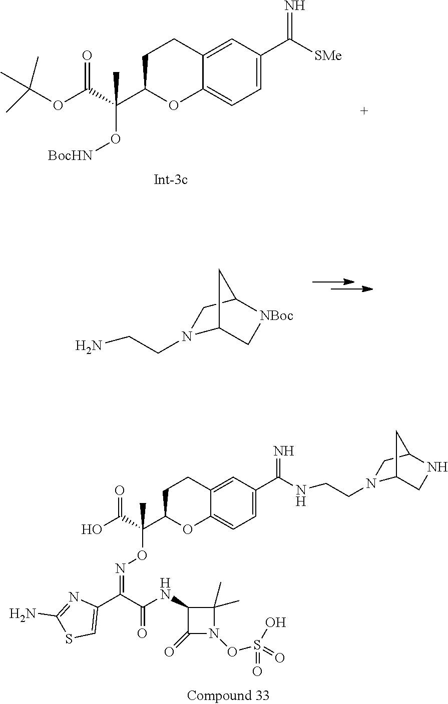 [SMILES: CSC(=N)c1ccc2c(c1)CC[C@H]([C@](C)(ONC(=O)OC(C)(C)C)C(=O)OC(C)(C)C)O2 ; CC(C)(C)OC(=O)N1CC2CC1CN2CCN ; CC1(C)[C@H](NC(=O)/C(=N\O[C@](C)(C(=O)O)[C@H]2CCc3cc(C(=N)NCCN4CC5CC4CN5)ccc3O2)c2csc(N)n2)C(=O)N1OS(=O)(=O)O]

Compound 33 was analogously prepared as its formic acid salt from intermediate 3c, using the method described in Steps A to F in Example 23 with the exception of substituting tert-butyl 2-(aminomethyl)morpholine-4-carboxylate with (1R, 4R) tert-butyl 5-(2-aminoethyl)-2,5-diaza-bicyclo[2.2.1]heptane-2-carboxylate in Step A. LC-MS (ESI) m/z: 749.9 [M+H]⁺. ¹H NMR (400 MHz, CD₃CN+D₂O) δ: 7.43-7.35 (m, 2H), 6.88 (d, J=8.6 Hz, 1H), 6.80 (s, 1H), 4.59 (s, 1H), 4.37-4.35 (m, 1H), 4.19 (s, 1H), 3.71 (s, 1H), 3.43 (br t, J=6.1 Hz, 2H), 3.37 (br d, J=12.1 Hz, 1H), 3.13 (br d, J=10.2 Hz, 1H), 2.99-2.89 (m, 2H), 2.87-2.73 (m, 4H), 2.11-1.98 (m, 2H), 1.78 (br d, J=11.0 Hz, 1H), 1.74-1.60 (m, 1H), 1.49 (s, 3H), 1.41 (s, 3H), 1.23 (s, 3H).

### Example 25: Preparation of Intermediate 17d

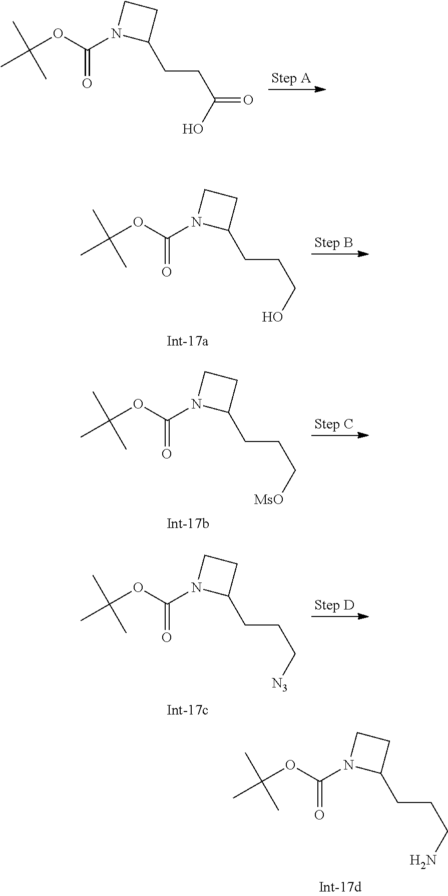 [SMILES: CC(C)(C)OC(=O)N1CCC1CCC(=O)O ; CC(C)(C)OC(=O)N1CCC1CCCO ; CC(C)(C)OC(=O)N1CCC1CCCOS(C)(=O)=O ; CC(C)(C)OC(=O)N1CCC1CCCN=[N+]=[N-] ; CC(C)(C)OC(=O)N1CCC1CCCN]

Step A—Synthesis of Intermediate 17a To a solution of 3-(1-(tert-butoxycarbonyl)azetidine-2-yl)propanic acid (500 mg, 2.181 mmol) in THF (15 mL) stirred at −12° C. (ice/acetone bath) was added a solution of 2 M lithium aluminum hydride in THF (1.636 mL, 3.27 mmol). The mixture was stirred at −10° C. for 1 h. Then the reaction was quenched with 1 N NaOH (10 mL), and extracted with EtOAc (3×50 mL). The combined organic layers were washed with brine (50 mL), and dried over anhydrous MgSO₄. The drying agent was removed via filtration, and the filtrate was concentrated under vacuum to give intermediate 17a, which was used in the next reaction without further purification. TLC: Rf=0.2 EtOAc/Hexane (1/1), KMnO₄ Stain.

Step B—Synthesis of Intermediate 17b To a solution of intermediate 17a (534 mg, 2.480 mmol) and triethylamine (0.688 mL, 4.96 mmol) in THF (20 mL) stirred at 0° C., was added methane sulfonyl chloride (0.288 mL, 3.72 mmol). The mixture was warmed to room temperature and stirred for 1 h. Then the reaction was quenched with saturated aqueous NaHCO₃ solution (20 mL), and extracted with EtOAc (2×50 mL). The combined organic layers were washed with brine (50 mL), and dried over anhydrous MgSO₄. The drying agent was removed via filtration, and the filtrate was concentrated under vacuum to give the crude intermediate 17b, which was used in the next reaction without further purification. TLC: Rf=0.5 EtOAc/Hexane (1/1), KMnO₄ Stain.

Step C—Synthesis of Intermediate 17c To a solution of intermediate 17b (730 mg, 2.488 mmol) in DMF (10 mL) stirred at room temperature was added sodium azide (243 mg, 3.73 mmol). The mixture was stirred at 70° C. for 16 h. Then the reaction was quenched with saturated aqueous NaHCO₃ solution (20 mL), and extracted with EtOAc (2×50 mL). The combined organic layers were washed with brine (50 mL), and dried over anhydrous MgSO₄. The drying agent was removed via filtration and the filtrate concentrated under vacuum. The resulting residue was purified by silica gel column eluting with 0-~50% EtOAc/hexanes (gradient) to give intermediate 17c. TLC: Rf=0.6 EtOAc/Hexane (3/7), KMnO₄ Stain.

Step D Synthesis of Intermediate 17d To a solution of intermediate 17c (212 mg, 0.882 mmol) and N,N-diisopropylethylamine (0.154 mL, 0.882 mmol) in THF (5 mL)/water (1.00 mL) stirred at room temperature, were added diphenylphosphinated resin (copolymer of styrene and divinylbenzene) (692 mg, 2.65 mmol). The mixture was stirred at 40° C. overnight. Then the reaction was filtered to remove the resin and the resin was further rinsed with MeOH. The filtrate was concentrated in vacuo, and the resulting residue was azeotroped with toluene (2×5 mL). The resulting oil was dried under high vacuum for 16 h to give the crude intermediate 17d, which was used without further purification. TLC: Rf=0.0 EtOAc/Hexane (1/1), KMnO₄ Stain.

### Example 26: Preparation of Compounds 34 to 37

The following compounds were analogously prepared as formic acid salts from the appropriate amines according to the method of Steps A to C of Example 23 and Steps B to D of Example 22:

| | | | LCMS |
|---|---|---|---|
| Compound | Structure | ¹H NMR | [M + H]⁺ |
| 34 | 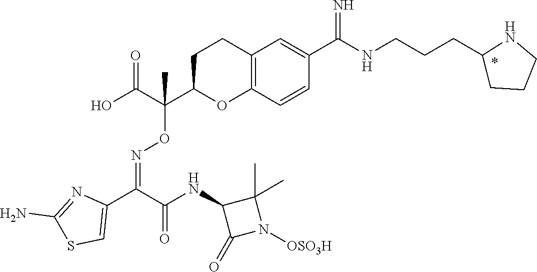 | ¹H NMR (500 MHz, 400 uL D₂O/100 uL CD₃CN) δ_{H} 7.32- 7.27 (m, 2H), 6.80-6.78 (m, 2H), 4.52 (s, 1H), 4.36-4.33 (m, 1H), 3.45-3.42 (m, 1H), 3.32 (br s, 2H), 3.17 (br s, 2H), 2.72 (br s, 2H), 2.14-2.06 (m, 1H), 2.02-1.79 (m, 3H), 1.74- 1.65 (m, 5H), 1.57-1.50 (m, 1H), 1.44 (s, 3H), 1.32 (s, 3H), 1.11 (s, 3H). | 737.5 |
| | (S)-2-((((Z)-1-(2-aminothiazol-4- | | |
| | yl)-2-(((S)-2,2-dimethyl-4-oxo-1- | | |
| | (sulfooxy)azetidin-3-yl)amino)-2- | | |
| | oxoethylidene)amino)oxy)-2- | | |
| | ((R)-6-(N-(3-((S)-pyrrolidin-2- | | |
| | yl)propyl)carbamimidoyl)chroman- | | |
| | 2-yl)propanoic acid or (S)-2- | | |
| | ((((Z)-1-(2-aminothiazol-4-yl)-2- | | |
| | (((S)-2,2-dimethyl-4-oxo-1- | | |
| | (sulfooxy)azetidin-3-yl)amino)-2- | | |
| | oxoethylidene)amino)oxy)-2- | | |
| | ((R)-6-(N-(3-((R)-pyrrolidin-2- | | |
| | yl)propyl)carbamimidoyl)chroman- | | |
| | 2-yl)propanoic acid | | |
| 35 | 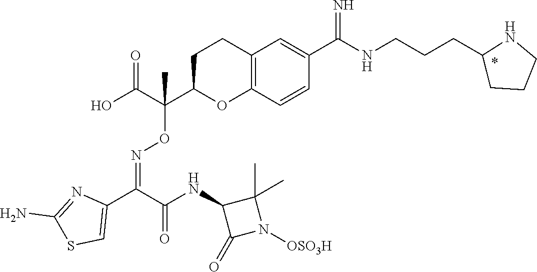 | ¹H NMR (500 MHz, 400 uL D₂O/100 uL CD₃CN) δ_{H} 7.30- 7.27 (m, 2H), 6.80-6.78 (m, 2H), 4.55 (s, 1H), 4.36-4.33 (m, 1H), 3.44-3.42 (m, 1H), 3.32 (br s, 2H), 3.17-3.14 (m, 2H), 2.71 (br s, 2H), 2.14-2.06 (m, 1H), 2.02-1.81 (m, 3H), 1.74-1.63 (m, 5H), 1.57-1.49 (m, 1H), 1.44 (s, 3H), 1.32 (s, 3H), 1.11 (s, 3H). | 737.5 |
| | (S)-2-((((Z)-1-(2-aminothiazol-4- | | |
| | yl)-2-(((S)-2,2-dimethyl-4-oxo-1- | | |
| | (sulfooxy)azetidin-3-yl)amino)-2- | | |
| | oxoethylidene)amino)oxy)-2- | | |
| | ((R)-6-(N-(3-((S)-pyrrolidin-2- | | |
| | yl)propyl)carbamimidoyl)chroman- | | |
| | 2-yl)propanoic acid or (S)-2- | | |
| | ((((Z)-1-(2-aminothiazol-4-yl)-2- | | |
| | (((S)-2,2-dimethyl-4-oxo-1- | | |
| | (sulfooxy)azetidin-3-yl)amino)-2- | | |
| | oxoethylidene)amino)oxy)-2- | | |
| | ((R)-6-(N-(3-((R)-pyrrolidin-2- | | |
| | yl)propyl)carbamimidoyl)chroman- | | |
| | 2-yl)propanoic acid | | |
| 36 | 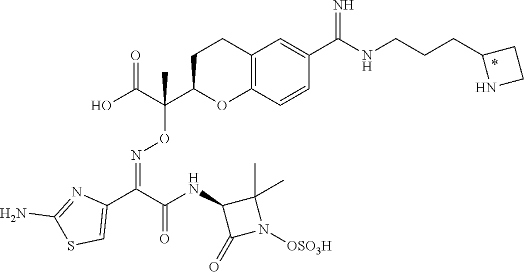 | ¹H NMR (500 MHz, 400 uL D₂O/100 uL CD₃CN) δ_{H} 7.49- 7.45 (m, 2H), 6.99-6.96 (m, 2H), 4.58-4.52 (m, 2H), 4.13- 4.07 (m, 1H), 3.93 (br s, 1H), 3.49 (br s, 2H), 2.89 (br s, 2H), 2.60 (br s, 1H), 2.42-2.37 (m, 1H), 2.19-2.17 (m, 1H), 2.07- 2.00 (m, 3H), 1.87 (br s, 1H), 1.76 (br s, 2H), 1.62 (s, 3H), 1.49 (s, 3H), 1.27 (s, 3H). | 723.7 |
| | (S)-2-((((Z)-1-(2-aminothiazol-4- | | |
| | yl)-2-(((S)-2,2-dimethyl-4-oxo-1- | | |
| | (sulfooxy)azetidin-3-yl)amino)-2- | | |
| | oxoethylidene)amino)oxy)-2- | | |
| | ((R)-6-(N-(3-((S)-azetidin-2- | | |
| | yl)propyl)carbamimidoyl)chroman- | | |
| | 2-yl)propanoic acid or (S)-2- | | |
| | ((((Z)-1-(2-aminothiazol-4-yl)-2- | | |
| | (((S)-2,2-dimethyl-4-oxo-1- | | |
| | (sulfooxy)azetidin-3-yl)amino)-2- | | |
| | oxoethylidene)amino)oxy)-2- | | |
| | ((R)-6-(N-(3-((R)-azetidin-2- | | |
| | yl)propyl)carbamimidoyl)chroman- | | |
| | 2-yl)propanoic acid | | |
| 37 | 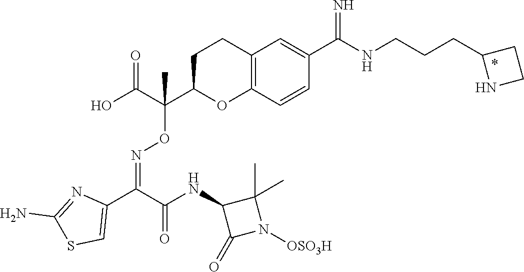 | ¹H NMR (500 MHz, 400 uL D₂O/100 uL CD₃CN) δ_{H} 7.48- 7.45 (m, 2H), 6.97 (s, 2H), 4.58-4.52 (m, 2H), 4.13-4.08 (m, 1H), 3.94 (br s, 1H), 3.49 (br s, 2H), 2.88 (br s, 2H), 2.60 (br s, 1H), 2.42-2.36 (m, 1H), 2.18-2.16 (m, 1H), 2.06-2.00 (m, 3H), 1.87 (br s, 1H), 1.76 (br s, 2H), 1.61 (s, 3H), 1.49 (s, 3H), 1.28 (s, 3H). | 723.7 |
| | (S)-2-((((Z)-1-(2-aminothiazol-4- | | |
| | yl)-2-(((S)-2,2-dimethyl-4-oxo-1- | | |
| | (sulfooxy)azetidin-3-yl)amino)-2- | | |
| | oxoethylidene)amino)oxy)-2- | | |
| | ((R)-6-(N-(3-((S)-azetidin-2- | | |
| | yl)propyl)carbamimidoyl)chroman- | | |
| | 2-yl)propanoic acid or (S)-2- | | |
| | ((((Z)-1-(2-aminothiazol-4-yl)-2- | | |
| | (((S)-2,2-dimethyl-4-oxo-1- | | |
| | (sulfooxy)azetidin-3-yl)amino)-2- | | |
| | oxoethylidene)amino)oxy)-2- | | |
| | ((R)-6-(N-(3-((R)-azetidin-2- | | |
| | yl)propyl)carbamimidoyl)chroman- | | |
| | 2-yl)propanoic acid | | |

### Example 27: Preparation of Compound 38

### (S)-2-((R)-6-(N-(((1s,4S)-4-(aminomethyl)cyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

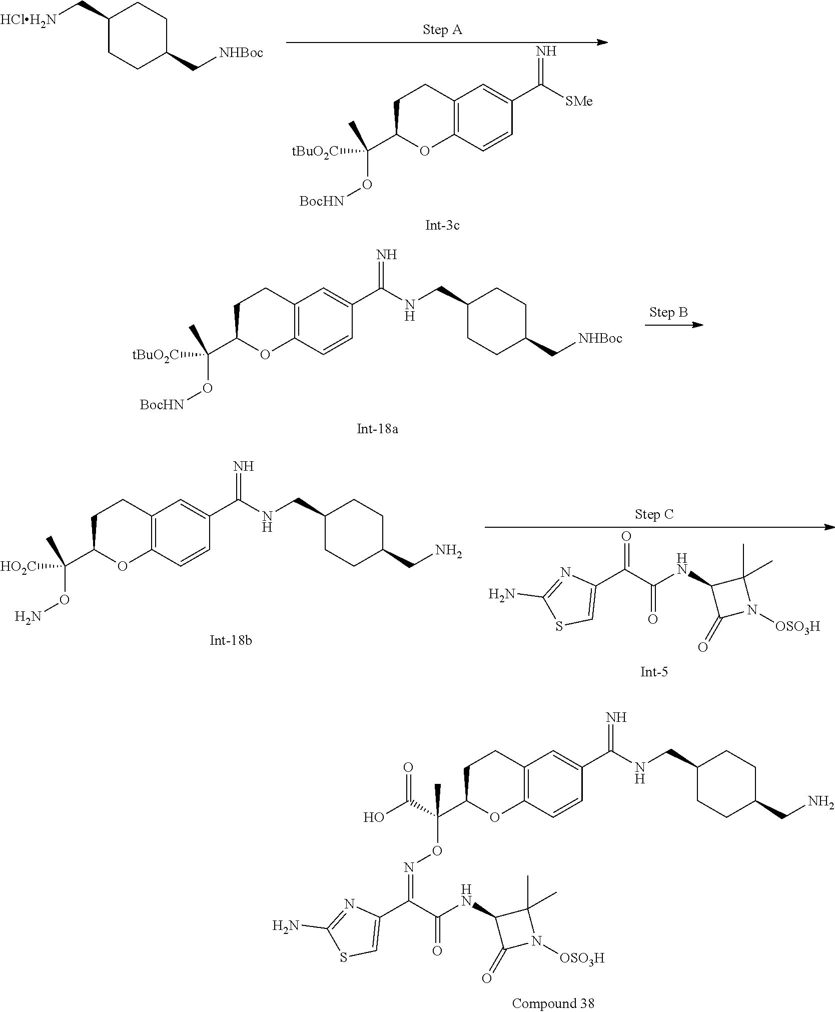

Step A—Synthesis of Intermediate 18a To a vial charged with tert-butyl (cis-4-(aminomethyl)-cyclohexyl)carbamate hydrochloride (143 mg, 0.51 mmol) were added a stock solution of acetic acid (118 μL, 2.1 mmol) in AcCN (3.4 mL), Hunig's Base (120 μl, 0.67 mmol), and intermediate 3c (160 mg, 0.34 mmol) sequentially. The reaction mixture was stirred at 70° C. for 1 h, then cooled to ambient temperature and purified directly by reverse phase MPLC (Column: Isco, C18 100 g; gradient elution with 10-100% AcCN (0.05% TFA)/water (0.05% TFA). The product fractions were collected and concentrated in vacuo to remove AcCN, and the remaining aqueous residue was lyophilized for 16 h to give the title compound. LC-MS [M+H]⁺: m/z 661.6.

Step B—Synthesis of Intermediate 18b A mixture of intermediate 18a (0.17 g, 0.22 mmol) and 2:1 trifluoroacetic acid/anhydrous dichloromethane (4.5 mL) was stirred at 40° C. for 2-3 hours. Then the reaction mixture was cooled to ambient temperature, and the solvent was removed under vacuum. The resulting residue was dried under high vacuum for 2 hours to give title compound as its TFA salt, which was used in the next reaction without further purification. LC-MS [M+H]⁺: m/z 405.5.

Step C—Synthesis of Compound 38 To a vial charged with intermediate 18b (0.12 g, 0.19 mmol), intermediate 5 (86 mg, 0.24 mmol), and oven-dried molecular sieves 4A (325 mesh particle; 0.12 g) was added anhydrous methanol (4.0 mL) at ambient temperature. The reaction mixture was stirred at room temperature for 16 h, then filtered through Celite™, and the Celite™ pad was rinsed with MeOH. The filtrate was concentrated under vacuum without heating. The resulting residue was dissolved in water and purified on reverse phase MPLC (Isco; Column: C18-Aq 150 g; gradient elution with 0-30% AcCN (0.05% TFA)/water (0.05% TFA)). The product fractions were collected and lyophilized to give title compound as its TFA salt. The TFA salt was converted to the formic acid salt by passing through a reverse phase MPLC (Column: Isco, C18-Aq 50 g; gradient elution with 0-50% AcCN (0.1% FA)/water (0.1% FA)). The product fractions were collected and lyophilized to give title compound as its formic acid salt. LC-MS [M+H]⁺: m/z 751.5. ¹H NMR (500 MHz, D₂O/CD₃CN 4:1) δ 7.85 (s, 1H), 7.90 (d, J=8.9 Hz, 1H), 7.42 (d, J=7.4 Hz, 1H), 7.31 (s, 1H), 5.12 (s, 1H), 4.96-4.84 (m, 1H), 3.77 (s, 2H), 3.37 (m, 2H), 3.30 (m, 2H), 2.59 (s, 1H), 2.31 (s, 1H), 2.21 (m, 2H), 2.05 (s, 3H), 2.02 (s, 3H), 1.94 (s, 3H), 1.88 (m, 4H), 1.76 (s, 3H).

### Example 28: Preparation of Compounds 39 to 45

The following compounds were analogously prepared as formic acid salts from the appropriate commercially available amines using the method described in Steps A to C of Example 27.

| Com- | | | LCMS |
|---|---|---|---|
| pound | Structure | ¹H NMR | [M + H]⁺ |
| 39 | 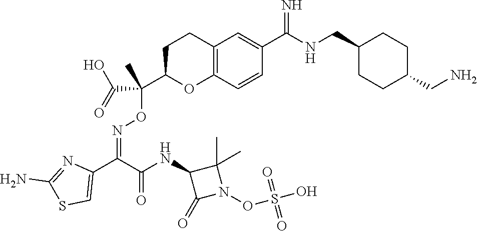 [SMILES: CC1(C)[C@H](NC(=O)/C(=N\O[C@](C)(C(=O)O)[C@H]2CCc3cc(C(=N)NC[C@H]4CC[C@H](CN)CC4)ccc3O2)c2csc(N)n2)C(=O)N1OS(=O)(=O)O] | ¹H NMR (500 MHz, D₂O/CD₃CN 4:1) δ: 7.85 (s, 1H), 7.84 (d, J = 9.3 Hz, 1H), 7.35 (s, 1H), 7.25 (s, 1H), 5.05 (s, 1H), 4.86 (m, 1H), 3.63 (s, 2H), 3.24 (m, 1H), 3.19 (s, 2H), 2.52 (s, 1H), 2.22 (m, 6H), 1.99 (s, 1H), 1.95 (s, 3H), 1.87 (s, 3H), 1.70 (s, 3H), 1.42 (m, 4H). | 751.5 |
| | (S)-2-((R)-6-(N-(((1r,4R)-4- | | |
| | (aminomethyl)cyclohexyl)methyl) | | |
| | carbamimidoyl)chroman-2-yl)- | | |
| | 2-((((Z)-1-(2-aminothiazol-4-yl)- | | |
| | 2-(((S)-2,2-dimethyl-4-oxo-1- | | |
| | (sulfooxy)azetidin-3-yl)amino)- | | |
| | 2-oxoethylidene)amino)- | | |
| | oxy )propanoic acid | | |
| 40 | 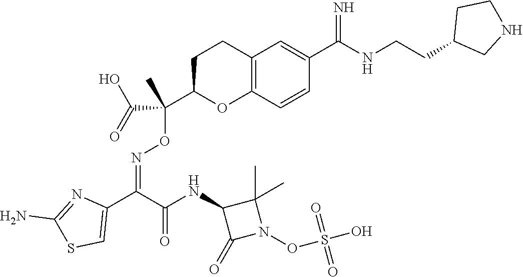 [SMILES: CC1(C)[C@H](NC(=O)/C(=N\O[C@](C)(C(=O)O)[C@H]2CCc3cc(C(=N)NCC[C@@H]4CCNC4)ccc3O2)c2csc(N)n2)C(=O)N1OS(=O)(=O)O] | ¹H NMR (500 MHz, D₂O) δ: 7.71 (s, 1H), 7.69 (d, J = 8.8 Hz, 1H), 7.20 (d, J = 8.5 Hz, 1H), 7.15 (s, 1H), 4.90 (s, 1H), 4.72 (d, J = 10.9 Hz, 1H), 3.79 - 3.74 (m, 1H), 3.74- 3.69 (m, 2H), 3.66 (t, J = 8.6 Hz, 1H), 3.50 (q, J = 10.8, 9.7 Hz, 1H), 3.12 (dt, J = 24.2, 12.4 Hz, 3H), 2.65 (dt, J = 15.4, 7.8 Hz, 1H), 2.49 (dd, J = 12.0, 4.6 Hz, 1H), 2.39 (d, J = 12.8 Hz, 1H), 2.17 (dq, J = 14.1, 7.1 Hz, 1H), 2.13-1.99 (m, 2H), 1.99-1.87 (m, 1H), 1.82 (s, 3H), 1.72 (s, 3H), 1.53 (s, 3H). | 723.5 |
| | (S)-2-((((Z)-1-(2-aminothiazol- | | |
| | 4-yl)-2-(((S)-2,2-dimethyl-4- | | |
| | oxo-1-(sulfooxy)azetidin-3- | | |
| | yl)amino)-2-oxoethylidene)- | | |
| | amino)oxy)-2-((R)-6-(N-(2-((S)- | | |
| | pyrrolidin-3-yl)ethyl)- | | |
| | carbamimidoyl)chroman-2- | | |
| | yl)propanoic acid | | |
| 41 | 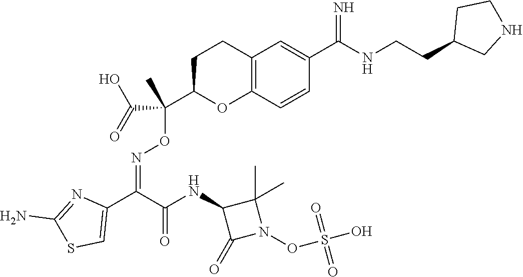 [SMILES: CC1(C)[C@H](NC(=O)/C(=N\O[C@](C)(C(=O)O)[C@H]2CCc3cc(C(=N)NCC[C@H]4CCNC4)ccc3O2)c2csc(N)n2)C(=O)N1OS(=O)(=O)O] | ¹H NMR (500 MHz, D₂O) δ: 7.75 (s, 1H), 7.72 (d, J = 8.7 Hz, 1H), 7.23 (d, J = 8.5 Hz, 1H), 7.17 (s, 1H), 4.94 (s, 1H), 4.74 (d, J = 11.1 Hz, 1H), 3.78 (dd, J = 11.4, 8.0 Hz, 1H), 3.73 (t, J = 7.1 Hz, 2H), 3.70- 3.65 (m, 1H), 3.53 (q, J = 11.0, 9.8 Hz, 1H), 3.15 (dt, J = 26.9, 12.4 Hz, 3H), 2.68 (dt, J = 15.7, 7.9 Hz, 1H), 2.52 (dt, J = 12.1, 5.8 Hz, 1H), 2.42 (d, J = 12.7 Hz, 1H), 2.34- 2.27 (m, 2H), 2.20 (dt, J = 13.9, 7.0 Hz, 1H), 2.10 (dtt, J = 23.4, 11.3, 5.3 Hz, 2H), 2.01-1.90 (m, 1H), 1.84 (s, 3H), 1.76 (s, 3H), 1.58 (s, 3H). | 723.4 |
| | (S)-2-((((Z)-1-(2-aminothiazol- | | |
| | 4-yl)-2-(((S)-2,2-dimethyl-4- | | |
| | ox0-1-(sulfooxy)azetidin-3- | | |
| | yl)amino)-2-oxoethylidene)- | | |
| | amino)oxy)-2-((R)-6-(N-(2-((R)- | | |
| | pyrrolidin-3-yl)ethyl)- | | |
| | carbamimidoyl)chroman-2- | | |
| | yl)propanoic acid | | |
| 42 | 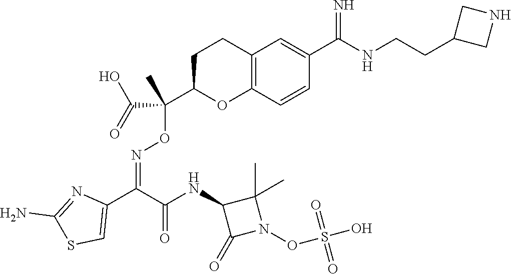 [SMILES: CC1(C)[C@H](NC(=O)/C(=N\O[C@](C)(C(=O)O)[C@H]2CCc3cc(C(=N)NCCC4CNC4)ccc3O2)c2csc(N)n2)C(=O)N1OS(=O)(=O)O] | ¹H NMR (500 MHz, D₂O/CD₃CN 4:1) δ: 7.74 (s, 1H), 7.71 (d, J = 9.1 Hz, 1H), 7.24 (d, J = 8.5 Hz, 1H), 7.17 (s, 1H), 4.94 (s, 1H), 4.75 (d, J = 11.3 Hz, 1H), 4.45 (t, J = 9.6 Hz, 2H), 4.16 (t, J = 9.2 Hz, 2H), 3.65 (t, J = 6.8 Hz, 2H), 3.33 (p, J = 8.1 Hz, 1H), 3.23-3.08 (m, 2H), 2.42 (d, J = 12.9 Hz, 1H), 2.35 (q, J = 7.1 Hz, 2H), 2.13-2.00 (m, 1H), 1.85 (s, 3H), 1.76 (s, 3H), 1.57 (s, 3H). | 709.8 |
| | (S)-2-((((Z)-1-(2-aminothiazol- | | |
| | 4-yl)-2-(((S)-2,2-dimethyl-4- | | |
| | oxo-1-(sulfooxy)azetidin-3- | | |
| | yl)amino)-2-oxo- | | |
| | ethylidene)amino)oxy)-2-((R)-6- | | |
| | (N-(2-(azetidin-3-yl)- | | |
| | ethyl)carbamimidoyl)chroman- | | |
| | 2-yl)propanoic acid | | |
| 43 | 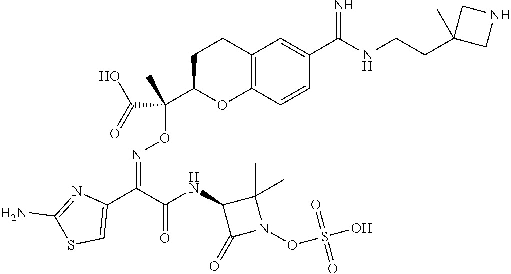 [SMILES: CC1(CCNC(=N)c2ccc3c(c2)CC[C@H]([C@](C)(O/N=C(\C(=O)N[C@@H]2C(=O)N(OS(=O)(=O)O)C2(C)C)c2csc(N)n2)C(=O)O)O3)CNC1] | ¹H NMR (500 MHz, D₂O/CD₃CN 4:1) δ: 7.77 (s, 1H), 7.75 (d, J = 8.6 Hz, 1H), 7.32 (d, J = 8.3 Hz, 1H), 7.25 (d, J = 8.5 Hz, 1H), 4.96 (s, 1H), 4.25 (d, J = 10.9 Hz, 2H), 4.11 (d, J = 10.8 Hz, 2H), 3.76- 3.66 (m, 2H), 3.26-3.16 (m, 2H), 2.52-2.45 (m, 1H), 2.46-2.38 (m, 2H), 2.35 (s, 1H), 2.09-2.18 (m, 1H), 2.01-1.91 (m, 3H), 1.77 (s, 3H), 1.69 (s, 3H), 1.54 (s, 3H). | 723.8 |
| | (S)-2-((((Z)-1-(2-aminothiazol- | | |
| | 4-yl)-2-(((S)-2,2-dimethyl-4- | | |
| | oxo-1-(sulfooxy)azetidin-3- | | |
| | yl)amino)-2-oxoethylidene)- | | |
| | amino)oxy)-2-((R)-6-(N-(2-(3- | | |
| | methylazetidin-3-yl)ethyl)- | | |
| | carbamimidoyl)chroman-2- | | |
| | yl)propanoic acid | | |
| 44 | 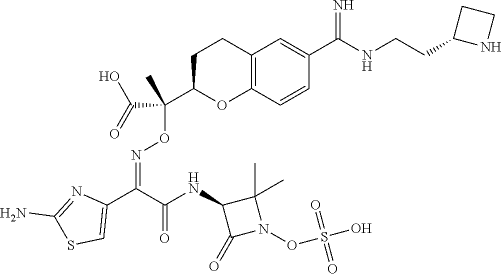 [SMILES: CC1(C)[C@H](NC(=O)/C(=N\O[C@](C)(C(=O)O)[C@H]2CCc3cc(C(=N)NCC[C@@H]4CCN4)ccc3O2)c2csc(N)n2)C(=O)N1OS(=O)(=O)O] | ¹H NMR (500 MHz, D₂O/CD₃CN 4:1) δ: 7.63 (s, 1H), 7.61 (d, J = 8.7 Hz, 1H), 7.14 (s, 1H), 7.11 (d, J = 8.6 Hz, 1H), 4.84 (s, 1H), 4.66 (d, J = 10.9 Hz, 1H), 4.25 (q, J = 9.5 Hz, 1H), 4.09 (td, J = 10.1, 5.7 Hz, 1H), 3.62 (t, J = 6.9 Hz, 2H), 3.13- 2.96 (m, 2H), 2.77 (dq, J = 13.9, 8.8, 6.7 Hz, 1H), 2.51 (dtq, J = 28.7, 14.1, 8.1, 7.0 Hz, 3H), 2.32 (d, J = 12.3 Hz, 1H), 2.05-1.92 (m, 1H), 1.76 (s, 3H), 1.64 (s, 3H), 1.43 (s, 3H). | 709.2 |
| | (S)-2-((((Z)-1-(2-aminothiazol- | | |
| | 4-yl)-2-(((S)-2,2-dimethyl-4- | | |
| | oxo-1-(sulfooxy)azetidin-3- | | |
| | yl)amino)-2-oxoethylidene)- | | |
| | amino)oxy)-2-((R)-6-(N-(2-((S)- | | |
| | azetidin-2-yl)ethyl)- | | |
| | carbamimidoyl)chroman-2- | | |
| | yl)propanoic acid | | |
| 45 | 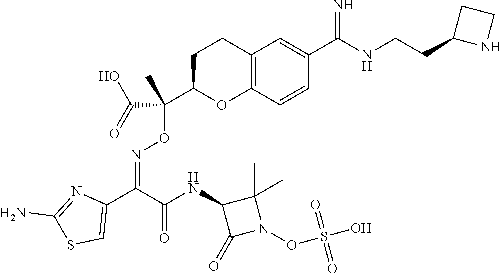 [SMILES: CC1(C)[C@H](NC(=O)/C(=N\O[C@](C)(C(=O)O)[C@H]2CCc3cc(C(=N)NCC[C@H]4CCN4)ccc3O2)c2csc(N)n2)C(=O)N1OS(=O)(=O)O] | ¹H NMR (500 MHz, D₂O/CD₃CN 4:1) δ: 7.72 (s, 1H), 7.70 (d, J = 8.9 Hz, 1H), 7.22 (d, J = 8.6 Hz, 1H), 7.13 (s, 1H), 4.91 (s, 1H), 4.71 (d, J = 11.0 Hz, 1H), 4.34 (q, J = 9.6 Hz, 1H), 4.18 (d, J = 5.3 Hz, 1H), 3.71 (t, J = 6.8 Hz, 2H), 3.19- 3.05 (m, 2H), 2.91-2.82 (m, 1H), 2.73-2.49 (m, 3H), 2.39 (d, J = 12.8 Hz, 1H), 2.06 (s, 1H), 1.81 (s, 3H), 1.73 (s, 3H), 1.54 (s, 3H). | 709.1 |
| | (S)-2-((((Z)-1-(2-aminothiazol- | | |
| | 4-y1)-2-(((S)-2,2-dimethyl-4- | | |
| | oxo-1-(sulfooxy)azetidin-3- | | |
| | yl)amino)-2-oxo- | | |
| | ethylidene)amino)oxy)-2-((R)-6- | | |
| | (N-(2-((R)-azetidin-2- | | |
| | yl)ethyl)carbamimidoyl)chroman- | | |
| | 2-yl)propanoic acid | | |

### Example 29: Preparation of Compounds 46 and 47

### (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N—((S)-1-(azetidin-3-yl)propan-2-yl)carbamimidoyl)chroman-2-yl)propanoic Acid and (S)-2-((((Z)-1-(2-aminothiazol-4-yl)-2-(((S)- 2,2-dimethyl-4-oxo-1-(sulfooxy)azetidin-3-yl)amino)-2-oxoethylidene)amino)oxy)-2-((R)-6-(N—((R)-1-(azetidin-3-yl)propan-2-yl)carbamimidoyl)chroman-2-yl)propanoic Acid

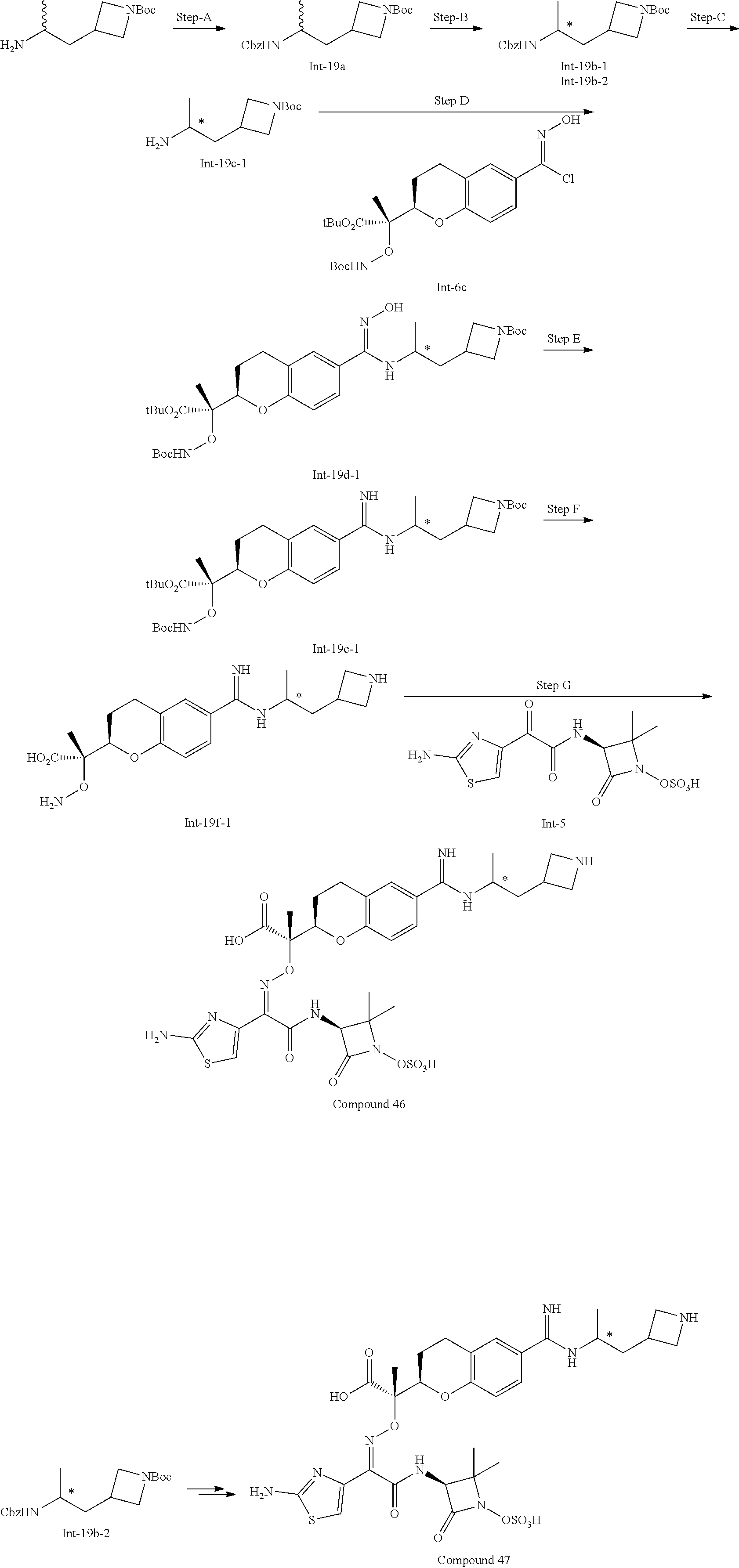

Step A—Synthesis of Intermediate 19a To a mixture of tert-butyl 3-(2-aminopropyl)azetidine-1-carboxylate TFA salt (lg, 2.9 mmol) and diisopropylethylamine (1.5 mL, 8.7 mmol) in DCM (29 mL), stirred for 10 minutes at 0° C. under N₂, was added dropwise a solution of benzylchloroformate (0.43 mL, 3.0 mmol) in 6 mL of DCM. The reaction was stirred at 0° C. for 30 minutes, then poured into ice-cold NH₄Cl solution (30 mL). The aqueous phase was separated, and extracted with DCM (20 mL). The organic layer was dried with anhydrous MgSO₄, filtered, and the filtrate was concentrated in vacuo. The resulting residue was purified by silica gel chromatography eluting with 0-100% MTBE/hexanes (gradient) to provide the title compound. LC-MS [M+Na]⁺: m/z 371.2.

Step B—Synthesis of Intermediate 19b-1 and 19b-2 The enantiomers of intermediate 19a were separated by chiral SFC (Column: Lux Cellulose-4, 2×25 cm; 17% MeOH/CO₂ (100 bar); 60 mL/min; 220 nm) to provide intermediates 19b-1 (the first eluting isomer, LC-MS [M+Na]⁺: m/z 371.2), and 19b-2 (the second eluting isomer, LC-MS [M+Na]⁺: m/z 371.2).

Step C—Synthesis of Intermediate 19c-1 A flask charged with intermediate 19b-1 (330 mg, 0.95 mmol) and Pd/C (50 mg, 10 wt %, 0.047 mmol), was evacuated and refilled with N₂. Then MeOH (9.5 mL) was added via a syringe, and the vial was evacuated and refilled with H₂ (3×). The reaction was stirred under a hydrogen balloon (1 atm) until completion, then filtered through Celite™. The filter cake was rinsed with MeOH, and the filtrate was concentrated under vacuum to provide intermediate 19c-1. LC-MS [M+H]⁺: m/z 215.2.

Step D—Synthesis of Intermediate 19d-1 To a mixture of intermediate 6c (300 mg, 0.64 mmol), and intermediate 19c-1 (205 mg, 0.96 mmol) in DMF (6.4 mL) was added triethylamine (0.89 mL, 6.4 mmol) The reaction was stirred at room temperature for 1 h, then the crude reaction mixture was directly purified by reverse phase MPLC (Column: Biotage, Sfar C18D 120 g, 50 mL/min, gradient elution with 0-100% MeCN+0.05% TFA/H₂O+0.05% TFA). The product-containing fractions were combined, concentrated under vacuum without heating, and the resulting aqueous residue was lyophilized for 16 h to provide intermediate 19d-1. LC-MS [M+H]⁺: m/z 649.4.

Step E—Synthesis of Intermediate 19e-1 To a mixture of potassium carbonate (373 mg, 2.7 mmol) in MeOH (6 mL) was added formic acid (0.20 mL, 5.4 mmol) at room temperature under N₂. The reaction was stirred at room temperature for 10 minutes, then added to a solution of intermediate 19d-1 (350 mg, 0.54 mmol) in AcOH (5.4 mL) and acetic anhydride (0.066 mL, 0.70 mmol). Pd/C (230 mg, 0.22 mmol) was added to the reaction, and the reaction was stirred at room temperature for 16 h, then filtered. The filtrate was concentrated under vacuum. The resulting residue was purified by reverse phase MPLC (Column: Biotage, Sfar C18D, 120 g; 50 m/min, 0-100% MeCN+0.05% TFA/H₂O+0.05% TFA). The product-containing fractions were combined, concentrated under vacuum without heating, and the resulting aqueous residue was lyophilized for 16 h to provide intermediate 19e-1. LC-MS [M+H]⁺: m/z 633.4.

Step F Synthesis of Intermediate 19f-1 A solution of intermediate 19e-1 (200 mg, 0.27 mmol) in DCM (2.7 mL) and TFA (2.7 mL) was stirred at room temperature for 16 h. Then the reaction was concentrated under vacuum without heating, and the resulting residue was dried under high vacuum for 2 hours to provide crude intermediate 19f-1, which was used without further purification. LC-MS [M+H]⁺: m/z 649.4.

Step G—Synthesis of Compounds 46 and 47 A mixture of intermediate 19f-1 (193 mg, 0.27 mmol), intermediate 5 (107 mg, 0.30 mmol), and oven-dried molecular sieves (4A, 500 mg) in MeOH (5.4 mL) was stirred at room temperature for 16 h. Then the reaction mixture was filtered through Celite™, and the filtrate was concentrated under vacuum without heating. The resulting residue was dissolved in water and purified on reverse phase MPLC (Column: Isco, Gold AqC18, 150 g; gradient elution with 0-30% MeCN+0.05% TFA/H₂O+0.05% TFA). The product fractions were collected and lyophilized to give compound 46 as its TFA salt. The TFA salt was converted to the formic acid salt by passing through reverse phase MPLC (Column: Gold C18-Aq, 50 g; gradient elution with 0-25% MeCN+0.1% FA/H₂O+0.1% FA). The product-containing fractions were collected and lyophilized to give compound 46 as its formic acid salt.

LC-MS [M+1]: m/z 723.2. ¹H NMR (500 MHz, D₂O/CD₃CN 4:1): 7.54 (s, 1H), 7.52 (d, J=9.0 Hz, 1H), 7.05 (d, J=8.6 Hz, 2H), 5.01 (s, 2H), 4.60 (d, J=11.4 Hz, 1H), 4.28 (t, J=9.6 Hz, 2H), 4.05 (d, J=7.6 Hz, 2H), 3.94 (d, J=5.9 Hz, 1H), 3.25 (dt, J=15.5, 7.4 Hz, 1H), 2.97 (s, 2H), 2.26 (d, J=13.3 Hz, 1H), 2.17 (t, J=7.1 Hz, 2H), 1.96 (s, 1H), 1.70 (s, 3H), 1.59 (s, 3H), 1.41 (d, J=5.8 Hz, 3H), 1.36 (s, 3H).

Compound 47 was analogously prepared as its formic acid salt starting from intermediate 19b-2 using the method described in Steps C to G of Example 29. LC-MS [M+H]⁺: m/z 723.3. ¹H NMR (500 MHz, D₂O/CD₃CN 4:1): 7.55 (s, 1H). 7.51 (d, J=8.8 Hz, 1H), 7.06 (d, J=8.3 Hz, 1H), 7.01 (s, 1H), 5.11 (s, 2H), 4.61 (d, J=11.8 Hz, 2H), 4.28 (t, J=9.1 Hz, 2H), 4.05 (s, 2H), 3.95 (s, 1H), 3.43 (s, 1H), 3.37-3.16 (m, 1H), 2.97 (s, 2H), 2.26 (d, J=14.2 Hz, 1H), 2.16 (d, J=9.4 Hz, 3H), 1.96 (s, 1H), 1.69 (s, 3H), 1.59 (s, 3H), 1.41 (d, J=6.2 Hz, 3H), 1.38 (s, 3H).

### Example 30: Preparation of Compounds 48 and 49

### (S)-2-((R)-6-(N—(((S)-1-amino-2,3-dihydro-1H-inden-5-yl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid and (S)-2-((R)-6-(N—(((R)-1-amino-2,3-dihydro-1H-inden-5-yl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

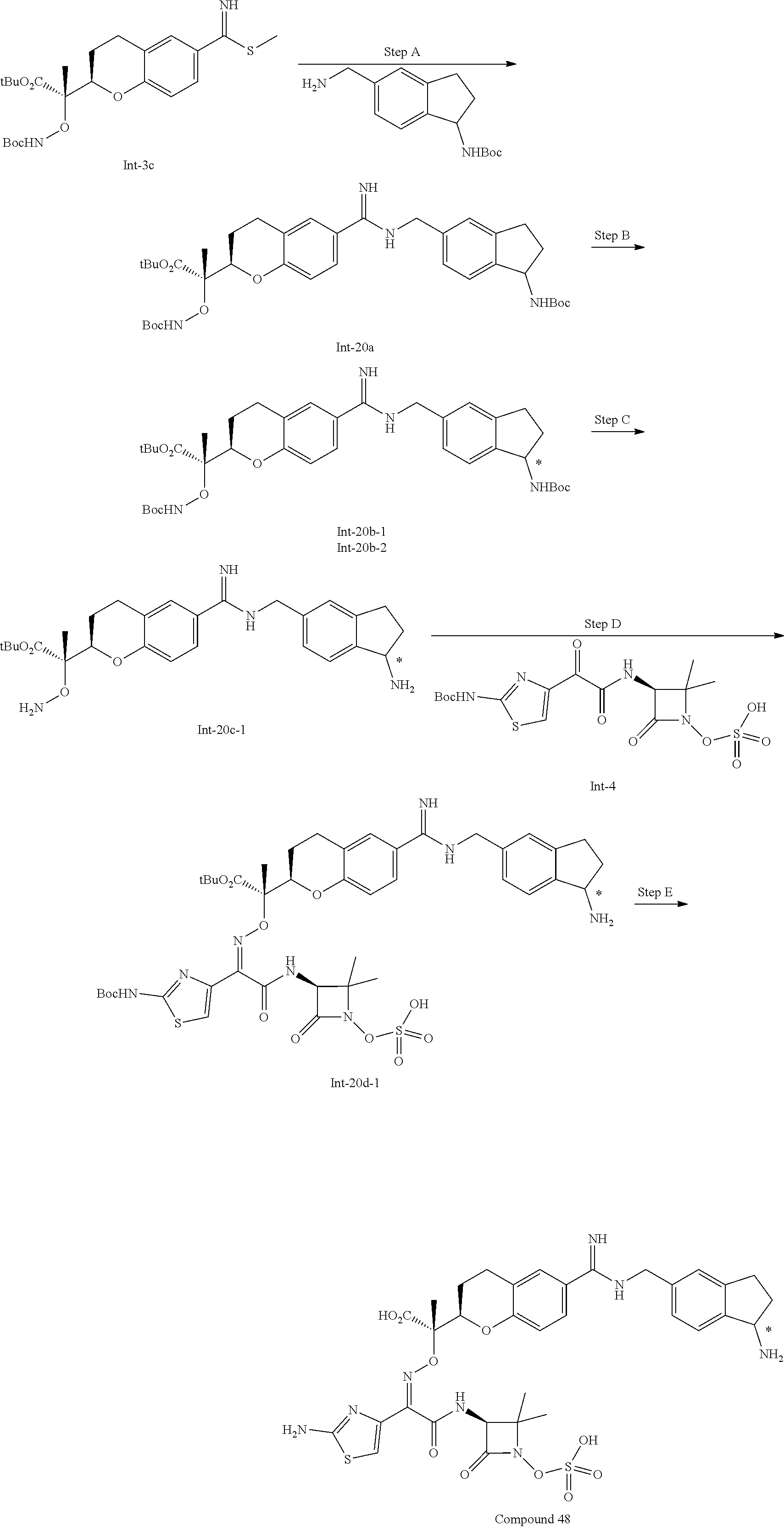

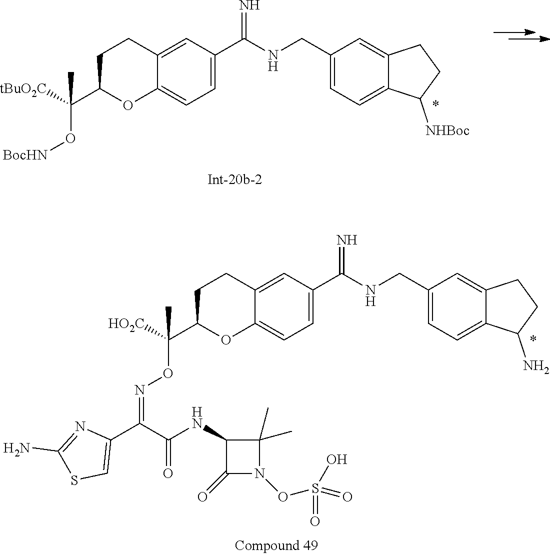 [SMILES: CC(C)(C)OC(=O)NO[C@](C)(C(=O)OC(C)(C)C)[C@H]1CCc2cc(C(=N)NCc3ccc4c(c3)CCC4NC(=O)OC(C)(C)C)ccc2O1 ; CC1(C)[C@H](NC(=O)/C(=N\O[C@](C)(C(=O)O)[C@H]2CCc3cc(C(=N)NCc4ccc5c(c4)CCC5N)ccc3O2)c2csc(N)n2)C(=O)N1OS(=O)(=O)O]

Step A—Synthesis of Intermediate 20a To a stirred mixture of tert-butyl (5-(aminomethyl)-2,3-dihydro-1H-inden-1-yl)carbamate (202 mg, 0.772 mmol), potassium acetate (189 mg, 1.929 mmol), and intermediate 3c (300 mg, 0.643 mmol) in MeOH (8 mL) was added acetic acid (0.147 mL, 2.57 mmol) at room temperature. The reaction was then stirred at 80° C. for 20 minutes. After cooling to room temperature, the reaction mixture was concentrated under reduced pressure, and the resulting residue was purified by flash silica gel chromatography (ISCO; 20 g Agela Silica Flash Column; Eluent of 0-8% MeOH/CH₂Cl₂ gradient; flow rate=20 mL/min) to give intermediate 20a. LC-MS (ESI) m/z: 681.3 [M+H]⁺.

Step B—Synthesis of Intermediates 20b-1 and 20b-2 The two stereoisomers of intermediate 20a (310 mg, 0.455 mmol) were separated by SFC (DAICEL CHIRALCEL OD (250 mm*30 mm, 10 um); Condition: Heptane/EtOH; Begin B, 10 End B 50; Gradient Time (min) 20; 100% B Hold Time (min) 5; FlowRate (mL/min) 20; Injections: 6) to give intermediates 20b-1 (faster eluting isomer; LC-MS (ESI) m/z: 681.4 [M+H]⁺) and 20b-2 (slower eluting isomer; LC-MS (ESI) m/z: 681.4 [M+H]⁺).

Step C—Synthesis of Intermediate 20c-1 To a solution of intermediate 20b-1 (270 mg, 0.397 mmol) in DCM (4 mL) was added TFA (2 mL, 26.0 mmol) dropwise at 0° C. The reaction was stirred at 25° C. for 60 min. Then the resulting mixture was concentrated under vacuum to give crude intermediate 20c-1, which was used without further purification. LC-MS (ESI) m/z: 481.3 [M+H]⁺.

Step D—Synthesis of Intermediate 2dc-1 To a solution of intermediate 20c-1 (200 mg, 0.416 mmol) in MeOH (4 mL) was added intermediate 4 (147 mg, 0.316 mmol). The reaction was stirred at 25° C. for 2 hours, then concentrated under vacuum to give crude intermediate 20d-1, which was used without further purification. LC-MS (ESI) m/z: 927.3 [M]⁺.

Step E Synthesis of Compounds 48 and 49 To a solution of intermediate 20d-1 (300 mg, 0.324 mmol) in DCM (0.75 mL) was added TFA (1.496 mL, 19.42 mmol) dropwise at 0° C. The resulting solution was stirred at 25° C. for 60 min, then concentrated under vacuum. The resulting residue was purified by a reverse phase HPLC (Boston Uni, C18, 40*150*5 um; Condition: water (0.1% TFA)/ACN; Begin B 1, End B 31; Gradient Time (min) 10; 100% B Hold Time (min) 2; FlowRate (mL/min) 60; Injections: 1) to give compound 48 as its TFA salt. The TFA salt was converted to its formic acid salt by passing through a reverse phase HPLC (Welch Xtimate, C18, 150*25 mm*5 um; Condition: water (0.225% FA)/ACN; Begin B 0, End B 20; Gradient Time (min) 10; 100% B Hold Time (min) 2; FlowRate (mL/min) 25; Injections: 3) to give compound 48 as its formic acid salt. LC-MS (ESI) m/z: 771.2 [M+H]⁺. ¹H NMR (400 MHz, DMSO-d₆) δ: ¹H NMR (400 MHz, DMSO-d₆) δ: 7.56-7.44 (m, 3H), 7.33 (s, 1H), 7.29 (br d, J=8.2 Hz, 1H), 6.97 (d, J=8.6 Hz, 1H), 6.75 (s. 1H), 4.66 (br t, J=6.8 Hz, 1H), 4.60 (s, 3H), 4.45 (br d, J=11.7 Hz, 1H), 3.10-2.98 (m, 1H), 2.90-2.67 (m, 3H), 2.49-2.37 (m, 1H), 2.10-1.90 (m, 2H), 1.66-1.50 (m, 1H), 1.49 (s, 3H), 1.39 (s, 3H), 1.23 (s, 3H).

Compound 49 was analogously prepared as its formic acid salt from intermediate 20b-2, following the method described in Steps C to E of Example 30. LC-MS (ESI) m/z: 771.2 [M+H]⁺. ¹H NMR (400 MHz, CD₃CN) δ: 7.49-7.41 (s, 3H), 7.36-7.24 (m, 2H), 6.93 (d, J=8.6 Hz, 1H), 6.78 (s, 1H), 4.77-4.72 (m, 1H), 4.59-4.52 (m, 3H), 4.45-4.40 (m, 1H), 3.13-3.01 (m, 1H), 2.91-2.75 (m, 3H), 2.58-2.45 (m, 1H), 2.13-2.02 (m, 2H), 1.80-1.62 (m, 1H), 1.52 (s, 3H), 1.41 (s, 3H), 1.25 (s, 3H).

### Example 31: Preparation of Intermediate 21e

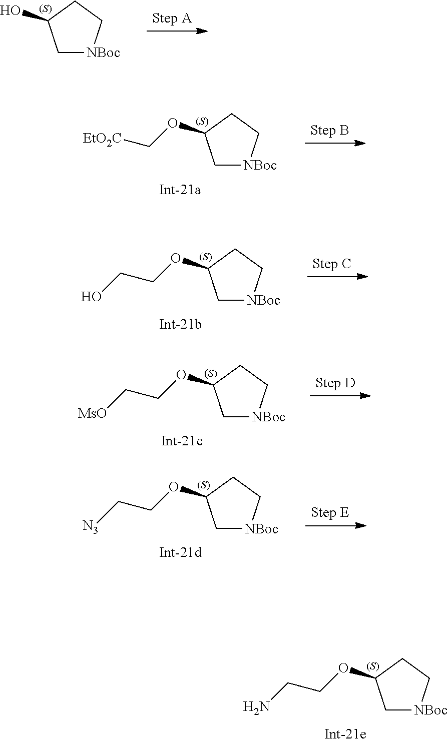

Step A—Synthesis of Intermediate 21a To a solution of sodium hydride (0.961 g, 40.1 mmol, 60%) in THF (50 mL) was added (S)-1-N-Boc-3-hydroxypyrrolidine (2.5 g, 13.35 mmol). The reaction mixture was stirred at 0° C. for 1 h before adding ethyl 2-chloroacetate (3.46 mL, 40.1 mmol) dropwise. Then the reaction mixture was stirred at 25° C. for 16 h, quenched with H₂O (200 mL), and extracted with EtOAc (3×200 mL). The combined organic layers were washed with brine (200 mL), dried over Na₂SO₄, filtered, and the filtrate was concentrated under reduced pressure. The resulting residue was purified by flash silica gel chromatography (ISCO®; Agela® Flash Column Silica-CS (80 g), Eluent of 0~20% Ethyl acetate/Petroleum ether gradient; flow rate=30 mL/min) to give intermediate 21a (2.3 g, 8.41 mmol). ¹H NMR (400 MHz, CDCl3) δ: 4.08-4.21 (m, 3H), 3.97-4.08 (m, 2H), 3.34-3.50 (m, 4H), 1.97-2.08 (m, 1H), 1.92 (d, J=8.22, 12.52 Hz, 1H), 1.42 (s, 9H), 1.26 (t, J=7.24 Hz, 3H).

Step B—Synthesis of Intermediate 21b To a solution of LiAlH₄ (0.383 g, 10.10 mmol) in THF (5 mL) was added dropwise intermediate 21a (2.3 g, 8.41 mmol) in THF (25 mL) over 0.5 h at 0° C. The reaction mixture was stirred at 0° C. for 1 h before quenching with H₂O (100 mL) and 10% NaOH (10 mL) at 0° C. The aqueous layer was extracted with EtOAc (3×100 mL). The combined organic layers were washed with brine (100 mL), dried over Na₂SO₄, filtered, and the filtrate was concentrated under reduced pressure. The resulting residue was purified by flash silica gel chromatography (ISCO®; Agela® Flash Column Silica-CS (40 g), Eluent of 0~50% ethyl acetate/petroleum ether gradient; flow rate=30 mL/min) to give intermediate 21b (1.3 g, 5.62 mmol). ¹H NMR (400 MHz, CDCl3) δ: 4.05 (s, 1H), 3.68-3.75 (m, 2H), 3.47-3.58 (m, 2H), 3.29-3.46 (m, 4H), 1.81-1.99 (m, 2H), 1.42 (s, 9H). LC-MS (ESI) m/z: 254.2 [M+Na]⁺.

Step C—Synthesis of Intermediate 21c To a solution of intermediate 21b (2.3 g, 9.94 mmol) in DCM (25 mL) were added dropwise triethylamine (4.16 mL, 29.8 mmol) and methanesulfonyl chloride (1.045 mL, 13.42 mmol) over 0.5 h at 0° C. The reaction mixture was stirred at 0° C. for 12 h. Then the reaction mixture was quenched with H₂O (60 mL) at 0° C. The aqueous layer was extracted with DCM (3×100 mL). The combined organic layers were washed with brine (60 mL), dried over Na₂SO₄, filtered and the filtrate was concentrated in vacuo. The resulting residue was purified by column chromatography (SiO₂, Pet. ether:EtOAc=20:1 to 10:1) to give intermediate 21c (3 g, 8.73 mmol). ¹H NMR (400 MHz, CDCl3) δ: 4.37-4.30 (m, 2H), 4.07 (br s, 1H), 3.73-3.65 (m, 2H), 3.49-3.32 (m, 4H), 3.02 (s, 3H), 2.01-1.87 (m, 2H), 1.44 (s, 9H).

Step D—Synthesis of Intermediate 21d To a solution of intermediate 21c (1.5 g, 4.85 mmol) in DMF (30 mL) were added NH₄Cl (1.556 g, 29.1 mmol) and sodium azide (1.261 g, 19.39 mmol). The reaction mixture was stirred at 60° C. for 16 h. Then the reaction mixture was diluted with water (40 mL) and extracted with ethyl acetate (100 mL×2). The combined organic layers were washed with brine (80 mL×2), dried over Na₂SO₄, filtered, and the filtrate was concentrated under N₂ gas flow to give crude intermediate 21d, which was used without further purification. LC-MS (ESI) m/z: 279.2 [M+Na]⁺.

Step E Synthesis of Intermediate 21e To a solution of intermediate 21d (4.85 mmol) in MeOH (30 mL) was added 10% Pd/C (0.581 g, 0.546 mmol). The reaction was stirred under 15 psi of H₂ at 20° C. for 12 h. Then the reaction mixture was filtered, and the filtrate was concentrated in vacuo to give intermediate 21e (1 g, 3.91 mmol), which was used in the next step without further purification. ¹H NMR (400 MHz, CDCl₃) δ: 4.03 (br s, 1H), 3.57-3.50 (m, 1H), 3.47-3.32 (m, 5H), 2.86-2.73 (m, 2H), 2.02-1.86 (m, 2H), 1.45 (d, J=1.2 Hz, 9H).

### Example 32: Preparation of Compound 50

### (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(2-(((S)-pyrrolidin-3-yl)oxy)ethyl)carbamimidoyl)chroman-2-yl)propanoic Acid

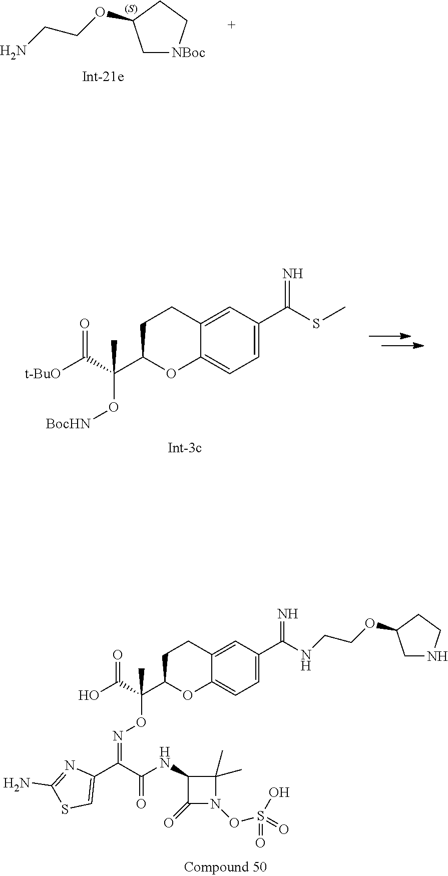

Compound 50 was prepared as its formic acid salt starting from intermediate 21e, using the procedure described in Steps A, C to E of Example 30. LC-MS (ESI) m/z: 739.2 [M+H]⁺. ¹H NMR (400 MHz, DMSO-D6) δ: 7.45-7.37 (m, 2H), 6.91 (d, J=8.5 Hz, 1H), 6.74 (s, 1H), 4.58 (s, 1H), 4.29 (br d, J=11.9 Hz, 1H), 4.21 (br s, 1H), 3.66-3.53 (m, 2H), 3.47 (br s, 2H), 3.32-3.07 (m, 4H), 2.84-2.65 (m, 2H), 2.07-1.83 (m, 3H), 1.60-1.48 (m, 1H), 1.46 (s, 3H), 1.38 (s, 3H), 1.22 (s, 3H).

### Example 33: Preparation of Intermediates 22d-trans-1, 22d-trans-2, 22d-cis-1 and 22d-cis-2

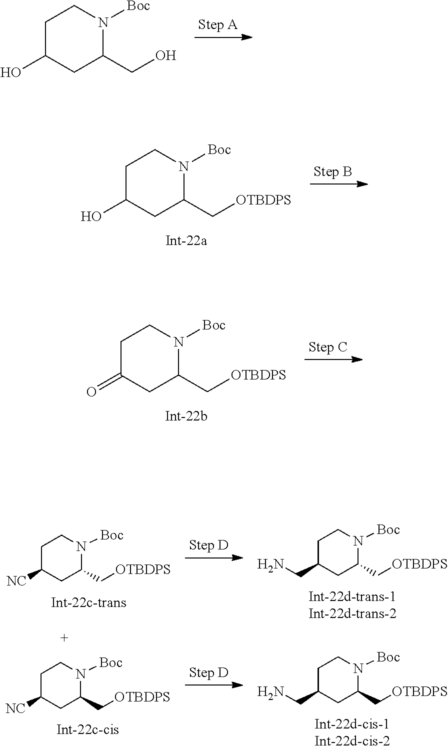

Step A—Synthesis of Intermediate 22a To a stirred solution of tert-butyl 4-hydroxy-2-(hydroxy-methyl)-piperidine-1-carboxylate (3.95 g, 17.08 mmol), imidazole (0.116 g, 1.708 mmol) and TEA (3.57 mL, 25.6 mmol) in DMF (50 mL) was added TBDPSCl (5.26 mL, 20.49 mmol) at 0° C. The reaction mixture was stirred at 20° C. for 12 h. Then the reaction content was diluted with water (250 mL) and extracted with EtOAc (80 mL×3). The organic layers were combined, washed with water (80 mL), brine (80 mL), dried over anhydrous Na₂SO₄, filtered, and the filtrate was concentrated under reduced pressure. The resulting residue was purified by flash silica gel chromatography (ISCO; 12 g Agela Silica Flash Column, Eluent of 0~12% EtOAc/Petroleum ether gradient; flow rate=30 mL/min) to give intermediate 22a. LC-MS (ESI) m/z: 470.2 [M+H]⁺.

Step B—Synthesis of Intermediate 22b To a solution of intermediate 22a (5.6 g, 11.92 mmol) in DCM (114 mL) stirred at 0° C., was added Dess Martin periodinane (7.59 g, 17.88 mmol) portion wise. The reaction mixture was stirred at 20° C. for 1 h, then quenched by addition of 10% Na₂S₂O₃ (200 mL) and stirred until the biphasic system turned clear. To the resulting mixture was added saturated NaHCO₃ (200 mL). The organic phase was isolated, and the aqueous layer was back-extracted with DCM (100 mL×3). The combined organic layers were washed with brine (100 mL), dried over anhydrous Na₂SO₄, filtered, and the filtrate was concentrated under reduced pressure. The resulting residue was purified by flash silica gel chromatography (ISCO; 20 g Agela Silica Flash Column, Eluent of 0~18% EtOAc/Petroleum ether gradient; flow rate=35 mL/min) to give intermediate 22b. LC-MS (ESI) m/z: 468.2 [M+H]⁺.

Step C—Synthesis of Intermediates 22c-trans and 22c-cis To a mixture of intermediate 22b (2.687 g, 5.75 mmol) and toluenesulfonylmethyl isocyanide (1.234 g, 6.32 mmol) in dimethoxyethane (38 mL) stirred at −15° C., was added a 1 M solution of t-BuOK in t-BuOH (8.62 mL, 8.62 mmol) dropwise under N₂. The reaction mixture was stirred at −15° C. for 2 h, then warmed to 28° C. and stirred for 12 h. The mixture was diluted with saturated aqueous NH₄Cl solution (150 mL), and extracted with EtOAc (50 mL×3). The organic layers were combined, washed with brine (50 mL), dried over anhydrous Na₂SO₄, filtered, and the filtrate was concentrated under reduced pressure. The resulting residue was purified by flash silica gel chromatography (ISCO; 12 g Agela Silica Flash Column, Eluent of 0~12% EtOAc/Petroleum ether gradient; flow rate=30 mL/min) to individually give intermediate 22c-trans (the first eluting isomer, LC-MS (ESI) m/z: 501.2 [M+Na]⁺) and intermediate 22c-cis (the second eluting isomer, LC-MS (ESI) m/z: 479.2 [M+H]⁺).

Step D—Preparation of Intermediates 22d-trans-1, 22d-trans-2, 22d-cis-1 and 22d-cis-2 A solution of intermediate 22c-trans (2.34 g, 4.89 mmol) in 7 N NH₃/MeOH (50 mL) was hydrogenated (30 psi) at 27° C. with wet Raney Nickel (1 g) for 12 h. Then the reaction mixture was filtered, and the filtrate was concentrated under reduced pressure. The resulting residue was separated by chiral SFC (DAICEL CHIRALPAK IG 250 mm*50 mm, 10 um; Condition: 0.1% NH₃·H₂O/IPA; Begin B 25%, End B 25%; FlowRate (mL/min) 200; Injections 100) to give intermediates 22d-trans-1 (the first eluting isomer, LC-MS (ESI) m/z: 483.3 [M+H]⁺), and 22d-trans-2 (the second eluting isomer, LC-MS (ESI) m/z: 483.3 [M+H]⁺).

Intermediates 22d-cis-1 and 22d-cis-2 were prepared from intermediate 22c-cis by using the procedure illustrated in Step D of Example 33, and the enantiomers were separated by chiral SFC (Column: REGIS (s,s) WHELK-01 (250 mm*50 mm, 10 um; Condition: 0.1% NH₃·H₂O/EtOH; Begin B 25%, End B 25%; FlowRate (mL/min) 200; Injections 300) to give intermediates 22d-cis-1 (the first eluting isomer, LC-MS (ESI) m/z: 483.3 [M+H]⁺), and 22d-cis-2 (the second eluting isomer, LC-MS (ESI) m/z: 483.3 [M+H]⁺).

### Example 34: Preparation of Compounds 51 to 54

### (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4S)-2-(hydroxymethyl)piperidin-4-yl)methyl)carbamimidoyl)chroman-2-yl)propanoic Acid, (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4R)-2-(hydroxymethyl)piperidin-4-yl)methyl)carbamimidoyl)chroman-2-yl)propanoic acid, (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4R)-2 -(hydroxymethyl)piperidin-4-yl)methyl)carbamimidoyl)chroman-2-yl)propanoic acid, and (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4S)-2-(hydroxymethyl)piperidin-4-yl)methyl)carbamimidoyl)chroman-2-yl)propanoic Acid

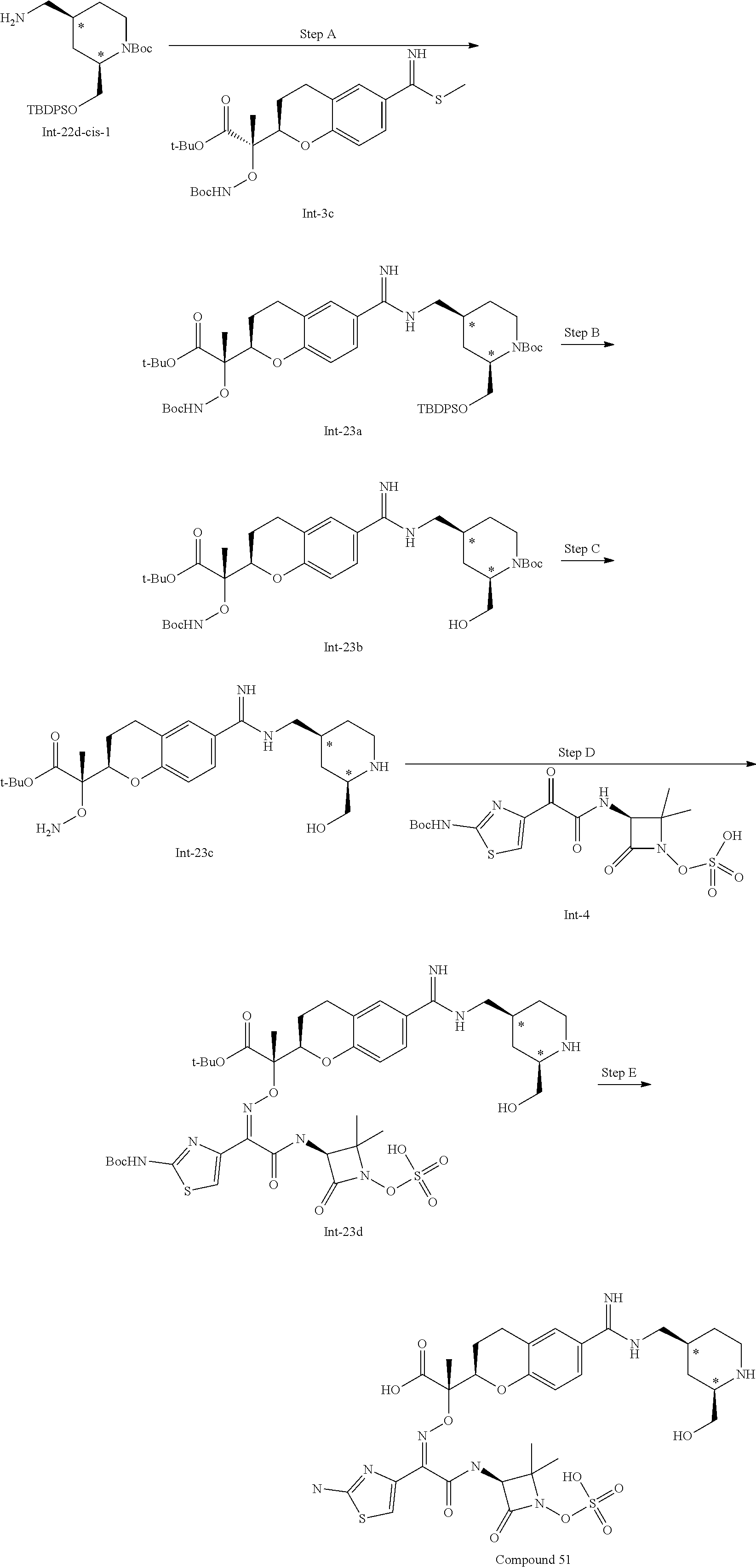

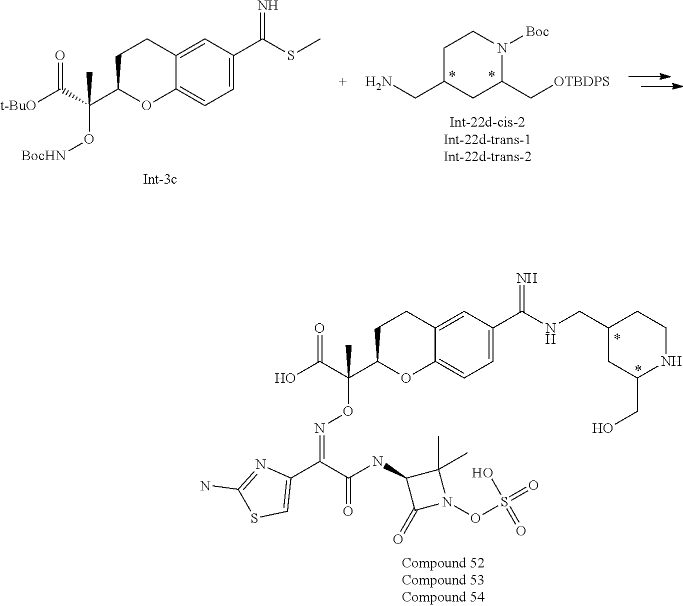

Step A—Synthesis of Intermediate 23a To a stirred solution of intermediate 3c (130 mg, 0.279 mmol) and intermediate 22d-cis-1 (130 mg, 0.269 mmol) in EtOH (4 mL) was added acetic acid (0.08 mL, 1.397 mmol) and potassium acetate (55 mg, 0.560 mmol) sequentially at 25° C. The reaction was stirred at 85° C. for 1 h. Then the reaction mixture was diluted with water (20 mL), extracted with EtOAc (8 mL×4), and dried over anhydrous Na₂SO₄. After filtration, the filtrate was concentrated in vacuo to afford crude intermediate 23a, which was used in the next step without further purification. LC-MS (ESI) m/z: 901.4 [M+H]⁺.

Step B—Synthesis of Intermediate 23b A solution of intermediate 23a (290 mg, 0.322 mmol) and tetrabutylammonium fluoride (1.5 mL, 1.500 mmol, 1 N in THF) in THF (6 mL) was stirred at 25° C. for 30 min. Then the reaction solution was concentrated under vacuum, and the resulting residue was purified by reverse phase HPLC (Column Boston Uni, C18, 40*150*5 um; Condition: water (0.1% TFA)-ACN; Begin B 30, End B 60; Gradient Time (min) 11; 100% B Hold Time (min) 2; FlowRate (mL/min) 60; Injections 1) to afford intermediate 23b. LC-MS (ESI) m/z: 663.5 [M+H]⁺.

Step C—Synthesis of Intermediate 23c A solution of intermediate 23b (85 mg, 0.128 mmol) in 3 mL of DCM/TFA (2:1) was stirred at 27° C. for 30 min. Then the solvent was removed via nitrogen gas flow to afford crude intermediate 23c, which was used in the next step without further purification. LC-MS (ESI) m/z: 463.3 [M+H]⁺.

Step D—Synthesis of Intermediate 23d A solution of intermediate 23c (59 mg, 0.128 mmol) and intermediate 4 (60 mg, 0.129 mmol) in MeOH (4 mL) was stirred at 25° C. for 45 min. Then the reaction mixture was concentrated under vacuum to afford crude intermediate 23d, which was used in the next step without further purification. LC-MS (ESI) m/z: 909.4 [M+H]⁺.

Step E—Synthesis of Compound 51 A solution of intermediate 23d (120 mg, 0.066 mmol) in TFA (3 mL) and DCM (1 mL) was stirred at 25° C. for 45 min. Then the reaction mixture was dried under a nitrogen gas flow. The resulting residue was dissolved in 2.5 mL of DMSO/MeCN (3:1), and directly purified by reverse phase HPLC (Column: Boston Uni C18 40×150×5 um; Condition: water (0.1% TFA)-ACN; Begin B 1, End B 31; Gradient Time (min) 11; 100% B Hold Time (min) 2; FlowRate (mL/min) 60; Injections 1) to afford the desired product as its TFA salt, which was converted to its formic acid salt by passing through a reverse phase HPLC (Column: YMC-Actus Triart C18 150×30 mm×5 um; Condition: water (0.225% FA)-ACN; Begin B 0, End B 20; Gradient Time (min) 19; 100% B Hold Time (min) 2; FlowRate (mL/min) 25; Injections 3) to give compound 51 as its formic acid salt. LC-MS (ESI) m/z: 753.2 [M+H]⁺. ¹H NMR (400 MHz, DMSO-d₆): δ 7.49-7.37 (m, 2H), 6.95 (d, J=8.2 Hz, 1H), 6.74 (s, 1H), 4.61 (s, 1H), 4.43 (br d, J=12.9 Hz, 1H), 3.59-3.52 (m, 1H), 3.47-3.35 (m, 1H), 3.32-3.18 (m, 3H), 3.12-3.02 (m, 1H), 2.91-2.73 (m, 3H), 2.02 (br s, 2H), 1.91-1.79 (m, 2H), 1.65-1.51 (m, 1H), 1.48 (s, 3H), 1.38 (s, 3H), 1.30-1.26 (m, 1H), 1.22 (s, 3H), 1.09-1.16 (m, 1H).

Compounds 52 to 54 were analogously prepared as formic acid salts starting from intermediate 3c and their respective amines prepared in Example 33, using the method described in Steps A to E in Example 34.

Compound 52 (from intermediate 22d-cis-2): LC-MS (ESI) m/z: 753.2 [M+H]⁺. ¹H NMR (400 MHz, DMSO-d₆) δ: 7.48-7.38 (m, 2H), 6.93 (d, J=8.6 Hz, 1H), 6.74 (s, 1H), 4.59 (s, 1H), 4.40 (br d, J=12.9 Hz, 1H), 3.63-3.56 (m, 1H), 3.46-3.37 (m, 1H), 3.32-3.24 (m, 3H), 3.13-3.03 (m, 1H), 2.93-2.71 (m, 3H), 2.11-1.91 (m, 2H), 1.90-1.76 (m, 2H), 1.65-1.51 (m, 1H), 1.47 (s, 3H), 1.38 (s, 3H), 1.31-1.26 (m, 1H), 1.22 (s, 3H), 1.20-1.05 (m, 1H).

Compound 53 (from intermediate 22d-trans-1): LC-MS (ESI) m/z: 752.8 [M+H]⁺. ¹H NMR (400 MHz, D₂O+CD₃CN) δ: 7.42-7.34 (m, 2H), 6.88 (d, J=8.6 Hz, 1H), 6.84 (s, 1H), 4.60 (s, 1H), 4.40 (br d, J=9.4 Hz, 1H), 3.69 (d, J=6.7 Hz, 2H), 3.48-3.43 (m, 1H), 3.36 (d, J=7.8 Hz, 2H), 3.22-3.13 (m, 2H), 2.87-2.74 (m, 2H), 2.30-2.21 (m, 1H), 2.11-2.01 (m, 1H), 1.88-1.56 (m, 5H), 1.50 (s, 3H), 1.40 (s, 3H), 1.21 (s, 3H).

Compound 54 (from intermediate 22d-trans-2): LC-MS (ESI) m/z: 753.1 [M+H]⁺. ¹H NMR (400 MHz, D₂O+CD₃CN) δ: 7.45-7.33 (m, 2H), 6.88 (d, J=8.6 Hz, 1H), 6.84 (s, 1H), 4.60 (s, 1H), 4.40 (br d, J=9.0 Hz, 1H), 3.68 (d, J=6.7 Hz, 2H), 3.51-3.44 (m, 1H), 3.35 (d, J=7.8 Hz, 2H), 3.19-3.11 (m, 2H), 2.88-2.69 (m, 2H), 2.31-2.20 (m, 1H), 2.12-2.04 (m, 1H), 1.87-1.55 (m, 5H), 1.50 (s, 3H), 1.41 (s, 3H), 1.22 (s, 3H).

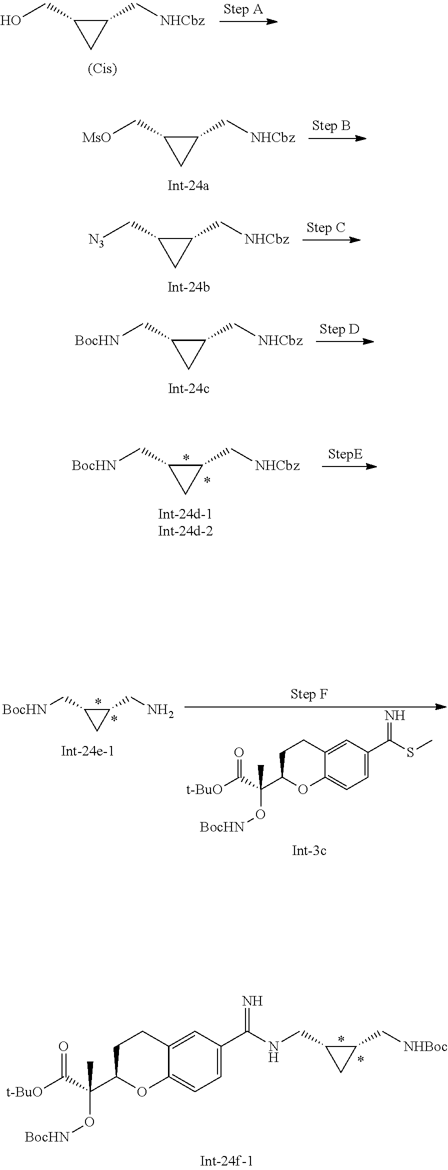

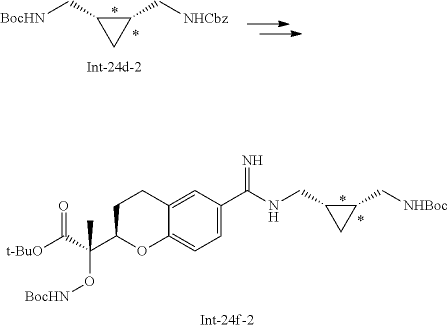 [SMILES: CC(C)(C)OC(=O)NC[C@H]1C[C@H]1CNC(=O)OCc1ccccc1 ; CC(C)(C)OC(=O)NC[C@@H]1C[C@@H]1CNC(=N)c1ccc2c(c1)CC[C@H]([C@](C)(ONC(=O)OC(C)(C)C)C(=O)OC(C)(C)C)O2]

Step A—Synthesis of Intermediate 24a To a solution of (cis)-benzyl ((2-(hydroxymethyl)-cyclopropyl)-methyl)carbamate (2.5 g, 10.63 mmol) and triethylamine (2.96 mL, 21.25 mmol) in THF (85 mL) stirred at 0° C., was added dropwise a solution of methanesulfonyl chloride (1.970 g, 17.20 mmol) in THF (5 mL). The reaction was warmed to 20° C. and stirred for 1.5 h, then cooled at 0° C. and quenched by the slow addition of saturated aqueous NaHCO₃ solution (120 mL). The resulting mixture was extracted with MTBE (80 mL×3). The organic layers were combined, dried over anhydrous Na₂SO₄, filtered, and the filtrate was concentrated under reduced pressure to give crude intermediate 24a, which was used in the next step without further purification. ¹H NMR (400 MHz, CDCl3) δ: 7.44-7.30 (m, 5H), 5.12 (s, 2H), 4.55 (br dd, J=11.0, 6.1 Hz, 1H), 4.02 (br t, J=10.4 Hz, 1H), 3.66-3.51 (m, 1H), 3.07-2.93 (m, 3H), 1.43-1.23 (m, 3H), 0.99-0.90 (m, 1H), 0.33 (q, J=5.5 Hz, 1H).

Step B—Synthesis of Intermediate 24b To a solution of intermediate 24a (3.33 g. 10.63 mmol) in DMF (45 mL) stirred at 20° C. was added NaN₃ (1.440 g, 22.15 mmol) in one portion. The reaction mixture was warmed to 70° C., and stirred for 12 h. Then the reaction mixture was partitioned between water (200 mL) and EtOAc (80 mL) and the organic layer was separated. The aqueous layer was further extracted with EtOAc (80 mL×2). The combined organic layers were washed with brine (80 mL×2), dried over anhydrous Na₂SO₄, filtered and the filtrate was concentrated in vacuo to give crude intermediate 24b, which was used in the next step without further purification. ¹H NMR (400 MHz, CDCl3) δ: 7.45-7.29 (m, 5H), 5.12 (s, 3H), 3.58 (dt, J=14.2, 6.5 Hz, 2H), 3.07 (br dd, J=12.9, 9.0 Hz, 1H), 3.00-2.91 (m, 1H), 1.31-1.14 (m, 2H), 0.87 (dt, J=8.4, 5.5 Hz, 1H), 0.23 (q, J=5.3 Hz, 1H).

Step C—Synthesis of Intermediate 24c To a solution of intermediate 24b (2.77 g, 10.64 mmol) in water (10 mL) and THF (40 mL) stirred at 20° C. was added triphenylphosphine (4.19 g, 15.96 mmol). The reaction was stirred at 20° C. for 12 h, then the reaction mixture was concentrated under reduced pressure. The resulting residue was dissolved in DCM (83 mL). and cooled to 0° C. Then triethylamine (4.45 mL, 31.9 mmol) was added, followed by the dropwise addition of (Boc)₂O (4.94 mL, 21.28 mmol). The reaction mixture was stirred at 25° C. for 12 h, then diluted with DCM (250 mL), washed with brine (100 mL), dried over anhydrous Na₂SO₄, and filtered. The filtrate was concentrated under reduced pressure. The resulting residue was purified by flash silica gel chromatography (ISCO; 80 g Agela Silica Flash Column, Eluent of 0-16% EtOAc/Pet. Ether gradient @ 60 mL/min) to give intermediate 24c. LC-MS (ESI) m/z: 357.2 [M+Na]⁺.

Step D—Synthesis of Intermediate 24d-1 and 24d-2 Enantiomers of intermediate 24c (1.6 g, 4.78 mmol) were further separated by SFC (DAICEL CHIRALPAK IG, 250 mm*30 mm, 10 um; Condition: 0.1% NH₃·H₂O MeOH; Begin B 30%, End B 30%; Flow Rate (mL/min) 220; Injections 200) to give intermediate 24d-1 (the first eluting isomer, LC-MS (ESI) m/z: 357.2 [M+Na]⁺), and intermediate 24d-2 (the second eluting isomer, LC-MS (ESI) m/z: 357.2 [M+Na]⁺).

Step E—Synthesis of Intermediate 24e-1 A solution of intermediate 24d-1 (200 mg, 0.598 mmol), AcOH (0.103 mL, 1.794 mmol) and 10 wt % Pd—C (100 mg, 0.094 mmol) in MeOH (15 mL) was stirred at 25° C. under hydrogen atmosphere (15 psi) for 1.5 h. Then the reaction mixture was filtered and the filtrate was concentrated under vacuum to give intermediate 24e-1, which was used in the next step without further purification. ¹H NMR (400 MHz, CDCl3) δ: 3.39 (br d, J=13.3 Hz, 1H), 3.22 (br d, J=8.6 Hz, 1H), 2.88 (br s, 1H), 2.73 (dd, J=13.5, 10.0 Hz, 1H), 1.44 (s, 9H), 1.30-1.06 (m, 2H), 0.93-0.75 (m, 1H), 0.16-0.00 (m, 1H).

Step F—Synthesis of Intermediate 24f-1 and 24f-2 To a stirred solution of intermediate 3c (280 mg, 0.600 mmol) and intermediate 24e-1 (120 mg, 0.600 mmol) in MeOH (5 mL) were added acetic acid (0.137 mL, 2.400 mmol) and potassium acetate (177 mg, 1.800 mmol) sequentially at 25° C. The reaction was stirred at 85° C. for 25 min. Then the reaction mixture was diluted with water (20 mL), and extracted with EtOAc (20 mL×4). The organic layers were combined, dried over anhydrous Na₂SO₄, and filtered. The filtrate was concentrated under reduced pressure to give crude intermediate 24f-1, which was used in the next step without further purification. LC-MS (ESI) m/z: 619.4 [M+H]⁺.

Intermediate 24f-2 was prepared from intermediate 24d-2 using a procedure similar to the method illustrated in Step E and Step F of Example 35. LC-MS (ESI) m/z: 619.4 [M+H]⁺.

### Example 36: Preparation of Compounds 55 and 56

### (S)-2-((R)-6-(N-(((1S,2R)-2-(aminomethyl)cyclopropyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid and (S)-2-((R)-6-(N-(((1R,2S)-2-(aminomethyl)cyclopropyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

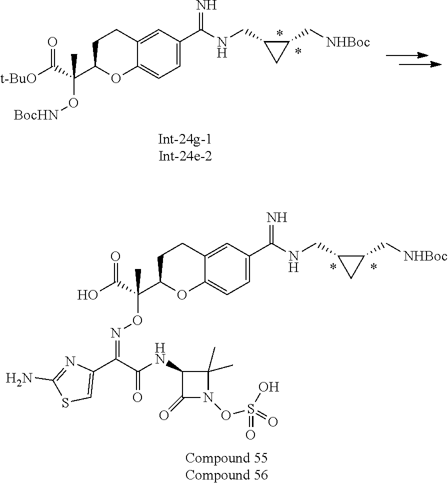 [SMILES: CC(C)(C)OC(=O)NC[C@@H]1C[C@@H]1CNC(=N)c1ccc2c(c1)CC[C@H]([C@](C)(ONC(=O)OC(C)(C)C)C(=O)OC(C)(C)C)O2 ; CC(C)(C)OC(=O)NC[C@@H]1C[C@@H]1CNC(=N)c1ccc2c(c1)CC[C@H]([C@](C)(O/N=C(\C(=O)N[C@@H]1C(=O)N(OS(=O)(=O)O)C1(C)C)c1csc(N)n1)C(=O)O)O2]

Compounds 55 and 56 were prepared from the corresponding intermediate 24f-1 and 24f-2 using similar method as illustrated in Step C to Step E of Example 34.

Compound 55 (from intermediate 24f-1): LC-MS (ESI) m/z: 709.2 [M+H]⁺. ¹H NMR (400 MHz, D₂O+CD₃CN) δ: 7.44-7.35 (m, 2H), 6.89 (d, J=8.2 Hz, 1H), 6.81 (s, 1H), 4.60 (s, 1H), 4.42-4.38 (m, 1H), 3.43-3.33 (m, 1H), 3.32-3.20 (m, 2H), 2.91-2.63 (m, 3H), 2.12-2.02 (m, 1H), 1.80-1.62 (m, 1H), 1.50 (s, 3H), 1.47-1.31 (m, 4H), 1.30-1.12 (m, 4H), 1.00-0.87 (m, 1H), 0.44-0.32 (m, 1H).

Compound 56 (from intermediate 24f-2): LC-MS (ESI) m/z: 709.1 [M+H]⁺. ¹H NMR (400 MHz, D₂O+CD₃CN) δ: 7.46-7.33 (m, 2H), 6.89 (d, J=8.2 Hz, 1H), 6.81 (s, 1H), 4.60 (s, 1H), 4.39 (br d, J=11.3 Hz, 1H), 3.43-3.34 (m, 1H), 3.33-3.20 (m, 2H), 2.89-2.62 (m, 3H), 2.10-2.02 (m, 1H), 1.79-1.60 (m, 1H), 1.49 (s, 3H), 1.45-1.35 (m, 4H), 1.30-1.15 (m, 4H), 1.02-0.89 (m, 1H), 0.43-0.33 (m, 1H).

### Example 37: Preparation of Intermediates 25d-1 and 25d-2

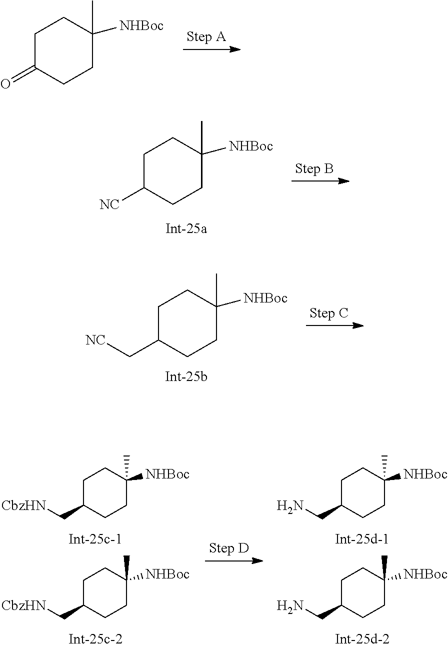

Step A—Synthesis of Intermediate 25a To a solution of tert-butyl (1-methyl-4-oxocyclohexyl)-carbamate (2 g, 8.80 mmol) and 1-((isocyanomethyl)sulfonyl)-4-methylbenzene (1.890 g, 9.68 mmol) in DME (20 mL), stirred at −20° C., was added dropwise potassium 2-methylpropan-2-olate (1 M in t-BuOH, 15.84 mL, 15.84 mmol) under N₂. The reaction was warmed to 20° C. and stirred for 12 h. Then the reaction mixture was diluted with saturated aqueous NH₄Cl solution (60 mL), and extracted with EtOAc (90 mL×3). The organic layers were combined, washed with brine (60 mL), dried over anhydrous Na₂SO₄, and filtered. The filtrate was concentrated under reduced pressure. The resulting residue was purified by silica gel chromatography eluting with petroleum ether/EtOAc (20:1 to 10:1 gradient) to give intermediate 25a. ¹H NMR (400 MHz, CDCl3) δ: 4.27 (br s, 1H), 2.77 (br t, J=4.7 Hz, 0.5H), 2.50-2.39 (m, 0.5H), 2.17-2.08 (m, 1H), 2.05-1.96 (m, 1H), 1.93-1.73 (m, 4H), 1.71-1.55 (m, 2H), 1.63-1.43 (m, 9H), 1.33-1.31 (m, 3H).

Step B—Synthesis of Intermediate 25b To a solution of intermediate 25a (2.9 g, 12.17 mmol) in THF (25 mL) was added LiAlH₄ (0.748 g, 19.71 mmol) at 0° C. The reaction was stirred at 25° C. for 2.5 h. Then the reaction was quenched with 1 M aqueous NaOH solution (20 mL) and H₂O (10 mL) at 0° C. The resulting mixture was extracted with ethyl acetate (3×150 mL). The combined organic layer was dried over anhydrous MgSO₄, filtered, and the filtrate was concentrated in vacuo to give intermediate 25b, which was used in the next reaction without further purification. LC-MS (ESI) m/z: 243.2 [M+H]⁺.

Step C—Synthesis of Intermediates 25c-1 and 25c-2 To a solution of intermediate 25b (1.4 g, 5.78 mmol) in THF (16 mL) and water (8 mL) were added Na₂CO₃ (1.837 g, 17.33 mmol) and benzyl chloroformate (1.024 mL, 7.51 mmol) at 0° C. The reaction mixture was stirred at 20° C. for 12 h, then diluted with water (50 mL) and extracted with ethyl acetate (130 mL×3). The combined organic layer was washed with brine (60 mL), dried over anhydrous Na₂SO₄, and filtered. The filtrate was concentrated in vacuo and the resulting residue was purified by silica gel chromatography eluting with petroleum ether:EtOAc (8:1 to 5:1 gradient) to give the product as a mixture of cis and trans isomers. The cis/trans mixture was further separated by SFC (Column: DAICEL CHIRALCEL OJ (250 mm*50 mm, 10 um); Conditions: 0.1% NH₃·H₂O/EtOH; Begin: B 30%, End: B 30%; FlowRate (mL/min): 200; Injections: 300) to individually give intermediates 25c-1 (the first eluting isomer) and 25c-2 (the second eluting isomer).

Intermediate 25c-1: ¹H NMR (400 MHz, CDCl3) δ: 7.42-7.28 (m, 5H), 5.13-5.06 (m, 2H), 4.82 (br s, 1H), 4.26 (br s, 1H), 3.07 (br t, J=6.5 Hz, 2H), 2.09 (br d, J=12.1 Hz, 2H), 1.65 (br d, J=11.0 Hz, 1H), 1.56 (br d, J=13.7 Hz, 2H), 1.43 (s, 9H), 1.29 (s, 3H), 1.21-1.06 (m, 4H).

Intermediate 25c-2: ¹H NMR (400 MHz, CDCl3) δ: 7.40-7.29 (m, 5H), 5.09 (s, 2H), 4.77 (br s, 1H), 4.44 (br s, 1H), 3.09 (br t, J=6.5 Hz, 2H), 1.83 (br d, J=12.5 Hz, 2H), 1.63 (br t, J=11.9 Hz, 5H), 1.43 (s, 9H), 1.29 (s, 3H), 1.19-1.07 (m, 2H).

Step D—Synthesis of Intermediate 25d-1 and 25d-2 To a solution of intermediate 25c-1 (150 mg, 0.398 mmol) in MeOH (20 mL) was added 10 wt % Pd/C (12.72 mg, 0.120 mmol). The reaction was stirred at 20° C. under 15 psi of H₂ for 13 hours. Then the reaction mixture was filtered and the filtrate was concentrated in vacuo to give crude intermediate 25d-1, which was used in the next step without further purification. LC-MS (ESI) m/z: 243.2 [M+H]⁺.

Intermediate 25d-2 was analogously prepared from intermediate 25c-2 using the method described in Step D of Example 37. LC-MS (ESI) m/z: 243.2 [M+H]⁺.

### Example 38: Preparation of Compounds 57 and 58

### (S)-2-((R)-6-(N-(((1s,4S)-4-amino-4-methylcyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid and (S)-2-((R)-6-(N-(((1r,4R)-4-amino-4-methylcyclohexyl)methyl)carbamimidoyl)chroman-2-yl)-2-((((Z)-1-(2-aminothiazol-4-yl)-2 -(((S)-2,2-dimethyl-4-oxo-1-(sulfooxy)azetidin-3-yl)amino)-2-oxoethylidene)amino)oxy)propanoic Acid

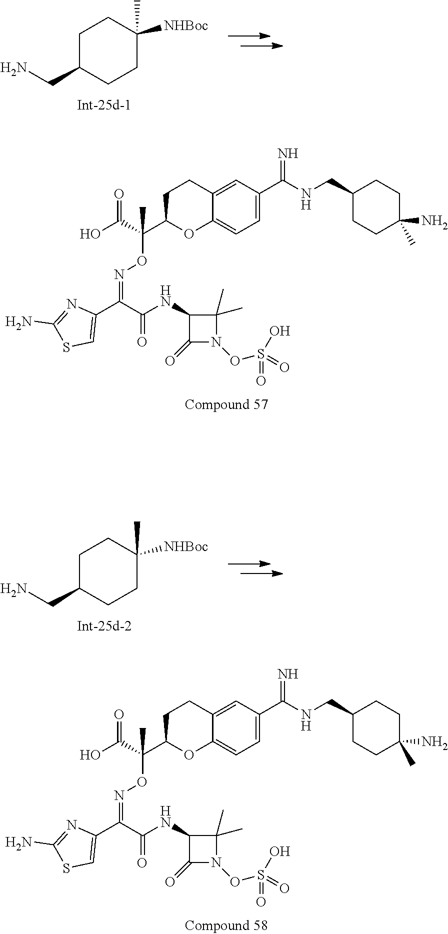 [SMILES: CC(C)(C)OC(=O)N[C@]1(C)CC[C@H](CN)CC1 ; CC1(C)[C@H](NC(=O)/C(=N\O[C@](C)(C(=O)O)[C@H]2CCc3cc(C(=N)NC[C@H]4CC[C@@](C)(N)CC4)ccc3O2)c2csc(N)n2)C(=O)N1OS(=O)(=O)O ; CC(C)(C)OC(=O)N[C@]1(C)CC[C@@H](CN)CC1 ; CC1(C)[C@H](NC(=O)/C(=N\O[C@](C)(C(=O)O)[C@H]2CCc3cc(C(=N)NC[C@H]4CC[C@](C)(N)CC4)ccc3O2)c2csc(N)n2)C(=O)N1OS(=O)(=O)O]

Compounds 57 and 58 were analogously prepared as formic acid salts starting from their corresponding intermediates 25d-1 and 25d-2 according to the method described in Steps A to D of Example 12.

Compound 57: LC-MS (ESI) m/z: 751.2 [M+H]⁺. ¹H NMR (400 MHz, CD₃CN) β: 7.42-7.34 (m, 2H), 6.89 (d, J=8.2 Hz, 1H), 6.78 (s, 1H), 4.60 (s, 1H), 4.39 (br d, J=11.3 Hz, 1H), 3.23 (br d, J=6.7 Hz, 2H), 2.87-2.70 (m, 2H), 2.12-2.02 (m, 1H), 1.85-1.60 (m, 6H), 1.57-1.48 (m, 5H), 1.42 (s, 3H), 1.31-1.15 (m, 8H).

Compound 58: LC-MS (EST) m/z: 751.2 [M+H]⁺. ¹H NMR (400 MHz, CD₃CN) β: 7.42-7.35 (m, 2H), 6.90 (d, J=8.2 Hz, 1H), 6.78 (s, 1H), 4.60 (s, 1H), 4.40 (br d, J=9.8 Hz, 1H), 3.21 (br d, J=6.7 Hz, 2H), 2.88-2.70 (m, 2H), 2.12-2.02 (m, 1H), 1.80-1.61 (m, 6H), 1.60-1.52 (m, 2H), 1.50 (s, 3H), 1.42 (s, 3H), 1.32-1.11 (m, 8H).

### Example 39: Preparation of Compound 59

### (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-((4-(2-methoxyethyl)piperidin-4-yl)methyl)carbamimidoyl)chroman-2-yl)propanoic Acid

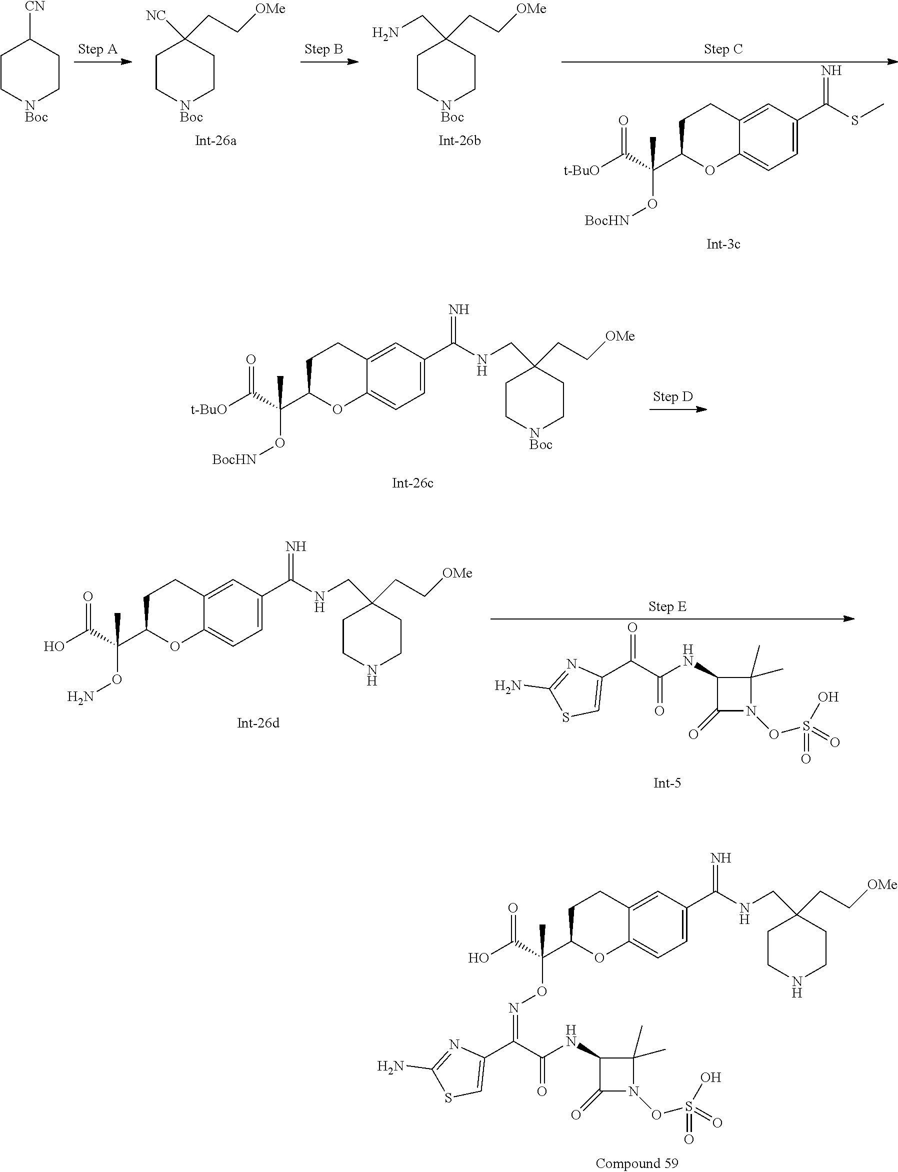

Step A—Synthesis of Intermediate 26a To a solution of 1-boc-4-cyanopiperidine (5 g, 23.78 mmol) in THF (50 mL), stirred at −10° C., was added dropwise a solution of 1.0 M LiHMDS in THF (35.7 mL, 35.7 mmol). Then a solution of 2-bromoethyl methyl ether (3.35 mL, 35.7 mmol) in THF (20 mL) was added dropwise while maintaining the temperature at −10° C. After the addition, the reaction mixture was allowed to warm to 28° C. and stirred for an additional 2 h. Then the reaction mixture was diluted with EtOAc (60 mL). The organic layer was separated and washed with H₂O (100 mL). The aqueous layer was back-extracted with EtOAc (60 mL×2). The combined organic layers were washed with brine (100 mL) and dried over anhydrous Na₂SO₄. After filtration, the filtrate was concentrated under reduced pressure to give the crude intermediate 26a, which was used in the next step without further purification. LC-MS (ESI) m/z: 291.0 [M+Na]⁺.

Step B—Synthesis of Intermediate 26b To a solution of intermediate 26a (6.38 g, 23.77 mmol) in 7 M NH₃ in MeOH (60 mL) was added Raney Nickel (1.395 g, 23.77 mmol). The reaction mixture was stirred at 30° C. under H₂ (45 psi) for 16 h. Then the reaction mixture was filtered, and the filtrate was concentrated under reduced pressure to give crude intermediate 26b, which was used in the next reaction without further purification. LC-MS (ESI) m/z: 273.2 [M+H]⁺.

Step C—Synthesis of Intermediate 26c To a solution of intermediate 3c (0.3 g, 0.643 mmol) in MeCN (3 mL) were added potassium acetate (0.189 g, 1.929 mmol), intermediate 26b (0.263 g, 0.964 mmol) and acetic acid (0.147 mL, 2.57 mmol) sequentially under N₂ atmosphere. The reaction mixture was stirred at 80° C. for 15 min, then filtered, and the filtrate was concentrated under reduced pressure. The resulting residue was purified by reverse phase MPLC (Biotage; 20 g Agela C18, 20-35 pm, Eluent of 30% MeCN/H₂O+0.5‰ TFA gradient, flow rate=50 m/min) to give intermediate 26c. LC-MS (ESI) m/z: 692.0 [M+H]⁺.

Step D—Synthesis of Intermediate 26d A mixture of intermediate 26c (280 mg, 0.405 mmol) in 37% aqueous HCl solution (2 mL) and DCM (1 mL) was stirred at 25° C. for 50 min. Then the reaction mixture was concentrated in vacuo. The resulting residue was purified by reverse phase HPLC (Column: Boston Uni C18 40×150×5 um; Condition: water (0.1% TFA)-ACN; Begin B 0, End B 30; Gradient Time (min) 10; 100% B Hold Time (min) 2; Flow Rate (mL/min) 60; Injections 1) to afford intermediate 26d. LC-MS (ESI): m/z: 435.2 [M+H]⁺.

Step E—Synthesis of Compound 59 To a solution of intermediate 26d (160 mg, 0.368 mmol) in MeOH (3 mL) was added intermediate 5 (192 mg, 0.368 mmol). The reaction mixture was stirred at 25° C. for 16 h. Then the resulting mixture was directly purified by reverse phase HPLC (Column: Boston Uni C18 40×150×5 um; Condition: water (0.1% TFA)-ACN; Begin B 0, End B 30; Gradient Time (min) 10; 100% B Hold Time (min) 2; FlowRate (mL/min) 60; Injections 1) to afford compound 59 as its TFA salt form. The TFA salt was dissolved in 4 mL of water and the resulting solution was further purified by reverse phase HPLC (Column: Welch Xtimate C18 150×25 mm×5 um; Condition: water (0.225% FA)-ACN; Begin B 0, End B 19; Gradient Time (min) 15; 100% B Hold Time (min) 2; FlowRate (mL/min) 25; Injections 2). The product-containing fractions were combined and lyophilized to afford compound 59 as its formic acid salt. LC-MS (ESI) m/z: 781.1 [M+H]⁺. ¹H NMR (400 MHz, D₂O+CD₃CN) δ: 7.41-7.34 (m, 2H), 6.91-6.83 (m, 2H), 4.60 (s, 1H), 4.44-4.36 (m, 1H), 3.52 (apparent t, J=6.1 Hz, 2H), 3.39 (s, 2H), 3.27-3.19 (m, 5H), 3.16-3.03 (m, 2H), 2.83-2.70 (m, 2H), 2.10-2.01 (m, 1H), 1.86-1.65 (m, 7H), 1.51 (s, 3H), 1.39 (s, 3H), 1.19 (s, 3H).

### Example 40: Preparation of Intermediates 27b-cis-1, 27b-cis-2, 27b-trans-1 and 27b-trans-2

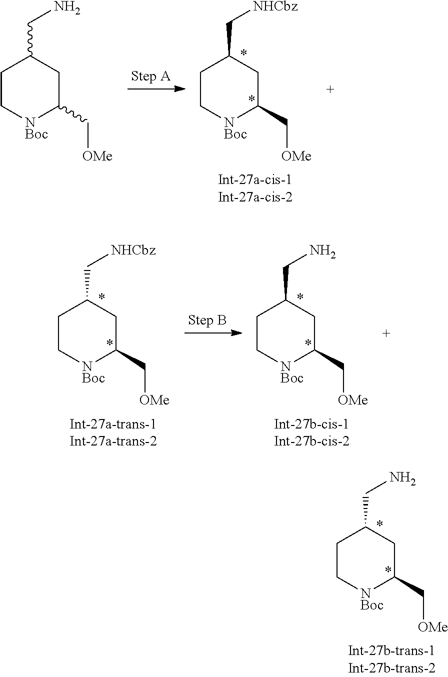 [SMILES: COCC1CC(CN)CCN1C(=O)OC(C)(C)C ; COC[C@@H]1C[C@H](CNC(=O)OCc2ccccc2)CCN1C(=O)OC(C)(C)C ; COC[C@@H]1C[C@@H](CNC(=O)OCc2ccccc2)CCN1C(=O)OC(C)(C)C ; COC[C@@H]1C[C@H](CN)CCN1C(=O)OC(C)(C)C ; COC[C@@H]1C[C@@H](CN)CCN1C(=O)OC(C)(C)C]

Step A—Synthesis of Intermediate 27a-cis-1, 27a-cis-2, 27a-trans-1, and 27a-trans-2 To a solution of tert-butyl 4-(aminomethyl)-2-(methoxymethyl)piperidine-1-carboxylate (3 g, 11.61 mmol) in DCM (300 mL) stirred at 0° C., was added triethylamine (4.86 mL, 34.8 mmol), followed by the dropwise addition of benzyl chloroformate (1.961 mL, 13.93 mmol). The reaction was stirred at 25° C. for 12 h, then diluted with water (50 mL) and extracted with ethyl acetate (130 mL×3). The combined organic layers were washed with brine (50 mL), dried over anhydrous Na₂SO₄, filtered, and the filtrate was concentrated in vacuo. The resulting residue was purified by flash silica gel chromatography MPLC (ISCO®; 40 g SepaFlash® Silica Flash Column, Eluent of 0~30% ethyl acetate/petroleum ether gradient; flow rate=56 mL/min) to give the title compound as a mixture of stereoisomers. The mixture of stereoisomers was separated by SFC (Column: Phenomenex-Cellulose-2, 250 mm×50 mm×10 um; Condition: 0.1% NH₃·H₂O/EtOH; Begin B 20%, End B 20%; Flow Rate (mL/min) 200; Injections 200) to give intermediate 27a-cis-1 and a mixture of the remaining three stereoisomers. The mixture was further separated by a second SFC (Column: DAICEL CHIRALPAK IC 250 mm×50 mm×10 um; Condition: 0.1% NH₃·H₂O IPA; Begin B 40%, End B 40%; Flow Rate (m/min) 200; Injections 300) to individually give intermediates 27a-cis-2 (the first eluting stereoisomer) and 27a-trans-1 (the second eluting stereoisomer) and 27a-trans-2 (the third eluting stereoisomer).

Intermediate 27a-cis-1: ¹H NMR (400 MHz, CD₃OD) δ: 7.39-7.26 (m, 5H), 5.07 (s. 2H), 4.59 (s, 1H), 3.96-3.87 (m, 1H), 3.70-3.62 (m, 1H), 3.52-3.40 (m, 2H), 3.33 (s, 3H), 3.21-3.08 (m, 2H), 3.05-2.98 (m, 1H), 1.85-1.67 (m, 3H), 1.45 (s, 9H), 1.40-1.28 (m, 1H), 1.27-1.14 (m, 1H).

Intermediate 27a-cis-2: ¹H NMR (400 MHz, CDCl3) δ: 7.41-7.28 (m, 5H), 5.09 (s, 2H), 4.88 (br s, 1H), 3.94-3.84 (m, 1H), 3.70-3.62 (m, 1H), 3.52-3.40 (m, 2H), 3.34 (s, 3H), 3.24-3.03 (m, 3H), 1.87-1.73 (m, 3H), 1.45 (s, 9H), 1.41-1.31 (m, 1H), 1.20 (br d, J=5.9 Hz, 1H).

Intermediate 27a-trans-1: ¹H NMR (400 MHz, CDCl3) δ: 7.40-7.28 (m, 5H), 5.09 (s, 2H), 4.86 (br s, 1H), 4.35 (br s, 1H), 4.15-3.89 (m, 1H), 3.40 (br d, J=5.5 Hz, 2H), 3.32 (s, 3H), 3.05 (br s, 2H), 2.76 (br s, 1H), 1.79 (br d, J=11.3 Hz, 2H), 1.65 (br s, 1H), 1.45 (s, 9H), 1.26-1.15 (m, 1H), 1.12-0.98 (m, 1H).

Intermediate 27a-trans-2: ¹H NMR (400 MHz, CDCl3) δ: 7.39-7.28 (m, 5H), 5.16-5.05 (m, 2H), 4.88 (br s, 1H), 4.58-4.29 (m, 1H), 4.14-3.93 (m, 1H), 3.40 (br d, J=5.9 Hz, 2H), 3.32 (s, 3H), 3.04 (br s, 2H), 2.76 (br s, 1H), 1.79 (br d, J=11.7 Hz, 2H), 1.65 (br s, 1H), 1.45 (s, 9H), 1.22-1.19 (m, 1H), 1.12-0.99 (m, 1H).

Step B—Synthesis of Intermediates 27b-cis-1, 27b-cis-2, 27b-trans-1, and 27b-trans-2 To a solution of intermediate 27a-cis-1 (160 mg, 0.408 mmol) in EtOAc (80 mL) was added 10 wt % Pd/C (217 mg, 0.204 mmol). The reaction was stirred at 25° C. under 15 psi of H₂ for 2.5 h. Then the reaction mixture was filtered and the filtrate was concentrated in vacuo to give crude intermediate 27b-cis-1, which was used in the next reaction without further purification. LC-MS (ESI) m/z: 280.9 [M+Na]⁺.

Intermediate 27b-cis-2 (LC-MS (ESI) m/z: 281.0 [M+Na]⁺), 27b-trans-1 (LC-MS (ESI) m/z: 259.3 [M+H]⁺), and 27b-tans-2 (LC-MS (ESI) m/z: 259.2 [M+H]⁺) were analogously prepared from the corresponding intermediates 27a-cis-2, 27a-trans-1, and 27a-trans-2 using the method described in Example 40, Step B.

### Example 41: Preparation of Compounds 60 to 63

### (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4R)-2-(methoxymethyl)piperidin-4-yl)methyl)carbamimidoyl)chroman-2-yl)propanoic acid, (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4S)-2-(methoxymethyl)piperidin-4-yl)methyl)carbamimidoyl)chroman-2-yl)propanoic acid, (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4R)-2-(methoxymethyl)piperidin-4-yl)methyl)carbamimidoyl)chroman-2-yl)propanoic acid, and (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4S)-2-(methoxymethyl)piperidin-4-yl)methyl)carbamimidoyl)chroman-2-yl)propanoic Acid

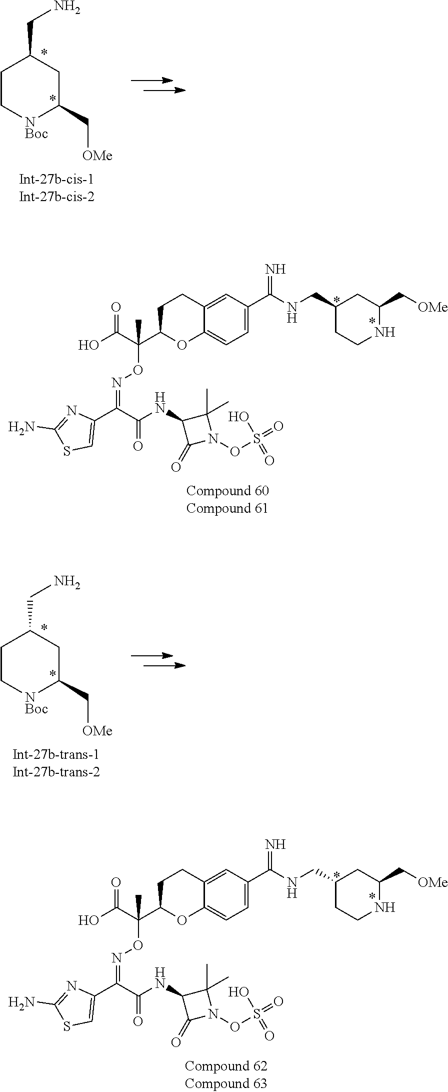 [SMILES: COC[C@@H]1C[C@H](CN)CCN1C(=O)OC(C)(C)C ; COC[C@@H]1C[C@H](CNC(=N)c2ccc3c(c2)CC[C@H]([C@](C)(O/N=C(\C(=O)N[C@@H]2C(=O)N(OS(=O)(=O)O)C2(C)C)c2csc(N)n2)C(=O)O)O3)CCN1 ; COC[C@@H]1C[C@@H](CN)CCN1C(=O)OC(C)(C)C ; COC[C@@H]1C[C@@H](CNC(=N)c2ccc3c(c2)CC[C@H]([C@](C)(O/N=C(\C(=O)N[C@@H]2C(=O)N(OS(=O)(=O)O)C2(C)C)c2csc(N)n2)C(=O)O)O3)CCN1]

Compounds 60 and 61 were analogously prepared as formic acid salts from the corresponding intermediates 27b-cis-1 and 27b-cis-2, according to the method described in Steps C to E of Example 39.

Compound 60: LC-MS (ESI) m/z: 767.1 [M+H]⁺. ¹H NMR (400 MHz, D₂O+CD₃CN) δ: 7.43-7.36 (m, 2H), 6.90 (d, J=8.6 Hz, 1H), 6.79 (s, 1H), 4.60 (s, 1H), 4.39 (br d, J=9.8 Hz, 1H), 3.57-3.49 (m, 1H), 3.45-3.35 (m, 2H), 3.32-3.25 (m, 6H), 2.97-2.87 (m, 1H), 2.82-2.72 (m, 2H), 2.12-2.01 (m, 2H), 1.90-1.85 (m, 2H), 1.79-1.65 (m, 1H), 1.50 (s, 3H), 1.42 (s, 3H), 1.40- 1.34 (m, 1H), 1.30-1.22 (m, 4H).

Compound 61: LC-MS (ESI) m/z: 767.1 [M+H]⁺. ¹H NMR (400 MHz, D₂O+CD₃CN) δ: 7.42-7.36 (m, 2H), 6.90 (d, J=8.6 Hz, 1H), 6.78 (s, 1H), 4.60 (s, 1H), 4.38 (br d, J=11.3 Hz, 1H), 3.56-3.50 (m, 1H), 3.45-3.36 (m, 2H), 3.33-3.24 (m, 6H), 2.98-2.88 (m, 1H), 2.83-2.69 (m, 2H), 2.09-2.02 (m, 2H), 1.93-1.86 (m, 2H), 1.78-1.61 (m, 1H), 1.49 (s, 3H), 1.42 (s, 3H), 1.39-1.31 (m, 1H), 1.24 (s, 4H).

Compounds 62 and 63 were analogously prepared as formic acid salts from the corresponding intermediates 27b-trans-1 and 27b-trans-2, according to the method described in Steps C to E of Example 39.

Compound 62: LC-MS (ESI) m/z: 767.1 [M+H]⁺. ¹H NMR (400 MHz, D₂O+CD₃CN) δ: 7.42-7.35 (m, 2H), 6.90 (d, J=8.2 Hz, 1H), 6.78 (s, 1H), 4.59 (s, 1H), 4.39 (br d, J=9.8 Hz, 1H), 3.61-3.48 (m, 3H), 3.36-3.29 (m, 5H), 3.18-3.09 (m, 2H), 2.84-2.73 (m, 2H), 2.28-2.18 (m, 1H), 2.09-2.02 (m, 1H), 1.93-1.87 (m, 1H), 1.84-1.75 (m, 1H), 1.75-1.55 (m, 3H), 1.49 (s, 3H), 1.42 (s, 3H), 1.24 (s, 3H).

Compound 63: LC-MS (ESI) m/z: 767.1 [M+H]⁺. ¹H NMR (400 MHz, D₂O+CD₃CN) δ: 7.42-7.35 (m, 2H), 6.90 (d, J=8.2 Hz, 1H), 6.78 (s, 1H), 4.60 (s, 1H), 4.39 (br d, J=11.0 Hz, 1H), 3.61-3.47 (m, 3H), 3.36-3.29 (m, 5H), 3.17-3.09 (m, 2H), 2.89-2.72 (m, 2H), 2.27-2.18 (m, 1H), 2.09-2.02 (m, 1H), 1.92-1.86 (m, 1H), 1.78 (br s, 1H), 1.74-1.55 (m, 3H), 1.50 (s, 3H), 1.42 (s, 3H), 1.24 (s, 3H).

### Example 42: Preparation of Compounds 64 and 65

### (S)-2-((R)-6-(N-(((1R,2S)-2-(2-aminoethyl)cyclopropyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid and (S)-2-((R)-6-(N-(((1S,2R)-2-(2-aminoethyl)cyclopropyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

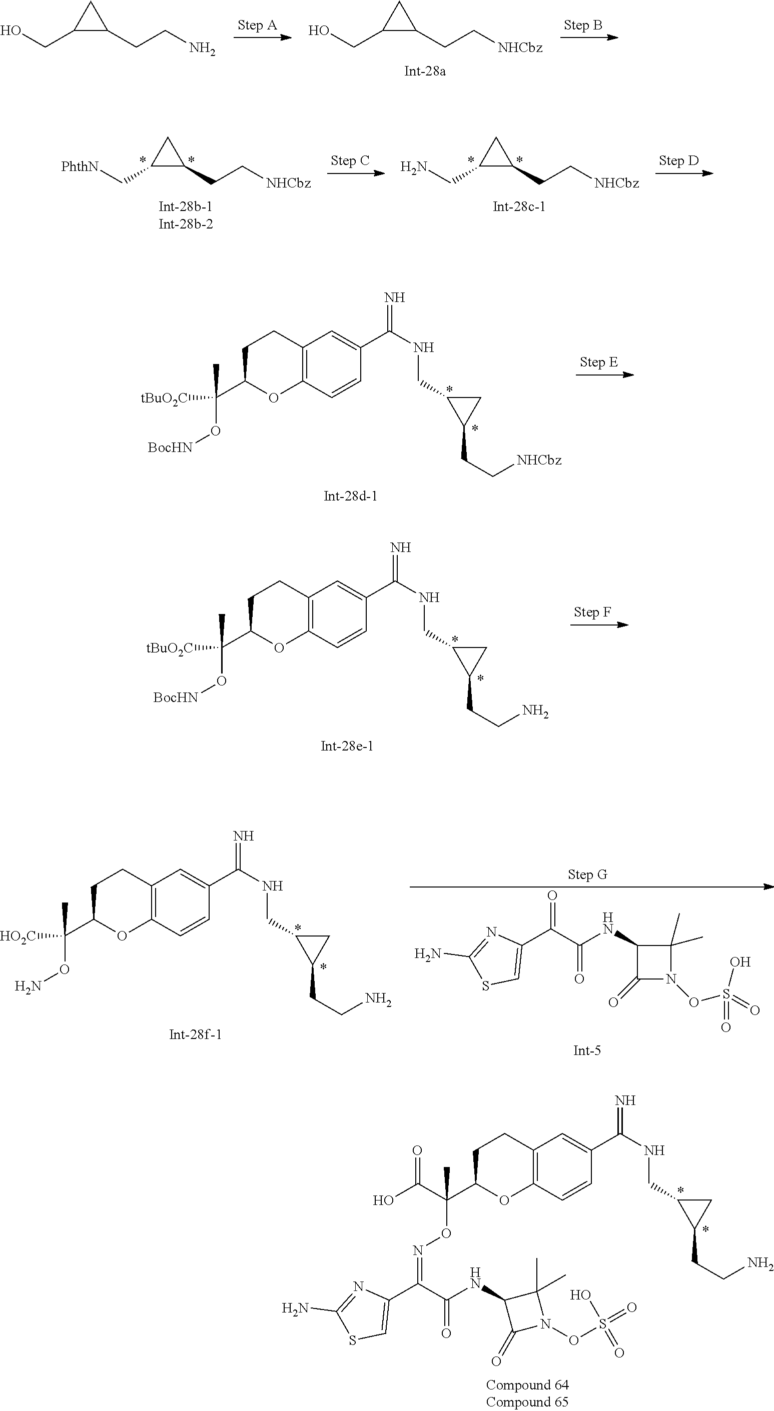

Step A—Synthesis of Intermediate 28a To a stirred suspension of (2-(2-aminoethyl)cyclopropyl)-methanol (2.4 g, 20.84 mmol) in DCM (180 mL) were added triethylamine (5.81 mL, 41.7 mmol) and benzyl chloroformate (3.23 mL, 22.92 mmol) dropwise at 0° C. under N₂. The reaction mixture was allowed to warm to 25° C. and stirred for 16 h, then diluted with water (20 mL) and extracted with DCM (50 mL×3). The combined organic layer was washed with brine (20 mL), dried over anhydrous Na₂SO₄, filtered, and the filtrate was concentrated in vacuo. The resulting crude product was purified by flash silica gel chromatography (ISCO®; 4 g SepaFlash® Silica Flash Column. Eluent of 0-90% ethyl acetate/petroleum ether (gradient); flow rate=35 mL/min) to give intermediate 28a. ¹H NMR (400 MHz, CDCl₃) δ: 7.40-7.28 (m, 5H), 5.10 (br d, J=3.1 Hz, 2H), 3.75 (br dd, J=5.1, 11.0 Hz, 1H), 3.51-3.38 (m, 1H), 3.24-3.13 (m, 1H), 3.04 (br t, J=10.2 Hz, 1H), 1.76-1.67 (m, 1H), 1.13-1.03 (m, 1H), 0.92-0.81 (m, 1H), 0.65-0.54 (m, 1H), 0.41-0.29 (m, 2H).

Step B—Synthesis of Intermediates 28b-1 and 28b-2 To a solution of intermediate 28a (2.8 g, 11.23 mmol) and phthalimide (1.818 g, 12.35 mmol) in toluene (55 mL) were added triphenylphosphine (5.89 g, 22.46 mmol) and di-tert-butyl azodicarboxylate (5.17 g, 22.46 mmol) dropwise at 25° C. under N₂. After stirring for 5 min, the reaction mixture was heated to 80° C. and stirred at this temperature for 16 h. Then the reaction mixture was diluted with water (90 mL) and extracted with ethyl acetate (130 mL×3). The combined organic layers were washed with brine (80 mL), dried over anhydrous Na₂SO₄, filtered, and the filtrate was concentrated in vacuo. The resulting crude product was purified by flash silica gel chromatography (ISCO®; 40 g SepaFlash® Silica Flash Column, Eluent of 15%-20% ethyl acetate/petroleum ether (gradient); flow rate=56 mL/min) to give the desired product as a racemic mixture. The racemic mixture was separated into its individual enantiomers by SFC (Daicel Chiralpak IC (250 mm*50 mm, 10 um); Condition: 0.1% NH₃·H₂O/IPA; Begin B, 40% End B 40%; Flow Rate (mL/min) 200; Injections 300) to give intermediates 28b-1 (the first eluting enantiomer) and 28b-2 (the second eluting enantiomer).

Intermediate 28b-1: LC-MS (ESI) m/z: 379.0 [M+H]⁺. ¹H NMR (400 MHz, CDCl3) δ: 7.82 (dd, J=2.9, 5.3 Hz, 2H), 7.69 (dd, J=2.9, 5.3 Hz, 2H), 7.38-7.27 (m, 5H), 5.03 (s, 2H), 4.94 (br s, 1H), 3.74-3.59 (m, 1H), 3.50-3.39 (m, 1H), 3.27-3.10 (m, 2H), 1.45 (td, J=6.8, 13.7 Hz, 1H), 1.32 (qd, J=6.9, 14.0 Hz, 1H), 1.00-0.89 (m, 1H), 0.82 (br d, J=6.7 Hz, 1H), 0.56 (td, J=4.6, 8.8 Hz, 1H), 0.42-0.28 (m, 1H).

Intermediate 28b-2: LC-MS (ESI) m/z: 401.0 [M+Na]⁺. ¹H NMR (400 MHz, CDCl3) δ: 7.87-7.77 (m, 2H), 7.69 (dd, J=2.9, 4.9 Hz, 2H), 7.38-7.27 (m, 5H), 5.12-5.00 (m, 2H), 4.95 (br s, 1H), 3.73-3.60 (m, 1H), 3.44 (br dd, J=7.4, 14.1 Hz, 1H), 3.26-3.10 (m, 2H), 1.51 (d, J=2.7 Hz, 1H), 1.36-1.28 (m, 1H), 0.98-0.91 (m, 1H), 0.83 (br d, J=7.8 Hz, 1H), 0.61-0.51 (m, 1H), 0.40-0.28 (m, 1H).

Step C—Synthesis of Intermediate 28c-1 To a solution of intermediate 28b-1 (300 mg, 0.793 mmol) in EtOH (7 mL) was added 85% hydrazine hydrate (55 mg, 0.793 mmol). The reaction mixture was stirred at 25° C. for 16 h. Then the reaction content was filtered and the filtrate was concentrated in vacuo to give crude intermediate 28c-1, which was used in the next reaction without further purification. LC-MS (ESI) m/z: 249.0 [M+H]⁺.

Step D—Synthesis of Intermediate 28d-1 To a solution of intermediate 28c-1 (176 mg, 0.707 mmol) and acetic acid (116 mg, 1.929 mmol) in acetonitrile (5 mL) were added intermediate 3c (300 mg, 0.643 mmol) and potassium acetate (252 mg, 2.57 mmol). The reaction was stirred at 85° C. under N₂ for 30 min. Then the reaction mixture was diluted with water (20 mL) and extracted with ethyl acetate (15 mL×3). The combined organic layer was washed with brine (20 mL), dried over anhydrous Na₂SO₄, filtered, and the filtrate was concentrated in vacuo. The resulting residue was purified by reverse phase MPLC (Biotage; 20 g Agela C18 20~35 gm column, Eluent of 0-40% MeCN/H₂O+0.5‰ TFA (gradient) flow rate=50 mL/min) to give intermediate 28d-1. LC-MS (ESI) m/z: 667.2 [M+H]⁺.

Step E—Synthesis of Intermediate 28e-1 To a solution of intermediate 28d-1 (280 mg, 0.420 mmol) in EtOAc (30 mL) was added 10% Pd/C (44.7 mg, 0.042 mmol). The reaction was stirred at 25° C. for 30 min under 15 psi of H₂. Then the reaction mixture was filtered and the filtrate was concentrated in vacuo to give crude intermediate 28e-1, which was used in the next reaction without further purification. LC-MS (ESI) m/z: 533.1 [M+H]⁺.

Step F Synthesis of Intermediate 28f-1 To a solution of intermediate 28e-1 (210 mg, 0.394 mmol) in DCM (1.5 mL) was added 12 N HCl (1.5 mL) at 0° C. The reaction was stirred at 25° C. for 15 minutes. Then the reaction solution was dried under nitrogen gas flow, and the resulting residue was purified by Prep-HPLC (Boston Uni C18 40×150×5 um column; Condition: water (with 0.1% TFA)-ACN; Begin B 0, End B 30; Gradient Time (min) 10; 100% B Hold Time (min) 2; Flow Rate (mL/min) 60; Injections 1) to give intermediate 28f-1 after lyophilization. LC-MS (ESI) m/z: 417.1 [M+CH₃CN]⁺.

Step G—Synthesis of Compounds 64 and 65 To a solution of intermediate 5 (85 mg, 0.234 mmol) in DMA (2 mL) was added intermediate 28f-1 (80 mg, 0.213 mmol). The reaction mixture was stirred at 25° C. for 16 h, then diluted with MeOH (0.5 mL) and purified by reverse phase HPLC (Boston Uni C18 40×150×5 um column; Condition: water (with 0.1% TFA)-ACN; Begin B 0, End B 30; Gradient Time (min) 10; 100% B Hold Time (min) 2; Flow Rate (mL/min) 60; Injections 1) to give compound 64 as its TFA salt after lyophilization. The TFA salt was dissolved in H₂O (2 mL) and purified by reverse phase HPLC (Welch Xtimate C18 150×25 mm×5 um column; Condition: water (with 0.225% FA)-ACN; Begin B 0, End B 20; Gradient Time (min) 15; 100% B Hold Time (min) 2; Flow Rate (mL/min) 25; Injections 2) to give compound 64 as its formic acid salt after lyophilization. LC-MS (ESI) m/z: 723.2 [M+H]⁺. ¹H NMR (400 MHz, D₂O+CD₃CN) δ: 7.43-7.35 (m, 2H), 6.90 (d, J=8.6 Hz, 1H), 6.80 (s, 1H), 4.60 (s, 1H), 4.39 (br d, J=11.0 Hz, 1H), 3.20 (d, J=7.0 Hz, 2H), 2.95 (apparent t, J=7.6 Hz, 2H), 2.86-2.70 (m, 2H), 2.13-2.03 (m, 1H), 1.76-1.59 (m, 2H), 1.51 (s, 3H), 1.45-1.35 (m, 4H), 1.24 (s, 3H), 1.02-0.91 (m, 1H), 0.76-0.65 (m, 1H), 0.56-0.41 (m, 2H).

Compound 65 was analogously prepared as its formic acid salt from intermediate 28b-2 using the method described in Steps C to G of Example 42. LC-MS (ESI) m/z: 723.2 [M+H]⁺. ¹H NMR (400 MHz, D₂O+CD₃CN) δ: 7.43-7.36 (m, 2H), 6.90 (d, J=8.6 Hz, 1H), 6.79 (s, 1H), 4.60 (s, 1H), 4.39 (apparent d, J=11.3 Hz, 1H), 3.27-3.15 (m, 2H), 2.95 (apparent t, J=7.4 Hz, 2H), 2.85-2.73 (m, 2H), 2.13-2.03 (m, 1H), 1.75-1.58 (m, 2H), 1.50 (s, 3H), 1.47-1.37 (m, 4H), 1.25 (s, 3H), 1.04-0.92 (m, 1H), 0.76-0.68 (m, 1H), 0.56-0.42 (m, 2H).

### Example 43: Preparation of Intermediates 29c-1 and 29c-2

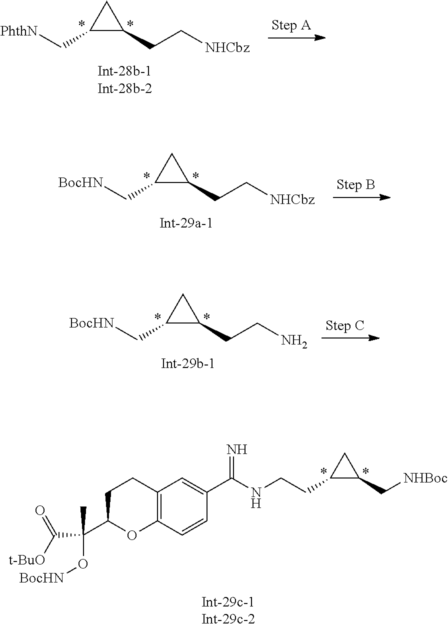

Step A—Synthesis of Intermediate 29a-1 To a solution of intermediate 28b-1 (650 mg, 1.718 mmol) in EtOH (16 mL) was added 85% hydrazine hydrate (151.7 mg, 2.58 mmol). The reaction was stirred at 25° C. for 16 h, then filtered and the filtrate was concentrated in vacuo. The resulting residue was dissolved in DCM (16 mL), cooled at 0° C., followed by the addition of triethylamine (0.719 mL, 5.16 mmol) and di-tert-butyl dicarbonate (525 mg, 2.407 mmol). The reaction was stirred at 25° C. for 16 h, then diluted with water (30 mL) and extracted with DCM (15 mL×3). The combined organic layer was washed with brine (20 mL), dried over anhydrous Na₂SO₄, filtered, and the filtrate was concentrated in vacuo. The resulting residue was purified by flash silica gel chromatography MPLC (ISCO®; 4 g SepaFlash® Silica Flash Column; Eluent of 0-30% ethyl acetate/petroleum ether (gradient); flow rate=35 mL/min) to give intermediate 29a-1. LC-MS (ESI) m/z: 248.9 [M+H-100]⁺.

Step B—Synthesis of Intermediate 29b-1 To a solution of intermediate 29a-1 (540 mg, 1.550 mmol) in EtOAc (50 mL) was added 10 wt % Pd/C (165 mg, 0.155 mmol). The reaction was stirred at 25° C. under 15 psi of H₂ for 12 h. Then the reaction mixture was filtered and the filtrate was concentrated in vacuo to give intermediate 29b-1, which was used in the next reaction without further purification. ¹H NMR (500 MHz, CDCl3) δ: 4.91 (br s, 1H), 3.12-3.01 (m, 1H), 2.96-2.87 (m, 1H), 2.83-2.73 (m, 2H), 1.44 (s, 10H), 1.28 (dt, J=6.9, 13.9 Hz, 1H), 0.75-0.67 (m, 1H), 0.60 (br s, 1H), 0.41-0.27 (m, 2H).

Step C—Synthesis of Intermediates 29c-1 and 29c-2 To a solution of intermediate 29b-1 (126 mg, 0.589 mmol) in acetonitrile (5.5 mL) were added acetic acid (97 mg, 1.607 mmol), intermediate 3c (250 mg, 0.536 mmol) and potassium acetate (210 mg, 2.143 mmol) sequentially. The reaction was stirred at 85° C. for 20 minutes. Then the reaction mixture was diluted with water (20 mL) and extracted with ethyl acetate (15 mL×3). The combined organic layer was washed with brine (20 mL), dried over anhydrous Na₂SO₄, filtered, and the filtrate was concentrated in vacuo. The resulting residue was purified by reverse phase MPLC (Biotage; 20 g Agela C18, 20~35 pm column; Eluent of 0-40% MeCN/H₂O (0.5‰ TFA) gradient; flow rate=50 mL/min) to give intermediate 29c-1. LC-MS (ESI) m/z: 633.2 [M+H]⁺.

Intermediate 29c-2 was analogously prepared from intermediate 28b-2 using the method described in Steps A to C of Example 43. LC-MS (ESI) m/z: 633.2 [M+H]⁺.

### Example 44: Preparation of Compounds 66 and 67

### (S)-2-((R)-6-(N-(2-((1S,2R)-2-(aminomethyl)cyclopropyl)ethyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid and (S)-2-((R)-6-(N-(2-((1R,2S)-2-(aminomethyl)cyclopropyl)ethyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

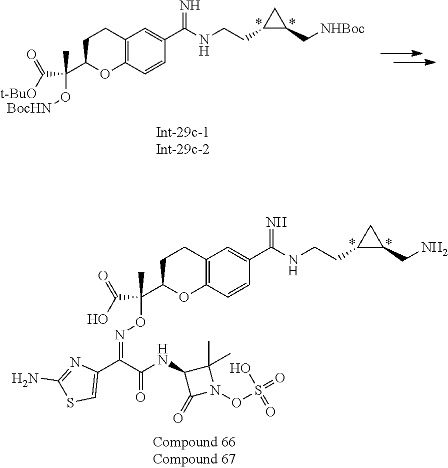 [SMILES: CC(C)(C)OC(=O)NC[C@@H]1C[C@H]1CCNC(=N)c1ccc2c(c1)CC[C@H]([C@](C)(ONC(=O)OC(C)(C)C)C(=O)OC(C)(C)C)O2 ; CC1(C)[C@H](NC(=O)/C(=N\O[C@](C)(C(=O)O)[C@H]2CCc3cc(C(=N)NCC[C@@H]4C[C@H]4CN)ccc3O2)c2csc(N)n2)C(=O)N1OS(=O)(=O)O]

Compounds 66 and 67 were analogously prepared as formic acid salts from the corresponding intermediates 29c-1 and 29c-2, using the method described in Steps F and G of Example 42.

Compound 66: LC-MS (ESI) m/z: 723.2 [M+H]⁺. ¹H NMR (400 MHz, D₂O+CD₃CN) δ: 7.41-7.35 (m, 2H), 6.90 (d, J=8.2 Hz, 1H), 6.78 (s, 1H), 4.60 (s, 1H), 4.38 (apparent d, J=9.8 Hz, 1H), 3.40 (apparent t, J=6.7 Hz, 2H), 2.89-2.77 (m, 3H), 2.73-2.64 (m, 1H), 2.13-2.03 (m, 1H), 1.76-1.65 (m, 2H), 1.53-1.44 (m, 4H), 1.42 (s, 3H), 1.25 (s, 3H), 0.90-0.75 (m, 2H), 0.53-0.44 (m, 2H).

Compound 67: LC-MS (ESI) m/z: 723.2 [M+H]⁺. ¹H NMR (400 MHz, D₂O+CD₃CN) δ: 7.43-7.35 (m, 2H), 6.90 (d, J=8.2 Hz, 1H), 6.78 (s, 1H), 4.60 (s, 1H), 4.38 (apparent d, J=11.3 Hz, 1H), 3.40 (apparent t, J=6.8 Hz, 2H), 2.89-2.76 (m, 3H), 2.73-2.62 (m, 1H), 2.14-2.03 (m, 1H), 1.77-1.62 (m, 2H), 1.53-1.44 (m, 4H), 1.42 (s, 3H), 1.25 (s, 3H), 0.90-0.74 (m, 2H), 0.55-0.44 (m, 2H).

### Example 45: Preparation of Intermediates 30 g-1 and 30 g-2

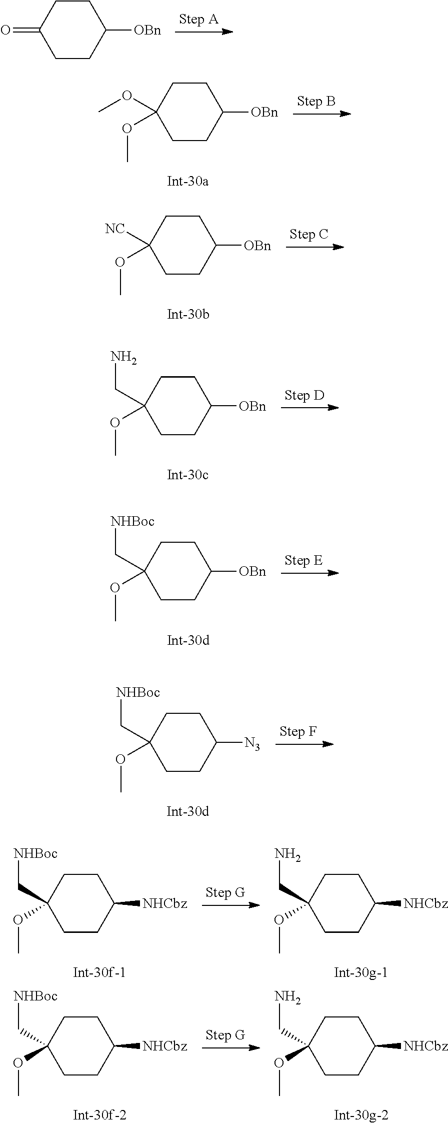

Step A—Synthesis of Intermediate 30a To a solution of 4-(benzyloxy)cyclohexan-1-one (7.8 g, 38.2 mmol) in MeOH (218 mL) were added trimethoxymethane (32.8 mL, 299 mmol) and 4-methylbenzenesulfonic acid (0.197 g, 1.146 mmol). The reaction mixture was stirred at 25° C. for 16 h, then filtered, and the filtrate was concentrated in vacuo. The resulting residue was diluted with saturated aqueous NaHCO₃ solution (150 mL) and extracted with EtOAc (100 mL×3). The aqueous layer was back-extracted with EtOAc (150 mL×3). The organic layers were combined, dried over anhydrous Na₂SO₄, filtered, and the filtrate was concentrated in vacuo. The resulting residue was purified by flash silica gel chromatography (ISCO; 40 g Agela Silica Flash Column, Eluent of 0-10% EtOAc/Petroleum ether (gradient); flow rate=30 mL/min) to afford intermediate 30a. ¹H NMR (400 MHz, CDCl3) δ: 7.39-7.22 (m, 5H), 4.53 (s, 2H), 3.50 (tt, J=3.6, 7.3 Hz, 1H), 3.19 (d, J=2.0 Hz, 6H), 1.92 (ddd, J=4.1, 7.8, 11.9 Hz, 2H), 1.84-1.74 (m, 2H), 1.73-1.63 (m, 2H), 1.59-1.48 (m, 2H).

Step B—Synthesis of Intermediate 30b To a stirred solution of intermediate 30a (3 g, 11.98 mmol) in DCM (42.2 mL) was added dropwise trimethylsilanecarbonitrile (6.00 mL, 47.9 mmol) at 0° C. The reaction was stirred at 0° C. for 2 minutes, then trimethylsilyl trifluoromethanesulfonate (2.99 mL, 16.54 mmol) was added dropwise. The reaction was stirred at 0° C. for 2 h, then quenched by the slow addition of saturated aqueous sodium bicarbonate solution (40 mL). The organic layers were combined, and the aqueous layer was extracted with dichloromethane (3×40 mL). The combined organic layers were dried over anhydrous sodium sulfate, filtered, and the filtrate was concentrated under reduced pressure. The resulting residue was purified by flash silica gel chromatography (ISCO; 12 g Agela Silica Flash Column, Eluent of 0-10% EtOAc/Petroleum ether (gradient), flow rate=30 mL/min) to give intermediate 30b. LC-MS (ESI) m/z: 246.2 [M+H]⁺.

Step C—Synthesis of Intermediate 30c To a mixture of aluminum(III) lithium hydride (1 g, 26.3 mmol) in THF (60 mL) stirred at 0° C. under N₂, was added dropwise a solution of intermediate 30b (3.147 g, 12.83 mmol) in THF (20 mL). The reaction was stirred for 2 h at 25° C., then quenched by adding water (1 mL), 10% NaOH (2 mL) and water (3 mL) sequentially. The resulting mixture was stirred for 15 min, then filtered. The filtrate was concentrated under reduced pressure to give crude intermediate 30c, which was used in the next reaction without further purification. LC-MS (ESI) m/z: 250.2 [M+H]⁺.

Step D—Synthesis of Intermediate 30d To a solution of intermediate 30c (3.011 g, 12.08 mmol) in DCM (46 mL), stirred at 0° C., was added triethylamine (5.87 mL, 42.3 mmol), followed by di-tert-butyl dicarbonate (5.61 mL, 24.15 mmol). The reaction was stirred for 16 hours at 25° C. Then the reaction mixture was concentrated under reduced pressure, and the resulting residue was purified by flash silica gel chromatography (ISCO; 40 g Agela Silica Flash Column; Eluent of 0-30% EtOAc/Petroleum ether gradient; flow rate=40 m/min) to give intermediate 30d, which was used in the next reaction without further purification. LC-MS (ESI) m/z: 350.2 [M+H]⁺.

Step E—Synthesis of Intermediate 30e A mixture of intermediate 30d (5.734 g. 16.41 mmol) and dihydroxypalladium/C (20 wt %, 2.304 g, 3.28 mmol) in MeOH (115 mL) was stirred at 25° C. under a hydrogen atmosphere (35 psi) for 20 h. The reaction mixture was filtered and the filtrate was concentrated in vacuo. The resulting residue was dissolved in THF (140 mL), and the mixture was cooled to 0° C., followed by the addition of triethylamine (4.55 mL, 32.6 mmol). A solution of methanesulfonyl chloride (5.990 g, 52.3 mmol) in THF (23.30 mL) was added dropwise to the reaction mixture. The reaction mixture was warmed to 25° C. and stirred for 1.5 h, then cooled to 0° C. and quenched by the slow addition of a saturated aqueous NaHCO₃ solution (100 mL). The mixture was then extracted with MTBE (40 mL×3). The organic layers were combined, dried over anhydrous Na₂SO₄, and filtered. The filtrate was concentrated under reduced pressure. The resulting residue was dissolved in 100 mL of DMF and then treated with sodium azide (3.50 g, 53.8 mmol) at 25 CC. Then the reaction mixture was warmed to 80° C. and stirred for 16 h. After cooling to room temperature, the reaction mixture was diluted with water (300 mL), and extracted with EtOAc (70 mL×3). The organic layers were combined, washed with brine (300 mL×2), dried over anhydrous Na₂SO₄, and filtered. The filtrate was concentrated under reduced pressure to give crude intermediate 30e, which was used in the next reaction without further purification. ¹H NMR (400 MHz, CDCl₃) δ: 4.82-4.58 (m, 1H), 3.75 (br s, 1H), 3.47 (s, 1H), 3.23-3.17 (m, 1H), 1.99-1.71 (m, 4H), 1.66-1.49 (m, 5H), 1.43 (s, 10H).

Step F—Synthesis of Intermediates 30f-1 and 30I-2 To a stirred solution of intermediate 30e (4.63 g, 16.28 mmol) in H₂O (32.6 mL) and THF (130 mL) was added portionwise triphenylphosphine (6.41 g, 24.42 mmol) at 25° C. The reaction mixture was stirred at 25° C. for 16 h. Then the solvent was removed under reduced pressure, the resulting residue was dissolved in DCM (51.5 mL) and treated with diisopropylethylamine (2.74 g, 21.18 mmol). The mixture was cooled to 0° C., and a solution of benzylchloroformate (3.34 g, 19.55 mmol) in DCM (57.2 mL) was added dropwise. The reaction mixture was stirred at 25° C. for 16 h, then poured into water (150 mL), extracted with DCM (50 mL×3), washed with brine (80 mL), dried over anhydrous Na₂SO₄, and filtered. The filtrate was concentrated under reduced pressure. The resulting residue was purified by flash silica gel chromatography (ISCO; 40 g Agela Silica Flash Column; Eluent of 0-30% EtOAc/Petroleum ether (gradient); flow rate=30 mL/min) to individually give intermediates 30f-1 and 30f-2.

Intermediate 30f-1: LC-MS (ESI) m/z: 415.2 [M+Na]⁺. ¹H NMR (500 MHz, CDCl3) δ: 7.34-7.18 (m, 5H), 5.08 (s, 2H), 4.74 (s, 2H), 3.76 (br s, 1H), 3.28 (br d, J=6.1 Hz, 2H), 3.25 (s, 3H), 1.80 (br s, 2H), 1.59-1.41 (m, 4H), 1.34 (s, 11H).

Intermediate 30f-2: LC-MS (ESI) m/z: 415.2 [M+Na]⁺. ¹H NMR (500 MHz, CDCl3) δ: 7.42-7.29 (m, 5H), 5.09 (s, 2H), 4.65 (br s, 2H), 3.52-3.47 (m, 1H), 3.15 (s, 5H), 1.95-1.77 (m, 4H), 1.44 (s, 9H), 1.41-1.19 (m, 4H).

Step G—Synthesis of Intermediates 30 g-1 and 30 g-2 A solution of intermediate 30f-1 (413 mg, 1.052 mmol) in HCl/EtOAc (4 M) (10 mL) was stirred at 45° C. for 3 hours. Then the reaction mixture was filtered, and the filtrate was concentrated under reduced pressure to give intermediate 30 g-1, which was used in the next step without further purification. LC-MS (ESI) m/z: 293.2 [M+H]⁺.

Intermediate 30 g-2 was analogously prepared from intermediate 30f-2 using the method described in the current step. LC-MS (ESI) m/z: 292.3 [M+H]⁺.

### Example 46: Preparation of Compounds 68 and 69

### (S)-2-((R)-6-(N-(((1r,4R)-4-amino-1-methoxycyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid and (S)-2-((R)-6-(N-(((1s,4S)-4-amino-1-methoxycyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

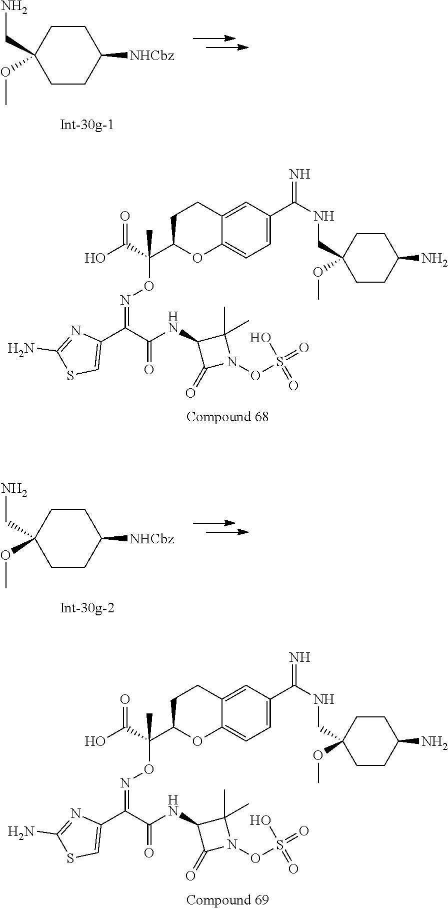 [SMILES: CO[C@]1(CN)CC[C@@H](NC(=O)OCc2ccccc2)CC1 ; CO[C@]1(CNC(=N)c2ccc3c(c2)CC[C@H]([C@](C)(O/N=C(\C(=O)N[C@@H]2C(=O)N(OS(=O)(=O)O)C2(C)C)c2csc(N)n2)C(=O)O)O3)CC[C@@H](N)CC1 ; CO[C@]1(CN)CC[C@H](NC(=O)OCc2ccccc2)CC1 ; CO[C@]1(CNC(=N)c2ccc3c(c2)CC[C@H]([C@](C)(O/N=C(\C(=O)N[C@@H]2C(=O)N(OS(=O)(=O)O)C2(C)C)c2csc(N)n2)C(=O)O)O3)CC[C@H](N)CC1]

Compounds 68 and 69 were analogously prepared as formic acid salts from the corresponding intermediates 30 g-1 and 30 g-2, using the method described in Steps D to G of Example 42.

Compound 68: LC-MS (ESI) m/z: 767.1 [M+H]⁺. ¹H NMR (400 MHz, D₂O+CD₃CN) δ: 7.57-7.44 (m, 2H), 7.02 (d, J=8.6 Hz, 1H), 6.96 (s, 1H), 4.75 (s, 1H), 4.56-4.51 (m, 1H), 3.67 (s, 2H), 3.45-3.34 (m, 1H), 3.31 (s, 3H), 2.95-2.84 (m, 2H), 2.25-2.17 (m, 1H), 2.16-2.05 (m, 2H), 1.90-1.86 (m, 5H), 1.65-1.61 (m, 5H), 1.55 (s, 3H), 1.36 (s, 3H).

Compound 69: LC-MS (ESI) m/z: 767.3 [M+H]⁺. ¹H NMR (D₂O+CD₃CN, 400 MHz) δ: 7.43-7.35 (m, 2H), 6.90 (d, J=8.6 Hz, 1H), 6.79 (s, 1H), 4.62 (s, 1H), 4.39 (apparent d, J=10.6 Hz, 1H), 3.39 (s, 2H), 3.16 (s, 3H), 3.15-3.03 (m, 1H), 2.87-2.72 (m, 2H), 2.12-2.03 (m, 1H), 1.98 (br s, 1H), 1.89-1.51 (m, 5H), 1.49 (s, 4H), 1.43 (s, 3H), 1.40-1.34 (m, 2H), 1.26 (s, 3H).

### Example 47: Preparation of Compounds 70 and 71

### (S)-2-((R)-6-(N-(((1s,3S)-3-amino-3-(hydroxymethyl)cyclobutyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid and (S)-2-((R)-6-(N-(((1r,3R)-3-amino-3-(hydroxymethyl)cyclobutyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic Acid

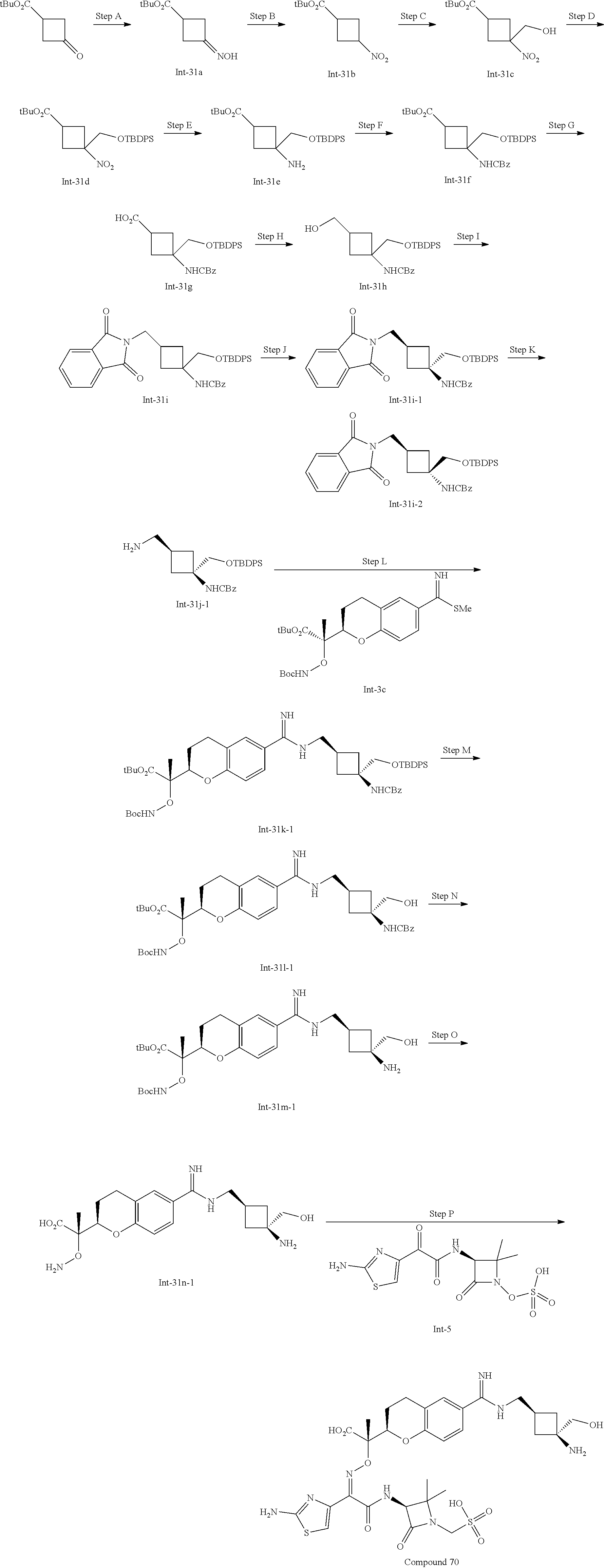

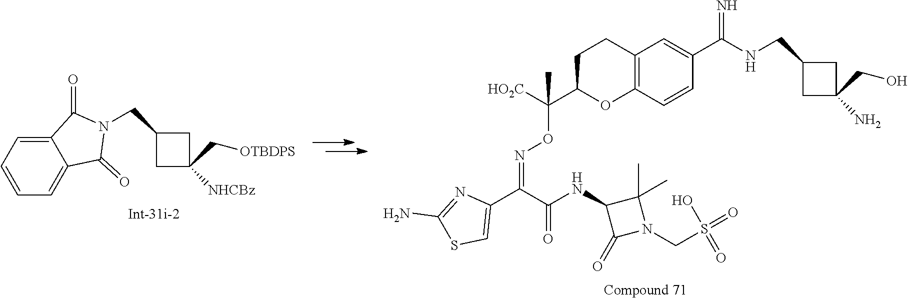

Step A—Synthesis of Intermediate 31a A solution of tert-butyl 3-oxocyclobutane-1-carboxylate (36.1 g, 212 mmol), hydroxylamine hydrochloride (58.1 g, 848 mmol) and sodium acetate (22.6 g, 276 mmol) in EtOH (370 mL) was stirred at 95° C. for 1 h. Then the reaction was filtered, and the filtrate was concentrated under vacuum. The resulting residue was diluted with water (100 mL), extracted with EtOAc (100 mL×3), washed with brine (200 mL×2), dried over anhydrous Na₂SO₄, and filtered. The filtrate was concentrated to afford crude intermediate 31a. was used in the next step without further purification. ¹H NMR (400 MHz, CDCl3) δ: 3.26-3.02 (m, 4H), 1.54-1.37 (m, 9H).

Step B—Synthesis of Intermediate 31b To a mixture of urea hydrogen peroxide (13.7 g, 146 mmol) in MeCN (80 mL) stirred at −10° C., was added dropwise a solution of 2,2,2-trifluoro-acetic anhydride (20.59 mL, 146 mmol) in MeCN (45 mL) over 30 min. It was stirred at −10° C. for 1 h. The resulting mixture was added dropwise over 60 min. to a mixture of intermediate 31a (9 g, 48.6 mmol) and sodium hydrogen phosphate (48.3 g, 340 mmol) in MeCN (80 mL). The reaction was stirred at 80° C. for 30 min, then filtered, and the filtrate was poured into water (400 mL). The solid was filtered off, and the filtrate was concentrated under vacuum to remove MeCN. The resulting mixture was extracted with EtOAc (200 mL×3), washed with brine (400 mL×2), dried over anhydrous Na₂SO₄, filtered, and the filtrate was concentrated under vacuum. The resulting residue was purified by flash silica gel chromatography (Biotage; 120 g Agela Silica Flash Column, Eluent of 0-9% EtOAc/Petroleum ether gradient; flow rate=50 mL/min) to give intermediate 31b. ¹H NMR (400 MHz, CDCl3) δ: 5.11-5.01 (m, 1H), 3.22-3.10 (m, 1H), 2.92-2.80 (m, 2H), 2.79-2.68 (m, 2H), 1.46 (s, 9H).

Step C—Synthesis of Intermediate 31c To a solution of intermediate 31b (2.3 g, 11.43 mmol) and formaldehyde (37% in water) (1.72 mL, 23.10 mmol) in MeCN (34 mL) stirred at 0° C., was added Et₃N (1.6 mL, 11.48 mmol) dropwise. The reaction was then stirred at 25° C. for 2 h, then diluted with EtOAc (50 mL), and washed with brine (50 mL×2). The organic phase was concentrated under vacuum to afford crude intermediate 31c, which was used in the next reaction without further purification. ¹H NMR (400 MHz, CDCl3) δ: 4.14-3.97 (m, 2H), 3.01-2.91 (m, 2H), 2.87-2.76 (m, 1H), 2.67-2.52 (m, 2H), 1.48-1.41 (m, 9H).

Step D—Synthesis of Intermediate 31d To a solution of intermediate 31c (2.55 g, 11.03 mmol) in MeCN (55 mL) stirred at 0° C., was added 1H-imidazole (2.25 g, 33.0 mmol), followed by tert-butylchlorodiphenylsilane (6 g, 21.83 mmol). The reaction was stirred at 25° C. for 16 h, then diluted with water (50 mL), and extracted with EtOAc (30 mL×3). The organic phase was washed with brine (50 mL), concentrated under vacuum, and the resulting residue was purified by flash silica gel chromatography (Biotage; 12 g Agela Silica Flash Column, Eluent of 0-10% EtOAc/Petroleum ether gradient; flow rate=35 mL/min) to give a cis/trans mixture of intermediate 31d. ¹H NMR (400 MHz, CDCl3) δ: 7.72-7.31 (m, 10H), 4.10-4.01 (m, 2H), 3.20-3.0 (m, 1H), (3.03-2.87 (m, 2H), 2.68-2.50 (m, 2H), 1.42 (d, J=17.2 Hz, 9H), 1.02 (d, J=6.3 Hz, 9H).

Step E—Synthesis of Intermediate 31e A mixture of intermediate 31d (1.78 g, 3.79 mmol) and Raney nickel (0.45 g, 1.516 mmol; 20% in water) in IPA (30 mL) was stirred at 70° C. under hydrogen atmosphere (40 psi) for 16 h. Then the reaction mixture was filtered, and the filtrate was concentrated under vacuum to afford a cis/trans mixture of intermediate 31e, which was used in the next reaction without further purification. ¹H NMR (400 MHz, CDCl3) δ: 7.73-7.56 (m, 4H), 7.46-7.32 (m, 6H), 3.65-3.60 (m, 2H), 2.62-2.43 (m, 1H), 2.36-2.22 (m, 3H), 2.18-2.07 (m, 1H), 1.44-1.34 (m, 9H), 1.13-1.03 (m, 9H).

Step F—Synthesis of Intermediate 31f To a mixture of intermediate 31e (1.59 g, 3.62 mmol) and Na₂CO₃ (0.77 g, 7.27 mmol) in THF/water (2:1; 36 mL), stirred at 0° C., was added benzylchloroformate (0.7 mL, 4.90 mmol). The reaction was stirred at 24° C. for 16 h, then diluted with water (100 mL) and extracted with EtOAc (50 mL×3). The organic layers were combined, washed with brine (80 mL), dried over anhydrous Na₂SO₄, filtered, and the filtrate was concentrated under reduced pressure. The resulting residue was purified by flash silica gel chromatography (Biotage; 20 g Agela Silica Flash Column, Eluent of 0-10% EtOAc/Petroleum ether gradient; flow rate=5 mL/min) to give a cis/trans mixture of intermediate 31f. ¹H NMR (400 MHz, CDCl3) δ: 7.65-7.59 (m, 4H), 7.45-7.27 (m, 11H), 5.05-5.02 (m, 2H), 3.75-3.72 (m, 2H), 3.14 (br s, 1H), 2.58-2.29 (m, 4H), 1.43-1.38 (m, 9H), 1.06-1.05 (m, 9H).

Step G—Synthesis of Intermediate 31 g To a solution of intermediate 31f (1.52 g, 2.65 mmol) in DCM (95 mL) stirred under N₂ at 0° C., was added 2,6-dimethylpyridine (3.1 mL, 26.5 mmol), followed by the dropwise addition of trimethylsilyl trifluoromethanesulfonate (4.8 mL, 26.5 mmol). The reaction was stirred at 22° C. under N₂ for 2 h, then diluted with water (300 mL), and extracted with DCM (150 mL×3). The combined organic layers were dried over anhydrous Na₂SO₄, filtered, and the filtrate was concentrated in vacuo. The resulting residue was purified by flash silica gel chromatography (Biotage; 12 g Agela Silica Flash Column, Eluent of Petroleum ether/EtOAc 0-50% gradient; flow rate=30 mL/min) to give intermediate 31 g. LC-MS (ESI) m/z: 518.2 [M+H]⁺.

Step H—Synthesis of Intermediate 31 h To a solution of intermediate 31 g (1.14 g, 2.202 mmol) in DME (13 mL), stirred at −22° C., was added N-methylmorpholine (0.334 g, 3.30 mmol), followed by isobutyl chloroformate (0.37 mL, 2.85 mmol). The reaction mixture was stirred at −22° C. for 30 minutes, then the solid was filtered off. The filtrate was cooled to −15° C., and NaBH₄ (0.42 g, 11.01 mmol) and H₂O (1 mL) were added sequentially. The mixture was stirred at −15° C. for 30 min, then diluted with saturated aqueous NH₄Cl solution (200 mL), and extracted with EtOAc (100 mL×3). The combined organic layers were washed with brine (120 mL), dried over anhydrous Na₂SO₄, filtered and the filtrate was concentrated under reduced pressure. The resulting residue was purified by flash silica gel chromatography (ISCO; 4 g Agela Silica Flash Column, Eluent of 0-20% EtOAc/Petroleum ether gradient; flow rate=30 mL/min) to give a cis/trans mixture of intermediate 31h. ¹H NMR (400 MHz, CDCl3) δ: 7.67-7.57 (m, 4H), 7.46-7.27 (m, 11H), 5.06 (s, 2H), 3.69-3.60 (m, 2H), 3.56-3.38 (m, 2H), 2.61 (br s, 1H), 2.45-2.17 (m, 2H), 2.14-1.85 (m, 3H), 1.09-1.03 (m, 9H).

Step I—Synthesis of Intermediate 31i To a solution of intermediate 31h (990 mg, 1.965 mmol) in THF (15 mL) stirred at 0° C., were added phthalimide (347 mg, 2.358 mmol) and Bu₃P (795 mg, 3.93 mmol), followed by the dropwise addition of a solution of DEAD (0.62 mL, 3.92 mmol) in THF (5 mL). The reaction mixture was stirred at 25° C. for 16 h. Then the solvent was removed under reduced pressure, and the resulting residue was purified by flash silica gel chromatography (ISCO; 4 g Agela Silica Flash Column, Eluent of 0-9% EtOAc/Petroleum ether gradient; flow rate=30 mL/min) to give a cis- and trans-mixture of intermediate 31i. LC-MS (ESI) m/z: 633.2 [M+H]⁺.

Step J Synthesis of Intermediate 31D-1 and Intermediate 31i-2 The cis- and trans-mixture of intermediate 31i (1.24 g, 1.959 mmol) was separated by SFC (Column: DAICEL CHIRALPAK AD, 250 mm×50 mm, 10 um; Condition: Neutral-IPA; Begin B 30%, End B 30%; FlowRate (mL/min) 200; Injections 120) to give intermediate 31i-1 (the first eluting isomer) and intermediate 31i-2 (the second eluting isomer).

Intermediate 31i-1: LC-MS (ESI) m/z: 633.2 [M+H]⁺. ¹H NMR (400 MHz, CDCl3) δ=7.89-7.80 (m, 2H), 7.78-7.68 (m, 2H), 7.65-7.56 (m, 4H), 7.40-7.29 (m, 11H), 5.12 (br s, 1H), 5.02 (s, 2H), 3.78 (br s, 2H), 3.70 (br s, 2H), 2.32-2.28 (m, 1H), 2.18 (br d, J=8.6 Hz, 4H), 1.03 (s, 9H).

Intermediate 31i-2: LC-MS (ESI) m/z: 633.2 [M+H]⁺. ¹H NMR (400 MHz, CDCl3) S: 7.83 (dd, J=3.1, 5.5 Hz, 2H), 7.70 (dd, J=3.1, 5.5 Hz, 2H), 7.67-7.60 (m, 4H), 7.45-7.28 (m, 11H), 5.01 (s, 3H), 3.69 (br s, 2H), 3.66 (d, J=7.4 Hz, 2H), 2.84 (br s, 1H), 2.25 (br s, 2H), 2.07 (br d, J=8.2 Hz, 2H), 1.07 (s, 9H).

Step K—Synthesis of Intermediate 31j-1 To a suspension of intermediate 31i-1 (360 mg, 0.569 mmol) in ethanol (3.7 mL) was added hydrazinium hydroxide (0.111 mL, 1.138 mmol). The reaction was refluxed at 82° C. for 1.5 h. After cooling, the insoluble material was filtered off, and the filtrate was concentrated in vacuo. To the resulting residue was added dichloromethane, and the insoluble material was filtered off. The filtrate was concentrated in vacuo to give crude intermediate 31j-1, which was used in the next step without further purification. LC-MS (ESI) m/z: 503.2 [M+H]⁺.

Step L—Synthesis of Intermediate 31k-1 To a stirred solution of intermediate 3c (312 mg, 0.668 mmol) and intermediate 31j-1 (280 mg, 0.557 mmol) in MeCN (6.5 mL) were added AcOH (0.11 mL, 1.922 mmol) and potassium acetate (219 mg, 2.228 mmol) at 20° C. The reaction was stirred at 80° C. for 2 h, then diluted with water (20 mL), and extracted with EtOAc (10 mL×3). The organic layers were combined, dried over anhydrous Na₂SO₄, filtered, and the filtrate was concentrated under reduced pressure. The resulting residue was purified by reversed phase MPLC (Biotage; 4 g Agela, C18, 20-35 pm, Eluent of 0-50% MeCN/H₂O (0.5% TFA) gradient; flow rate=30 mL/min) to give intermediate 31k-1. LC-MS (ESI) m/z: 921.4 [M+H]⁺.

Step M—Synthesis of Intermediate 31l-1 To a stirred solution of intermediate 31k-1 (600 mg, 0.651 mmol) in THF (6.6 mL) was added TBAF (1N in THF, 1.3 mL, 1.300 mmol) dropwise. The reaction was stirred at 28° C. for 20 h, then diluted with water (2 mL), and extracted with 10 vol % THF/EtOAc (1 mL×3). The organic layers were combined, dried over anhydrous Na₂SO₄, filtered, and the filtrate was concentrated under reduced pressure. The resulting residue was purified by reverse phase MPLC (Biotage; 20 g Agela, C18, 20-35 m, Eluent of 0-50% MeCN/H₂O (0.5‰ TFA) gradient; flow rate=50 mL/min) to give intermediate 31l-1. LC-MS (ESI) m/z: 683.6 [M+H]⁺.

Step N—Synthesis of Intermediate 31m-1 To a solution of intermediate 31l-1 (210 mg, 0.308 mmol) in EtOAc (23 mL) was added 10 wt % palladium on carbon (164 mg, 0.154 mmol). The resulting mixture was stirred at 30° C. under H₂ atmosphere (15 psi) for 20 h. Then the reaction mixture was filtered, and the filtrate was concentrated to give crude intermediate 31m-1, which was used in the next step without further purification. LC-MS (ESI) m/z: 549.2 [M+H]⁺.

Step O—Synthesis of Intermediate 31n-1 A solution of intermediate 31m-1 (90 mg, 0.164 mmol) in TFA (1.7 mL) was stirred at 40° C. for 1 h. Then the reaction mixture was concentrated in vacuo to give intermediate 31n-1, which was used in the next step without further purification. LC-MS (ESI) m/z: 392.9 [M+H]⁺.

Step P—Synthesis of Compounds 70 and 71 To a solution of intermediate 31n-1 (64 mg, 0.163 mmol) in MeOH (1.2 mL) was added intermediate 5 (64 mg, 0.176 mmol). The reaction was stirred at 30° C. for 16 h, then diluted with MeOH (3 mL). The resulting solution was directly purified by reverse phase HPLC (Column: Boston Uni C18 40×150×5 um; Condition water (0.1% TFA)-ACN; Begin B 0, End B 30; Gradient Time (min) 10; 100% B Hold Time (min) 2; FlowRate (mL/min) 60; Injections 1) to give compound 70 as its TFA salt. The TFA salt was dissolved in H₂O (3 mL) and purified by reverse phase HPLC (Column: Welch Xtimate C18 150×25 mm×5 um; Condition: water (0.225% FA)-ACN; Begin B 0, End B 18; Gradient Time (min) 15; 100% B Hold Time (min) 2; FlowRate (mL/min) 25; Injections 1) to give compound 70 as its formic acid salt. LC-MS (ESI) m/z: 739.3 [M+H]⁺. ¹H NMR (400 MHz, D₂O+CD₃CN) δ: 7.37-7.34 (m, 2H), 6.88 (d, J=8.4 Hz, 1H), 6.80 (s, 1H), 4.60 (s, 1H), 4.40-4.36 (m, 1H), 3.69 (s, 2H), 3.42 (apparent d, J=7.0 Hz, 2H), 2.84-2.70 (m, 2H), 2.57-2.43 (m, 1H), 2.32-2.21 (m, 2H), 2.13-1.93 (m, 3H), 1.72-1.68 (m, 1H), 1.48 (s, 3H), 1.41 (s, 3H), 1.23 (s, 3H).

Compound 71 was analogously prepared as its formic acid salt from intermediate 31i-2 using the method described in Steps J to P of Example 47.

Compound 71: LC-MS (ESI) m/z: 739.3 [M+H]⁺. ¹H NMR (400 MHz, D₂O+CD₃CN) δ: 7.37-7.34 (m, 2H), 6.87 (d, J=8.4 Hz, 1H), 6.82 (s, 1H), 4.60 (s, 1H), 4.37 (br d, J=9.8 Hz, 1H), 3.60 (s, 2H), 3.41 (d, J=7.4 Hz, 2H), 2.88-2.67 (m, 3H), 2.39-2.27 (m, 2H), 2.13-1.96 (m, 3H), 1.77-1.62 (m, 1H), 1.49 (s, 3H), 1.40 (s, 3H), 1.22 (s, 3H).

### Biological Assays

### Antibiotic Activity: Determination of Growth Inhibitory Concentration

The concentrations of compounds required to inhibit the growth of various strains of bacteria were determined in an assay that assessed bacterial growth by measuring optical density at 600 nm (OD600). The bacterial strains tested included the clinical strains Escherichia coli expressing NDM-1 (CLB30016), Klebsiella pneumoniae expressing KPC-1 (CL6569), Acinetobacter baumannii expressing TEM-1, AmpC, and Oxa-24/40 (CL6188) and Pseudomonas aeruginosa expressing AmpC (CL5701). All compounds were tested in the presence of a R lactamase inhibitor (BLi, Relebactam) in 384-well microplates.

The clinical strains were stored as frozen single use stocks, thawed and diluted into 1.1× cation-adjusted Mueller-Hinton II broth to achieve approximately 2×10⁵ CFU/mL. Test compounds were dissolved in DMSO and diluted 1:50 in the assay, resulting in a final concentration range of 100 μM to 0.098 μM. On the day of the assay, 1 μL of test compound was added to the plate followed by 4 μL of 50 μg/mL BLi in MOPS buffer and 45 μL of diluted bacteria. Plates were centrifuged at 1000 rpm for 30 seconds, shaken at approximately 800 rpm for 1 minute, and incubated at 35±2° C. for 22 hours. The concentration of BLi used in the assay was 4 μg/mL. At the end of the incubation, absorbance at 600 nm was determined using a spectrophotometer. Inhibition was quantitated by identifying the lowest concentration of test compound that was required to inhibit 95% of the growth of the bacteria. The results for Examples 1-39 are reported in Table I, expressed as the concentration of compound that inhibited 95% of bacterial growth (Minimum Inhibitory Threshold Concentration; MITC95).

Representative compounds of the present invention display a growth inhibitory effect. For example, representative Compounds 1-71 were determined to inhibit growth at concentrations of 100 μM or less.

TABLE I
Antibacterial activity of Compounds 1-71

| | AB_{—} | PA_{—} | KP_{—} | EC_{—} |
|---|---|---|---|---|
| | CL6188 | CL5701 | CL6569 | CLB30016 |
| Compound | MITC95 | MITC95 | MITC95 | MITC95 |
| # | (μM) | (μM) | (μM) | (μM) |
| 1 | 6.25 | 1.563 | 0.3906 | 3.125 |
| 2 | 6.25 | 1.563 | 0.3906 | 3.125 |
| 3 | 6.25 | 1.563 | 0.3906 | 3.125 |
| 4 | 4.688 | 1.563 | 0.3906 | 6.25 |
| 5 | 12.5 | 3.125 | 0.7813 | 12.5 |
| 6 | 12.5 | 3.125 | 0.7813 | 6.25 |
| 7 | 6.25 | 1.563 | 0.3906 | 6.25 |
| 8 | 10.94 | 1.563 | 0.3906 | 6.641 |
| 9 | 3.125 | 1.563 | 0.3906 | 6.25 |
| 10 | 6.25 | 3.125 | 0.7813 | 6.25 |
| 11 | 6.25 | 1.563 | 0.3906 | 3.125 |
| 12 | 6.25 | 1.563 | 0.3906 | 3.125 |
| 13 | 6.25 | 3.125 | 0.7813 | 6.25 |
| 14 | 12.5 | 3.125 | 0.3906 | 3.125 |
| 15 | 12.5 | 3.125 | 0.7813 | 6.25 |
| 16 | 6.25 | 3.125 | 0.7813 | 6.25 |
| 17 | 6.25 | 1.563 | 0.3906 | 3.125 |
| 18 | 6.25 | 1.563 | 0.3906 | 3.125 |
| 19 | 6.25 | 1.563 | 0.3906 | 3.125 |
| 20 | 6.25 | 3.125 | 0.3906 | 6.25 |
| 21 | 3.125 | 3.125 | 0.7813 | 6.25 |
| 22 | 6.25 | 3.125 | 0.3906 | 6.25 |
| 23 | 6.25 | 1.563 | 0.3906 | 6.25 |
| 24 | 6.25 | 3.125 | 0.3906 | 6.25 |
| 25 | 3.125 | 1.563 | 0.3906 | 3.125 |
| 26 | 6.25 | 3.125 | 0.7813 | 6.25 |
| 27 | 6.25 | 1.563 | 0.7813 | 6.25 |
| 28 | 3.125 | 1.563 | 0.3906 | 6.25 |
| 29 | 3.125 | 1.563 | 0.3906 | 6.25 |
| 30 | 6.25 | 3.125 | 0.7813 | 6.25 |
| 31 | 12.5 | 3.125 | 0.7813 | 6.25 |
| 32 | 12.5 | 3.125 | 0.7813 | 6.25 |
| 33 | 6.25 | 3.125 | 0.3906 | 6.25 |
| 34 | 6.25 | 1.563 | 0.3906 | 6.25 |
| 35 | 6.25 | 1.563 | 0.3906 | 3.125 |
| 36 | 3.125 | 1.563 | 0.3906 | 3.125 |
| 37 | 6.25 | 1.563 | 0.3906 | 3.125 |
| 38 | 6.25 | 1.563 | 0.3906 | 3.125 |
| 39 | 6.25 | 1.563 | 0.3906 | 6.25 |
| 40 | 3.125 | 1.563 | 0.3906 | 6.25 |
| 41 | 25 | 12.5 | 3.125 | 25 |
| 42 | 3.125 | 1.563 | 0.3906 | 6.25 |
| 43 | 6.25 | 1.563 | 0.7813 | 6.25 |
| 44 | 3.125 | 1.563 | 0.3906 | 3.125 |
| 45 | 3.125 | 1.563 | 0.3906 | 6.25 |
| 46 | 6.25 | 1.563 | 0.7813 | 12.5 |
| 47 | 12.5 | 6.25 | 1.563 | 25 |
| 48 | 6.25 | 1.563 | 0.3906 | 3.125 |
| 49 | 3.125 | 1.563 | 0.3906 | 3.125 |
| 50 | 12.5 | 1.563 | 0.3906 | 6.25 |
| 51 | 6.25 | 1.563 | 0.3906 | 12.5 |
| 52 | 9.375 | 2.344 | 0.7813 | 6.25 |
| 53 | 6.25 | 1.563 | 0.3906 | 6.25 |
| 54 | 4.688 | 1.563 | 0.3906 | 4.688 |
| 55 | 3.125 | 1.563 | 0.7813 | 6.25 |
| 56 | 3.125 | 1.563 | 0.7813 | 3.125 |
| 57 | 6.25 | 3.125 | 0.3906 | 3.125 |
| 58 | 6.25 | 3.125 | 0.3906 | 6.25 |
| 59 | 6.25 | 6.25 | 0.7813 | 6.25 |
| 60 | 6.25 | 3.125 | 0.7813 | 6.25 |
| 61 | 6.25 | 3.125 | 0.7813 | 6.25 |
| 62 | 6.25 | 3.125 | 0.7813 | 12.5 |
| 63 | 6.25 | 3.125 | 0.7813 | 6.25 |
| 64 | 6.25 | 1.563 | 0.3906 | 6.25 |
| 65 | 6.25 | 3.125 | 0.3906 | 3.125 |
| 66 | 3.125 | 1.563 | 0.3906 | 6.25 |
| 67 | 6.25 | 1.563 | 0.3906 | 6.25 |
| 68 | 6.25 | 3.125 | 0.7813 | 12.5 |
| 69 | 6.25 | 3.125 | 0.7813 | 12.5 |
| 70 | 6.25 | 1.563 | 0.3906 | 6.25 |
| 71 | 3.125 | 1.563 | 0.3906 | 6.25 |

What is claimed is:

1. A compound of Formula I 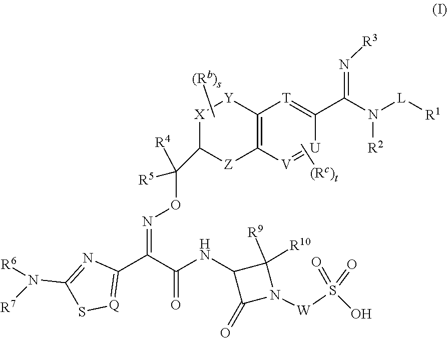
or a pharmaceutically acceptable salt thereof, wherein:
T is CH, or N, provided that no more than two of T, U and V are N;
U is CH, or N;
V is CH or N;
X is selected from the group consisting of:
1) O, and
2) CH₂;
Y is selected from the group consisting of:
1) O,
2) NR⁸,
3) S, and
4) CH₂,
provided that when Y is O, NR⁸ or S then X is not O;
Z is
1) O,
2) S,
3) CH₂, or
4) NH,
provided that when Z is O, S or NH, then X is not O;
W is selected from the group consisting of:
1) bond, and
2) O;
Q is selected from the group consisting of:
1) N, and
2) CR⁸;
L is selected from the group consisting of:
1) —C₁₋₆alkyl-,
2) —C₁₋₆alkyl-O—,
3) —C₁₋₆alkyl-O—C₁₋₆alkyl-,
4) —C₁₋₆alkyl-S—,
5) —C₁₋₆alkyl-S—C₁₋₆alkyl-,
6) —C₁₋₆alkyl-N(R^{m})—, and
7) —C₁₋₆alkyl-N(R^{m})—C₁₋₆alkyl-,
wherein alkyl is unsubstituted or substituted with one to three substituents selected from:
halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl;
R¹ is selected from the group consisting of:
1) —C₃₋₁₂cycloalkyl,
2) —C₃₋₁₂cycloalkenyl,
3) C₂₋₁₁cycloheteroalkyl,
4) C₂₋₁₁cycloheteroalkenyl,
5) aryl, and
6) heteroaryl,
wherein cycloalkyl, cycloalkenyl, cycloheteroalkyl, cycloheteroalkenyl, aryl and heteroaryl are unsubstituted or substituted with one to five substituents selected from R^{a};
R² is selected from the group consisting of:
1) hydrogen,
2) C₁₋₆alkyl,
3) C₁₋₆alkyl-OR⁴, and
4) C₁₋₆alkyl-NHR⁴,
wherein alkyl is unsubstituted or substituted with one to three halogens;
R³ is selected from the group consisting of:
1) hydrogen, and
2) OH;
R⁴ is selected from the group consisting of:
1) hydrogen,
2) C₁₋₃alkyl, and
3) C₃cycloalkyl,
wherein alkyl and cycloalkyl are unsubstituted or substituted with one to three halogens or OC₁₋₃alkyl;
R⁵ is selected from the group consisting of:
1) —CO₂H, and
2) tetrazole;
R⁶ and R⁷ are selected from the group consisting of:
1) hydrogen, and
2) C₁₋₆alkyl,
wherein alkyl is unsubstituted or substituted with one to three halogens, provided that at least one of R⁶ and R⁷ is hydrogen;
R⁸ is independently selected from the group consisting of:
1) hydrogen,
2) C₁₋₄alkyl,
3) halogen, and
4) C₃₋₇cycloalkyl,
wherein alkyl and cycloalkyl are unsubstituted or substituted with one to three substituents selected from: —OH, halogen, NH₂, and —OC₁₋₃alkyl;
R⁹ and R¹⁰ are selected from the group consisting of:
1) hydrogen, and
2) C₁₋₆alkyl,
wherein alkyl is unsubstituted or substituted with one to three substituents selected from:
halogen, OH, —OC₁₋₃alkyl, —NHC(O)C₁₋₃alkyl, —C(O)NHC₁₋₃alkyl, NHC₁₋₃alkyl, and SC₁₋₃alkyl, provided that one or both of R⁹ and R¹⁰ are C₁₋₆alkyl,
or alternatively R⁹ and R¹⁰ together with the carbon to which they are attached form a monocyclic C₃₋₅cycloalkyl or a monocyclic C₂₋₅cycloheteroalkyl, wherein cycloalkyl and cycloheteroalkyl are unsubstituted or substituted with one to three substituents independently selected from halogen, —OH and —OC₁₋₃alkyl;
each R^{a} is independently selected from the group consisting of:
1) halogen,
2) —C₁₋₆alkyl,
3) —C₀₋₆alkyl-O—C₁₋₆alkyl,
4) —C₀₋₆alkyl-OH,
5) —C₀₋₆alkyl S(O)ᵣR^{j},
6) —C₀₋₆alkyl S(O)ᵣNR^{k}R^{l},
7) —C₀₋₆alkyl C(O)Rⁱ,
8) —C₀₋₆alkyl OC(O)Rⁱ,
9) —C₀₋₆alkyl C(O)OR¹,
10) —C₀₋₆alkyl CN,
11) —C₀₋₆alkyl C(O)N^{k}R^{l},
12) —C₀₋₆alkyl C(NH)NR^{k}R^{l},
13) —C₀₋₆alkylNR^{k}R^{l},
14) —C₀₋₆alkyl N(R^{k})(C(O)Rⁱ),
15) —C₀₋₆alkyl N(R^{k})(C(O)OR^{h}),
16) —C₀₋₆alkyl N(R^{k})(C(O)NR^{f}R^{g}), and
17) —C₀₋₆alkyl N(R^{k})(S(O)ᵥR^{j}),
wherein alkyl is unsubstituted or substituted with one to three substituents selected from:
halogen, OH, —OC₁₋₃alkyl, —C₁₋₃alkyl, —CO₂C₁₋₃alkyl, —C(O)NH₂, —C₀₋₆alkylNH₂, and —C₀₋₆alkylNH(C₁₋₃alkyl);
each R^{b} is independently selected from the group consisting of:
1) hydrogen,
2) C₁₋₆alkyl,
3) C₀₋₆alkyl-O—C₁₋₆alkyl,
4) C₀₋₆alkyl-OH,
5) C₀₋₆alkyl-S(O)ᵤR^{d},
6) C₁₋₆alkyl-C(O—N(R^{c})₂,
7) C₁₋₆alkylN(R^{e})C(O)R^{e},
8) C₀₋₆alkyl-N(R^{e})₂, and
9) halogen,
wherein alkyl is unsubstituted or substituted with one to three halogens, or wherein two R^{b} substituents together with the atoms they are attached to can cyclize to form a 3 to 6 membered ring;
each R^{c} is independently selected from the group consisting of:
1) hydrogen,
2) C₁₋₆alkyl,
3) C₀₋₆alkyl-O—C₁₋₆alkyl,
4) C₀₋₆alkyl-OH,
5) C₀₋₆alkyl-S(O)ᵥR^{f},
6) C₀₋₆alkyl-S(O)ᵥN(R^{g})₂,
7) C₁₋₆alkyl C(O)—N(R^{g})₂,
8) C₁₋₆alkylN(R^{g})C(O)R^{g},
9) C₀₋₆alkyl-N(R^{g})₂, and
10) halogen,
wherein alkyl is unsubstituted or substituted with one to three halogens;
each R^{d} is independently selected from the group consisting of:
1) hydrogen, and
2) —C₁₋₆ alkyl,
wherein each alkyl is unsubstituted or substituted with one to three halogens;
each R^{e} is independently selected from the group consisting of:
1) hydrogen, and
2) —C₁₋₆ alkyl,
wherein each alkyl is unsubstituted or substituted with one to three halogens;
each R^{f} is independently selected from the group consisting of:
1) hydrogen, and
2) —C₁₋₆alkyl,
wherein each alkyl is unsubstituted or substituted with one to three halogens;
each R^{g} is independently selected from the group consisting of:
1) hydrogen, and
2) —C₁₋₆ alkyl,
wherein each alkyl is unsubstituted or substituted with one to three halogens;
each R^{h} is independently selected from the group consisting of:
1) hydrogen, and
2) —C₁₋₆ alkyl,
wherein each alkyl is unsubstituted or substituted with one to three halogens;
each Rⁱ is —C₁₋₆ alkyl, wherein each alkyl is unsubstituted or substituted with one to three halogens;
each R^{j} is independently selected from the group consisting of:
1) hydrogen,
2) OH, and
3) —C₁₋₆ alkyl,
wherein each alkyl is unsubstituted or substituted with one to three halogens;
each R^{k} is independently selected from the group consisting of:
1) hydrogen, and
2) —C₁₋₆alkyl,
wherein each alkyl is unsubstituted or substituted with one to three halogens;
each R^{l} is independently selected from the group consisting of:
1) hydrogen, and
2) —C₁₋₆alkyl,
wherein each alkyl is unsubstituted or substituted with one to three halogens;
each R^{m} is independently selected from the group consisting of:
1) hydrogen, and
2) —C₁₋₆alkyl,
wherein each alkyl is unsubstituted or substituted with one to three halogens;
each r is independently 0, 1 or 2;
each s is independently 0, 1, 2, 3, 4 or 5;
each t is independently 0, 1, 2 or 3;
each u is independently selected from 0, 1 or 2; and
each v is independently selected from 0, 1 or 2.

2. The compound of claim 1 wherein
T is CH;
U is CH; and
V is CH;
or a pharmaceutically acceptable salt thereof.

3. The compound of claim 1 wherein
X is CH₂;
or a pharmaceutically acceptable salt thereof.

4. The compound of claim 1 wherein
Y is O or CH₂; and
Z is O or CH₂;
or a pharmaceutically acceptable salt thereof.

5. The compound of claim 4 wherein
Y is CH₂; and
Z is O;
or a pharmaceutically acceptable salt thereof.

6. A compound of claim 1 wherein
W is O;
or a pharmaceutically acceptable salt thereof.

7. The compound of claim 1 wherein
Q is CR⁸; and
R⁸ is hydrogen;
or a pharmaceutically acceptable salt thereof.

8. The compound of claim 1 wherein
R² is hydrogen; and
R³ is hydrogen;
or a pharmaceutically acceptable salt thereof.

9. The compound of claim 5 wherein R⁵ is —CO₂H; or a pharmaceutically acceptable salt thereof.

10. The compound of claim 1 wherein R⁴ is selected from the group consisting of:
1) C₁₋₃alkyl, and
2) C₃cycloalkyl,
wherein alkyl and cycloalkyl are unsubstituted or substituted with one to three substituents selected from: halogen and OC₁₋₃alkyl; or a pharmaceutically acceptable salt thereof.

11. The compound of claim 1 wherein R⁴ is C₁₋₃alkyl, wherein alkyl is unsubstituted or substituted with one to three substituents selected from: halogen or OC₁₋₃alkyl;
or a pharmaceutically acceptable salt thereof.

12. The compound of claim 1 wherein
R⁶ is hydrogen; and
R⁷ is hydrogen;
or a pharmaceutically acceptable salt thereof.

13. The compound of claim 1 wherein
R⁹ is C₁₋₆alkyl, and
R¹⁰ is C₁₋₆alkyl;
or a pharmaceutically acceptable salt thereof.

14. The compound of claim 1 wherein L is selected from the group consisting of:
1) —C₁₋₆alkyl-, and
2) —C₁₋₆alkyl-O—,
wherein alkyl is unsubstituted or substituted with one to three substituents selected from:
halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl; or a pharmaceutically acceptable salt thereof.

15. The compound of claim 1 wherein L is —C₁₋₆alkyl-, wherein alkyl is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl; or a pharmaceutically acceptable salt thereof.

16. The compound of claim 1 wherein R¹ is selected from the group consisting of:
1) —C₃₋₁₂cycloalkyl,
2) C₂₋₁₁cycloheteroalkyl, and
3) aryl,
wherein cycloalkyl, cycloheteroalkyl and aryl are unsubstituted or substituted with one to five substituents selected from R^{a}; or a pharmaceutically acceptable salt thereof.

17. The compound of claim 1 wherein R¹ is selected from the group consisting of:
1) —C₃₋₁₂cycloalkyl, and
2) C₂₋₁₁cycloheteroalkyl,
wherein cycloalkyl and cycloheteroalkyl are unsubstituted or substituted with one to five substituents selected from R^{a}; or a pharmaceutically acceptable salt thereof.

18. The compound of claim 1 wherein
T is CH;
U is CH;
V is CH;
X is CH₂;
Y is O or CH₂;
Z is O or CH₂;
W is bond or O;
Q is CR⁸;
L is selected from the group consisting of:
1) —C₁₋₆alkyl-, and
2) —C₁₋₆alkyl-O—,
wherein alkyl is unsubstituted or substituted with one to three substituents selected from:
halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl;
R¹ is selected from the group consisting of:
1) —C₃₋₁₂cycloalkyl,
2) C₂₋₁₁cycloheteroalkyl, and
3) aryl,
wherein cycloalkyl, cycloheteroalkyl and aryl are unsubstituted or substituted with one to five substituents selected from R^{a};
R² is hydrogen;
R³ is hydrogen;
R⁴ is selected from the group consisting of:
1) C₁₋₃alkyl, and
2) C₃cycloalkyl,
wherein alkyl and cycloalkyl are unsubstituted or substituted with one to three substituents selected from: halogen and OC₁₋₃alkyl;
R⁵ is —CO₂H or tetrazole;
R⁶ is hydrogen;
R⁷ is hydrogen;
R⁸ is hydrogen;
R⁹ is C₁₋₆alkyl, and
R¹⁰ is C₁₋₆alkyl;
or a pharmaceutically acceptable salt thereof.

19. The compound of claim 1 wherein
T is CH;
U is CH;
V is CH;
X is CH₂;
Y is CH₂;
Z is O;
W is O;
Q is CR^{g};
L is —C₁₋₆alkyl-, wherein alkyl is unsubstituted or substituted with one to three substituents selected from: halogen, C₁₋₃alkyl, —OC₁₋₃alkyl, —NHC(O)C₁₋₃alkyl, —C(O)NHC₁₋₃alkyl, NHC₁₋₃alkyl, and SC₁₋₃alkyl;
R¹ is selected from the group consisting of:
1) —C₃₋₁₂cycloalkyl, and
2) C₂₋₁₁cycloheteroalkyl,
wherein cycloalkyl and cycloheteroalkyl are unsubstituted or substituted with one to five substituents selected from R^{a};
R² is hydrogen;
R³ is hydrogen;
R⁴ is C₁₋₃alkyl;
R⁵ is —CO₂H;
R⁶ is hydrogen;
R⁷ is hydrogen;
R⁸ is hydrogen;
R⁹ is C₁₋₆alkyl, and
R¹⁰ is C₁₋₆alkyl;
or a pharmaceutically acceptable salt thereof.

20. The compound of claim 1 which is selected from:
1) (S)-2-((R)-6-(N-(((1r,4R)-4-amino-1-fluorocyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
2) (S)-2-((R)-6-(N-(((1s,4S)-4-amino-1-fluorocyclohexyl)-methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)-oxy)propanoic acid;
3) (S)-2-((R)-6-(N-((3-amino-bicyclo[1.1.1]pentan-1-yl)-methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)-amino)oxy)propanoic acid;
4) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(piperidin-4-ylmethyl)-carbamimidoyl)chroman-2-yl)propanoic acid;
5) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N—(((R)-pyrrolidin-3-yl)methyl)-carbamimidoyl)chroman-2-yl)propanoic acid;
6) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N—(((S)-pyrrolidin-3-yl)methyl)-carbamimidoyl)chroman-2-yl)propanoic acid;
7) (S)-2-((R)-6-(N-(((1r,3R)-3-aminocyclobutyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)propanoic acid;
8) (S)-2-((R)-6-(N-(((1s,3S)-3-aminocyclobutyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)propanoic acid;
9) (S)-2-((((Z)-1-(2-aminothiazol-4-yl)-2-(((S)-2,2-dimethyl-4-oxo-1-(sulfooxy)azetidin-3-yl)amino)-2-oxo-ethylidene)-amino)oxy)-2-((R)-6-(N-(2-((S)-pyrrolidin-2-yl)-ethyl)-carbamimidoyl)chroman-2-yl)propanoic acid;
10) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N-(2-((R)-pyrrolidin-2-yl)ethyl)-carbamimidoyl)chroman-2-yl)propanoic acid;
11) (S)-2-((R)-6-(N-(3-(1-aminocyclopropyl)propyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)propanoic acid;
12) (S)-2-((R)-6-(N-(2-(1-aminocyclopropyl)ethyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-propanoic acid;
13) (S)-2-((R)-6-(N-(2-(1-aminocyclobutyl)ethyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)propanoic acid;
14) (S)-2-((R)-6-(N-(2-(4-aminopiperidin-1-yl)ethyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
15) (S)-2-((R)-6-(N-(((1r,4R)-4-aminocyclohexyl)methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)amino)oxy)propanoic acid;
16) (S)-2-((R)-6-(N-(((1s,4S)-4-aminocyclohexyl)methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)-amino)oxy)propanoic acid;
17) (S)-2-((R)-6-(N-(4-(aminomethyl)benzyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxo-ethylidene)amino)oxy)-propanoic acid;
18) (S)-2-((R)-6-(N-(3-(aminomethyl)benzyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxo-ethylidene)amino)oxy)-propanoic acid;
19) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(azetidin-3-ylmethyl)-carbamimidoyl)chroman-2-yl)propanoic acid;
20) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-((4-(methoxymethyl)-piperidin-4-yl)methyl)-carbamimidoyl)chroman-2-yl)propanoic acid;
21) (S)-2-((R)-6-(N-(((1S,3R)-3-amino-2,2-dimethylcyclobutyl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)propanoic acid;
22) (S)-2-((R)-6-(N-(((2R,4r,6R)-6-aminospiro[3.3]heptan-2-yl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
23) (S)-2-((R)-6-(N-(((2S,4s,6S)-6-aminospiro[3.3]heptan-2-yl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
24) (S)-2-((R)-6-(N-(((2R,4r,6R)-6-amino-2-fluorospiro[3.3]heptan-2-yl)methyl)-carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
25) (S)-2-((R)-6-(N-(((2S,4s,6S)-6-amino-2-fluorospiro[3.3]heptan-2-yl)methyl)-carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
26) (S)-2-((R)-6-(N-(((1s,3S)-3-amino-1-methylcyclobutyl)-methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)-amino)oxy)propanoic acid;
27) (S)-2-((R)-6-(N-(((1r,3R)-3-amino-1-methylcyclobutyl)methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)amino)oxy)propanoic acid;
28) (S)-2-((R)-6-(N-(((1s,3S)-3-(aminomethyl)cyclobutyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
29) (S)-2-((R)-6-(N-(((1r,3R)-3-(aminomethyl)-cyclobutyl)methyl)carbamimidoyl)chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)amino)oxy)propanoic acid;
30) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(2-(piperidin-4-yl)ethyl)-carbamimidoyl)chroman-2-yl)propanoic acid;
31) tert-butyl (S)-2-((((Z)-1-(2-((tert-butoxycarbonyl)amino)thiazol-4-yl)-2-(((S)-2,2-dimethyl-4-oxo-1-(sulfooxy)azetidin-3-yl)amino)-2-oxoethylidene)amino)oxy)-2-((R)-6-(N—(((R)-morpholin-2-yl)methyl)carbamimidoyl)chroman-2-yl)propanoate;
32) tert-butyl (S)-2-((((Z)-1-(2-((tert-butoxycarbonyl)amino)thiazol-4-yl)-2-(((S)-2,2-dimethyl-4-oxo-1-(sulfooxy)azetidin-3-yl)amino)-2-oxoethylidene)amino)oxy)-2-((R)-6-(N—(((S)-morpholin-2-yl)methyl)carbamimidoyl)chroman-2-yl)propanoate;
33) (S)-2-((R)-6-(N-(2-((1R,4R)-2,5-diazabicyclo[2.2.1]heptan-2-yl)ethyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
34) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(3-((S)-pyrrolidin-2-yl)propyl)-carbamimidoyl)chroman-2-yl)propanoic acid;
35) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(3-((R)-pyrrolidin-2-yl)propyl)-carbamimidoyl)chroman-2-yl)propanoic acid;
36) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(3-((S)-azetidin-2-yl)propyl)-carbamimidoyl)chroman-2-yl)propanoic acid;
37) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(3-((R)-azetidin-2-yl)propyl)-carbamimidoyl)chroman-2-yl)propanoic acid;
38) (S)-2-((R)-6-(N-(((1s,4S)-4-(aminomethyl)cyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
39) (S)-2-((R)-6-(N-(((1r,4R)-4-(aminomethyl)cyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)-oxy)propanoic acid;
40) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N-(2-((S)-pyrrolidin-3-yl)ethyl)-carbamimidoyl)chroman-2-yl)propanoic acid;
41) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N-(2-((R)-pyrrolidin-3-yl)ethyl)-carbamimidoyl)chroman-2-yl)propanoic acid;
42) (S)-2-((((Z)-1-(2-aminothiazol-4-yl)-2-(((S)-2,2-dimethyl-4-oxo-1-(sulfooxy)azetidin-3-yl)amino)-2-oxo-ethylidene)amino)oxy)-2-((R)-6-(N-(2-(azetidin-3-yl)-ethyl)-carbamimidoyl)chroman-2-yl)propanoic acid;
43) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N-(2-(3-methylazetidin-3-yl)ethyl)-carbamimidoyl)chroman-2-yl)propanoic acid;
44) (S)-2-((((Z)-1-(2-aminothiazol-4-yl)-2-(((S)-2,2-dimethyl-4-oxo-1-(sulfooxy)azetidin-3-yl)amino)-2-oxoethylidene)-amino)oxy)-2-((R)-6-(N-(2-((S)-azetidin-2-yl)ethyl)-carbamimidoyl)chroman-2-yl)propanoic acid;
45) (S)-2-((((Z)-1-(2-aminothiazol-4-yl)-2-(((S)-2,2-dimethyl-4-oxo-1-(sulfooxy)azetidin-3-yl)amino)-2-oxo-ethylidene)amino)oxy)-2-((R)-6-(N-(2-((R)-azetidin-2-yl)ethyl)-carbamimidoyl)chroman-2-yl)propanoic acid;
46) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N—((S)-1-(azetidin-3-yl)propan-2-yl)carbamimidoyl)chroman-2-yl)propanoic acid;
47) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N—((R)-1-(azetidin-3-yl)propan-2-yl)carbamimidoyl)chroman-2-yl)propanoic acid;
48) (S)-2-((R)-6-(N—(((S)-1-amino-2,3-dihydro-1H-inden-5-yl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
49) (S)-2-((R)-6-(N—(((R)-1-amino-2,3-dihydro-1H-inden-5-yl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
50) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(2-(((S)-pyrrolidin-3-yl)oxy)ethyl)-carbamimidoyl)chroman-2-yl)propanoic acid;
51) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4S)-2-(hydroxymethyl)-piperidin-4-yl)methyl)carbamimidoyl)chroman-2-yl)propanoic acid;
52) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4R)-2-(hydroxymethyl)-piperidin-4-yl)methyl)carbamimidoyl)chroman-2-yl)propanoic acid;
53) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4R)-2-(hydroxymethyl)-piperidin-4-yl)methyl)carbamimidoyl)chroman-2-yl)propanoic acid;
54) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4S)-2-(hydroxymethyl)-piperidin-4-yl)methyl)carbamimidoyl)chroman-2-yl)propanoic acid;
55) (S)-2-((R)-6-(N-(((1S,2R)-2-(aminomethyl)cyclopropyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
56) (S)-2-((R)-6-(N-(((1R,2S)-2-(aminomethyl)cyclopropyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
57) (S)-2-((R)-6-(N-(((1s,4S)-4-amino-4-methylcyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
58) (S)-2-((R)-6-(N-(((1r,4R)-4-amino-4-methylcyclohexyl)methyl)carbamimidoyl)chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
59) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-((4-(2-methoxyethyl)piperidin-4-yl)methyl)carbamimidoyl)chroman-2-yl)propanoic acid;
60) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4R)-2-(methoxymethyl)-piperidin-4-yl)methyl)carbamimidoyl)chroman-2-yl)propanoic acid;
61) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4S)-2-(methoxymethyl)-piperidin-4-yl)methyl)carbamimidoyl)chroman-2-yl)propanoic acid;
62) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2R,4R)-2-(methoxymethyl)-piperidin-4-yl)methyl)carbamimidoyl)chroman-2-yl)propanoic acid;
63) (S)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)-2-((R)-6-(N-(((2S,4S)-2-(methoxymethyl)piperidin-4-yl)methyl)carbamimidoyl)chroman-2-yl)propanoic acid;
64) (S)-2-((R)-6-(N-(((1R,2S)-2-(2-aminoethyl)cyclopropyl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
65) (S)-2-((R)-6-(N-(((1S,2R)-2-(2-aminoethyl)cyclopropyl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
66) (S)-2-((R)-6-(N-(2-((1S,2R)-2-(aminomethyl)cyclopropyl)ethyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
67) (S)-2-((R)-6-(N-(2-((1R,2S)-2-(aminomethyl)cyclopropyl)ethyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
68) (S)-2-((R)-6-(N-(((1r,4R)-4-amino-1-methoxycyclohexyl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
69) (S)-2-((R)-6-(N-(((1s,4S)-4-amino-1-methoxycyclohexyl)methyl)carbamimidoyl)-chroman-2-yl)-2-((((Z)-1-(2-aminothiazol-4-yl)-2-(((S)-2,2-dimethyl-4-oxo-1-(sulfooxy)azetidin-3-yl)amino)-2-oxoethylidene)amino)oxy)propanoic acid;
70) (S)-2-((R)-6-(N-(((1s,3S)-3-amino-3-(hydroxymethyl)cyclobutyl)methyl)-carbamimidoyl)-chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)amino)oxy)propanoic acid; and
71) (S)-2-((R)-6-(N-(((1r,3R)-3-amino-3-(hydroxymethyl)cyclobutyl)methyl)-carbamimidoyl)-chroman-2-yl)-2-((((Z)-1-(2-aminothiazol-4-yl)-2-(((S)-2,2-dimethyl-4-oxo-1-(sulfooxy)-azetidin-3-yl)amino)-2-oxoethylidene)amino)oxy)propanoic acid;
or a pharmaceutically acceptable salt thereof.

21. A pharmaceutical composition comprising a therapeutically effective amount of a compound of claim 1, or a pharmaceutically acceptable salt thereof, and a pharmaceutically acceptable carrier.

22. A pharmaceutical composition according to claim 21, which further comprises a therapeutically effective amount of a beta-lactamase inhibitor compound.

23. A pharmaceutical composition according to claim 22 wherein the beta-lactamase inhibitor compound is selected from the group consisting of relebactam, tazobactam, clavulanic acid, sulbactam, and avibactam.

24. A method for treating a bacterial infection which comprises administering to a subject in need of such treatment a therapeutically effective amount of a compound of claim 1, or a pharmaceutically acceptable salt thereof.

25. The method of claim 24 further comprising administering to a subject in need of such treatment a therapeutically effective amount of a beta-lactamase inhibitor compound.

26. The method of claim 25 wherein the beta-lactamase inhibitor compound is selected from the group consisting of relebactam, tazobactam, clavulanic acid, sulbactam, and avibactam.

27. The method of claim 24 wherein the bacterial infection is due to Pseudomonas spp., Klebsiella spp., Enterobacter spp., Escherichia spp., Morganella spp., Citrobacter spp., Serratia spp. or Acintetobacter spp.

28-31. (canceled)
